(19) United States
(12) Patent Application Publication
(10) Pub. No.: US 2013/0064770 A1
(43) Pub. Date: Mar. 14, 2013

(54) RADIOLABELED COMPOUNDS AND METHODS THEREOF

(71) Applicants: Ian Martin Newington, Amersham (GB); Duncan George Wynn, Amersham (GB); Robert James Domett Nairne, Amersham (GB); Benedicte Guilbert, Amersham (GB); Subrata Mandal, Bangalore (IN); Jinto Jose, Bangalore (IN); Sunderaraman Varadarajan, Bangalore (IN); Chitralekha Rangaswamy, Bangalore (IN); Helen Betts, Sheffield (GB); Rebecca Davis, Oxfordshire (GB)

(72) Inventors: Ian Martin Newington, Amersham (GB); Duncan George Wynn, Amersham (GB); Robert James Domett Nairne, Amersham (GB); Benedicte Guilbert, Amersham (GB); Subrata Mandal, Bangalore (IN); Jinto Jose, Bangalore (IN); Sunderaraman Varadarajan, Bangalore (IN); Chitralekha Rangaswamy, Bangalore (IN); Helen Betts, Sheffield (GB); Rebecca Davis, Oxfordshire (GB)

(73) Assignee: GE HEALTHCARE LIMITED, BUCKINGHAMSHIRE (GB)

(21) Appl. No.: 13/699,155

(22) PCT Filed: May 26, 2011
(86) PCT No.: PCT/US11/38099

§ 371 Date: Nov. 20, 2012

(30) Foreign Application Priority Data
May 28, 2010 (IN) 1231/DEL/2010
May 28, 2010 (IN) 1232/DEL/2010

(51) Int. Cl. A61K 51/04; C07D 403/06; C07D 327/04; C07D 401/14; C07D 417/14; B01J 19/00; C07D 295/096; C07D 417/04; C07D 403/12; C07D 417/12; C07D 401/04; C07D 403/04

(52) U.S. Cl. CPC C07D 401/04; C07D 401/14; C07D 417/14; C07D 403/06; C07D 295/096; C07D 417/04; C07D 403/12; A61K 51/0461; A61K 51/0459; C07D 417/12; C07D 327/04; B01J 19/00
USPC 424/1.89; 544/182; 544/364; 544/370; 544/360; 544/394; 544/368; 544/373; 424/1.81; 544/369; 549/30; 422/159

(57) ABSTRACT

The present invention relates to radiodiagnostic compounds, methods of making those compounds, and methods of use thereof as imaging agents for preferably a HA serotonin 5-HT1A receptor for use in PET or SPECT, preferably PET. Compositions comprising an imaging-effective amount of radiolabeled compounds are also disclosed. The present invention also relates to non-radiolabeled compounds, methods of making those compounds, and methods of use thereof to treat various neurological and/or psychiatric disorders.

### CROSS-REFERENCE TO RELATED APPLICATIONS

This application claims the benefit of Indian Provisional Appl. Nos. 1232/DEL/2010 and 1231/DEL/2010, both of which were filed on May 28, 2010, the disclosures of each of which are incorporated herein by reference in their entireties as if fully set forth herein.

### FIELD OF INVENTION

The present invention relates to radiodiagnostic compounds (and precursors thereof), methods of making those compounds, and methods of use thereof as imaging agents for a serotonin receptor (e.g., the 5-HT_{1A} receptor) which would preferably have high affinity for use in PET or SPECT, preferably PET. Compositions comprising an imaging-effective amount of radiolabeled compounds are also disclosed. The present invention also relates to non-radiolabeled compounds, methods of making those compounds, and methods of use thereof to treat various neurological and/or psychiatric disorders.

### BACKGROUND OF THE INVENTION

Serotonin (5-hydroxytryptamine; 5-HT) plays a role in several neurological and psychiatric disorders. It has been linked with major depression, bipolar disorder, eating disorders, alcoholism, pain, anxiety, obsessive-compulsive disorders, Alzheimer's Disease, Parkinsons's disease and other psychiatric maladies. It is also involved in mediating the action of many psychotropic drugs including antidepressants, antianxiety drugs and antipsychotics. There are more than a dozen known subtypes of serotonin receptors. Among these serotonin receptors, 5-HT_{1A} receptors play a role as a presynaptic autoreceptor in the dorsal raphe nucleus and as a postsynaptic receptor for 5-HT in terminal field areas. The serotonin system in the brain is an important neurotransmission network regulating various physiological functions and behaviour including anxiety and mood states. (See Rasmussen et al., “Chapter 1. Recent Progress in Serotonin 5HT_{1A} Receptor Modulators”, in Annual Reports in Medicinal Chemistry, Vol. 30, Section I, pp. 1-9, 1995, Academic Press, Inc.).

WO00/16777 discloses that a 5-HT1A receptor agonist, buspirone is efficacious in treating a variety of symptoms associated with ADHD (attention deficit hyperactivity disorder), and that combined use of a D2 receptor agonist and 5-HT1A agonist provides effective treatments for ADHD and Parkinson's disease.

5-HT_{1A} agonists are effective in the treatment of cognitive impairment in Alzheimer's disease, Parkinson's disease or senile dementia. U.S. Pat. No. 5,824,680 discloses that a 5-HT_{1A} agonist, ipsapirone, is effective in treating Alzheimer's disease by improving memory. U.S. Pat. No. 4,687,772 describes that a 5-HT_{1A} partial agonist, buspirone, is useful for improving short term memory in patients in need of treatment. WO 93/04681 discloses that use of 5-HT_{1A} partial agonists have been used for the treatment or prevention of cognitive disorders associated with Alzheimer's disease, Parkinson's disease or senile dementia.

5-HT_{1A} agonists are also effective in the treatment of depression. U.S. Pat. No. 4,771,053 describes that a 5-HT_{1A} receptor partial agonist, gepirone, is useful in alleviation of certain primary depressive disorders, such as severe depression, endogenous depression, major depression with melancholia, and atypical depression. WO 01/52855 discloses that the combined use of the 5-HT_{1A} receptor partial agonist gepirone with an antidepressant can effectively treat depression.

However, the aforementioned patents/publications do not utilize radioligands.

The most successful radioligands studied so far for 5-HT_{1A} receptors are antagonists tracers which bind with both the G-protein-coupled high affinity (HA) state and uncoupled low affinity (LA) state of 5-HT_{1A} receptors disclosed in U.S. Pat. No. 6,056,942. U.S. Pat. No. 6,056,942 describes selective 5-HT_{1A} antagonists radiolabelled with ³H or ¹¹C ligands which are useful, for example, in pharmacological screening procedures and in positron emission tomography (PET) studies. In contrast, agonists bind preferentially to the HA state of the 5-HT_{1A} receptor. Therefore, having a radioligand agonist tracer may provide a more meaningful functional measure of 5-HT_{1A} receptors.

There have only been a few studies performed on select 5-HT_{1A} agonist radiotracers in a living brain. These studies unfortunately have resulted in low radiochemical yield (less than 2%) and purity, WO 2009006227. Thus, there is still a need in the art for radiolabeled serotonin receptor agonist, partial agonist, inverse agonist, or antagonist modulators that are highly selective for imaging 5-HT_{1A} receptors. There also remains a need in the art for selective radioactive tracers, which are useful for imaging 5-HT_{1A} receptors in vivo by powerful imaging methods like PET (Positron Emission Tomography) or SPECT (Single Photon Emission Computed Tomography). There is also a need for a more efficient method of obtaining these selective radioactive tracers that yields a higher radiochemical yield and purity.

Imaging methods currently exist which enable one to assess the living brain and body in vivo and thereby monitor the effectiveness of treatments that affect brain chemistry and function. PET is a dynamic, non-invasive imaging technique used in nuclear medicine to study various biochemical and biological process in vivo. In PET, radiolabeled and non-radiolabeled compounds may be administered in nanomolar or picomolar concentrations, allowing imaging studies to be performed without perturbing the biological system being studied. These labeled compounds may generally be radioisotopes that give off positrons. The emitted positrons may then collide with electrons to generate gamma rays. The emitted gamma rays may then be detected by scanners and be processed to obtain images of the living brain and body. Like other dynamic imaging protocols, PET has the ability collect images repeatedly over time and provide information about regional distribution of the tracer as well as the change in compartmental distribution as a function of time. As such, PET lends itself directly to measuring kinetic processes, such as rate of tracer uptake by cells, substrate metabolic rates, receptor density/affinity, and regional blood flow.

SPECT imaging though is performed by using a gamma camera to acquire multiple 2-D images (also called projections), from multiple angles. A computer is then used to apply a tomographic reconstruction algorithm to the multiple projections, yielding a 3-D dataset. This dataset may then be manipulated to show thin slices along any chosen axis of the body, similar to those obtained from other tomographic techniques, such as MRI, CT, and PET.

SPECT is similar to PET in its use of radioactive tracer material and detection of gamma rays. In contrast with PET, however, the tracer used in SPECT emits gamma radiation that is measured directly, whereas PET tracer emits positrons which annihilate with electrons up to a few millimeters away, causing two gamma photons to be emitted in opposite directions. A PET scanner detects these emissions “coincident” in time, which provides more radiation event localization information and thus higher resolution images than SPECT (which has about 1 cm resolution). SPECT scans, however, are significantly less expensive than PET scans, in part because they are able to use longer-lived more easily-obtained radioisotopes than PET.

The basic technique of SPECT requires injection of a gamma-emitting radioisotope into the bloodstream of a subject. Occasionally the radioisotope is a simple soluble dissolved ion, such as a radioisotope of gallium(III), which happens to also have chemical properties which allow it to be concentrated in ways of medical interest for disease detection. However, most of the time in SPECT, a marker radioisotope, which is of interest only for its radioactive properties, has been attached to a special radioligand, which is of interest for its chemical binding properties to certain types of tissues. This combination allows both the ligand and radioisotope (the radiopharmaceutical) to be carried together and bound to a place of interest in the body, which then (due to the gamma-emission of the isotope) allows the ligand concentration to be seen by a gamma-camera.

### SUMMARY OF THE INVENTION

Viewed from one aspect the present invention provides radiolabeled and non-radiolabeled compounds of Formula I:

Z—Y-L₂-N(R₁)-L₁-X(R₂)—Ar (I)

or a pharmaceutically acceptable salt thereof,
wherein:
Ar is -aryl or a 3- to 9-membered aromatic heterocycle;

### X is —N, —CH—, O, or S;

R₁ is absent, H, Me or with R₂ forms a heterocycloalkyl group

### L₁ is a (—CH₂)₂—

L₂ is —(CH₂)ₙ— or —(CH₂)ᵣ-L₃-(CH₂)ₛ— where n is an integer ranging from 1 to 5; r and s are independently integers ranging from 0 to 2
L₃ is a 3-9-membered cycloalkyl or heterocycloalkyl
Y is absent or a bond, S, O, NH, CONH, NHCO or SO₂NH;
Z is selected from a group comprising a 3-to 9-membered aromatic heterocycle, aryl, an alkyl, cycloalkyl or a heterocycloalkyl;
wherein said compound of formula (I) is not a compound of the formula:

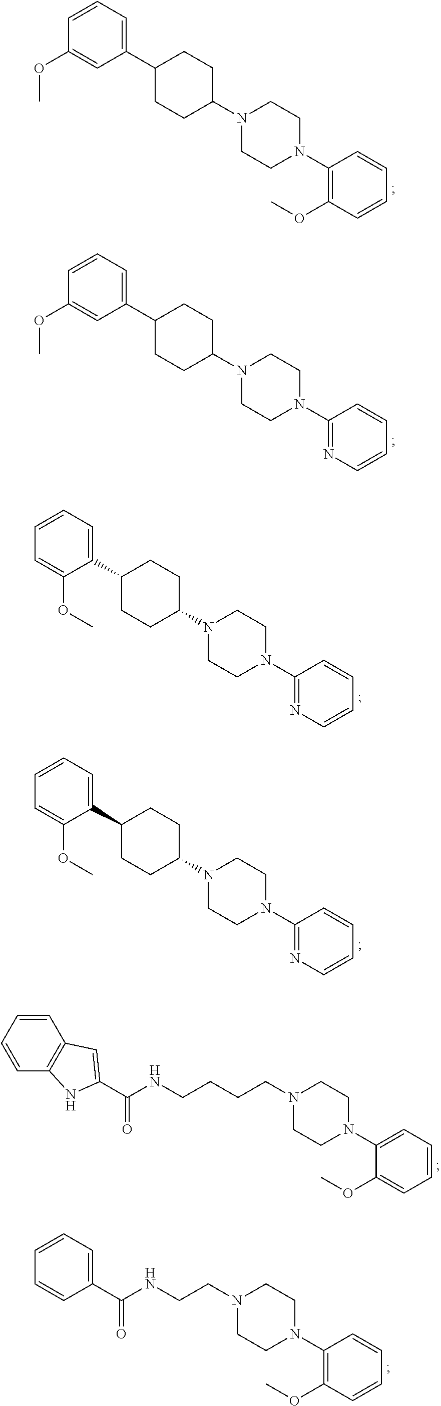 [SMILES: COc1cccc(C2CCC(N3CCN(c4ccccc4OC)CC3)CC2)c1 ; COc1cccc(C2CCC(N3CCN(c4ccccn4)CC3)CC2)c1 ; COc1ccccc1[C@H]1CC[C@@H](N2CCN(c3ccccn3)CC2)CC1 ; COc1ccccc1[C@H]1CC[C@H](N2CCN(c3ccccn3)CC2)CC1 ; COc1ccccc1N1CCN(CCCCNC(=O)c2cc3ccccc3[nH]2)CC1 ; COc1ccccc1N1CCN(CCNC(=O)c2ccccc2)CC1]

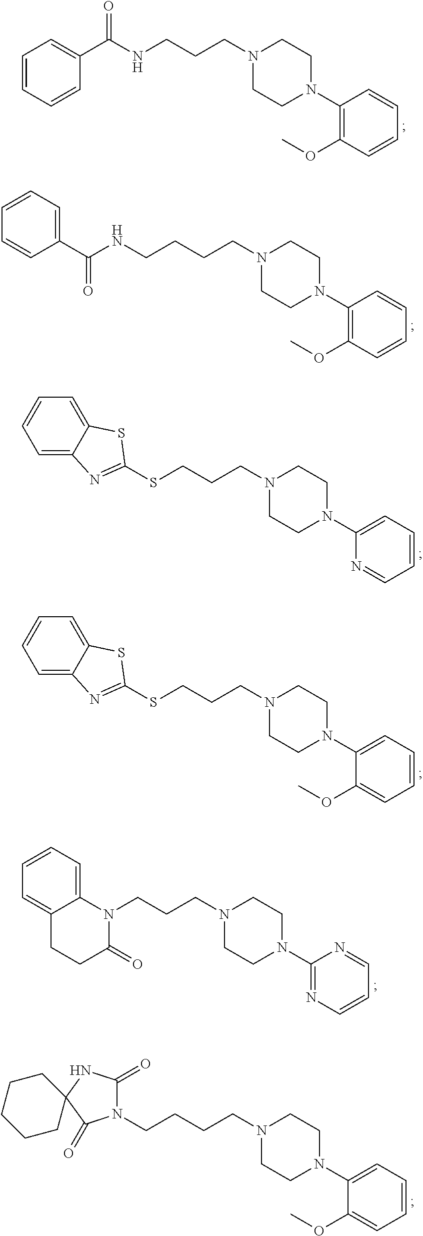 [SMILES: COc1ccccc1N1CCN(CCCNC(=O)c2ccccc2)CC1 ; COc1ccccc1N1CCN(CCCCNC(=O)c2ccccc2)CC1 ; c1ccc(N2CCN(CCCSc3nc4ccccc4s3)CC2)nc1 ; COc1ccccc1N1CCN(CCCSc2nc3ccccc3s2)CC1 ; O=C1CCc2ccccc2N1CCCN1CCN(c2ncccn2)CC1 ; COc1ccccc1N1CCN(CCCCN2C(=O)NC3(CCCCC3)C2=O)CC1]

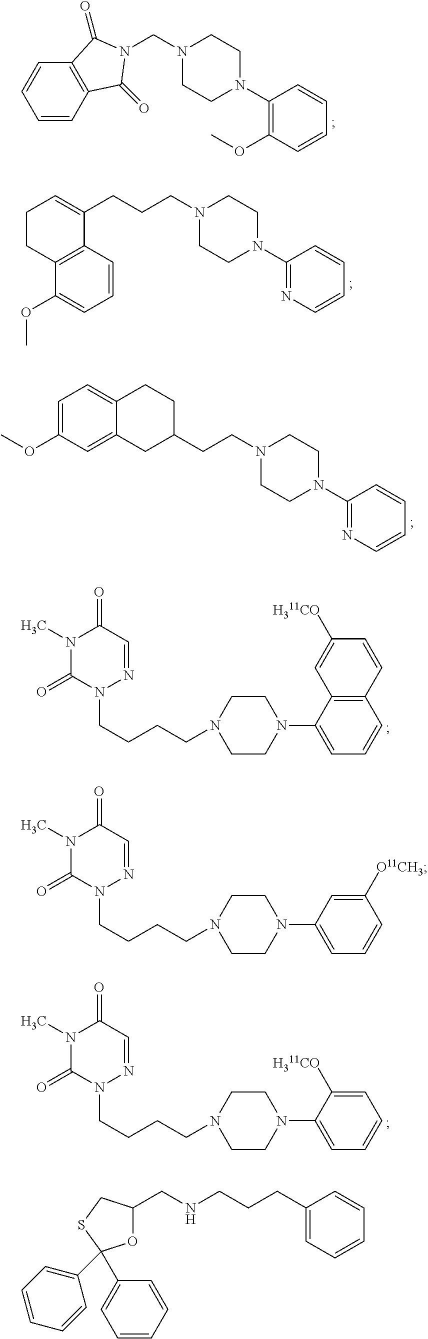

The compounds of formula (I) have angular and distance requirements specifically for the agonists. These requirements are found in MF Hibbert et al., Eur. J. Med. Chem. 1989, 24, 31. and in M L Lopez-Rodriguez et al., Current Med Chem. 2002, 9, 443.

A model around requirements for hydrogen bond acceptor and other pharmacophore requirements is indicated in P Gaillard et al., J. Med. Chem. 1996, 39, 126. A much more recent pharmacophore model that seems to work across classes is K C Weber et al., Eur J Med Chem., 2010, 45, 1508.

In one embodiment of the present invention, the compounds of formula (I) may be used in therapeutically effective treatments as well as for imaging purposes.

In yet another aspect, the present invention provides a method for detecting in vivo 5-HT_{1A} receptors in a subject such as a human or animal, the method comprising:

(a) administering to the subject an imaging-effective amount of radiolabeled compounds of Formula (I), or a pharmaceutically acceptable salt
thereof, and
(b) detecting the radioactive emission of the compound or salt thereof administered to the subject.

In the present methods, the radioactive emissions from the ¹¹C and/or ¹⁸F-atom of a radiolabeled compound can be detected using PET for imaging one or more 5-HT_{1A} serotonin receptors in a subject. The radioactive emission can be detected anywhere in the body of the subject. In one embodiment, the radioactive emission is detected in the brain of the subject. In a further embodiment, the subject can be known or suspected to have a psychiatric or neurological disorder.

In another aspect, a radiolabeled compound or a pharmaceutically acceptable salt thereof is useful for: (i) diagnosing, treating or preventing a psychiatric disorder, or (ii) stabilizing the mood of a subject having a mood disorder.

The invention also relates to compositions comprising a physiologically acceptable carrier or vehicle and an amount of a radiolabeled compound that is effective to: (i) diagnose, treat or prevent a psychiatric disorder in a subject; or (ii) stabilize the mood of a subject having a mood disorder. The compositions are useful for diagnosing, treating or preventing a psychiatric disorder in a subject, or for stabilizing the mood of a subject having a mood disorder.

In one aspect, the invention relates to a method of making a compound of the formula (VII):

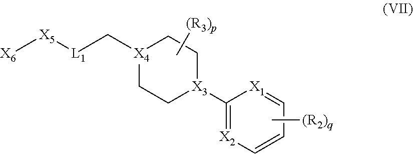

the method comprising:
(i) reacting a compound of the formula

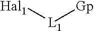

where Hal₁ is a halogen; Gp is a different halogen than Hal₁, an amine or a protected amine; and L₁ is optionally substituted alkyl or optionally substituted cycloalkyl; with an optionally substituted heterocycloalkyl compound to give a compound of the formula:

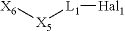

where:
X₅ is a bond; and
X₆ is optionally substituted heterocycloalkyl; and
(ii) reacting the compound of formula

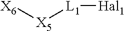

with a compound of the formula:

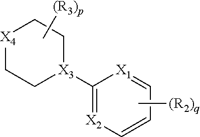

wherein X₁ and X₂ are the same or different, independently one from the other, and each is N or CR₁, where R₁ is H, hydroxy, alkoxy, halo, haloalkyloxy, nitro, haloalkylamido, or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ is N or CR₄, wherein R₄ is H or halogen;
X₄ is N; and
p and q are the same or different, independently one from the other, and each is 0, 1 or 2;
   to give a compound of the formula (VII):

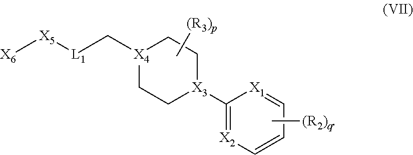

In another aspect, the invention relates to a method of making a compound of the formula (VII):

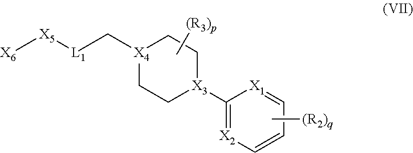

the method comprising:
(i) reacting a compound of the formula

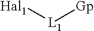

where Hal₁ is a halogen; Gp is a different halogen than Hal₁, an amine or a protected amine; and L₁ is optionally substituted alkyl or optionally substituted cycloalkyl; with a compound of the formula:

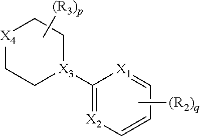

wherein X₁ and X₂ are the same or different, independently one from the other, and each is N or CR₁, where R₁ is H, hydroxy, alkoxy, halo, haloalkyloxy, nitro, haloalkylamido, or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ is N or CR₄, wherein R₄ is H or halogen;
X₄ is N; and
p and q are the same or different, independently one from the other, and each is 0, 1 or 2;
   to give a compound of the formula:

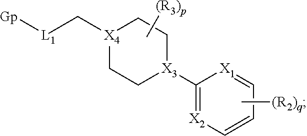

and
(ii) reacting a compound of the formula:

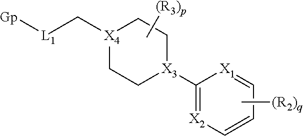

with a compound of the formula:

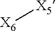

where:
X₅′ comprises a group that reacts with Gp; and
X₆ is optionally substituted heterocycloalkyl;
   to give a compound of the formula (VII):

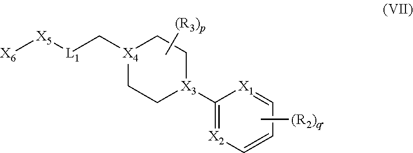

In some embodiments, the group that reacts with Gp, comprised in X₅′, comprises a thiol, an amine or a hydroxyl.

In still another aspect, the invention relates to a method of preparing a compound of formula (VIII):

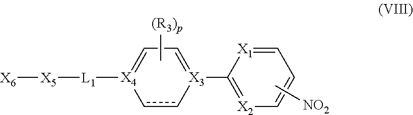

the method comprising reacting a compound of the formula

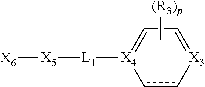

with a compound of the formula

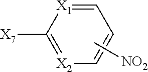

wherein X₁ and X₂ are the same or different, independently one from the other, and each is N or CR₁, where R₁ is H, hydroxy, alkoxy, halo, haloalkyloxy, nitro, haloalkylamido;
X₇ is halo;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

In yet another aspect, the invention relates to a method of preparing a compound of formula (IX):

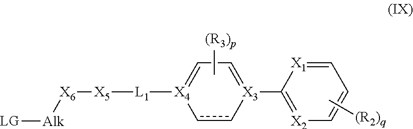

the method comprising reacting a compound of the formula

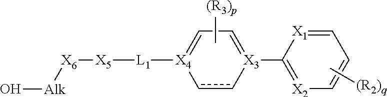

with a reagent which transforms the hydroxyl group attached to Alk into a leaving group;
wherein Alk is an alkyl group;
LG is a leaving group;
X₁ and X₂ are the same or different, independently one from the other, and each is N or CR₁, where R₁ is H, hydroxy, alkoxy, halo, haloalkyloxy, nitro, haloalkylamido or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl;
p and q are the same or different, independently one from the other, and each is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

In still another aspect, the invention relates to a method of preparing a compound of formula (X):

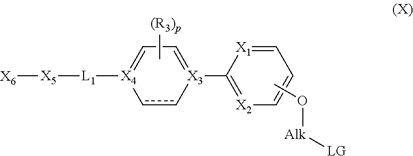

the method comprising reacting a compound of the formula

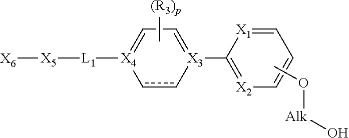

with a reagent which transforms the hydroxyl group attached to Alk into a leaving group;
wherein Alk is an alkyl group;
LG is a leaving group;
wherein X₁ and X₂ are the same or different, independently one from the other, and each is N or CR₁, where R₁ is H, hydroxy, alkoxy, halo, haloalkyloxy, nitro, haloalkylamido;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

In a further aspect, the invention relates to a compound of formula (VIII):

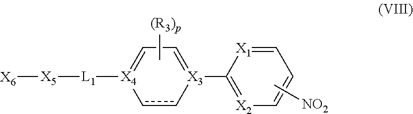

wherein X₁ and X₂ are the same or different, independently one from the other, and each is N or CR₁, where R₁ is H, hydroxy, alkoxy, halo, haloalkyloxy, nitro, haloalkylamido;
X₇ is halo;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

In still a further aspect, the invention relates to a compound of formula (IX):

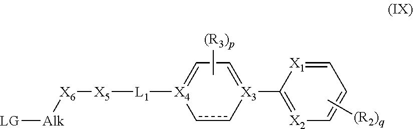

wherein Alk is an alkyl group;
LG is a leaving group;
X₁ and X₂ are the same or different, independently one from the other, and each is N or CR₁, where R₁ is H, hydroxy, alkoxy, halo, haloalkyloxy, nitro, haloalkylamido or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl;
p and q are the same or different, independently one from the other, and each is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

In yet a further aspect, the invention relates to a method of preparing a compound of formula (X):

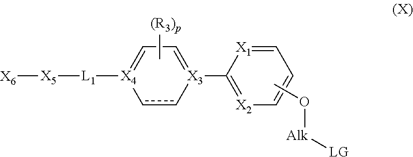

the method comprising reacting a compound of the formula

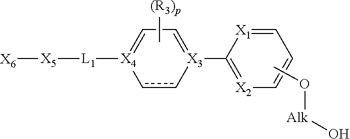

with a reagent which transforms the hydroxyl group attached to Alk into a leaving group;
wherein Alk is an alkyl group;
LG is a leaving group;
wherein X₁ and X₂ are the same or different, independently one from the other, and each is N or CR₁, where R₁ is H, hydroxy, alkoxy, halo, haloalkyloxy, nitro, haloalkylamido;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

### DETAILED DESCRIPTION OF THE DRAWINGS

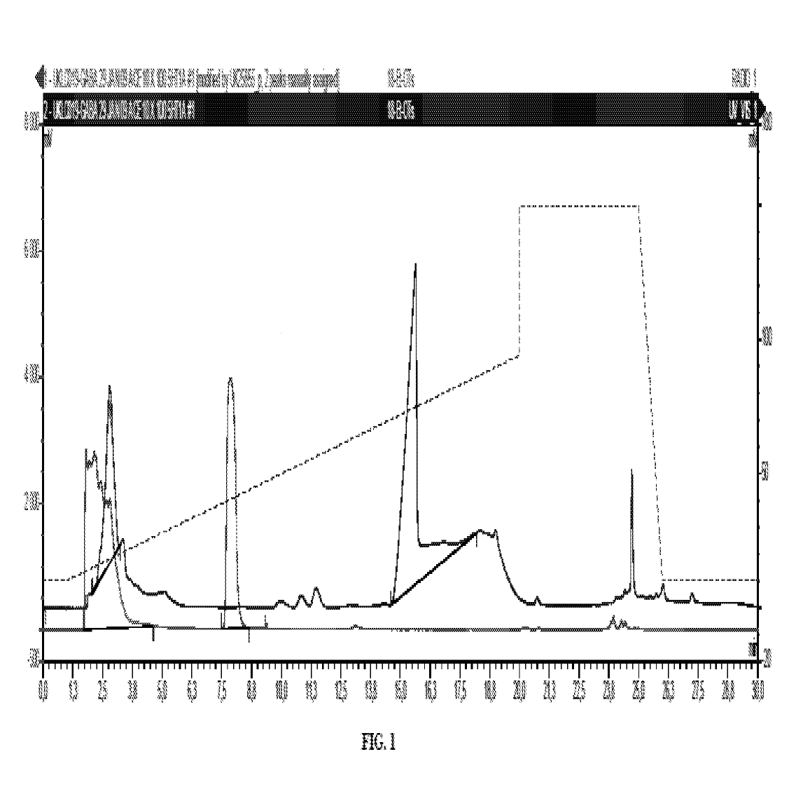

FIG. 1 is an HPLC trace obtained from the preparative HPLC purification of [¹⁸F]fluoroethyl tosylate.

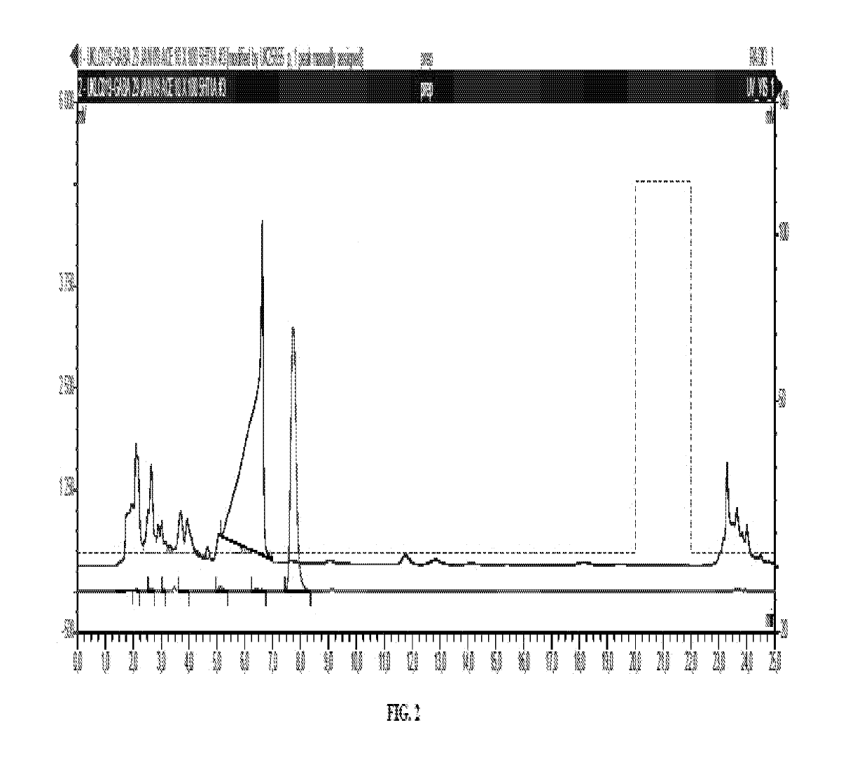

FIG. 2 is an HPLC trace obtained from the preparative purification of [¹⁸F]2-(4-(4-(2-(2-fluoroethoxy)phenyl)piperazin-1-yl)butyl)-4-methyl-1,2,4-triazine-3,5(2H,4H)-dione.

### DETAILED DESCRIPTION OF THE INVENTION

The radiolabeled compounds of Formula (I) are useful as imaging agents for one or more high affinity (HA) serotonin (5-HT_{1A}) receptors. In certain embodiments, the radiolabeled compounds have one or more of the following characteristics: (i) HA and selectivity for the serotonin (5-HT_{1A}) receptor compared to the other known transporters, receptors, enzymes and proteins; and (ii) sufficient lipophilicity to allow rapid blood-brain-barrier penetration and generation of polar metabolites that do not cross the blood-brain-barrier; and (iii) high specific activity of the compounds of Formula (I). It is possible for the radiolabeled and non-radiolabeled compounds to have one or more chiral centers and as such the radiolabeled and non-radiolabeled compounds can exist in various stereoisomeric forms. Accordingly, the compounds of formula (I), although not depicting specific stereoisomers of the radiolabeled and non-radiolabeled compounds, are understood to encompass all possible stereoisomers.

As stated above, the present invention encompasses radiolabeled and non-radiolabeled compounds having the Formula (I):

Z—Y-L₂-N(R₁)-L₁-X(R₂)—Ar (I)

or a pharmaceutically acceptable salt thereof,
wherein:
Ar is -aryl or a 3- to 9-membered aromatic heterocycle;

### X is —N, —CH—, O, or S;

R₁ is absent, H, Me or with R₂ forms a heterocycloalkyl group

### L₁ is a (—CH₂)₂—

L₂ is —(CH₂)ₙ— or —(CH₂)ᵣ-L₃-(CH₂)ₛ— where n is an integer ranging from 1 to 5; r and s are independently integers ranging from 0 to 2
L₃ is a 3-9-membered cycloalkyl or heterocycloalkyl
Y is absent or a bond, S, O, NH, CONH, NHCO or SO₂NH; and
Z is selected from a group comprising a 3-to 9-membered aromatic heterocycle, aryl, an alkyl, cycloalkyl or a heterocycloalkyl;
wherein said compound of formula (I) is not a compound of the formula:

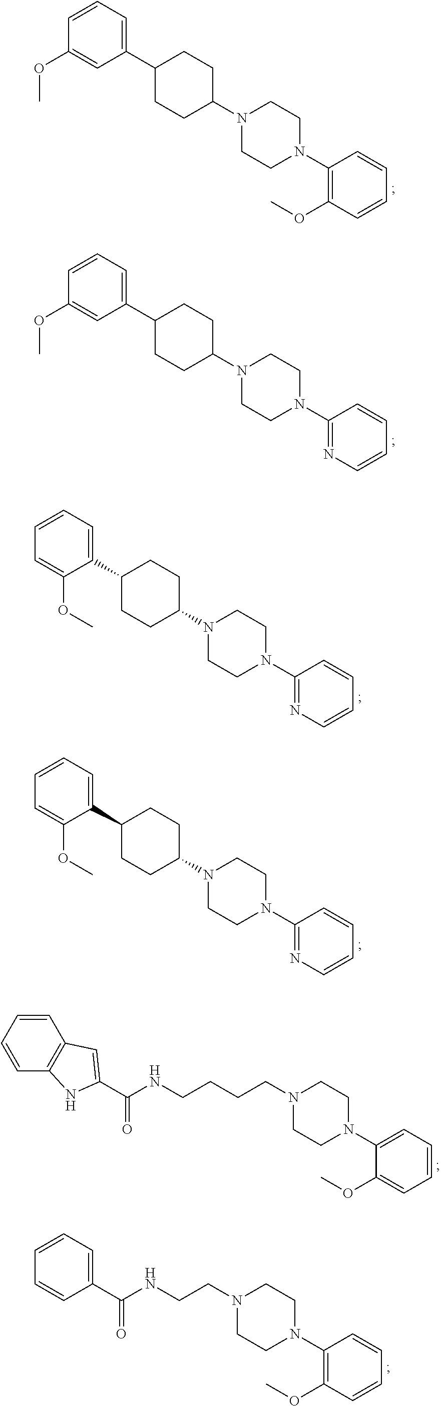 [SMILES: COc1cccc(C2CCC(N3CCN(c4ccccc4OC)CC3)CC2)c1 ; COc1cccc(C2CCC(N3CCN(c4ccccn4)CC3)CC2)c1 ; COc1ccccc1[C@H]1CC[C@@H](N2CCN(c3ccccn3)CC2)CC1 ; COc1ccccc1[C@H]1CC[C@H](N2CCN(c3ccccn3)CC2)CC1 ; COc1ccccc1N1CCN(CCCCNC(=O)c2cc3ccccc3[nH]2)CC1 ; COc1ccccc1N1CCN(CCNC(=O)c2ccccc2)CC1]

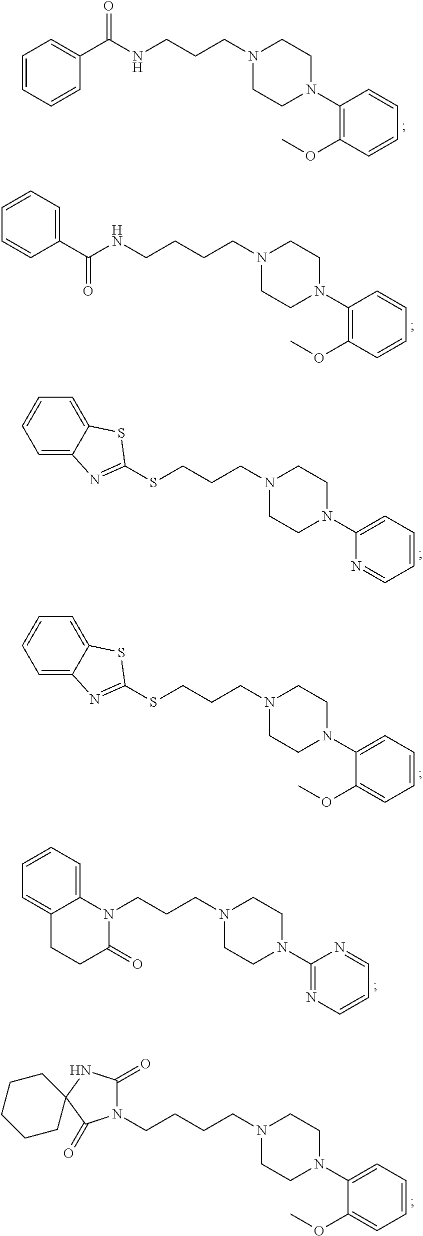 [SMILES: COc1ccccc1N1CCN(CCCNC(=O)c2ccccc2)CC1 ; COc1ccccc1N1CCN(CCCCNC(=O)c2ccccc2)CC1 ; c1ccc(N2CCN(CCCSc3nc4ccccc4s3)CC2)nc1 ; COc1ccccc1N1CCN(CCCSc2nc3ccccc3s2)CC1 ; O=C1CCc2ccccc2N1CCCN1CCN(c2ncccn2)CC1 ; COc1ccccc1N1CCN(CCCCN2C(=O)NC3(CCCCC3)C2=O)CC1]

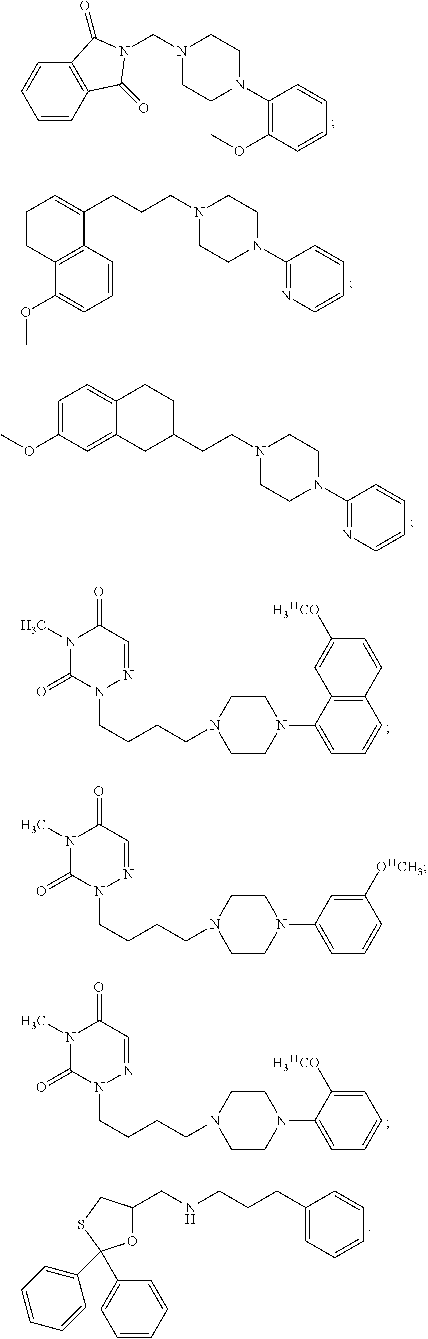

In some embodiments, the compounds of the formula (I) are radiolabeled. In some embodiments, the compounds of formula (I) are not radiolabeled.

In some embodiments, the compound of formula (I) comprises an ¹⁸F or a ¹¹C atom. In some embodiments, Ar is directly (e.g., covalently) attached to the ¹⁸F or the ¹¹C atom. In other embodiments, the ¹⁸F or ¹¹C atom is attached to the Ar group via a —OCₙHₘ¹⁸F group or via a —OC¹¹H₃ group, wherein n is 1 to 4 and m is 2 to 8, respectively. In still other embodiments, the ¹⁸F or ¹¹C atom is attached to compounds of the formula (I) directly to Z or to a suitable group on Z. In yet other embodiments, the ¹⁸F or ¹¹C atom is attached to compounds of the formula (I) directly to L₂ or L₃ or to a suitable group on L₂ or L₃.

In some embodiments of the present invention, N(R₁)-L₁-X(R₂) combine to form a piperazine group with the following connectivity:

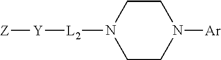

The compounds of formula (I) were designed to have angular and distance requirements characteristic of agonists. These requirements are found in MF Hibbert et al., Eur. J. Med. Chem. 1989, 24, 31. and in M L Lopez-Rodriguez et al., Current Med Chem. 2002, 9, 443.

A model around requirements for hydrogen bond acceptor and other pharmacophore requirements is indicated in P Gaillard et al., J. Med. Chem. 1996, 39, 126. A much more recent pharmacophore model that seems to work across classes is K C Weber et al., Eur J Med Chem., 2010, 45, 1508.

An embodiment of the present invention is wherein the compounds of formula (I) may be used in therapeutically effective treatments as well as for imaging purposes.

An “aryl” is a phenyl, napthyl, benzyl or anthracenyl. If the aryl contains one or more heteroatoms, the aryl group is referred to as a “heteroaryl” group. Representative heteroaryl groups include pyridinyl, pyrimidinyl, triazinyl, thiophenyl, thiazolyl, furanyl, pyrrolyl, oxazolyl, imidazolyl, triazyolyl, tetrazolyl, pyrazinyl or pyrazolyl that fall under the 5-7 membered heteroaromatics or could also be fused (e.g., naphthyl, indolyl, benzoxazolyl, benzthiazolyl, carbazolyl, benzimidazolyl, and quinolinyl) to another benzene ring or heterocycle and optionally aromatic. The aryl may be optionally substituted. Further, one or more carbon atoms on the aryl group can be ¹¹C.

As used herein, the term “radiolabeled compound” means a compound comprising at least one radioactive atom. Exemplary radioactive atoms for PET imaging include ¹¹C, ¹³N, ¹⁵O, ¹⁷F, ¹⁸F, ⁷⁵Br, ⁷⁶Br or ¹²⁴I, especially ¹¹C and ¹⁸F, most especially ¹⁸F. Exemplary radioactive atoms for SPECT imaging include ¹²³I, ¹³¹I or ⁷⁷Br, especially ¹²³I.

The term “alkyl” as used herein, refers to a straight chain or branched non-cyclic hydrocarbon having from 1 to 6 carbon atoms, from 1 to 4 carbon atoms, from 1 to 3 carbon atoms or from 1 to 2 carbon atoms, wherein one of the hydrocarbon's hydrogen atoms has been replaced with a single bond. Representative straight chain alkyls include-methyl, -ethyl, -n-propyl, -n-butyl, -n-pentyl, and -n-hexyl. Representative branched alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 5, 1,1-dimethylbutyl, 1,2-dimethylbutyl, 1,3-dimethylbutyl, 2,2-dimethylbutyl, 2,3-dimethylbutyl, 3,3-dimethylbutyl, -isopropyl, -sec-butyl, -isobutyl, -neohexyl, -isohexyl, and the like. The alkyl may be optionally substituted. Further, one or more carbon atoms on the alkyl group can be ¹¹C.

The term “cycloalkyl” as used herein is a 3-, 4-, 5-, 6-, 7-, 8-, 9- or 10-membered saturated non-aromatic monocyclic, bicyclic (e.g., bicyclo[2.2.1]heptyl and bicyclo[2.2.2]octanyl) or tricyclic (e.g., tricyclo[3.3.1.1^{3,7}]decyl, otherwise known as adamantyl)cycloalkyl ring. Representative C₃-C₇ monocyclic cycloalkyl groups include, but are not limited to, cyclopropyl, cyclobutyl, cyclopentyl, cyclohexyl and cycloheptyl. The non-aromatic monocyclic, bicyclic or tricyclic cycloalkyl ring optionally contains one (e.g., cyclohexenyl) or two double bonds (e.g., cyclopentadienyl). The cycloalkyl may be optionally substituted. Further, one or more carbon atoms on the cycloalkyl group can be ¹¹C

As used herein, the term “heterocycloalkyl” refers to a cycloalkyl group in which at least one of the carbon atoms in the ring is replaced by a heteroatom (e.g., O, S or N). Representative heterocycloalkyl groups include oxathiolanyl, pyrrolidinyl, piperidinyl, piperazinyl, morpholinyl, triazine dionyl (e.g., 1,2,4-triazine-3,5(2H,4H)-dion-yl), pyrimidine dionyl (e.g., pyrimidine-2,4(1H,3H)-dione), hydantoinyl and the like. The heterocycloalkyl may be optionally substituted. Further, one or more carbon atoms on the cycloalkyl group can be ¹¹C.

In some embodiments, the heterocycloalkyl group is fused with an aryl group. Examples of such heterocycloalkyl-aryl fused groups include quinazolinyl, quinazolinonyl (e.g., quinazolin-4(3H)-one), tetrahydroquinolyl (e.g., 1,2,3,4-tetrahydroquinolyl), dihydroquinolinonyl (e.g., 3,4-dihydroquinolin-2(1H)-one), 2H-benzoxazinonyl (e.g., 2H-benzo[b][1,4]oxazin-3(4H)-one), phenanthridinyl, phenanthridinonyl (e.g., phenanthridin-6(5H)-one), and the like. Such groups may be optionally substituted. Further, one or more carbon atoms on the heterocycloalkyl group can be ¹¹C.

The term “3- to 9-membered aromatic heterocycle” refers to a 3-9-membered aromatic monocyclic cycloalkyl in which 1-4 of the ring carbon atoms have been independently replaced with a N, S, or O atom or any combination of these atoms thereof. Examples of this combination of atoms include, but not limited to benzthiazole. The term 3- to 9-membered aromatic heterocycle also encompasses any heterocycles described which are fused to a benzene ring. The 3- to 9-membered aromatic heterocycles are attached via a ring carbon atom. The 2- or 3-membered aromatic heterocycle can optionally be fused with aryl group as well. Representative examples of a 3- to 9-membered aryl heterocycle group include, but are not limited to phenyl, napthyl, benzyl, pyrazinyl, pyrazolyl, pyridinyl, pyrimidinyl, adamantine or any combination thereof. In some embodiments, such heterocycles may be optionally substituted. In another embodiment, the 3- to 9-membered aromatic heterocycle group is substituted with one or more of the following groups: —F, —O, —OCnHmF, or —OCH₃ wherein n is 1 to 4 and m is 2 to 8. In a further embodiment, a tosyl group is optionally added to the 3- to 9-membered heterocycle group preferably attached or fused to an aryl in the Z position. Further, one or more carbon atoms on the 3- to 9-membered aromatic heterocycle group can be ¹¹C.

As used herein, the term “alkoxy” means an alkyl-O— group.

As used herein, the term “halo” or “halogen” means refers to chlorine, bromine, fluorine or iodine. In some embodiments, when the halogen is fluorine, the fluorine is ¹⁸F.

As used herein, the term “haloalkyloxy” refers to halo-alkyl-O—.

As used herein, the term “haloalkylamido” refers to halo-alkyl-C(O)NH—. Further, the amide carbonyl carbon atom can be ¹¹C.

As used herein, “optionally substituted” specifically envisions and allows for one or more substitutions that are common in the art. However, it is generally understood by those skilled in the art that the substituents should be selected so as to not adversely affect the useful characteristics of the compound or adversely interfere with its function. Suitable substituents may include, for example, halo groups, including ¹⁸F, perfluoroalkyl groups, perfluoroalkoxy groups, alkyl groups, haloalkyl groups (including haloalkyl groups having an ¹⁸F group), haloalkoxy groups, haloalkylamido groups, alkylamido groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, amino groups, alkyl- and dialkylamino groups, carbamoyl groups, alkylcarbonyl groups, carboxyl groups, alkoxycarbonyl groups, alkylaminocarbonyl groups, dialkylamino carbonyl groups, arylcarbonyl groups, aryloxycarbonyl groups, alkylsulfonyl groups, arylsulfonyl groups, cycloalkyl groups, cyano groups, C₁-C₆ alkylthio groups, arylthio groups, nitro groups, keto groups, acyl groups, boronate or boronyl groups, phosphate or phosphonyl groups, sulfamyl groups, sulfonyl groups, sulfinyl groups, and combinations thereof. Additionally, in some cases, suitable substituents may combine to form one or more rings as known to those of skill in the art.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

As used herein, the term “heteroatom” refers to atoms such as N, O, P, B, S, Se or Si.

The term “leaving group” refers to a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

Agonists bind (have affinity for) and activate a receptor, displaying full efficacy at that receptor. One example of a drug that acts as a full agonist is isoproterenol, which mimics the action of adrenaline at β adrenoreceptors. Another example is morphine, which mimics the actions of endorphins at μ-opioid receptors throughout the central nervous system.

Partial agonists (such as buspirone, aripiprazole, buprenorphine, or norclozapine) also bind and activate a given receptor, but have only partial efficacy at the receptor relative to a full agonist. One study of benzodiazepine active sedative hypnotics found that partial agonists have just under half the strength of full agonist. Partial agonists such as abecarnil have demonstrated a reduced rate and reduced severity of dependence and withdrawal syndromes.

An inverse agonist is an agent that binds to the same receptor binding-site as an agonist for that receptor and reverses constitutive activity of receptors. Inverse agonists exert the opposite pharmacological effect of a receptor agonist.

An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is, at least in theory, irreversible. This causes the compound to produce a brief burst of agonist activity, followed by desensitisation and internalisation of the receptor, which, with long-term treatment, produces an effect more like that of an antagonist.

A selective agonist is selective for one certain type of receptor. It can be of any of the aforementioned types.

New findings that broaden the conventional definition of pharmacology demonstrate that ligands can concurrently behave as agonist and antagonists at the same receptor, depending on effector pathways or tissue type. Terms that describe this phenomenon are “functional selectivity”, “protean agonism”, or selective receptor modulators.

An antagonist is a type of receptor ligand or drug that does not provoke a biological response itself upon binding to a receptor, but blocks or dampens agonist-mediated responses. In pharmacology, antagonists have affinity but no efficacy for their cognate receptors, and binding will disrupt the interaction and inhibit the function of an agonist or inverse agonist at receptors. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding. The majority of drug antagonists achieve their potency by competing with endogenous ligands or substrates at structurally-defined binding sites on receptors.

A further embodiment of the present invention illustrates radiolabeled and non-radiolabeled compounds of Formula (I) include but are not limited to:

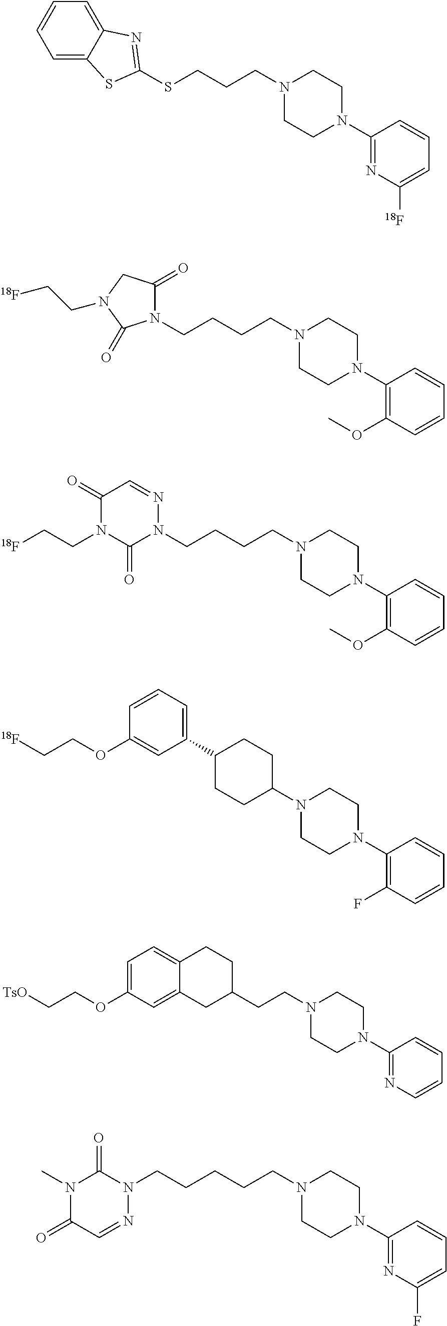

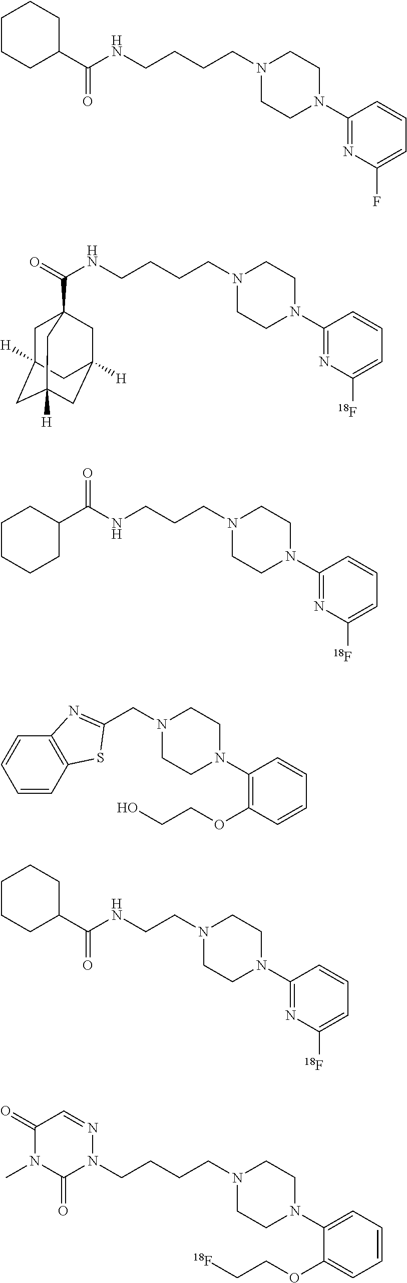 [SMILES: O=C(NCCCCN1CCN(c2cccc(F)n2)CC1)C1CCCCC1 ; O=C(NCCCCN1CCN(c2cccc([18F])n2)CC1)[C@]12C[C@H]3C[C@H](C[C@H](C3)C1)C2 ; O=C(NCCCN1CCN(c2cccc([18F])n2)CC1)C1CCCCC1 ; OCCOc1ccccc1N1CCN(Cc2nc3ccccc3s2)CC1 ; O=C(NCCN1CCN(c2cccc([18F])n2)CC1)C1CCCCC1 ; Cn1c(=O)cnn(CCCCN2CCN(c3ccccc3OCC[18F])CC2)c1=O]

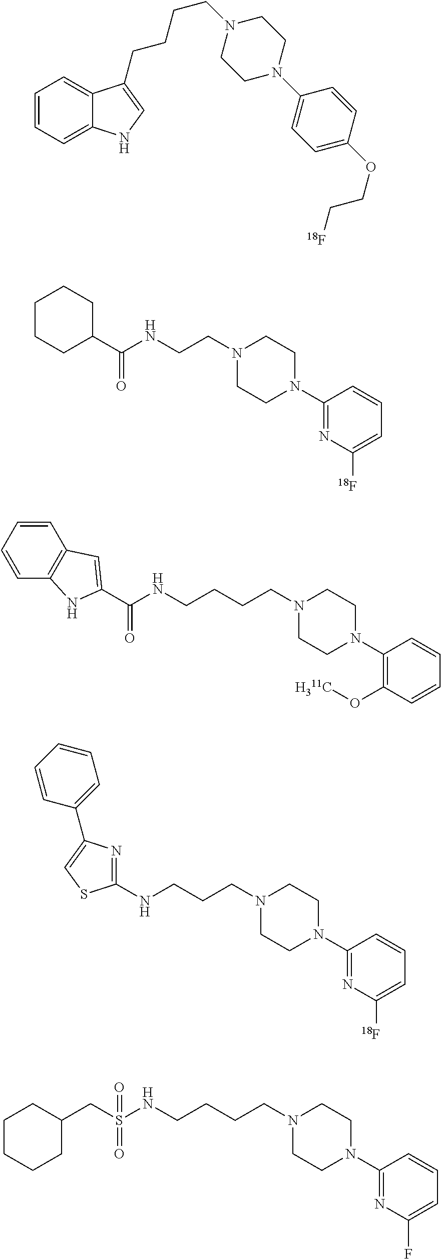 [SMILES: [18F]CCOc1ccc(N2CCN(CCCCc3c[nH]c4ccccc34)CC2)cc1 ; O=C(NCCN1CCN(c2cccc([18F])n2)CC1)C1CCCCC1 ; [11CH3]Oc1ccccc1N1CCN(CCCCNC(=O)c2cc3ccccc3[nH]2)CC1 ; [18F]c1cccc(N2CCN(CCCNc3nc(-c4ccccc4)cs3)CC2)n1 ; O=S(=O)(CC1CCCCC1)NCCCCN1CCN(c2cccc(F)n2)CC1]

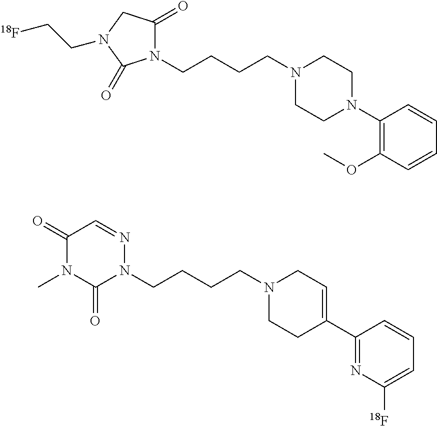 [SMILES: COc1ccccc1N1CCN(CCCCN2C(=O)CN(CC[18F])C2=O)CC1 ; Cn1c(=O)cnn(CCCCN2CC=C(c3cccc([18F])n3)CC2)c1=O]

and pharmaceutically acceptable salts thereof.

A further embodiment of the present invention illustrates radiolabeled compounds of Formula (I) include, but are not limited to:

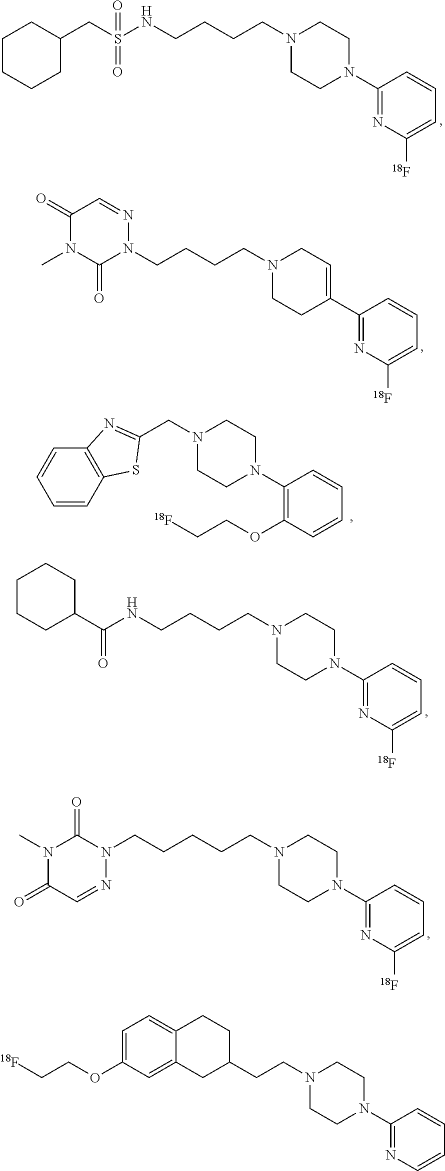 [SMILES: O=S(=O)(CC1CCCCC1)NCCCCN1CCN(c2cccc([18F])n2)CC1 ; Cn1c(=O)cnn(CCCCN2CC=C(c3cccc([18F])n3)CC2)c1=O ; [18F]CCOc1ccccc1N1CCN(Cc2nc3ccccc3s2)CC1 ; O=C(NCCCCN1CCN(c2cccc([18F])n2)CC1)C1CCCCC1 ; Cn1c(=O)cnn(CCCCCN2CCN(c3cccc([18F])n3)CC2)c1=O ; [18F]CCOc1ccc2c(c1)CC(CCN1CCN(c3ccccn3)CC1)CC2]

or a pharmaceutically acceptable salt thereof.

In one embodiment, a radiolabeled and non-radiolabeled compound of Formula (I) is an agonist, partial agonist, or inverse agonist of the 5-HT_{1A} receptor.

The radiolabeled compounds of Formula (I) can be used as imaging agents to image one or more 5-HT_{1A} receptors in a subject.

In another embodiment, the present invention relates to the use of radiolabeled compounds for detecting one or more 5-HT_{1A} receptors in vivo. In particular, the present methods for detecting 5-HT_{1A} receptors in vivo contemplate the use of PET, where the imaging probe is a radiolabeled compound of the present invention.

In yet another embodiment, the invention provides a method for imaging one or more 5-HT_{1A} receptors in a subject in vivo comprising the steps: (a) administering to the subject an imaging-effective amount of a compound having Formula (I) or a pharmaceutically acceptable salt thereof, and (b) detecting the radioactive emission of the compound or salt thereof administered in step (a).

In another embodiment the invention provides a method for imaging one or more 5-HT_{1A} receptors in a subject in vivo, the method comprising:
(a) administering to the subject an imaging-effective amount of a compound of formula (I), or a pharmaceutically acceptable salt thereof; and
(b) detecting the radioactive emission of the radiolabel on the compound of formula (I), or salt thereof, following its administration to the subject.

In one embodiment, the detecting of step (b) is carried out using PET. Yet in another embodiment the detecting of step (b) is carried out using SPECT.

Such methods are applied to compounds of the formula (I) that are radiolabeled. In some embodiments, such methods can be applied to a compound of formula (I) comprising an ¹⁸F or a ¹¹C atom where, e.g., Ar is directly (e.g., covalently) attached to the ¹⁸F or the ¹¹C atom; the ¹⁸F or ¹¹C atom is attached to the Ar group via a —OCₙHₘ¹⁸F group or via a —OC¹¹H₃ group, wherein n is 1 to 4 and m is 2 to 8, respectively; where the ¹⁸F or ¹¹C atom is attached to compounds of the formula (I) directly to Z or to a suitable group on Z; or where the ¹⁸F or ¹¹C atom is attached to compounds of the formula (I) directly to L₂ or L₃ or to a suitable group on L₂ or L₃.

In another embodiment, the 5-HT_{1A} receptors being imaged are in the brain of the subject. Accordingly, the radioactive emission is detected in the brain of the subject. Methods for imaging, and thereby detecting, 5-HT_{1A} receptors in vivo are desirable in order to screen individuals for psychiatric neurological disorders or for diseases, disorders, states or conditions that are related to the binding of serotonin to 5-HT_{1A} receptors. For example, the following list of processes, diseases or disorders may involve alterations in normal binding of serotonin to 5-HT_{1A} receptors: mood disorders, such as a major depressive disorder or bipolar disorder; an eating disorder, such as anorexia nervosa or bulemia; drug addiction, alcoholism, or sexual addiction; a sleep disorder, such as insomnia or narcolepsy; a disease associated with cognitive dysfunction, such as Alzheimer's disease; a neurodegenerative disease, such as stroke; a pain disorder, including neuropathic pain or cancer pain; psychotic disorders such as schizophrenia; a movement disorder, such as Parkinson's disease; an anxiety disorder such as panic disorder, or obsessive-compulsive disorder or social phobia; a seizure disorder, such as temporal lobe epilepsy. Further, radiolabeled compounds of the present invention which are selective for the 5-HT_{1A} receptor can be used to screen for individuals who are more likely to respond to drugs that act on these receptors or susceptible to side effects of drugs which bind to the 5-HT_{1A} receptor, as manifested by an increased detection of radiolabeled 5-HT_{1A} selective agents in specified tissue compartments. These compounds can be used to identify the dose range of drugs to treat illnesses and disorders that work by binding to this receptor.

Further, the radiolabeled and non-radiolabeled compounds have a preferred high affinity and specificity to the 5-HT_{1A} receptor. In one embodiment, the radiolabeled and non-radiolabeled compounds have a HA for a 5-HT_{1A} receptor wherein the receptor's binding affinity is in the range of about 10 picomolar to about 10 nanomolar, wherein the most prefereable binding affinity is less than 1 nanomolar. This binding affinity is greater than the binding affinity for any of the other known transporters, receptors, enzymes, and peptides. The radiolabeled compounds of the present invention can be used to detect and/or quantitatively measure the HA state of 5-HT_{1A} receptor levels in subjects, including humans. The radiolabeled compounds of the present invention can also be used to measure and/or detect HA states of 5-HT_{1A} receptors in 5-HT_{1A} receptor related diseases, conditions and disorders, including but not limited to, mood disorders, such as a major depressive disorder or bipolar disorder; an eating disorder, such as anorexia nervosa or bulemia; drug addiction, alcoholism, or sexual addiction; a sleep disorder, such as insomnia or narcolepsy; a disease associated with cognitive dysfunction, such as Alzheimer's disease; a neurodegenerative disease, such as stroke; a pain disorder, including neuropathic pain or cancer pain; psychotic disorders such as schizophrenia; a movement disorder, such as Parkinson's disease; an anxiety disorder such as panic disorder, or obsessive-compulsive disorder or social phobia; a seizure disorder, such as temporal lobe epilepsy.

The ability to quantitatively measure the HA state of 5-HT_{1A} receptor levels in a subject is useful for pre-screening subjects and in one embodiment, a radiolabeled compound of Formula I disclosed in the present invention can be administered to a subject to help determine whether the subject is likely to be a responder or non-responder to medicinal agents which bind to HA 5-HT_{1A} receptors. The ability to quantitatively measure HA state 5-HT_{1A} receptor levels in a subject is useful for pre-screening clinical trial patient populations.

The radiolabeled compounds of the present invention can be used to detect or monitor processes, diseases or disorders that may involve the binding of serotonin to HA 5-HT_{1A} receptors, including but not limited to, a mood disorder, such as a major depressive disorder or bipolar disorder; an eating disorder, such as anorexia nervosa or bulemia; drug addiction, alcoholism, or sexual addiction; a sleep disorder, such as insomnia or narcolepsy; a disease associated with cognitive dysfunction, such as Alzheimer's disease; a neurodegenerative disease, such as stroke; a pain disorder, including neuropathic pain or cancer pain; a psychotic disorder, such as schizophrenia; a movement disorder, such as Parkinson's disease; an anxiety disorder such as panic disorder, or obsessive-compulsive disorder or social phobia; a seizure disorder, such as temporal lobe epilepsy.

The radiolabeled compounds of the present invention can also be used to help determine the capacity that one or more HA 5-HT_{1A} receptors have for signaling. In this embodiment, the present methods for imaging HA 5-HT_{1A} receptors can be used to determine the percentage of HA 5-HT_{1A} receptors. In a specific embodiment, the radiolabeled compound of the present invention being administered for imaging one or more HA 5-HT_{1A} receptors are agonist, partial agonists, or inverse agonists of the 5-HT_{1A} receptor.

Further, the radiolabeled compounds of the present invention can be used to screen for subjects who are more susceptible to side effects of agents which bind to HA 5-HT_{1A} receptors, as manifested by an increased detection of the radiolabeled compounds of the present invention in specified tissue compartments.

Additionally, the radiolabeled compounds of the present invention are useful in drug discovery programs and in one embodiment, can be used to determine the efficacy of agents that bind to HA 5-HT_{1A} receptors when such agents are administered to a subject to treat a disorder whose etiology involves the binding of serotonin to one or more 5-HT_{1A} receptors.

In another embodiment, the radiolabeled compounds of the present invention can be used to monitor the occupancy and occupancy rate of HA 5-HT_{1A} receptors in a subject after the subject has been administered an agent which binds to HA 5-HT_{1A} receptors.

In one embodiment, the occupancy and occupancy rate of HA 5-HT_{1A} receptors for experimental drugs can be used to help determine optimal dosage levels of such drugs. In so far as the radiolabeled and non-radiolabeled compounds of the present invention are either an agonist, partial agonist, or inverse agonists these type of compounds have special advantages in quantifying the receptor occupancy of potential new therapeutic agents that are also agonists and therefore in determining the optimal dose to use for those agents as part of an Investigational New Drug (IND) application process and thereby shorten the time period to acquire data for regulatory approval for marketing and general use in treatment. When the radiolabeled compound of the present invention is an agonist it will also aid the study and diagnosis of disease by being more sensitive to the quantification of serotonin release and depletion.

Alternatively, the methods for detection can be used to monitor the course of a HA 5-HT_{1A} receptor related disease in an individual. Thus, whether a particular therapeutic regimen aimed at ameliorating the cause of the disease, or the disease process itself, is effective, can be determined by measuring the decrease of HA 5-HT_{1A} receptors at suspected sites of disease.

In a further embodiment, the present methods for imaging one or more HA 5-HT_{1A} receptors can provide images of the location of HA 5-HT_{1A} receptors and serve as a guide to surgeons who are operating in the area of such receptors. In one embodiment, the surgeon is a neurosurgeon operating on the brain of a subject.

In one embodiment, the present invention provides a method for treating a disease associated with abnormal 5-HT_{1A} receptor function comprising administering to the subject in need thereofan effective amount of a compound of formula (I), or a pharmaceutically acceptable salt thereof. Such diseases include, but are not limited to, neurological disorders and psychiatric disorders.

A psychiatric disorder can be treated or prevented by administration of a therapeutically effective amount of a non-radiolabelled or radiolabeled compound of the present invention. Psychiatric disorders that can be treated or prevented by administering a therapeutically effective amount of a non-radiolabelled or radiolabeled compound of the present invention include, but are not limited to, a mood disorder, such as a major depressive disorder, bipolar disorder, manic depression, depression, cyclothymia, dysthymia, or borderline personality disorder; an eating disorder, such as anorexia nervosa or bulemia; an addictive disorder, such as drug addiction, alcoholism, or sexual addiction; a sleep disorder, such as insomnia or narcolepsy; a disease associated with cognitive dysfunction, such as Alzheimer's disease; a neurodegenerative disease, such as stroke; a pain disorder, including neuropathic pain or cancer pain; psychotic disorders such as schizophrenia; a movement disorder, such as Parkinson's disease; an anxiety disorder such as panic disorder, or obsessive-compulsive disorder or social phobia; a seizure disorder, such as temporal lobe epilepsy.

In one embodiment, the psychiatric disorder is a mood disorder.

In another embodiment, the psychiatric disorder is an eating disorder.

In another embodiment, the psychiatric disorder is an addictive disorder.

In another embodiment, the psychiatric disorder is a disease associated with cognitive dysfunction.

In a specific embodiment, the psychiatric disorder is Alzheimer's disease.

In still another embodiment, the psychiatric disorder is a neurodegenerative disease.

In yet another embodiment, the psychiatric disorder is a pain disorder.

In another embodiment, the psychiatric disorder is a psychotic disorder.

In one embodiment, the psychiatric disorder is a movement disorder.

In another embodiment, the psychiatric disorder is an anxiety disorder.

In still another embodiment, the psychiatric disorder is a seizure disorder.

In yet another embodiment, the psychiatric disorder is an obsessive-compulsive disorder.

The mood of a subject having a mood disorder can be stabilized by administration of a therapeutically effective amount of both a non-radiolabelled and radiolabeled compound of the present invention. Mood disorders in which the radiolabeled and non-radiolabeled compounds of the present invention are useful for stabilizing the mood include, but are not limited to, a major depressive disorder, bipolar disorder, manic depression, depression, cyclothymia, dysthymia, and borderline personality disorder.

In one embodiment, the mood disorder is a major depressive disorder.

In another embodiment, the mood disorder is bipolar disorder.

Examples of conditions treatable or preventable using the radiolabeled and non-radiolabeled compounds of the present invention include, but are not limited to, an eating disorder, such as anorexia nervosa or bulemia; drug addiction, alcoholism, or sexual addiction; a sleep disorder, such as insomnia or narcolepsy; a disease associated with cognitive dysfunction, such as Alzheimer's disease; a neurodegenerative disease, such as stroke; a pain disorder, including neuropathic pain or cancer pain; psychotic disorders such as schizophrenia; a movement disorder, such as Parkinson's disease; an anxiety disorder such as panic disorder, or obsessive-compulsive disorder or social phobia; or a seizure disorder, such as temporal lobe epilepsy.

Yet in another embodiment, the present application discloses precursors of making compounds of formula (I). Examples of making these precursors and the precursors are as follows. These precursors are not limited in any way to these examples:

A method of preparing a precursor compound of formula (II) wherein said method comprises

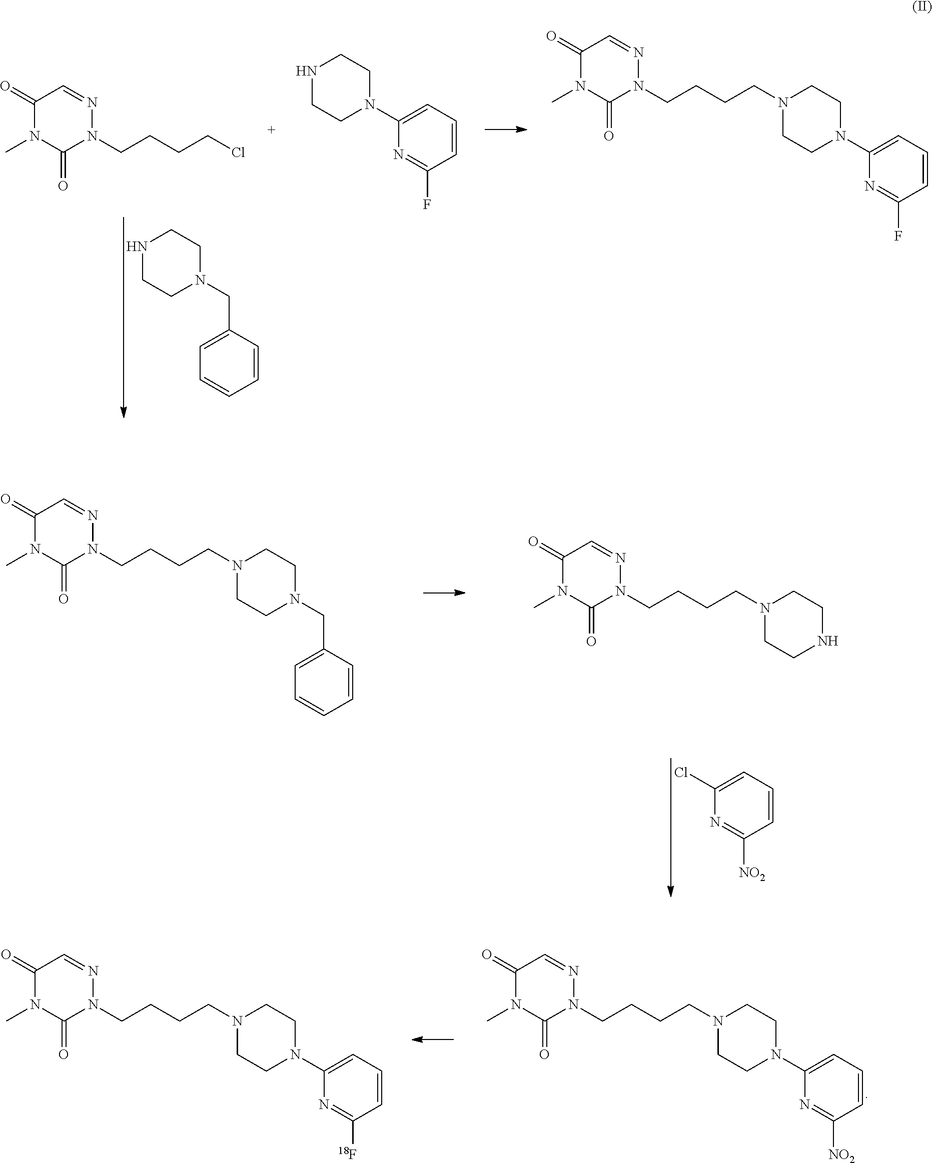

A method of preparing a precursor compound of formula (III) wherein said method comprises

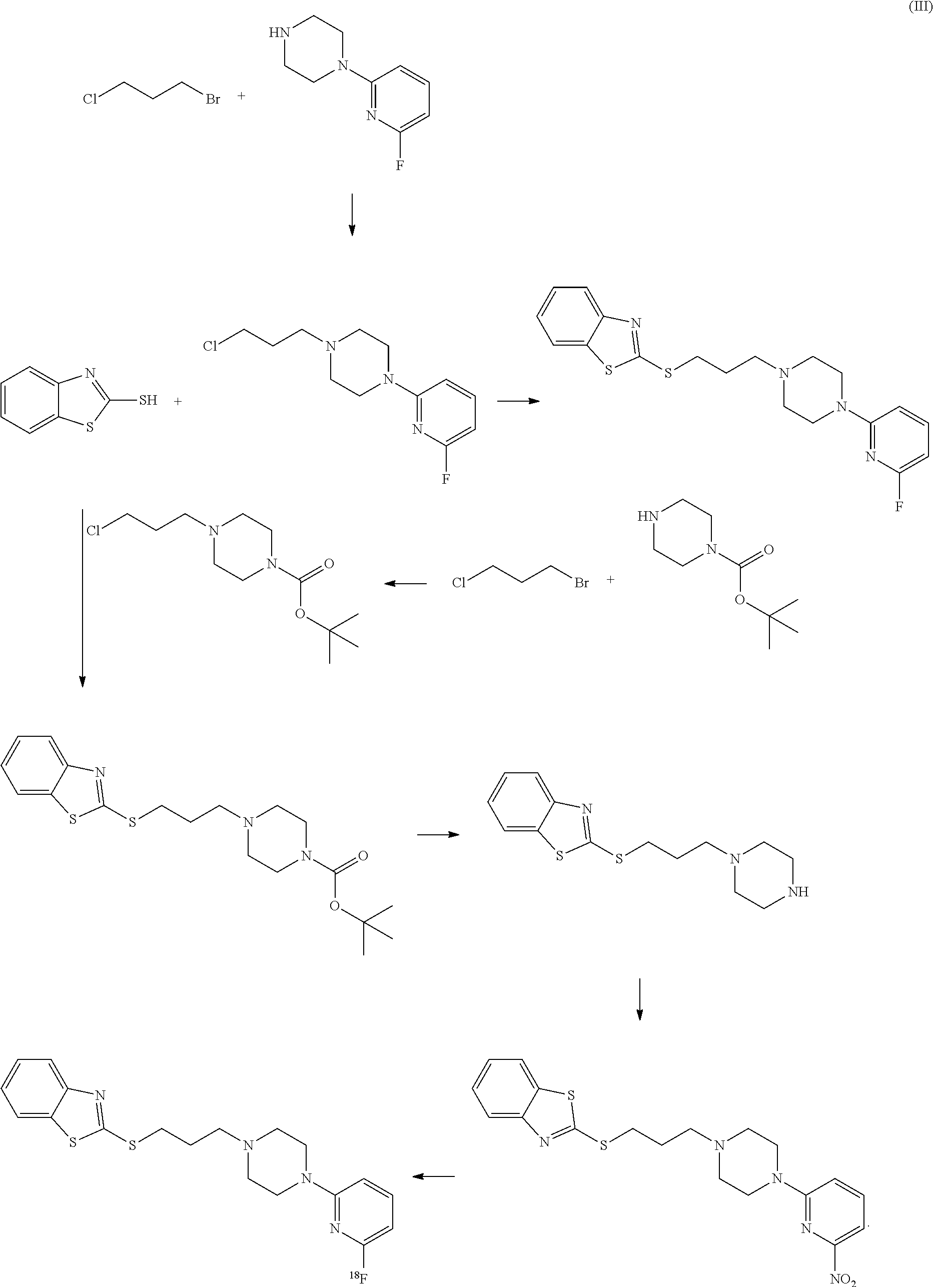

A method of preparing a precursor compound of formula (IV) wherein said method comprises

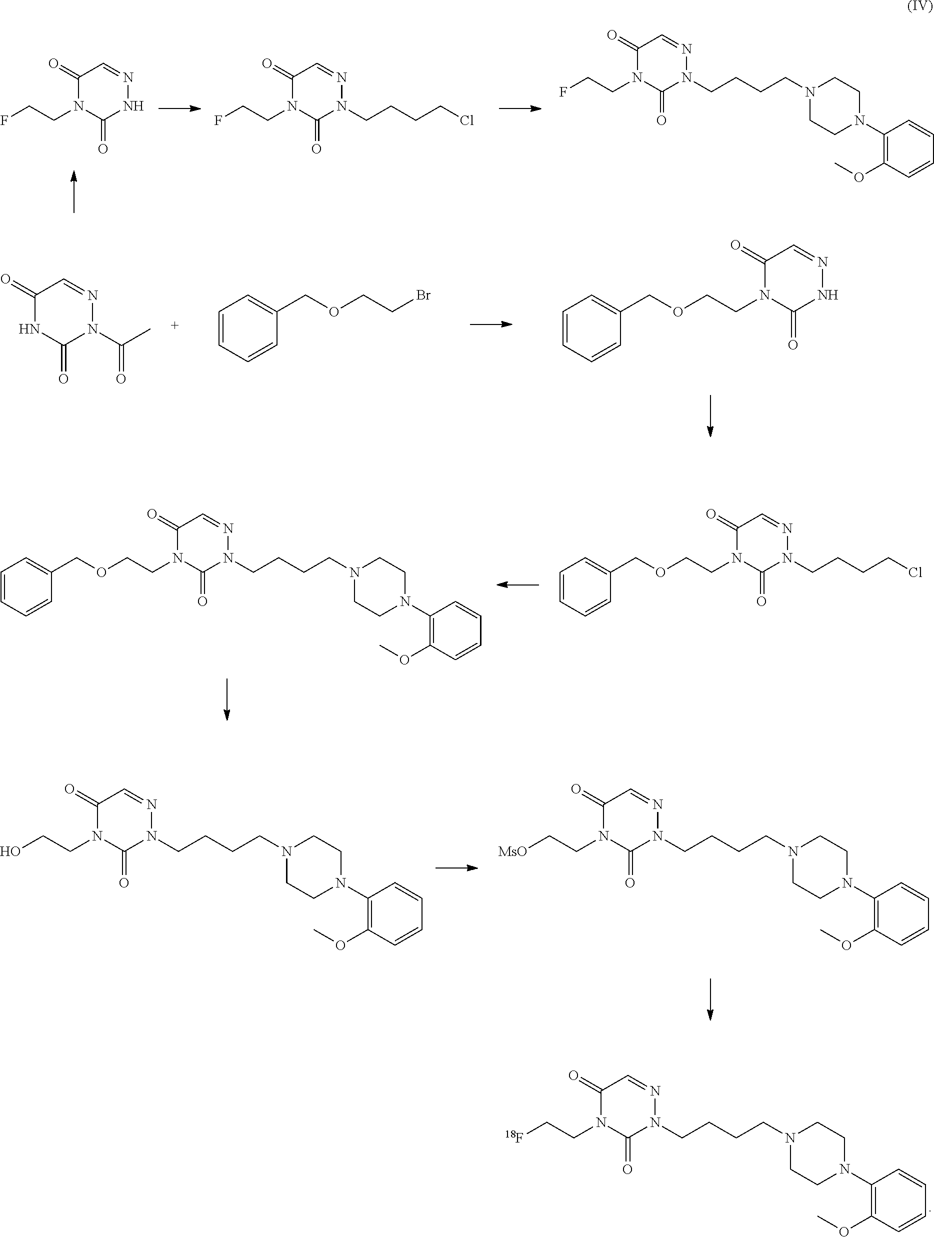

A method of preparing a precursor compound of formula (V) wherein said method comprises

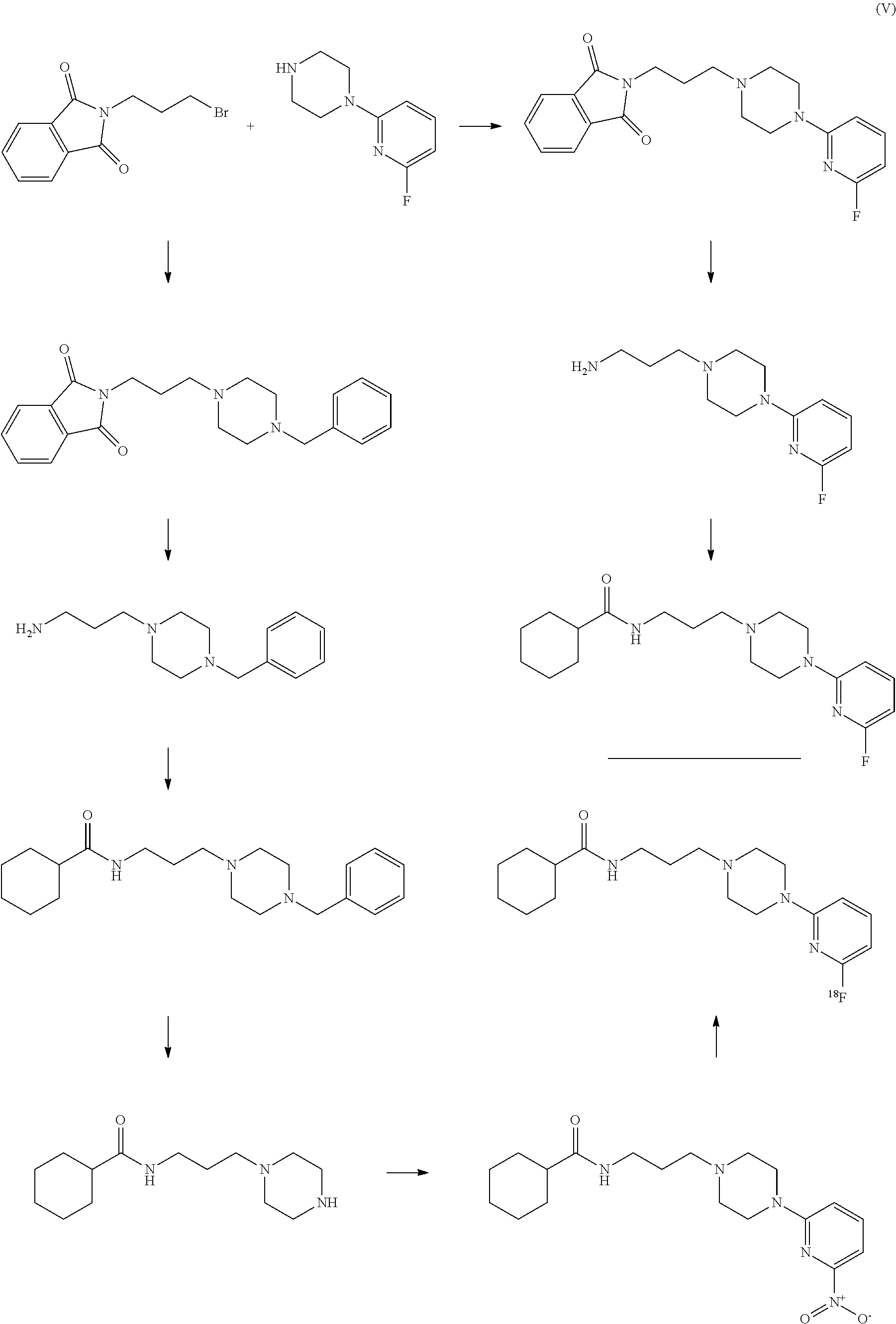

A method of preparing a precursor compound of formula (VI) wherein said method comprises

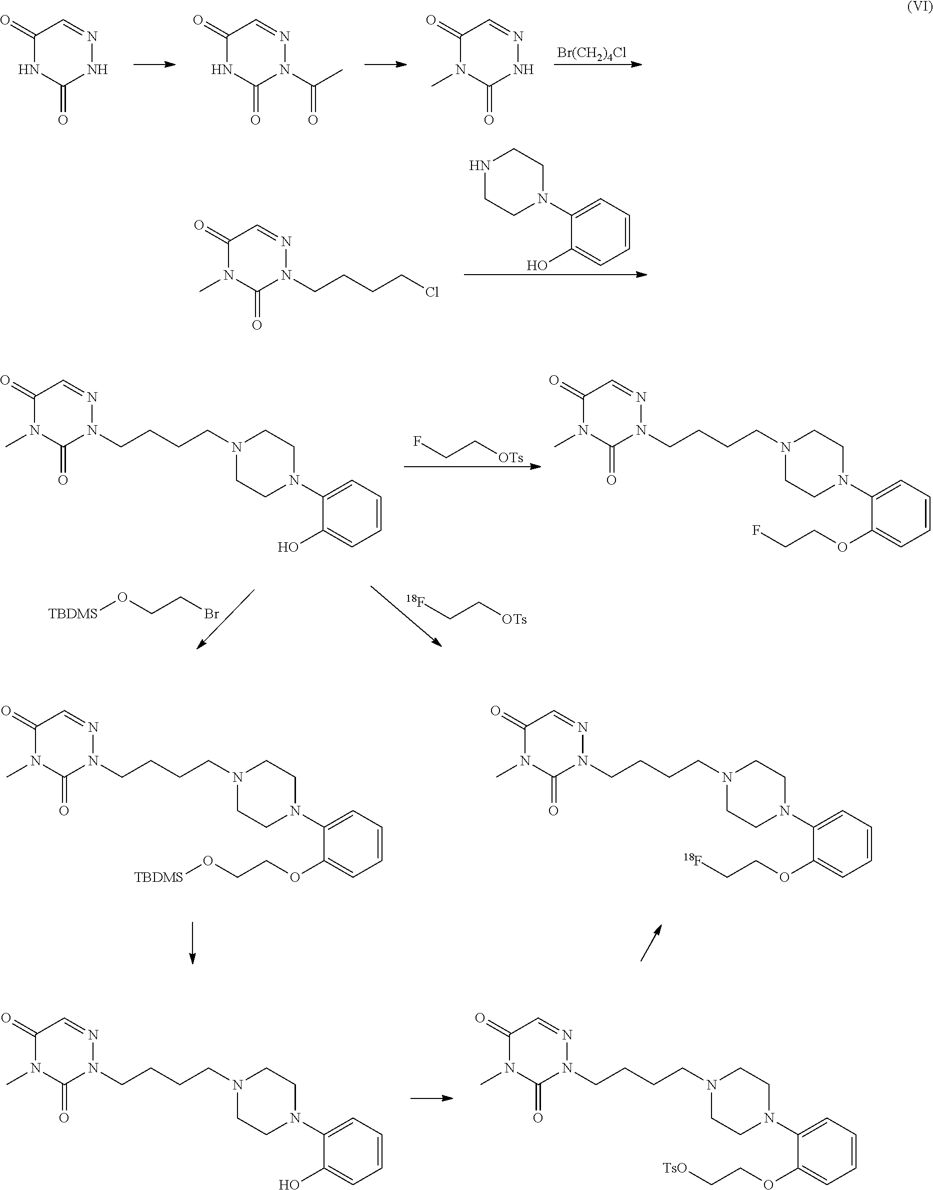

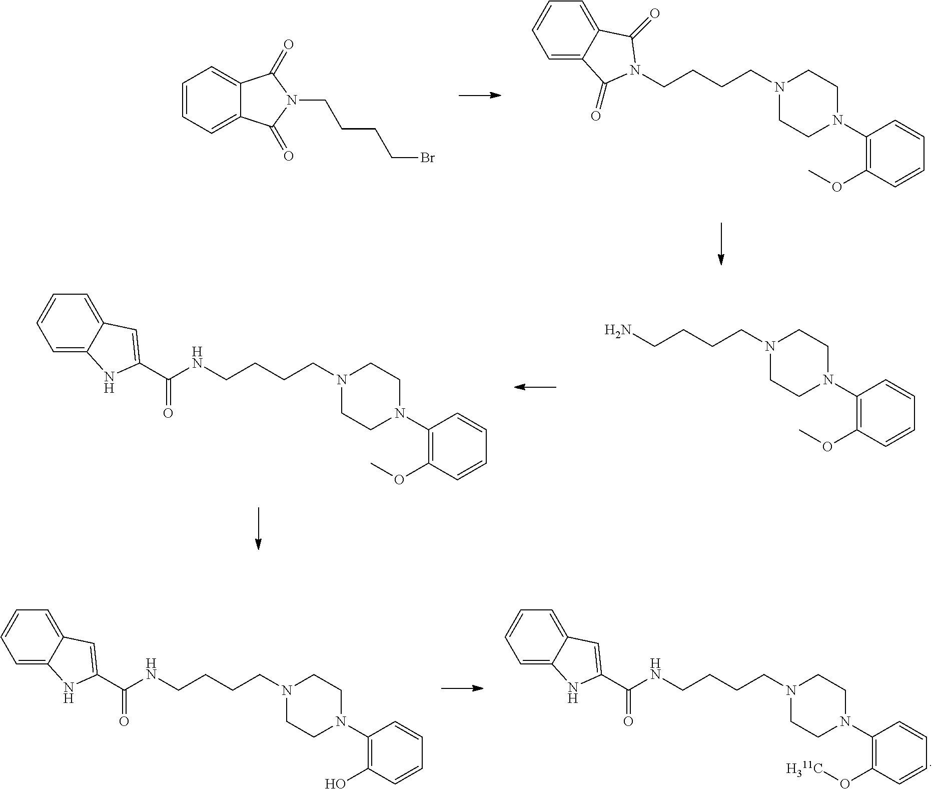 [SMILES: O=C1c2ccccc2C(=O)N1CCCCBr ; COc1ccccc1N1CCN(CCCCN2C(=O)c3ccccc3C2=O)CC1 ; COc1ccccc1N1CCN(CCCCNC(=O)c2cc3ccccc3[nH]2)CC1 ; COc1ccccc1N1CCN(CCCCN)CC1 ; O=C(NCCCCN1CCN(c2ccccc2O)CC1)c1cc2ccccc2[nH]1 ; [11CH3]Oc1ccccc1N1CCN(CCCCNC(=O)c2cc3ccccc3[nH]2)CC1]

The term “precursor” is defined herein as a substance, such as an intermediate compound in a chain of reactions, from which a more stable or definitive product is formed such as radiolabeled and non-radiolabeled compounds of formula (I).

Administration of the Radiolabeled and Non-Radiolabeled Compounds The radiolabeled and non-radiolabeled compounds of the present invention are advantageously useful in veterinary and human medicine. As described above, the radiolabeled and non-radiolabeled compounds of the present invention are useful for imaging HA 5-HT_{1A} receptors in a subject.

When administered to a subject, the radiolabeled and non-radiolabeled compounds of the present invention can be administered as a component of a composition that comprises a physiologically acceptable carrier or vehicle. The present compositions, which comprise a radiolabeled and non-radiolabeled compound of the present invention, can be administered orally or by any other convenient route, for example, by infusion or bolus injection, or by absorption through epithelial or mucocutaneous linings (e.g., oral, rectal, and intestinal mucosa, etc.) and can be administered together with another biologically active agent. Administration can be systemic or local. Various delivery systems are known, e.g., encapsulation in liposomes, microparticles, microcapsules, capsules, etc., and can be administered.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the radiolabeled and non-radiolabeled compounds of the present invention into the bloodstream. The mode of administration is left to the discretion of the practitioner.

In one embodiment, the radiolabeled and non-radiolabeled compounds of the present invention are administered orally.

In another embodiment, the radiolabeled and non-radiolabeled compounds of the present invention are administered intravenously.

In another embodiment, the radiolabeled and non-radiolabeled compounds of the present invention are administered transdermally.

In other embodiments, it can be desirable to administer the radiolabeled and non-radiolabeled compounds of the present invention locally. This can be achieved, for example, and not by way of limitation, by local infusion during surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the radiolabeled and non-radiolabeled compounds of the present invention into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant.

In another embodiment the radiolabeled and non-radiolabeled compounds of the present invention can be delivered in a vesicle, in particular a liposome (see Langer, Science 249:1527-1533 (1990) and Liposomes in the Therapy of Infectious Disease and Cancer, pp. 317-327 and 353-365 (1989)).

In yet another embodiment the radiolabeled and non-radiolabeled compounds of the present invention can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J Med. 321:574 (1989)).

In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

The present compositions can optionally comprise a suitable amount of a physiologically acceptable excipient so as to provide the form for proper administration of a radiolabeled compound of the present invention to the subject.

Such physiologically acceptable excipients can be liquids, such as water for injection, bactereostatic water for injection, sterile water for injection, and oils, including those of petroleum, subject, vegetable, or synthetic origin, such as peanut oil, soybean oil, mineral oil, sesame oil and the like. The pharmaceutical excipients can be saline, gum acacia; gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a subject. Water is a particularly useful excipient when the radiolabeled compound of the present invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents. The present compositions can take the form of solutions, suspensions, emulsion, tablets, pills; pellets, capsules, capsules containing liquids, powders, sustained-release formulations, suppositories, emulsions. aerosols, sprays, suspensions, or any other form suitable for use.

In one embodiment the composition is in the form of a capsule (see e.g. U.S. Pat. No. 5,698,155). Other examples of suitable physiologically acceptable excipients are described in Remington's Pharmaceutical Sciences 1447-1676 (Alfonso R. Gennaro eds., 19th ed. 1995), incorporated herein by reference.

In one embodiment the radiolabeled and non-radiolabeled compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. A time-delay material such as glycerol monostearate or glycerol stearate can also be used. Oral compositions can include standard excipients such as mannitol, lactose, starch, magnesium stearate, sodium saccharin, cellulose, and magnesium carbonate.

In one embodiment the excipients are of pharmaceutical grade.

In one embodiment, when a radiolabeled compound is orally administered, the radiolabeled compound is administered in combination with an additional therapeutic agent that can increase the oral bioavailability of the radiolabeled compound, as described, for example, in U.S. Pat. No. 6,008,222. The additional therapeutic agent may be administered separately from the radiolabeled compound or the additional agent and the radiolabeled compound may be co-administered as part of the same composition. In a specific embodiment, the additional agent that increases the oral bioavailability of a radiolabeled compound is nefazodone.

In another embodiment the radiolabeled and non-radiolabeled compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized-powder or water free concentrate in a hermetically sealed container such as an ampule or sachette indicating

the quantity of active agent. Where the radiolabeled and non-radiolabeled compounds are to be administered by infusion, they can be dispensed, for example, with an infusion bottle containing sterile pharmaceutical grade water or saline. Where the radiolabeled and non-radiolabeled compounds are administered by injection, an ampule of sterile water for injection or saline can be provided so that the ingredients can be mixed prior to administration.

The radiolabeled and non-radiolabeled compounds can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,431,922; 5,354,556; and 5,733,556, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the radiolabeled and non-radiolabeled compounds of the invention. The invention thus encompasses single unit dosage forms suitable for oral administration such as, but not limited to, tablets, capsules, gelcaps, and caplets that are adapted for controlled- or sustained-release. The invention also encompasses transdermal delivery devices, including but not limited to, a transdermal patch and other devices, such as those described in U.S. Pat. No. 5,633,009.

In one embodiment a controlled- or sustained-release composition comprises a minimal amount of a radiolabeled compound to image one or more HA serotonin (5-HT_{1A}) receptors in a subject. Advantages of controlled- or sustained-release compositions include extended activity of the drug, reduced dosage frequency, and increased subject compliance. In addition, controlled- or sustained-release compositions can favorably affect the time of onset of action or other characteristics, such as blood levels of the radiolabeled compound, and can thus reduce the occurrence of adverse side effects. Controlled- or sustained-release compositions can initially release an amount of a radiolabeled compound that promptly produces the desired diagnostic effect, and gradually and continually release other amounts of the radiolabeled compound to maintain this level of diagnostic effect over an extended period of time. To maintain a constant level of the radiolabeled compound in the body, the radiolabeled compound can be released from the dosage form at a rate that will replace the amount of radiolabeled compound being metabolized and excreted from the body. Controlled- or sustained-release of an active ingredient can be stimulated by various conditions, including but no t limited to, changes in pH, changes in temperature, concentration or availability of enzymes, concentration or availability of water, or other physiological conditions.

The amount of the radiolabeled compound that is effective as an imaging agent to detect one or more HA serotonin (5-HT_{1A}) receptors in a subject can be determined using standard clinical and nuclear medicine techniques. In addition, in vitro or in vivo testing can optionally be employed to help identify optimal dosage ranges. The precise dose to be employed will also depend on certain factors—the route of administration, the identity of the subject and the identity of the particular radionuclide being detected- and should be decided according to the judgment of the practitioner and each subject's circumstances in view of, e.g., published clinical studies. The imaging-effective dosage amounts are in the range of about 170 to 380 MBq (megabecquerel) compared to the total amounts administered; that is, if more than one dose of a radiolabeled compound is administered, the imaging-effective dosage amounts correspond to the total amount administered.

The invention encompasses kits that can simplify the administration of a radiolabeled compound to a subject.

A typical kit of the invention comprises a unit dosage form of a radiolabeled compound. In one embodiment the unit dosage form is within a container, which can be sterile, containing a therapeutically effective amount of a radiolabeled compound and a physiologically acceptable carrier or vehicle. The kit can further comprise a label or printed instructions instructing the use of the radiolabeled compound as an imaging agent in order to image one or more HA 5-HT_{1A} receptors in a subject.

Kits of the invention can further comprise a device that is useful for administering the unit dosage forms. Examples of such a device include, but are not limited to, a syringe, a drip bag, a patch, an inhaler, and an enema bag.

Conveniently, the precursor of formula (I) could be provided as part of a kit to a radiopharmacy. The kit may contain a cartridge which can be plugged into a suitably adapted automated synthesiser such as FastLab® or TracerLab®. The cartridge may contain, apart from the precursor, a column to remove unwanted fluoride ion, and an appropriate vessel connected so as to allow the reaction mixture to be evaporated and allow the product to be formulated as required. The reagents and solvents and other consumables required for the synthesis may also be included together with a compact disc carrying the software which allows the synthesiser to be operated in a way so as to meet the customers requirements for radioactive concentration, volumes, time of delivery, etc.

Conveniently, all components of the kit are disposable to minimise the possibilities of contamination between runs and may be sterile and quality assured.

Accordingly, the present invention provides a radiopharmaceutical kit for the preparation of a compound of formula (I) for use in PET which comprises:

(i) a vessel containing a compound of formula (I); and
(ii) means for eluting the vessel with a source of ¹⁸F⁻: and
(iii) an ion-exchange cartridge for removal of excess ¹⁸F⁻.

The invention further provides a cartridge for a radiopharmaceutical kit for the preparation of a compound of formula (I) for use in PET which comprises:

(i) a vessel containing a compound of formula (I); and
(ii) means for eluting the vessel with a source of ¹⁸F⁻.

In a further aspect of the invention, there is provided a method for obtaining a diagnostic PET image which comprises the step of using a radiopharmaceutical kit or a cartridge for a radiopharmaceutical kit as described above.

The term “imaging-effective amount” when used in connection with radiolabeled compounds of the present invention or pharmaceutically acceptable salts thereof, is an amount of the compound that is sufficient to produce a visible image when the compound is administered to a subject and the radiation emitted by the compound is detected using PET or autoradiography.

The phrase “pharmaceutically acceptable salt,” as used herein, is a salt of an acid and a basic nitrogen group of a radiolabeled compound of the present invention. Illustrative salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt of a radiolabeled compound of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohe xylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” also includes a hydrate of a radiolabeled compound of the present invention.

As used herein, a 5-HT_{1A} receptor agent refers to a compound that can selectively interact with the 5-HT_{1A} receptor relative to the other known transporters, receptors, enzymes and proteins. 5-HT_{1A} selective receptor agents include agonists, partial agonists, inverse agonists and antagonists that specifically bind to 5-HT_{1A} receptors.

The term “subject,” as used herein, includes, but is not limited to, a non-human animal, such as a cow, monkey, horse, sheep, pig, chicken, turkey, quail, cat, dog, mouse, rat, rabbit, or guinea pig; and a human. In one embodiment, a subject is a human.

The term “vessel” as used herein is a duct or tube.

The term “therapeutically effective amount” when used in connection with radiolabeled compounds of the present invention or pharmaceutically acceptable salts thereof is an amount that is effective to treat or prevent a psychiatric disorder in a subject, or to stabilize the mood of a subject having a mood disorder.

### EXAMPLES

The following examples are set forth to assist in understanding the invention and should not, of course, be construed as specifically limiting the invention described and claimed herein.

Such variations of the invention, including the substitution of all equivalents now known or later developed, which would be within the purview of those skilled in the art, and changes in formulation or minor changes in experimental design, are to be considered to fall within the scope of the invention incorporated herein.

In some instances, the Examples describe methods for making radiolabeled compounds of the formula (I). These methods, however, are equally applicable to making non-radiolabeled compounds of the formula (I).

### Example 1

### Preparation for Making the Radiolabled Compounds of Formula (I)

Scheme 1 depicts a method for making radiolabeled compounds of formula (I) for preparation of the (6-fluoropyridin-2-yl)piperazine radio-labeled compounds.

Specifically, the nitro-pyridinyl and N,N,N-trimethylpyridin-2-aminium precursors are utilized to make (6-fluoropyridin-2-yl)piperazine radio-labeled compounds.

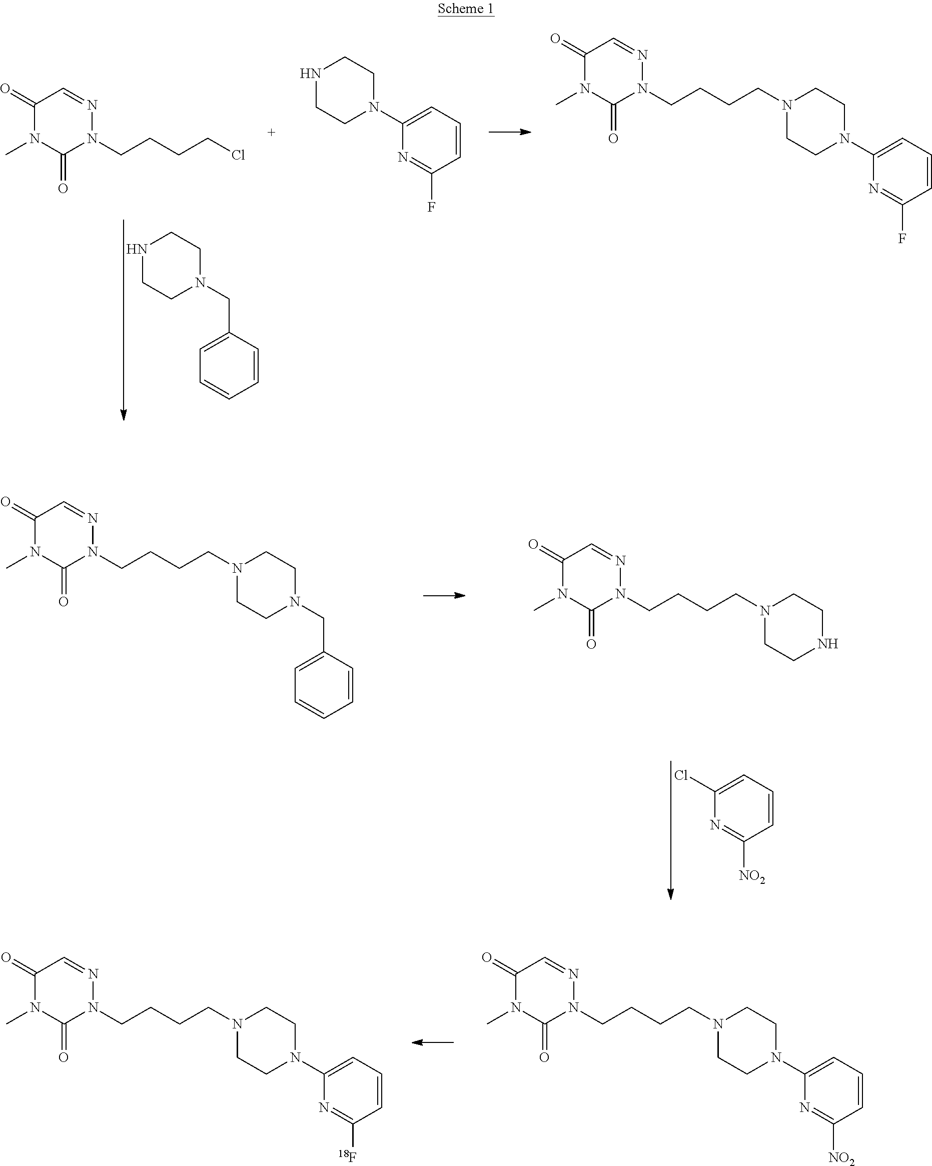

1-(6-fluoropyridin-2-yl)piperazine (0.22 g, 1.21 mmol) and 2-(4-chlorobutyl)-4-methyl-1,2,4-triazine-3,5(2H,4H)-dione (0.25 g, 1.15 mmol) were dissolved in n-butanol (10 mL) and triethylamine (1 mL) added. The mixture was heated to 145° Celcius at reflux for 24 hours, cooled and evaporated. Added water (25 milliLiter) and extracted with EtOAc (3×20 milliLiter). Combined organics were washed with water (20 mL), brine (20 milliLiter, dried (Na2SO4), filtered and evaporated to give 240 mg crude brown oil. This was purified by column chromatography on a 10 g silica cartidge 0.5-10% MeOH-DICHLOROMETHANE over 28 CV to give 2-(4-(4-(6-fluoropyridin-2-yl)piperazin-1-yl)butyl)-4-methyl-1,2,4-triazine-3,5(2H,4H)-dione (180 mg, 41%) as a slightly coloured oil. ¹H and ¹³C NMR were consistent and showed high purity.

### Preparation of 2-(4-(4-benzylpiperazin-1-yl)butyl)-4-methyl-1,2,4-triazine-3,5(2H,4H)-dione

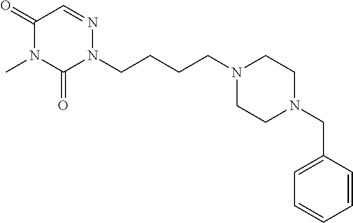 [SMILES: Cn1c(=O)cnn(CCCCN2CCN(Cc3ccccc3)CC2)c1=O]

2-(4-chlorobutyl)-4-methyl-1,2,4-triazine-3,5(2H,4H)-dione (520 mg, 2.389 mmol) and 1-benzylpiperazine (421 mg, 2.389 mmol) were dissolved in BuOH (24 ml) in a 50 mL rb flask to which was added TRIETHYLAMINE (2.5 mL). The mixture was heated at 145° C. for 17 hs. Evaporated solvent, partitioned between DICHLOROMETHANE and water and separated with a phase separator. Evaporated solvent and purified by chromatography on 50 g silica gel cartridge in 0.5-10% MeOH-DICHLOROMETHANE gradient to give product as a viscous oil (480 mg, 56%).

LCMS expected for C₁₉H₂₇N₅O2 357.2; found 358.2 [M+H]⁺.

¹H NMR (300 MHz, d6-DMSO): δ 1.44-1.57 (2H, m, CH₂CH₂), 1.68-1.81 (2H, m, CH₂CH₂), 2.35 (2H, t, J=7.5 Hz, CH₂N), 2.45 (8H, br s, pip-CH₂), 3.31 (3H, s, N—CH₃), 3.49 (2H, s, PhCH₂), 3.97 (2H, t, J=7.0 Hz, CH₂—N), 7.19-7.31 (5H, m, phenyl-H) and 7.36 (1H, s, N═CH). ¹³C NMR (75 MHz, d6-DMSO): δ 28.9 (CH₂CH₂), 26.3 (CH₂CH₂), 27.0 (N—CH₃), 51.8 (NCH₂), 53.1 (pip-CH₂), 53.3 (pip-CH₂), 58.1 (NCH₂), 63.1 (PhCH₂), 127.1 (phenyl-C4), 128.3 (phenyl-C3 & 5), 129.3 (phenyl-C2 & 6), 133.8 (phenyl-C1), 138.1 (N═CH), 148.9 (NMe-C═O) and 156.3 (N—C(═O)—N).

### Preparation of 4-methyl-2-(4-(piperazin-1-yl)butyl)-1,2,4-triazine-3,5(2H,4H)-dione

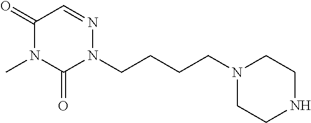 [SMILES: Cn1c(=O)cnn(CCCCN2CCNCC2)c1=O]

2-(4-(4-benzylpiperazin-1-yl)butyl)-4-methyl-1,2,4-triazine-3,5(2H,4H)-dione (475 mg, 1.329 mmol) was dissolved in acetic acid (15 ml) and passed through the H-Cube (80° C., 80 bar, 1 mL/min, 20% Pd(OH)₂). Evaporated to dryness. Dissolved in DICHLOROMETHANE and washed with sat NaHCO₃aq to remove residual acetic acid. 4-methyl-2-(4-(piperazin-1-yl)butyl)-1,2,4-triazine-3,5(2H,4H)-dione was isolated as a waxy solid on standing (166 mg, 47%).

LCMS cal'd for C₁₂H₂₁N₅O₂ 267.2; found 268.1 [M−H]⁺ in ES⁺.

¹H NMR (300 MHz, CDCl₃): δ 1.45-1.58 (2H, m, CH₂CH₂), 1.69-1.83 (2H, m, CH₂CH₂), 2.45 (2H, t, J=7.5 Hz, CH₂N), 2.66 (4H, br s, pip-CH₂), 3.13 (4H, br t, pip-CH₂), 3.33 (3H, s, N—CH₃), 3.98 (2H, t, J=7.0 Hz, CH₂—N) and 7.38 (1H, s, N═CH).

### Preparation of precursor 4-methyl-2-(4-(4-(6-nitropyridin-2-yl)piperazin-1-yl)butyl)-1,2,4-triazine-3,5(2H,4H)-dione

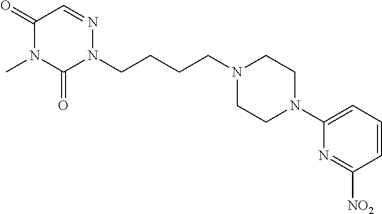 [SMILES: Cn1c(=O)cnn(CCCCN2CCN(c3cccc([N+](=O)[O-])n3)CC2)c1=O]

4-methyl-2-(4-(piperazin-1-yl)butyl)-1,2,4-triazine-3,5(2H,4H)-dione (166 mg, 0.62 mmol), and 2-chloro-6-nitropyridine (98.6 mg, 0.62 mmol) were dissolved in dr yMeCN (8 ml) in 2×10 mL microwave tubes and 6 drops DIPEA added to each. The mixture was stirred during heating to dissolve and heated in microwave at 120° C. for 20 min. The tubes were reheated because it seemed likely that starting material remained. Combined and evaporated. Partitioned between DICHLOROMETHANE-water and separated with a phase-separator. Evaporated and columned on 10 g silica using 0.5-10% MeOH-EtOAc gradient to give incomplete separation of nitro from chloro products. Evaporated product-containing fractions and further purified by semi-prep HPLC (150 mm×15 mm, Phenomenex Gemini column in acetonitrile water eluant) to give 4-methyl-2-(4-(4-(6-nitropyridin-2-yl)piperazin-1-yl)butyl)-1,2,4-triazine-3,5(2H,4H)-dione (35 mg, 15%).

LCMS calc'd for C₁₇H₂₃N₇O₄: 389.2; found 390.1 [M+H]⁺.

¹H NMR (300 MHz, d6-DMSO): δ 1.50-1.63 (2H, m, CH₂CH₂), 1.73-1.86 (2H, m, CH₂CH₂), 2.42 (2H, t, J=7.5 Hz, CH₂N), 2.53 (4H, t, J=5.0 Hz, pip-CH₂), 3.33 (3H, s, N—CH₃), 3.65 (4H, t, J=5.0 Hz, pip-CH₂), 4.01 (2H, t, J=7.0 Hz, CH₂—N), 6.89 (1H, d, J=8.5 Hz, pyridylC3-H), 7.39 (1H, s, N═CH), 7.43 (1H, d, J=7.5 Hz, pyridylC5-H) and 7.68 (1H, t, J=8.0 Hz, pyridylC4-H). ¹³C NMR (75 MHz, d6-DMSO): δ 23.6 (CH₂CH₂), 26.1 (CH₂CH₂), 26.9 (N—CH₃), 44.7 (pip-CH₂), 51.6 (N—CH₂), 52.7 (pip-CH₂), 57.9 (NCH₂), 105.4 (pyridyl-C5), 111.7 (pyridyl-C3), 133.8 (N═CH), 140.1 (pyridyl-C4), 148.8 (NMe-C═O), 155.8 (pyridyl-C2), 156.2 (MeN-C(═O)N) and 157.7 (C—NO₂).

### Preparation of [¹⁸F]2-(4-(4-(6-fluoropyridin-2-yl)piperazin-1-yl)butyl)-4-methyl-1,2,4-triazine-3,5(2H,4H)-dione

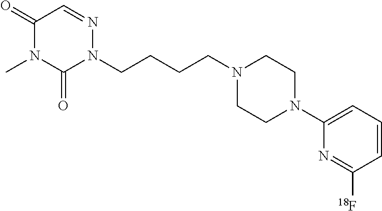 [SMILES: Cn1c(=O)cnn(CCCCN2CCN(c3cccc([18F])n3)CC2)c1=O]

The synthesis was performed using a GE Tracerlab system, inside a lead shielded enclosure. [¹⁸F]-Fluoride (aq., 10.75 GBq) was trapped to a Sep-pak QMA carbonate Light cartridge, and eluted into a reaction vessel with a solution of kryptofix (9.5 mg), K₂CO₃ (80 μL of a 0.10 M solution) and anhydrous MeCN (1.92 mL). The vessel was heated to 100° C. for 30 min under a stream of N₂ to azeotropically dry the fluoride. The nitropyridyl precursor, 4-methyl-2-(4-(4-(6-nitropyridin-2-yl)piperazin-1-yl)butyl)-1,2,4-triazine-3,5(2H,4H)-dione (3.3 mg) in anhydrous DMF (0.5 mL) was added to the vessel and heated at 110° C. for 30 min. The crude reaction mixture was added to H₂O (20 mL) and loaded to a tC18 Light Sep-pak cartridge, which was washed with H₂O (2 mL). The product was eluted from the cartridge with MeCN (0.5 mL) and H₂O (1 mL). The eluant contained 1663 MBq. A portion of the eluant (1.0 mL) was removed from the enclosure and manually injected onto a semi-preparative HPLC system. The product was collected using a manual switch (731 MBq). Approximately 1 mL of the collected volume was separated, diluted in H₂O (15 mL) and loaded to a primed (1 mL EtOH, 2 mL H₂O) tC18 Light Sep-pak cartridge. The cartridge was washed with H₂O (2 mL), and product eluted in EtOH (0.5 mL) and phosphate buffered saline (4.5 mL). 170 MBq was formulated. A portion of this sample (3 mL) was used for biodistribution studies. Estimated total yield ˜7% (˜750 MBq). RCP >99%. SA=25 GBq/μmol.

| | Semi-preparative | Analytical |
|---|---|---|
| Column | Phenomenex Luna C18, | Phenomenex Luna C18, |
| | 10 × 100 mm | 5μ, 4.6 × 150 mm |
| Eluant | A = 0.8% NEt₃ in H₂O, | A = H₂O |
| | adjusted to pH = 7.5 with | B = MeCN |
| | H₃PO₄ | |
| | B = MeOH | |
| Gradient | 0-1 min 45% B | 0-1 min 35% B |
| | 1-30 min 45-65% B | 1-20 min 35-95% B |
| | 30-35 min 65% B | 20-30 min 95% B |
| Flow rate | 3.0 | 1.0 |
| (mL/min) | | |
| Retention | 24.2 | 10.1 |
| time (min) | | |

Compounds that were synthesized following procedures that were similar or identical as described those in Examples 1, 4, 7 or 12 include the following:

| Structure | Name | [M + H]⁺ |
|---|---|---|
| 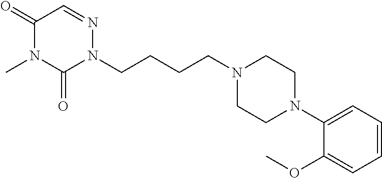 [SMILES: COc1ccccc1N1CCN(CCCCn2ncc(=O)n(C)c2=O)CC1] | 2-(4-(4-(2- methoxyphenyl)piperazin-1- yl)butyl)-4-methyl-1,2,4- triazine-3,5(2H,4H)-dione | 374.4 |
| 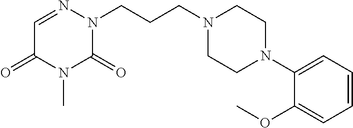 [SMILES: COc1ccccc1N1CCN(CCCn2ncc(=O)n(C)c2=O)CC1] | 2-(3-(4-(2- methoxyphenyl)piperazin-1- yl)propyl)-4-methyl-1,2,4- triazine-3,5(2H,4H)-dione | 360.3 |
| 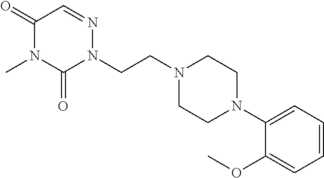 [SMILES: COc1ccccc1N1CCN(CCn2ncc(=O)n(C)c2=O)CC1] | 2-(2-(4-(2- methoxyphenyl)piperazin-1- yl)ethyl)-4-methyl-1,2,4- triazine-3,5(2H,4H)-dione | 346.4 |
| 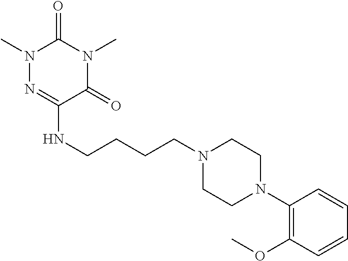 [SMILES: COc1ccccc1N1CCN(CCCCNc2nn(C)c(=O)n(C)c2=O)CC1] | 6-((4-(4-(2- methoxyphenyl)piperazin-1- yl)butyl)amino)-2,4-dimethyl- 1,2,4-triazine-3,5(2H,4H)-dione | 403.5 |
| 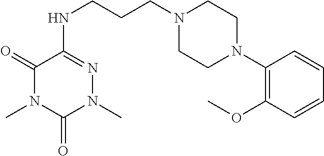 [SMILES: COc1ccccc1N1CCN(CCCNc2nn(C)c(=O)n(C)c2=O)CC1] | 6-((3-(4-(2- methoxyphenyl)piperazin-1- yl)propyl)amino)-2,4-dimethyl- 1,2,4-triazine-3,5(2H,4H)-dione | 389.4 |
| 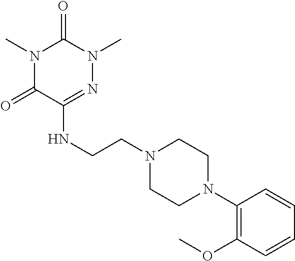 [SMILES: COc1ccccc1N1CCN(CCNc2nn(C)c(=O)n(C)c2=O)CC1] | 6-((2-(4-(2- methoxyphenyl)piperazin-1- yl)ethyl)amino)-2,4-dimethyl- 1,2,4-triazine-3,5(2H,4H)-dione | 375.4 |
| 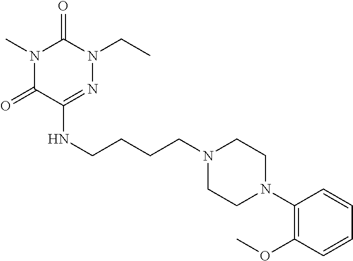 [SMILES: CCn1nc(NCCCCN2CCN(c3ccccc3OC)CC2)c(=O)n(C)c1=O] | 2-ethyl-6-((4-(4-(2- methoxyphenyl)piperazin-1- yl)butyl)amino)-4-methyl-1,2,4- triazine-3,5(2H,4H)-dione | 417.5 |
| 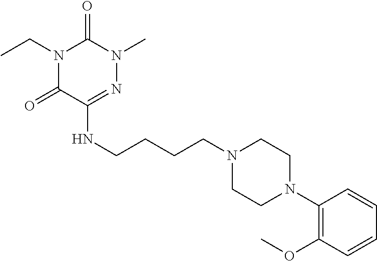 [SMILES: CCn1c(=O)c(NCCCCN2CCN(c3ccccc3OC)CC2)nn(C)c1=O] | 4-ethyl-6-((4-(4-(2- methoxyphenyl)piperazin-1- yl)butyl)amino)-2-methyl-1,2,4- triazine-3,5(2H,4H)-dione | 417.5 |
| 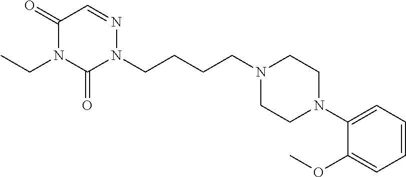 [SMILES: CCn1c(=O)cnn(CCCCN2CCN(c3ccccc3OC)CC2)c1=O] | 4-ethyl-2-(4-(4-(2- methoxyphenyl)piperazin-1- yl)butyl)-1,2,4-triazine- 3,5(2H,4H)-dione | 388.4 |
| 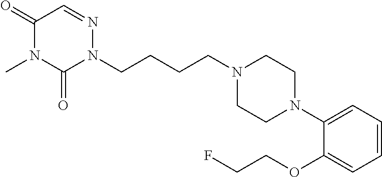 [SMILES: Cn1c(=O)cnn(CCCCN2CCN(c3ccccc3OCCF)CC2)c1=O] | 2-(4-(4-(2-(2- fluoroethoxy)phenyl)piperazin- 1-yl)butyl)-4-methyl-1,2,4- triazine-3,5(2H,4H)-dione | 406.4 |
| 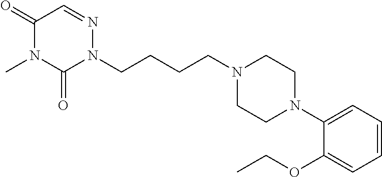 [SMILES: CCOc1ccccc1N1CCN(CCCCn2ncc(=O)n(C)c2=O)CC1] | 2-(4-(4-(2- ethoxyphenyl)piperazin-1- yl)butyl)-4-methyl-1,2,4- triazine-3,5(2H,4H)-dione | 388.4 |
| 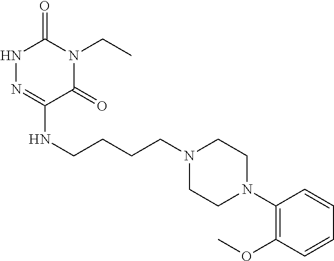 [SMILES: CCn1c(=O)[nH]nc(NCCCCN2CCN(c3ccccc3OC)CC2)c1=O] | 4-ethyl-6-((4-(4-(2- methoxyphenyl)piperazin-1- yl)butyl)amino)-1,2,4-triazine- 3,5(2H,4H)-dione | 403.5 |
| 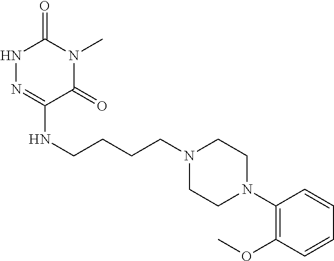 [SMILES: COc1ccccc1N1CCN(CCCCNc2n[nH]c(=O)n(C)c2=O)CC1] | 6-((4-(4-(2- methoxyphenyl)piperazin-1- yl)butyl)amino)-4-methyl-1,2,4- triazine-3,5(2H,4H)-dione | 389.4 |
| 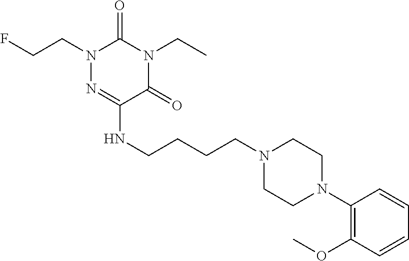 [SMILES: CCn1c(=O)c(NCCCCN2CCN(c3ccccc3OC)CC2)nn(CCF)c1=O] | 4-ethyl-2-(2-fluoroethyl)-6-((4- (4-(2-methoxyphenyl)piperazin- 1-yl)butyl)amino)-1,2,4-triazine- 3,5(2H,4H)-dione | 449.5 |
| 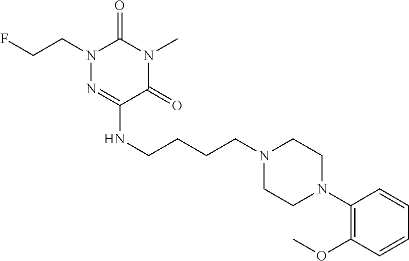 [SMILES: COc1ccccc1N1CCN(CCCCNc2nn(CCF)c(=O)n(C)c2=O)CC1] | 2-(2-fluoroethyl)-6-((4-(4-(2- methoxyphenyl)piperazin-1- yl)butyl)amino)-4-methyl-1,2,4- triazine-3,5(2H,4H)-dione | 435.5 |
| 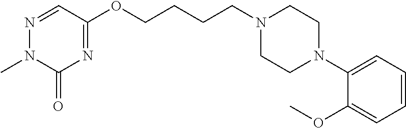 [SMILES: COc1ccccc1N1CCN(CCCCOc2cnn(C)c(=O)n2)CC1] | 5-(4-(4-(2- methoxyphenyl)piperazin-1- yl)butoxy)-2-methyl-1,2,4- triazin-3(2H)-one | 374.4 |
| 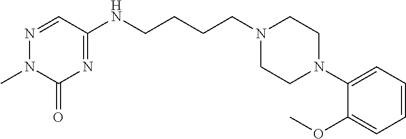 [SMILES: COc1ccccc1N1CCN(CCCCNc2cnn(C)c(=O)n2)CC1] | 5-((4-(4-(2- methoxyphenyl)piperazin-1- yl)butyl)amino)-2-methyl-1,2,4- triazin-3(2H)-one | 373.4 |
| 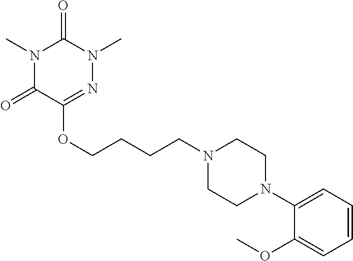 [SMILES: COc1ccccc1N1CCN(CCCCOc2nn(C)c(=O)n(C)c2=O)CC1] | 6-(4-(4-(2- methoxyphenyl)piperazin-1- yl)butoxy)-2,4-dimethyl-1,2,4- triazine-3,5(2H,4H)-dione | 404.4 |
| 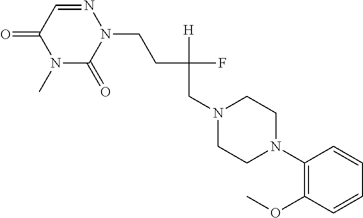 [SMILES: COc1ccccc1N1CCN(CC(F)CCn2ncc(=O)n(C)c2=O)CC1] | 2-(3-fluoro-4-(4-(2- methoxyphenyl)piperazin-1- yl)butyl)-4-methyl-1,2,4- triazine-3,5(2H,4H)-dione | 392.4 |
| 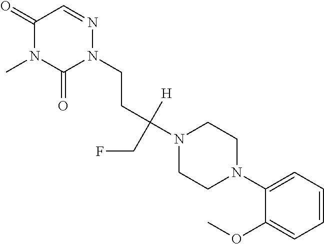 [SMILES: COc1ccccc1N1CCN(C(CF)CCn2ncc(=O)n(C)c2=O)CC1] | 2-(4-fluoro-3-(4-(2- methoxyphenyl)piperazin-1- yl)butyl)-4-methyl-1,2,4- triazine-3,5(2H,4H)-dione | 392.4 |
| 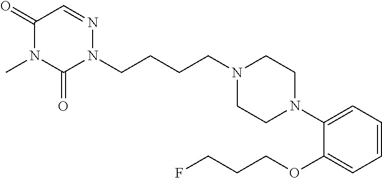 [SMILES: Cn1c(=O)cnn(CCCCN2CCN(c3ccccc3OCCCF)CC2)c1=O] | 2-(4-(4-(2-(3- fluoropropoxy)phenyl)piperazin- 1-yl)butyl)-4-methyl-1,2,4- triazine-3,5(2H,4H)-dione | 420.5 |
| 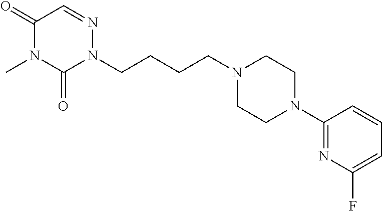 [SMILES: Cn1c(=O)cnn(CCCCN2CCN(c3cccc(F)n3)CC2)c1=O] | 2-(4-(4-(6-fluoropyridin-2- yl)piperazin-1-yl)butyl)-4- methyl-1,2,4-triazine- 3,5(2H,4H)-dione | 363.3 |
| 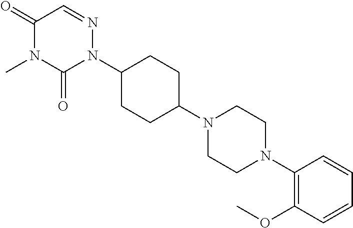 [SMILES: COc1ccccc1N1CCN(C2CCC(n3ncc(=O)n(C)c3=O)CC2)CC1] | 2-(4-(4-(2- methoxyphenyl)piperazin-1- yl)cyclohexyl)-4-methyl-1,2,4- triazine-3,5(2H,4H)-dione | 400.4 |
| 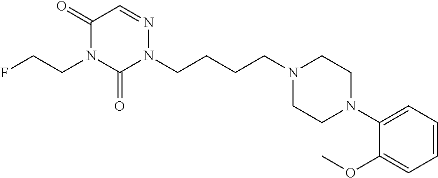 [SMILES: COc1ccccc1N1CCN(CCCCn2ncc(=O)n(CCF)c2=O)CC1] | 4-(2-fluoroethyl)-2-(4-(4-(2- methoxyphenyl)piperazin-1- yl)butyl)-1,2,4-triazine- 3,5(2H,4H)-dione | 406.4 |
| 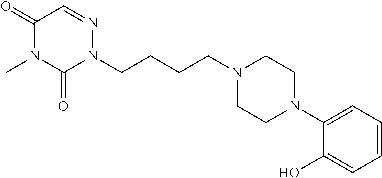 [SMILES: Cn1c(=O)cnn(CCCCN2CCN(c3ccccc3O)CC2)c1=O] | 2-(4-(4-(2- hydroxyphenyl)piperazin-1- yl)butyl)-4-methyl-1,2,4- triazine-3,5(2H,4H)-dione | 360.3 |
| 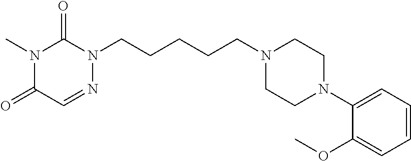 [SMILES: COc1ccccc1N1CCN(CCCCCn2ncc(=O)n(C)c2=O)CC1] | 2-(5-(4-(2- methoxyphenyl)piperazin-1- yl)pentyl)-4-methyl-1,2,4- triazine-3,5(2H,4H)-dione | 388.4 |
| 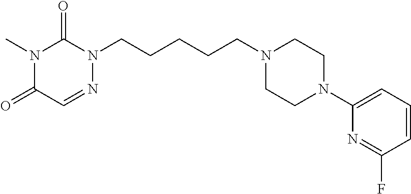 [SMILES: Cn1c(=O)cnn(CCCCCN2CCN(c3cccc(F)n3)CC2)c1=O] | 2-(5-(4-(6-fluoropyridin-2- yl)piperazin-1-yl)pentyl)-4- methyl-1,2,4-triazine- 3,5(2H,4H)-dione | 377.3 |
| 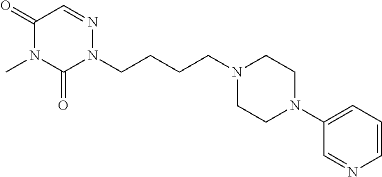 [SMILES: Cn1c(=O)cnn(CCCCN2CCN(c3cccnc3)CC2)c1=O] | 4-methyl-2-(4-(4-(pyridin-3- yl)piperazin-1-yl)butyl)-1,2,4- triazine-3,5(2H,4H)-dione | 345.3 |
| 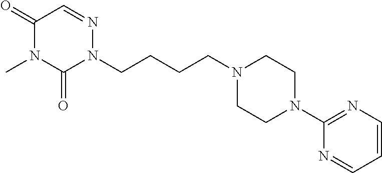 [SMILES: Cn1c(=O)cnn(CCCCN2CCN(c3ncccn3)CC2)c1=O] | 4-methyl-2-(4-(4-(pyrimidin-2- yl)piperazin-1-y)butyl)-1,2,4- triazine-3,5(2H,4H)-dione | 346.3 |
| 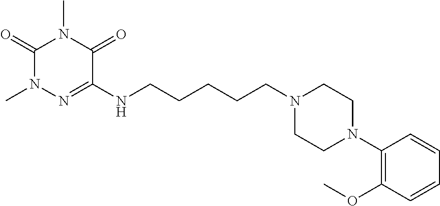 [SMILES: COc1ccccc1N1CCN(CCCCCNc2nn(C)c(=O)n(C)c2=O)CC1] | 6-((5-(4-(2- methoxyphenyl)piperazin-1- yl)pentyl)amino)-2,4-dimethyl- 1,2,4-triazine-3,5(2H,4H)-dione | 417.4 |
| 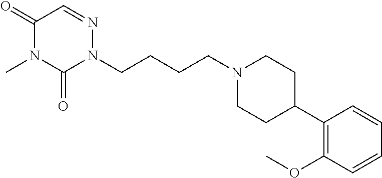 [SMILES: COc1ccccc1C1CCN(CCCCn2ncc(=O)n(C)c2=O)CC1] | 2-(4-(4-(2- methoxyphenyl)piperidin-1- yl)butyl)-4-methyl-1,2,4- triazine-3,5(2H,4H)-dione | 373.4 |
| 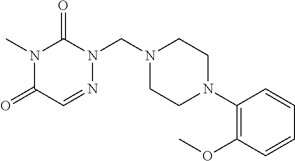 [SMILES: COc1ccccc1N1CCN(Cn2ncc(=O)n(C)c2=O)CC1] | 2-((4-(2- methoxyphenyl)piperazin-1- yl)methyl)-4-methyl-1,2,4- triazine-3,5(2H,4H)-dione | 332.3 |
| 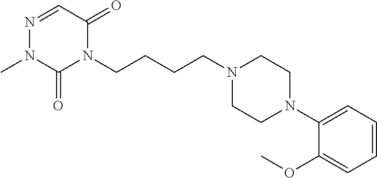 [SMILES: COc1ccccc1N1CCN(CCCCn2c(=O)cnn(C)c2=O)CC1] | 4-(4-(4-(2- methoxyphenyl)piperazin-1- yl)butyl)-2-methyl-1,2,4- triazine-3,5(2H,4H)-dione | 374.3 |
| 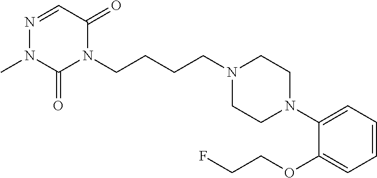 [SMILES: Cn1ncc(=O)n(CCCCN2CCN(c3ccccc3OCCF)CC2)c1=O] | 4-(4-(4-(2-(2- fluoroethoxy)phenyl)piperazin- 1-yl)butyl)-2-methyl-1,2,4- triazine-3,5(2H,4H)-dione | 406.4 |
| 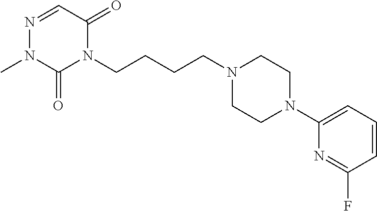 [SMILES: Cn1ncc(=O)n(CCCCN2CCN(c3cccc(F)n3)CC2)c1=O] | 4-(4-(4-(6-fluoropyridin-2- yl)piperazin-1-yl)butyl)-2- methyl-1,2,4-triazine- 3,5(2H,4H)-dione | 363.3 |
| 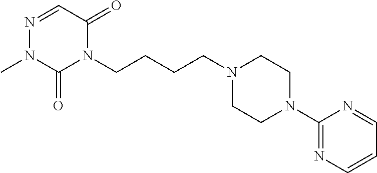 [SMILES: Cn1ncc(=O)n(CCCCN2CCN(c3ncccn3)CC2)c1=O] | 2-methyl-4-(4-(4-(pyrimidin-2- yl)piperazin-1-yl)butyl)-1,2,4- triazine-3,5(2H,4H)-dione | 346.3 |
| 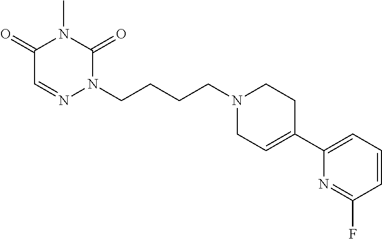 [SMILES: Cn1c(=O)cnn(CCCCN2CC=C(c3cccc(F)n3)CC2)c1=O] | 2-(4-(6-fluoro-5′,6′-dihydro- [2,4′-bipyridin]-1′(2′H)- yl)butyl)-4-methyl-1,2,4- triazine-3,5(2H,4H)-dione | 360.3 |
| 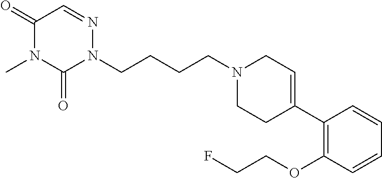 [SMILES: Cn1c(=O)cnn(CCCCN2CC=C(c3ccccc3OCCF)CC2)c1=O] | 2-(4-(4-(2-(2- fuoroethoxy)phenyl)-5,6- dihydropyridin-1(2H)-yl)butyl)- 4-methyl-1,2,4-triazine- 3,5(2H,4H)-dione | 403.4 |
| 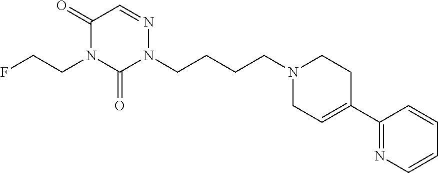 [SMILES: O=c1cnn(CCCCN2CC=C(c3ccccn3)CC2)c(=O)n1CCF] | 2-(4-(5′,6′-dihydro-[2,4′- bipyridin]-1′(2′H)-yl)butyl)-4- (2-fluoroethyl)-1,2,4-triazine- 3,5(2H,4H)-dione | 374.4 |
| 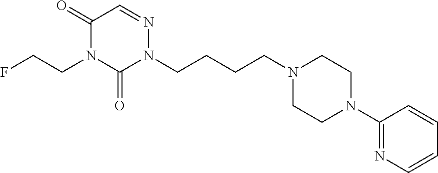 [SMILES: O=c1cnn(CCCCN2CCN(c3ccccn3)CC2)c(=O)n1CCF] | 4-(2-fluoroethyl)-2-(4-(4- (pyridin-2-yl)piperazin-1- yl)butyl)-1,2,4-triazine- 3,5(2H,4H)-dione | 377.4 |
| 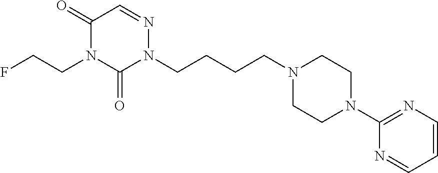 [SMILES: O=c1cnn(CCCCN2CCN(c3ncccn3)CC2)c(=O)n1CCF] | 4-(2-fluoroethyl)-2-(4-(4- (pyrimidin-2-yl)piperazin-1- yl)butyl)-1,2,4-triazine- 3,5(2H,4H)-dione | 378.3 |
| 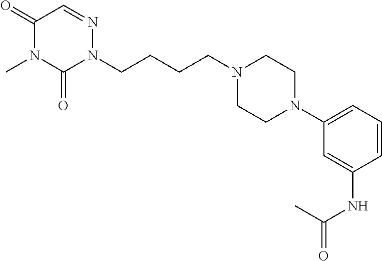 [SMILES: CC(=O)Nc1cccc(N2CCN(CCCCn3ncc(=O)n(C)c3=O)CC2)c1] | N-(3-(4-(4-(4-methyl-3,5-dioxo- 4,5-dihydro-1,2,4-triazin-2(3H)- yl)butyl)piperazin-1- yl)phenyl)acetamide | 401.4 |
| 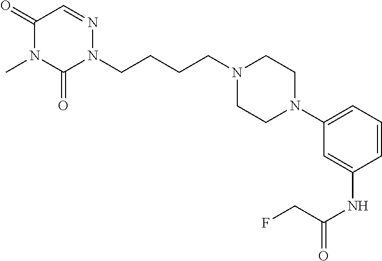 [SMILES: Cn1c(=O)cnn(CCCCN2CCN(c3cccc(NC(=O)CF)c3)CC2)c1=O] | 2-fluoro-N-(3-(4-(4-(4-methyl- 3,5-dioxo-4,5-dihydro-1,2,4- triazin-2(3H)-yl)butyl)piperazin- 1-yl)phenyl)acetamide | 419.4 |
| 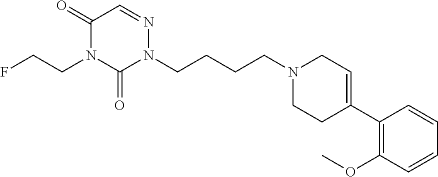 [SMILES: COc1ccccc1C1=CCN(CCCCn2ncc(=O)n(CCF)c2=O)CC1] | 4-(2-fluoroethyl)-2-(4-(4-(2- methoxyphenyl)-5,6- dihydropyridin-1(2H)-yl)butyl)- 1,2,4-triazine-3,5(2H,4H)-dione | 403.4 |
| 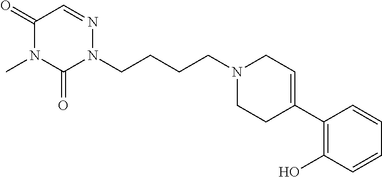 [SMILES: Cn1c(=O)cnn(CCCCN2CC=C(c3ccccc3O)CC2)c1=O] | 2-(4-(4-(2-hydroxyphenyl)-5,6- dihydropyridin-1(2H)-yl)butyl)- 4-methyl-1,2,4-triazine- 3,5(2H,4H)-dione | 357.3 |
| 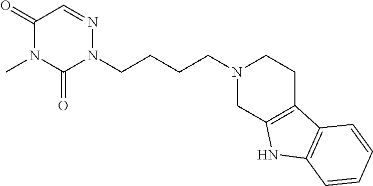 [SMILES: Cn1c(=O)cnn(CCCCN2CCc3c([nH]c4ccccc34)C2)c1=O] | 2-(4-(3,4-dihydro-1H- pyrido[3,4-b]indol-2(9H)- yl)butyl)-4-methyl-1,2,4- triazine-3,5(2H,4H)-dione | 354.3 |
| 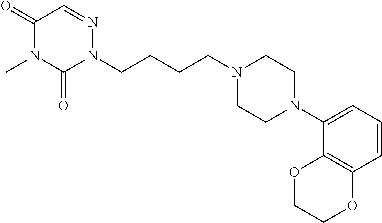 [SMILES: Cn1c(=O)cnn(CCCCN2CCN(c3cccc4c3OCCO4)CC2)c1=O] | 2-(4-(4-(2,3- dihydrobenzo[b][1,4]dioxin-5- yl)piperazin-1-yl)butyl)-4- methyl-1,2,4-triazine- 3,5(2H,4H)-dione | 402.4 |
| 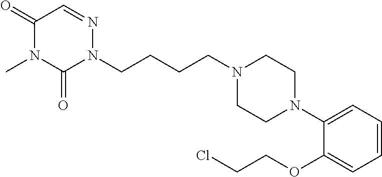 [SMILES: Cn1c(=O)cnn(CCCCN2CCN(c3ccccc3OCCCl)CC2)c1=O] | 2-(4-(4-(2-(2- chloroethoxy)phenyl)piperazin- 1-yl)butyl)-4-methyl-1,2,4- triazine-3,5(2H,4H)-dione | 422.8 |
| 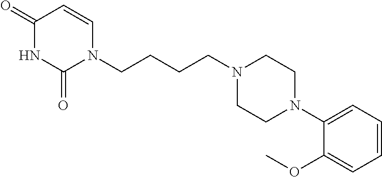 [SMILES: COc1ccccc1N1CCN(CCCCn2ccc(=O)[nH]c2=O)CC1] | 1-(4-(4-(2- methoxyphenyl)piperazin-1- yl)butyl)pyrimidine-2,4(1H,3H)- dione | 359.3 |
| 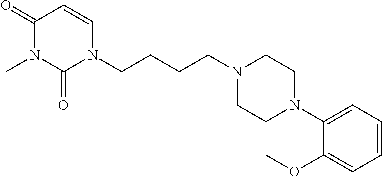 [SMILES: COc1ccccc1N1CCN(CCCCn2ccc(=O)n(C)c2=O)CC1] | 1-(4-(4-(2- methoxyphenyl)piperazin-1- yl)butyl)-3-methylpyrimidine- 2,4(1H,3H)-dione | 373.4 |
| 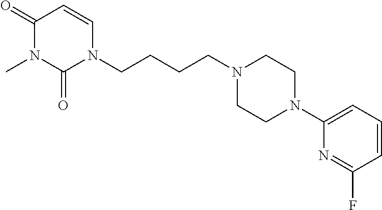 [SMILES: Cn1c(=O)ccn(CCCCN2CCN(c3cccc(F)n3)CC2)c1=O] | 1-(4-(4-(6-fluoropyridin-2- yl)piperazin-1-yl)butyl)-3- methylpyrimidine-2,4(1H,3H)- dione | 362.4 |
| 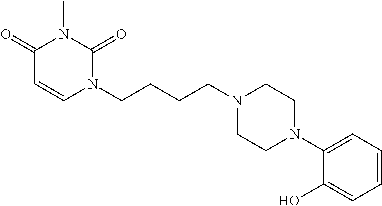 [SMILES: Cn1c(=O)ccn(CCCCN2CCN(c3ccccc3O)CC2)c1=O] | 1-(4-(4-(2- hydroxyphenyl)piperazin-1- yl)butyl)-3-methylpyrimidine- 2,4(1H,3H)-dione | 359.3 |
| 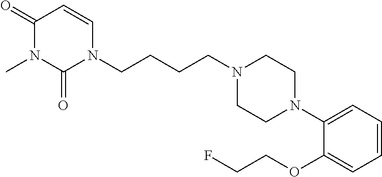 [SMILES: Cn1c(=O)ccn(CCCCN2CCN(c3ccccc3OCCF)CC2)c1=O] | 1-(4-(4-(2-(2- fluoroethoxy)phenyl)piperazin-1- yl)butyl)-3-methylpyrimidine- 2,4(1H,3H)-dione | 405.4 |
| 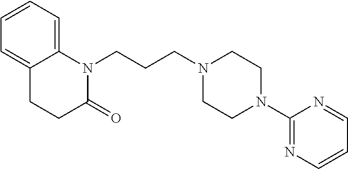 [SMILES: O=C1CCc2ccccc2N1CCCN1CCN(c2ncccn2)CC1] | 1-(3-(4-(pyrimidin-2-yl)piperazin- 1-yl)propyl)-3,4-dihydroquinolin- 2(1H)-one | 352.4 |
| 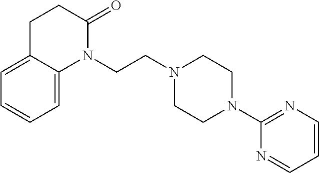 [SMILES: O=C1CCc2ccccc2N1CCN1CCN(c2ncccn2)CC1] | 1-(2-(4-(pyrimidin-2-yl)piperazin- 1-yl)ethyl)-3,4-dihydroquinolin- 2(1H)-one | 338.4 |
| 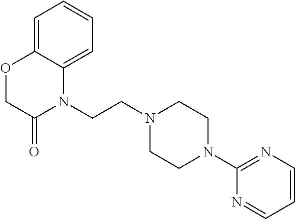 [SMILES: O=C1COc2ccccc2N1CCN1CCN(c2ncccn2)CC1] | 4-(2-(4-(pyrimidin-2-yl)piperazin- 1-yl)ethyl)-2H- benzo[b][1,4]oxazin-3(4H)-one | 340.8 |
| 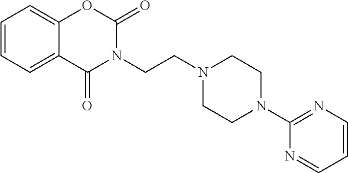 [SMILES: O=c1oc2ccccc2c(=O)n1CCN1CCN(c2ncccn2)CC1] | 3-(2-(4-(pyrimidin-2-yl)piperazin- 1-yl)ethyl)-2H- benzo[e][1,3]oxazine-2,4(3H)- dione | 354.3 |
| 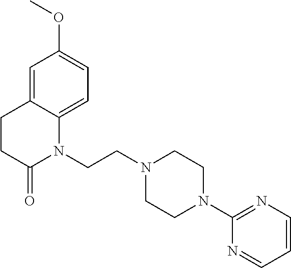 [SMILES: COc1ccc2c(c1)CCC(=O)N2CCN1CCN(c2ncccn2)CC1] | 6-methoxy-1-(2-(4-(pyrimidin-2- yl)piperazin-1-yl)ethyl)-3,4- dihydroquinolin-2(1H)-one | 368.4 |
| 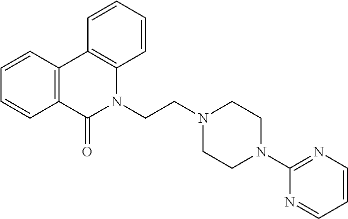 [SMILES: O=c1c2ccccc2c2ccccc2n1CCN1CCN(c2ncccn2)CC1] | 5-(2-(4-(pyrimidin-2-yl)piperazin- 1-yl)ethyl)phenanthridin-6(5H)- one | 386.4 |
| 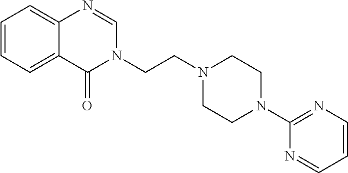 [SMILES: O=c1c2ccccc2ncn1CCN1CCN(c2ncccn2)CC1] | 3-(2-(4-(pyrimidin-2-yl)piperazin- 1-yl)ethyl)quinazolin-4(3H)-one | 337.3 |
| 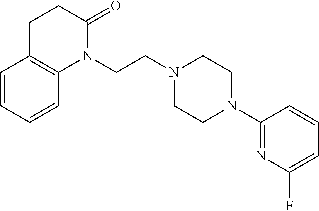 [SMILES: O=C1CCc2ccccc2N1CCN1CCN(c2cccc(F)n2)CC1] | 1-(2-(4-(6-fluoropyridin-2- yl)piperazin-1-yl)ethyl)-3,4- dihydroquinolin-2(1H)-one | 355.4 |
| 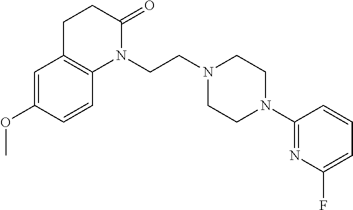 [SMILES: COc1ccc2c(c1)CCC(=O)N2CCN1CCN(c2cccc(F)n2)CC1] | 1-(2-(4-(6-fluoropyridin-2- yl)piperazin-1-yl)ethyl)-6- methoxy-3,4-dihydroquinolin- 2(1H)-one | 385.4 |
| 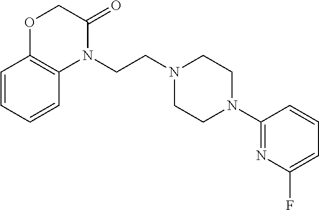 [SMILES: O=C1COc2ccccc2N1CCN1CCN(c2cccc(F)n2)CC1] | 4-(2-(4-(6-fluoropyridin-2- yl)piperazin-1-yl)ethyl)-2H- benzo[b][1,4]oxazin-3(4H)-one | 357.4 |
| 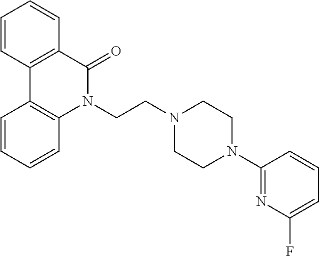 [SMILES: O=c1c2ccccc2c2ccccc2n1CCN1CCN(c2cccc(F)n2)CC1] | 5-(2-(4-(6-fluoropyridin-2- yl)piperazin-1- yl)ethyl)phenanthridin-6(5H)-one | 403.4 |
| 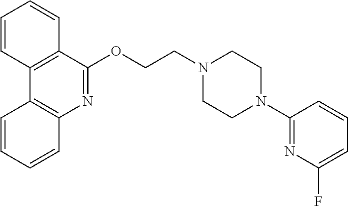 [SMILES: Fc1cccc(N2CCN(CCOc3nc4ccccc4c4ccccc34)CC2)n1] | 6-(2-(4-(6-fluoropyridin-2- yl)piperazin-1- yl)ethoxy)phenanthridine | 403.4 |
| 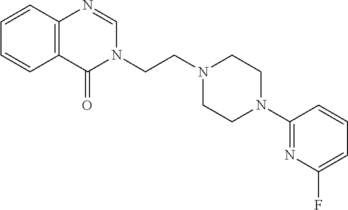 [SMILES: O=c1c2ccccc2ncn1CCN1CCN(c2cccc(F)n2)CC1] | 3-(2-(4-(6-fluoropyridin-2- yl)piperazin-1- yl)ethyl)quinazolin-4(3H)-one | 354.5 |
| 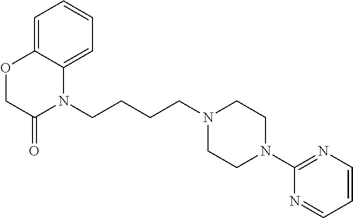 [SMILES: O=C1COc2ccccc2N1CCCCN1CCN(c2ncccn2)CC1] | 4-(4-(4-(pyrimidin-2-yl)piperazin- 1-yl)butyl)-2H- benzo[b][1,4]oxazin-3(4H)-one | 368.4 |
| 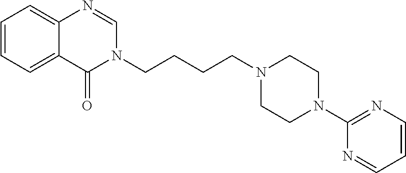 [SMILES: O=c1c2ccccc2ncn1CCCCN1CCN(c2ncccn2)CC1] | 3-(4-(4-(pyrimidin-2-yl)piperazin- 1-yl)butyl)quinazolin-4(3H)-one | 365.4 |
| 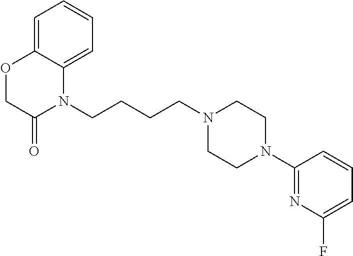 [SMILES: O=C1COc2ccccc2N1CCCCN1CCN(c2cccc(F)n2)CC1] | 4-(4-(4-(6-fluoropyridin-2- yl)piperazin-1-yl)butyl)-2H- benzo[b][1,4]oxazin-3(4H)-one | 385.5 |
| 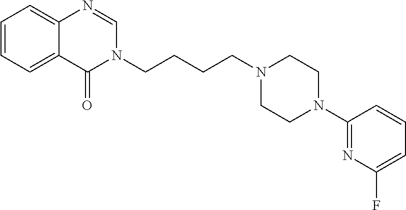 [SMILES: O=c1c2ccccc2ncn1CCCCN1CCN(c2cccc(F)n2)CC1] | 3-(4-(4-(6-fluoropyridin-2- yl)piperazin-1- yl)butyl)quinazolin-4(3H)-one | 382.5 |
| 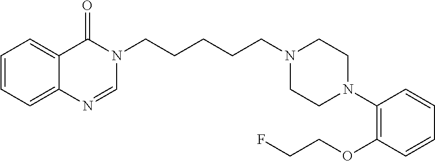 [SMILES: O=c1c2ccccc2ncn1CCCCCN1CCN(c2ccccc2OCCF)CC1] | 3-(5-(4-(2-(2- fluoroethoxy)phenyl)piperazin-1- yl)pentyl)quinazolin-4(3H)-one | 439.5 |

### Example 2

### Scheme 2 Depicts a Method for Making Radiolabeled Compounds of Formula (I) for Preparation of 2-((3-(4-(6-fluoropyridin-2-yl)piperazin-1-yl)propyl)thio)benzo[d]thiazole

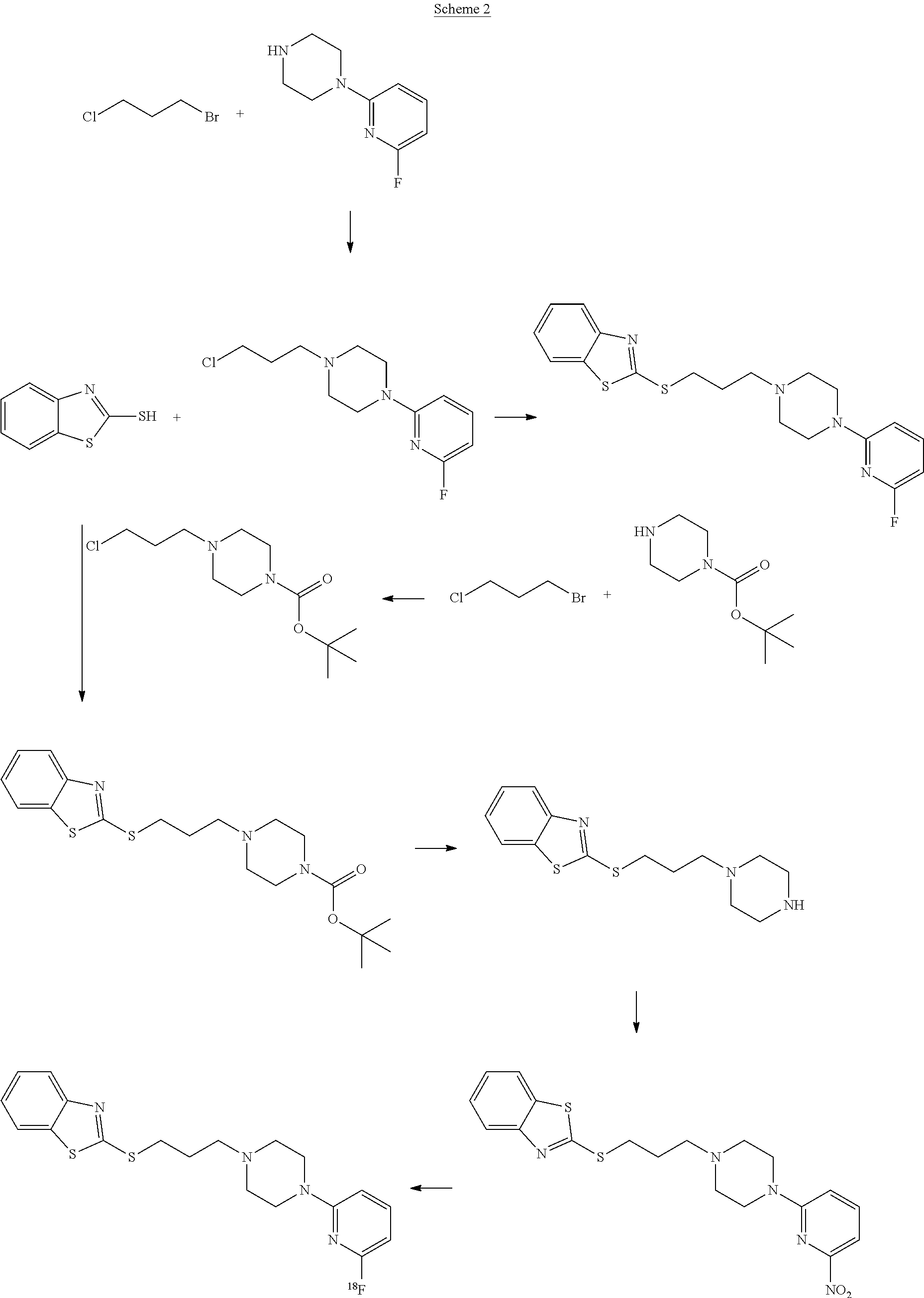

### Preparation of 1-(3-chloropropyl)-4-(6-fluoropyridin-2-yl)piperazine

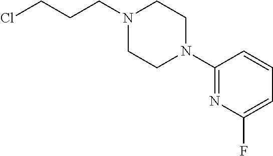 [SMILES: Fc1cccc(N2CCN(CCCCl)CC2)n1]

Dissolved 1-(6-fluoropyridin-2-yl)piperazine (0.25 g, 1.38 mmol) in dry DMF (5 mL) and added 1-bromo-3-chloropropane (0.19 mL, 0.3 g, 1.9 mmol) and potassium carbonate (0.3 g). Stirred vigorously under dry nitrogen for 18 h. Diluted with water (40 mL) ands extracted with ethyl acetate (3×20 mL). Combined organics were washed with water (20 mL), brine (20 mL), dried over sodium sulfate, filtered and evaporated to give an almost colourless oil (250 mg, 70% yield). TLC (6% methanol-dichloromethane).

### Preparation of 2-((3-(4-(6-fluoropyridin-2-yl)piperazin-1-yl)propyl)thio)benzo[d]thiazole

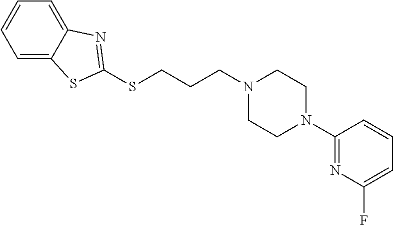 [SMILES: Fc1cccc(N2CCN(CCCSc3nc4ccccc4s3)CC2)n1]

1-(3-Chloropropyl)-4-(6-fluoropyridin-2-yl)piperazine (258 mg, 1.000 mmol) in acetone (7.5 mL) was added to a suspension of benzo[d]thiazole-2-thiol (167 mg, 1 mmol) in acetone (7.5 mL) with Potassium carbonate (138 mg, 1.000 mmol) and a catalytic quantity of potassium iodide. The mixture was heated to reflux for 24 h. On cooling, the solvent was evaporated and the solid residue suspended in brine (30 mL) and extracted into diethylether (4×15 mL). Combined organics were washed with brine (10 mL), dried over sodium sulfate, filtered and evaporated to a yellow oil which solidified on standing (433 mg). Purified by column chromatography on silica gel (10 g) in 5-100% ethyl acetate-petrol gradient over 28 CV to give 1 major peaks at 40%. Evaporated appropriate fractions to give a yellow viscous oil (220 mg). ¹H NMR showed about 10% residual 1-(3-chloropropyl)-4-(6-fluoropyridin-2-yl)piperazine. Repurified by column chromatography to give IMN05-087B (178 mg, 46%).

LCMS calcd for C₁₉H₂₁FN₄S₂: 388.1; found: 389.0 [M+H]⁺.

¹H, ¹³C and ¹⁹F NMR consistent with structure.

### Preparation of tert-Butyl 4-(3-chloropropyl)piperazine-1-carboxylate

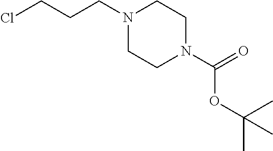 [SMILES: CC(C)(C)OC(=O)N1CCN(CCCCl)CC1]

tert-Butylpiperazine-1-carboxylate (500 mg, 2.68 mmol) was dissolved in acetonitrile (20 ml) and 1-bromo-3-chloropropane (423 mg, 2.68 mmol) and triethylamine (272 mg, 2.68 mmol) added. The mixture was put in 5×10 mL microwave tubes and each heated at 50° C. for 20 mins. Combined the tubes and evaporated to dryness. Dissolved in the minimum quantity of dichloromethane and purified by chromatography on silica gel (50 g column eluting with 10-100% EtOAc-petrol gradient over 20 CV at 85 mL/min. Appropriate fractions were identified by UV and permanganate visualized TLC (silica plate) and evaporated to give the product as a pale yellow oil (366 mg, 52%).

¹H NMR (300 MHz, d6-CDCl₃): δ 1.43 (9H, s, C(CH₃)₃), 1.87-1.97 (2H, m, CH₂CH₂), 2.35 (4H, t, J=5.0 Hz, pip-CH₂), 2.46 (24H, t, J=7.0 Hz, N—CH₂), 3.40 (4H, t, J=5.0 Hz, pip-CH₂) and 3.58 (2H, t, J=6.5 Hz, CH₂—Cl). ¹³C NMR (75 MHz, d6-CDCl₃): δ 28.4 (C(CH₃)₃), 29.7 (CH₂CH₂CH₂), 43.0 (CH₂—Cl), 43.8 (pip-CH₂), 53.0 (N—CH₂), 55.3 (pip-CH2), 79.6 (C(CH₃)₃) and 154.7 (C═O).

### Preparation of tert-Butyl 4-(3-(benzo[d]thiazol-2-ylthio)propyl)piperazine-1-carboxylate

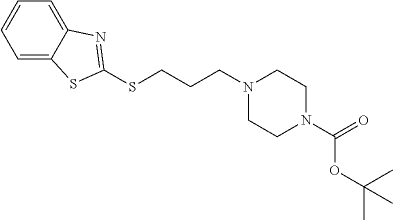 [SMILES: CC(C)(C)OC(=O)N1CCN(CCCSc2nc3ccccc3s2)CC1]

Benzo[d]thiazole-2-thiol (233 mg, 1.393 mmol) and tert-butyl 4-(3-chloropropyl)piperazine-1-carboxylate (366 mg, 1.393 mmol) were dissolved in acetone (40 ml) and potassium carbonate (386 mg, 2 equivs) and one small crystal of potassium iodide (catalytic) added. The vigorously stirred suspension was heated at reflux for 18 h. TLC (1:1 ethyl acetate:petrol) showed one major spot at ˜Rf 0.5.

Evaporated, partitioned between water (15 mL) and dichloromethane (25 mL) separated with a phase separator and evaporated. The product was purified by chromatography on silica gel (50 g cartridge) eluting with 5-100% ethyl acetate-petrol over 16 CV to give the product as a viscous oil (350 mg, 64%).

LCMS calcd for C₁₉H₂₇N₃O₂S₂: 393.2; found:394.1 [M+H]⁺ in ES+.

¹H NMR (300 MHz, CDCl₃): δ 1.45 (9H, s, tBu), 1.97-2.06 (1H, m, CH₂—CH₂), 2.34-2.32 (4H, pip-CH₂), 2.50 (2H, t, J=7.0 Hz, CH₂—N), 3.39 (2H, t, J=7.5 Hz, CH₂—N), 3.40-3.46 (4H, m, pip-CH₂), 7.24-7.31 (1H, m, benzo-C5/6-H), 7.36-7.43 (1H, m, benzo-C5/6-H), 7.74 (1H, d, J=8.0 Hz, benzoC4-H) and 7.83 (1H, d, J=7.5 Hz, benzoC7-H). ¹³C NMR (75 MHz, CDCl₃): δ 26.7 (CH₂CH₂CH₂), 28.6 (C(CH₃)₃), 31.1 (CH₂—S), 31.6 (pip-CH₂), 53.1 (CH₂—N), 57.1 (pip-CH₂), 79.8 (C—O), 121.1 (benzoC7), 121.6 (benzoC-4), 124.3 (benzoC6), 12126.2 (benzoC-5), 135.4 (C—S), 153.5 (C—N), 154.9 (C═O) and 167.2 (S—C═N).

### Preparation of 2-((3-(piperazin-1-yl)propyl)thio)benzo[d]thiazole

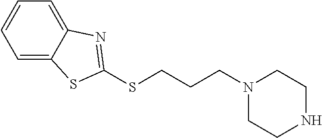 [SMILES: c1ccc2sc(SCCCN3CCNCC3)nc2c1]

tert-Butyl 4-(3-(benzo[d]thiazol-2-ylthio)propyl)piperazine-1-carboxylate (350 mg, 0.889 mmol) was dissolved in dichloromethane (10 mL) and cooled in an ice-bath and cold trifluoroacetic acid (10 mL) added. The mixture was stirred for 3 h and ice-bath removed after 10 mins. Evaporated to dryness to give the product as a sticky viscous oil that was used directly in the next step.

LCMS calc for C₁₄H₁₉N₃S₂ 293.1; found 294.0 [M+H]⁺ in ES+

### Preparation of precursor 2-((3-(4-(6-nitropyridin-2-yl)piperazin-1-yl)propyl)thio)benzo[d]thiazole

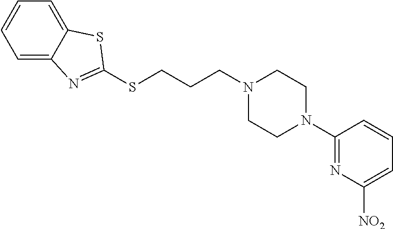 [SMILES: O=[N+]([O-])c1cccc(N2CCN(CCCSc3nc4ccccc4s3)CC2)n1]

2-(3-(piperazin-1-yl)propylthio)benzo[d]thiazole (150 mg, 0.5 mmol) was dissolved in anhydrous acetonitrile (4 mL) in a 10 mL microwave tube. To this solution was added 2-chloro-6-nitropyridine (80 mg, 0.5 mmol) and di-isopropylethylamine (0.2 mL) at ambient temperature. The reaction mixture was heated in a microwave at 120° C. for 30 min. The resultant solution was evaporated to dryness under reduced pressure, partitioned between dichloromethane and water and separated with a phase-separator cartridge. The organic portion was evaporated to dryness under reduced pressure to afford a dark yellow oil. Purification of the oil by flash column chromatography on a 50 g high performance silica cartridge, eluted with isocratic 2.5% methanol in ethyl acetate at a flow rate of 40 mL/min afforded 2-(3-(4-(6-nitropyridin-2-yl)piperazin-1-1)propylthio)benzo[d]thiazole as a bright yellow oil (22 mg, 11%).

LCMS calcd for C₁₉H₂₁N₅O₂S₂: 415.1; found: 415.9 [M+H]⁺.

¹H NMR (300 MHz, CDCl₃): δ 2.07 (p, 2H), 2.57 (t, 6H), 3.44 (t, 2H), 3.67 (t, 4H), 6.90 (d, 1H), 7.29 (t, 1H), 7.42 (t, 2H), 7.68 (t, 1H), 7.76 (d, 1H), 7.86 (d, 1H). ¹³C NMR (75.5 MHz, CDCl₃): δ 26.4, 31.3, 44.9, 52.7, 56.9, 105.3, 111.7, 120.9, 121.4, 124.1, 126.0, 135.1, 140.0, 153.3, 155.9, 157.8, 166.9.

### Preparation of [¹⁸F]2-((3-(4-(6-Fluoropyridin-2-yl)piperazin-1-yl)propyl)thio)benzo[d]thiazole

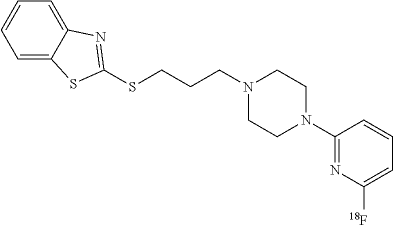 [SMILES: [18F]c1cccc(N2CCN(CCCSc3nc4ccccc4s3)CC2)n1]

[¹⁸F]-fluoride (7.99 GBq) was transferred to the GE TracerLab FX and trapped on a conditioned QMA cartridge. The [¹⁸F]-fluoride was then eluted as the Kryptofix (K222) complex using a solution formed of Kryptofix (10.0 mg) in acetonitrile (2 ml) and 0.1 M potassium carbonate (100 μl) into the reaction vessel. Azeotropic drying of the [¹⁸F]-fluoride commenced at 100° C. for 30 minutes under a stream of N₂ gas. The dried [¹⁸F]-fluoride/K222 vial was then cooled to 30° C. and N₂ flow ceased. 2-(3-(4-(6-nitropyridin-2-yl)piperazin-1-1)propylthio)benzo[d]thiazole (1.9 mg) in DMSO (1 ml) was added to the reaction vessel which was then sealed and heated to 150° C. for 10 minutes. The crude reaction was diluted with H₂O (2.5 ml) and transferred to a semi-preparative HPLC system (Phenomenex Luna C8 250×10 mm, 5 um, A=0.8% TRIETHYLAMINE adj. to pH 7.6 with phosphoric acid, B=acetonitrile, 50%-95% B over 20 minutes). The collected fraction was diluted with H₂O (10 ml) and trapped on a tC18 light cartridge (conditioned with ethanol (5 ml) and H₂O (10 ml)). The cartridge was washed with H₂O (4 ml) and eluted with ethanol (500 μl) before formulation with PBS (4.5 ml). The product consisted of 462 MBq (11% non-decay corrected yield). [¹⁸F]2-((3-(4-(6-Fluoropyridin-2-yl)piperazin-1-yl)propyl)thio)benzo[d]thiazole was analyzed by RP HPLC (Phenomenex Luna C8 150×4.6 mm, 5 um A=0.8% TRIETHYLAMINE, adj. to pH 7.6 with H₃PO₄ B=MeCN; 50%-95% B over 20 mins) and eluted simultaneously with a spike of cold standard at 12.3 minutes, total synthesis time 120 minutes, specific activity 16 GBq/umol.

Compounds that were synthesized following procedures that were similar or identical as described those in Examples 2, 11, and 17 include the following:

| Structure | Name | [M + H]⁺ |
|---|---|---|
| 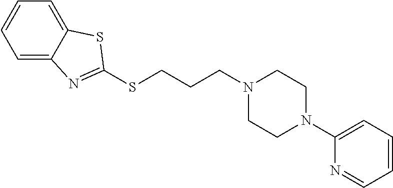 [SMILES: c1ccc(N2CCN(CCCSc3nc4ccccc4s3)CC2)nc1] | 2-((3-(4-(pyridin-2- yl)piperazin-1- yl)propyl)thio)benzo[d]thiazole | 371.5 |
| 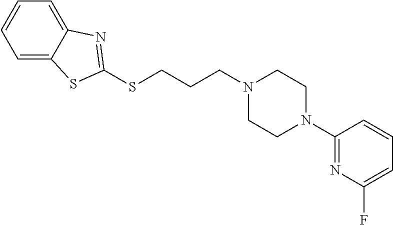 [SMILES: Fc1cccc(N2CCN(CCCSc3nc4ccccc4s3)CC2)n1] | 2-((3-(4-(6-fluoropyridin-2- yl)piperazin-1- yl)propyl)thio)benzo[d]thiazole | 389.5 |
| 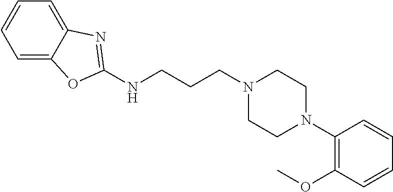 [SMILES: COc1ccccc1N1CCN(CCCNc2nc3ccccc3o2)CC1] | N-(3-(4-(2- methoxyphenyl)piperazin-1- yl)propyl)benzo[d]oxazol-2- amine | 367.4 |
| 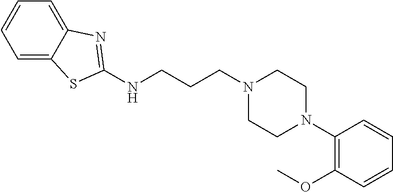 [SMILES: COc1ccccc1N1CCN(CCCNc2nc3ccccc3s2)CC1] | N-(3-(4-(2- methoxyphenyl)piperazin-1- yl)propyl)benzo[d]thiazol-2- amine | 383.4 |
| 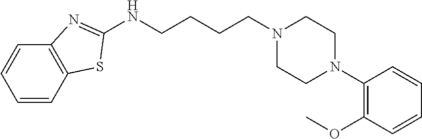 [SMILES: COc1ccccc1N1CCN(CCCCNc2nc3ccccc3s2)CC1] | N-(4-(4-(2- methoxyphenyl)piperazin-1- yl)butyl)benzo[d]thiazol-2- amine | 397.5 |
| 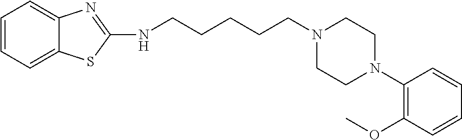 [SMILES: COc1ccccc1N1CCN(CCCCCNc2nc3ccccc3s2)CC1] | N-(5-(4-(2- methoxyphenyl)piperazin-1- yl)pentyl)benzo[d]thiazol-2- amine | 411.5 |
| 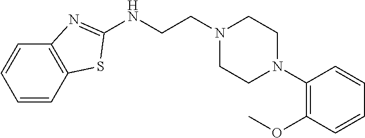 [SMILES: COc1ccccc1N1CCN(CCNc2nc3ccccc3s2)CC1] | N-(2-(4-(2- methoxyphenyl)piperazin-1- yl)ethyl)benzo[d]thiazol-2- amine | 369.4 |
| 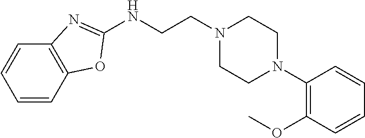 [SMILES: COc1ccccc1N1CCN(CCNc2nc3ccccc3o2)CC1] | N-(2-(4-(2- methoxyphenyl)piperazin-1- yl)ethyl)benzo[d]oxazol-2- amine | 353.2 |
| 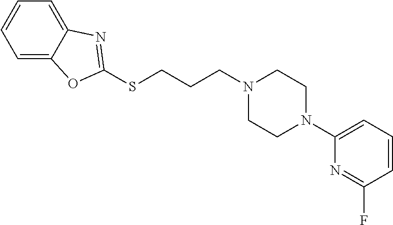 [SMILES: Fc1cccc(N2CCN(CCCSc3nc4ccccc4o3)CC2)n1] | 2-((3-(4-(6-fluoropyridin-2- yl)piperazin-1- yl)propyl)thio)benzo[d]oxazole | 373.4 |
| 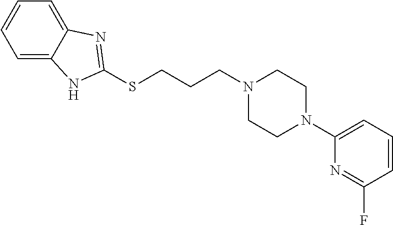 [SMILES: Fc1cccc(N2CCN(CCCSc3nc4ccccc4[nH]3)CC2)n1] | 2-((3-(4-(6-fluoropyridin-2- yl)piperazin-1-yl)propyl)thio)- 1H-benzo[d]imidazole | 372.4 |
| 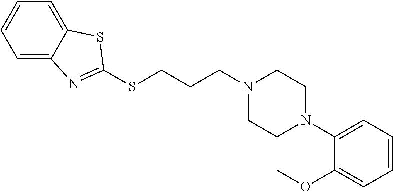 [SMILES: COc1ccccc1N1CCN(CCCSc2nc3ccccc3s2)CC1] | 2-((3-(4-(2- methoxyphenyl)piperazin-1- yl)propyl)thio)benzo[d]thiazole | 400.5 |
| 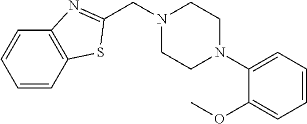 [SMILES: COc1ccccc1N1CCN(Cc2nc3ccccc3s2)CC1] | 2-((4-(2- methoxyphenyl)piperazin-1- yl)methyl)benzo[d]thiazole | 340.4 |
| 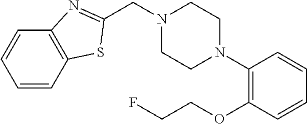 [SMILES: FCCOc1ccccc1N1CCN(Cc2nc3ccccc3s2)CC1] | 2-((4-(2-(2- fluoroethoxy)phenyl)piperazin- l-yl)methyl)benzo[d]thiazole | 372.4 |
| 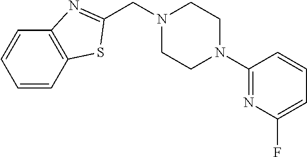 [SMILES: Fc1cccc(N2CCN(Cc3nc4ccccc4s3)CC2)n1] | 2-((4-(6-fluoropyridin-2- yl)piperazin-1- yl)methyl)benzo[d]thiazole | 329.3 |
| 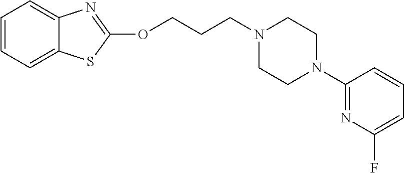 [SMILES: Fc1cccc(N2CCN(CCCOc3nc4ccccc4s3)CC2)n1] | 2-(3-(4-(6-fluoropyridin-2- yl)piperazin-1- yl)propoxy)benzo[d]thiazole | 373.4 |
| 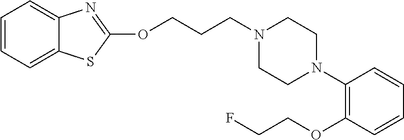 [SMILES: FCCOc1ccccc1N1CCN(CCCOc2nc3ccccc3s2)CC1] | 2-(3-(4-(2-(2- fluoroethoxy)phenyl)piperazin- 1-yl)propoxy)benzo[d]thiazole | 416.4 |
| 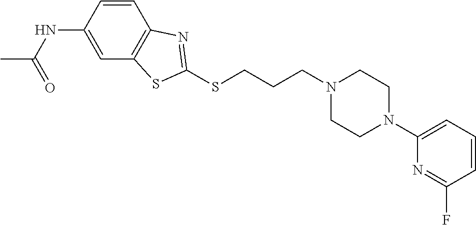 [SMILES: CC(=O)Nc1ccc2nc(SCCCN3CCN(c4cccc(F)n4)CC3)sc2c1] | N-(2-((3-(4-(6-fluoropyridin-2- yl)piperazin-1- yl)propyl)thio)benzo[d]thiazol- 6-yl)acetamide | 446.5 |
| 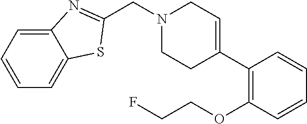 [SMILES: FCCOc1ccccc1C1=CCN(Cc2nc3ccccc3s2)CC1] | 2-((4-(2-(2- fluoroethoxy)phenyl)-5,6- dihydropyridin-1(2H)- yl)methyl)benzo[d]thiazole | 369.2 |
| 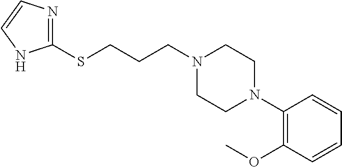 [SMILES: COc1ccccc1N1CCN(CCCSc2ncc[nH]2)CC1] | 1-(3-((1H-imidazol-2- yl)thio)propyl)-4-(2- methoxyphenyl)piperazine | 333.4 |
| 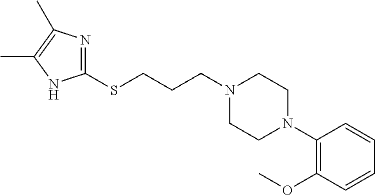 [SMILES: COc1ccccc1N1CCN(CCCSc2nc(C)c(C)[nH]2)CC1] | 1-(3-((4,5-dimethyl-1H- imidazol-2-yl)thio)propyl)-4- (2-methoxyphenyl)piperazine | 361.4 |
| 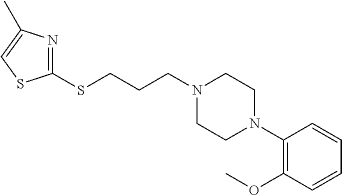 [SMILES: COc1ccccc1N1CCN(CCCSc2nc(C)cs2)CC1] | 2-((3-(4-(2- methoxyphenyl)piperazin-1- yl)propyl)thio)-4- methylthiazole | 364.4 |
| 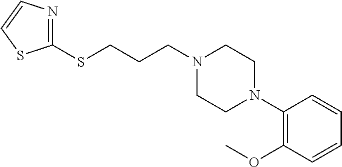 [SMILES: COc1ccccc1N1CCN(CCCSc2nccs2)CC1] | 2-((3-(4-(2- methoxyphenyl)piperazin-1- yl)propyl)thio)thiazole | 350.4 |
| 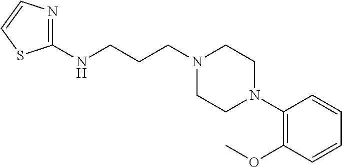 [SMILES: COc1ccccc1N1CCN(CCCNc2nccs2)CC1] | N-(3-(4-(2- methoxyphenyl)piperazin-1- yl)propyl)thiazol-2-amine | 333.4 |
| 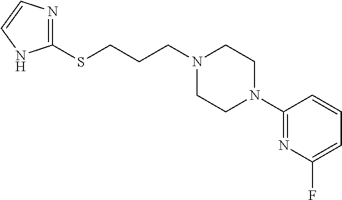 [SMILES: Fc1cccc(N2CCN(CCCSc3ncc[nH]3)CC2)n1] | 1-(3-((1H-imidazol-2- yl)thio)propyl)-4-(6- fluoropyridin-2-yl)piperazine | 322.3 |
| 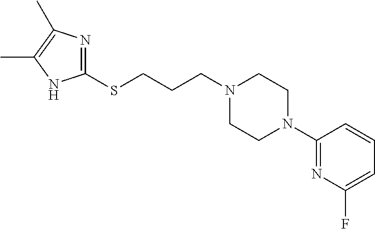 [SMILES: Cc1nc(SCCCN2CCN(c3cccc(F)n3)CC2)[nH]c1C] | 1-(3-((4,5-dimethyl-1H- imidazol-2-yl)thio)propyl)-4- (6-fluoropyridin-2- yl)piperazine | 350.4 |
| 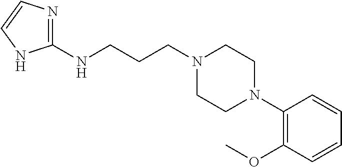 [SMILES: COc1ccccc1N1CCN(CCCNc2ncc[nH]2)CC1] | N-(3-(4-(2- methoxyphenyl)piperazin-1- yl)propyl)-1H-imidazol-2- amine | 316.3 |
| 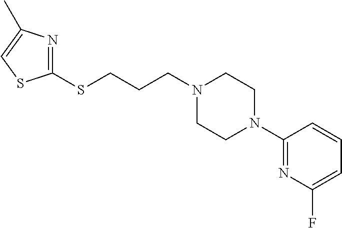 [SMILES: Cc1csc(SCCCN2CCN(c3cccc(F)n3)CC2)n1] | 2-((3-(4-(6-fluoropyridin-2- yl)piperazin-1-yl)propyl)thio)- 4-methylthiazole | 353.4 |
| 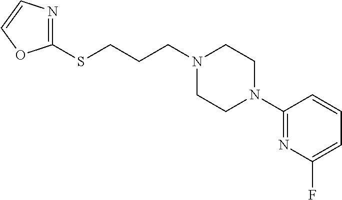 [SMILES: Fc1cccc(N2CCN(CCCSc3ncco3)CC2)n1] | 2-((3-(4-(6-fluoropyridin-2- yl)piperazin-1- yl)propyl)thio)oxazole | 323.3 |
| 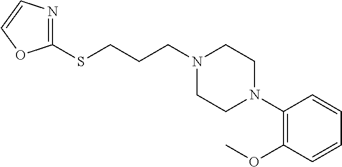 [SMILES: COc1ccccc1N1CCN(CCCSc2ncco2)CC1] | 2-((3-(4-(2- methoxyphenyl)piperazin-1- yl)propyl)thio)oxazole | 334.3 |
| 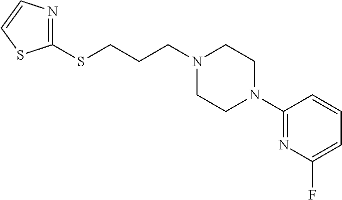 [SMILES: Fc1cccc(N2CCN(CCCSc3nccs3)CC2)n1] | 2-((3-(4-(6-fluoropyridin-2- yl)piperazin-1- yl)propyl)thio)thiazole | 339.4 |
| 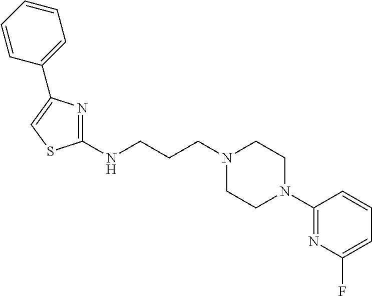 [SMILES: Fc1cccc(N2CCN(CCCNc3nc(-c4ccccc4)cs3)CC2)n1] | N-(3-(4-(6-fluoropyridin-2- yl)piperazin-1-yl)propyl)-4- phenylthiazol-2-amine | 398.4 |
| 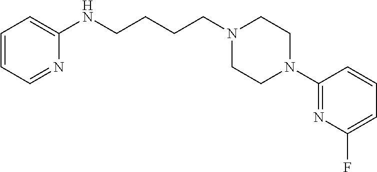 [SMILES: Fc1cccc(N2CCN(CCCCNc3ccccn3)CC2)n1] | N-(4-(4-(6-fluoropyridin-2- yl)piperazin-1- yl)butyl)pyridin-2-amine | 330.3 |
| 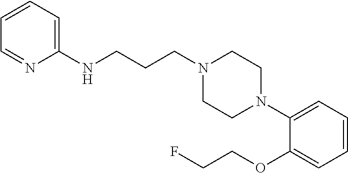 [SMILES: FCCOc1ccccc1N1CCN(CCCNc2ccccn2)CC1] | N-(3-(4-(2-(2- fluoroethoxy)phenyl)piperazin- 1-yl)propyl)pyridin-2-amine | 359.4 |
| 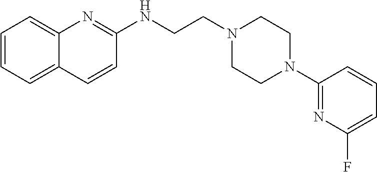 [SMILES: Fc1cccc(N2CCN(CCNc3ccc4ccccc4n3)CC2)n1] | N-(2-(4-(6-fluoropyridin-2- yl)piperazin-1- yl)ethyl)quinolin-2-amine | 352.3 |
| 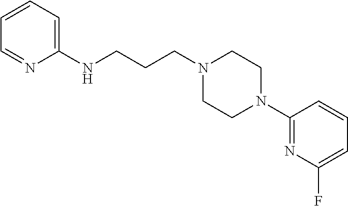 [SMILES: Fc1cccc(N2CCN(CCCNc3ccccn3)CC2)n1] | N-(3-(4-(6-fluoropyridin-2- yl)piperazin-1- yl)propyl)pyridin-2-amine | 316.3 |
| 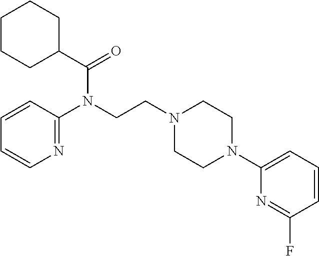 [SMILES: O=C(C1CCCCC1)N(CCN1CCN(c2cccc(F)n2)CC1)c1ccccn1] | N-(2-(4-(6-fluoropyridin-2- yl)piperazin-1-yl)ethyl)-N- (pyridin-2- yl)cyclohexanecarboxamide | 412.4 |
| 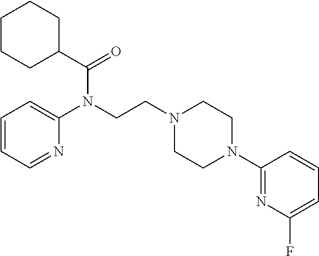 [SMILES: O=C(C1CCCCC1)N(CCN1CCN(c2cccc(F)n2)CC1)c1ccccn1] | N-(4-(4-(6-fluoropyridin-2- yl)piperazin-1-yl)butyl)-N- (pyridin-2- yl)cyclohexanecarboxamide | 440.5 |
| 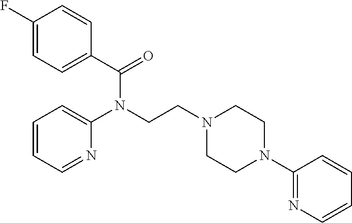 [SMILES: O=C(c1ccc(F)cc1)N(CCN1CCN(c2ccccn2)CC1)c1ccccn1] | 4-fluoro-N-(pyridin-2-yl)-N- (2-(4-(pyridin-2-yl)piperazin- 1-yl)ethyl)benzamide | 406.4 |
| 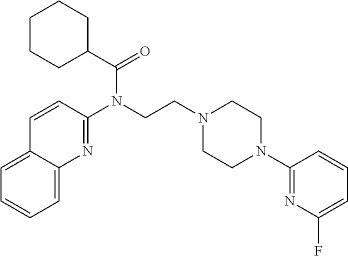 [SMILES: O=C(C1CCCCC1)N(CCN1CCN(c2cccc(F)n2)CC1)c1ccc2ccccc2n1] | N-(2-(4-(6-fluoropyridin-2- yl)piperazin-1-yl)ethyl)-N- (quinolin-2- yl)cyclohexanecarboxamide | 462.5 |

### Example 3

### Scheme 3 Shows a Method for Making Radiolabeled Compounds of Formula (I) for Preparation of 1-(2-fluoroethyl)-3-(4-(4-(2-methoxyphenyl)piperazin-1-yl)butyl)imidaz-olidine-2,4-dione

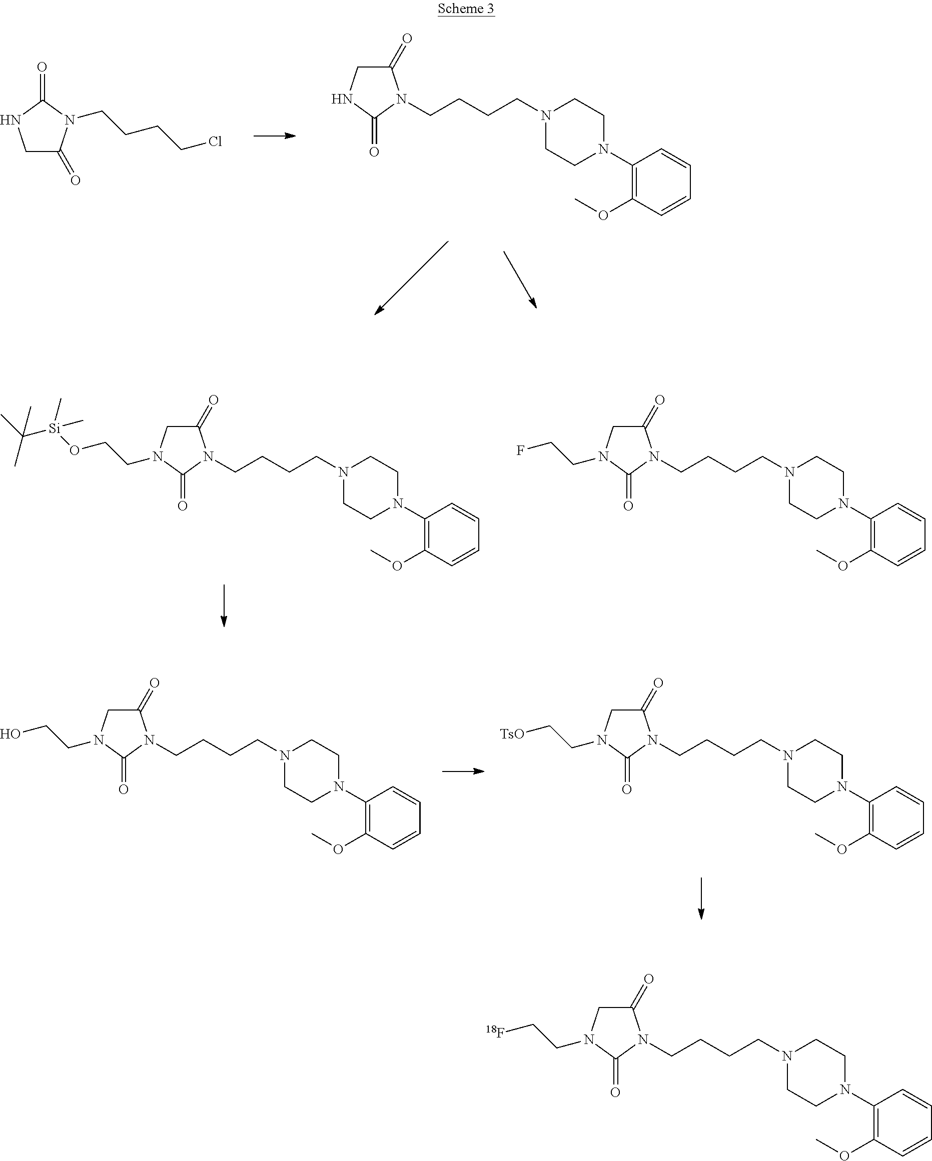

### Preparation of 3-(4-Chlorobutyl)imidazolidine-2,4-dione

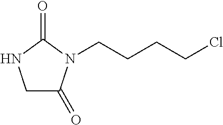 [SMILES: O=C1CNC(=O)N1CCCCCl]

To hydantoin (1.06 g, 10.5 mmol) in dimethylformamide (20 mL) at 50° C. was added a 60% suspension of sodium hydride (420 mg, 10.5 mmol). After stirring for 60 minutes 1-bromo-4-chlorobutane (4.5 g, 26.3 mmol) was added, and the mixture stirred for 18 h. The reaction was quenched with 1 N HCl (20 mL, 20 mmol) and concentrated to give a yellow oil. This was purified by chromatography on silica gel (40 g) eluting with ethyl acetate at 40 mL/min. The product eluted in fraction 3-7. These were concentrated to give a pale orange solid (1.9 g, 95%).

¹H (CDCl₃, 300 MHz): δ 1.65 (4H, m), 3.36 (2H, t), 3.62 (2H, t), 3.89 (2H, s), and 8.02 (1H, s).

### 3-(4-(4-(2-Methoxyphenyl)piperazin-1-yl)butyl)imidazolidine-2,4-dione

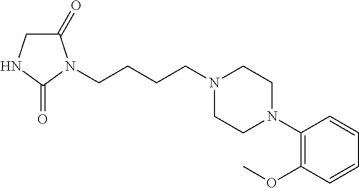 [SMILES: COc1ccccc1N1CCN(CCCCN2C(=O)CNC2=O)CC1]

To 3-(4-chlorobutyl)imidazolidine-2,4-dione (1.10 g, 5.7 mmol) in acetone (100 mL) was added potassium carbonate (2.5 g, 18 mmol) and 1-(2-methoxyphenyl)piperazine (1.0 g, 5.2 mmol). The mixture was heated at reflux for 24 hours. The mixture was cooled, filtered and concentrated at reduced pressure. The crude material was separated on silica (40 g) eluting with 15% methanol-dichloromethane at 40 mL/min. The product eluted between ˜4-8 CV. These fractions were concentrated at reduced pressure to yield a white solid (780 mg, 43%).

LCMS: calcd for C₁₈H₂₆N₄O₃: 346.2; found: 347.0 [M+H]⁺.

### Preparation of 1-(2-Fluoroethyl)-3-(4-(4-(2-methoxyphenyl)piperazin-1-yl)butyl)imidazolidine-2,4-dione

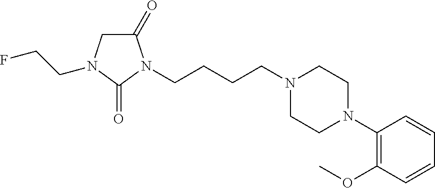 [SMILES: COc1ccccc1N1CCN(CCCCN2C(=O)CN(CCF)C2=O)CC1]

To 3-{4-[4-(2-methoxyphenyl)piperazin-1-yl]butyl}imidazolidine-2,4-dione (100 mg, 0.29 mmol) in DMF (1 mL) was added NaH (12 mg, 0.3 mmol) and fluoroethyltosylate (63 mg, 0.29 mmol). The mixture was stirred at room temperature for 30 minutes. The mixture was filtered and concentrated at reduced pressure. The crude material was purified by chromatography onsilica gel (40 g) eluting with 5% methanol—ethyl acetate for 5 CV then 5-15% methanol gradient over 15 CV at 30 mL/min. The product eluted between ˜2-6 CV. These fractions were concentrated at reduced pressure to yield an off white solid (30 mg, 29%).

¹H (CDCl₃, 300 MHz): δ 1.67 (4H, m), 2.56 (2H, t), 2.79 (4H, brm), 3.18 (4H, brm), 3.55 (2H, t), 3.70 (2H, dt), 3.84 (3H, s), 4.01 (2H, s), 4.60 (2H, dt), and 6.81-7.05 (4H, m).

### Preparation of 1-(2-(tert-butyldimethylsilyloxy)ethyl)-3-(4-(4-(2-methoxyphenyl)piperazin-1-yl)butyl)imidazolidine-2,4-dione

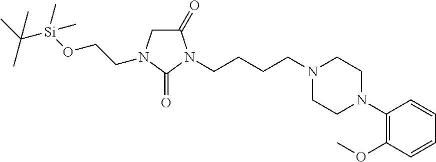 [SMILES: COc1ccccc1N1CCN(CCCCN2C(=O)CN(CCO[Si](C)(C)C(C)(C)C)C2=O)CC1]

To a solution of 3-{4-[4-(2-methoxyphenyl)piperazin-1-yl]butyl}imidazolidine-2,4-dione in DMF is added sodium hydride (1.1 equivalent) and (2-bromoethoxy)(tert-butyl)dimethylsilane (1 equivalent). The reaction mixture is stirred at 60° C. for 18 hours. On collong, the reaction is partitioned between dichloromethane and water. The organics are dried and concentrated. The reaction mixture will be purified on silica gel eluting with 10% methanol in ethyl acetate.

### Preparation of 1-(2-hydroxyethyl)-3-(4-(4-(2-methoxyphenyl)piperazin-1-yl)butyl)imidazolidine-2,4-dione

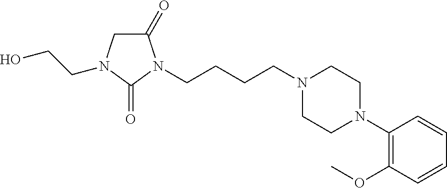 [SMILES: COc1ccccc1N1CCN(CCCCN2C(=O)CN(CCO)C2=O)CC1]

To 1-(2-(tert-butyldimethylsilyloxy)ethyl)-3-(4-(4-(2-methoxyphenyl)piperazin-1-yl)butyl)imidazolidine-2,4-dione is added 1M solution of tetrabutylammonium fluoride in tetrahydrofuran. After a short time, the reaction is partitioned between dichloromethane and water. The organics are dried and concentrated. The reaction mixture is purified on silica gel eluting with 5% methanol in ethyl acetate.

### Preparation of precursor 2-(3-(4-(4-(2-Methoxyphenyl)piperazin-1-yl)butyl)-2,4-dioxoimidazolidin-1-yl)ethyl 4-methylbenzenesulfonate

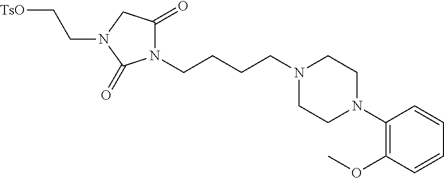 [SMILES: COc1ccccc1N1CCN(CCCCN2C(=O)CN(CCOS(=O)(=O)c3ccc(C)cc3)C2=O)CC1]

To a solution of 1-(2-hydroxyethyl)-3-(4-(4-(2-methoxyphenyl)piperazin-1-yl)butyl)imidazolidine-2,4-dione in dichloromethane is added triethylamine (1.1 equivalent) and tosyl chloride (1 equivalent) at 0° C. The reaction is partitioned between dichloromethane and water. The organics are dried and concentrated. The reaction mixture is purified on silica gel eluting with 5% methanol in ethyl acetate.

### Preparation of [¹⁸F]1-(2-fluoroethyl)-3-(4-(4-(2-methoxyphenyl)piperazin-1-yl)butyl)imidazolidine-2,4-dione

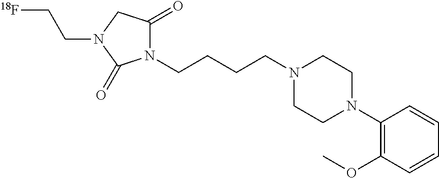 [SMILES: COc1ccccc1N1CCN(CCCCN2C(=O)CN(CC[18F])C2=O)CC1]

[¹⁸F]fluoride is transferred from a P6 vial into a 3 mL V-vial by suction. To the P6 vial is added a pre-prepared solution of kryptofix 2.2.2 (5 mg, 1.3×10⁻⁵ mol) in acetonitrile (0.5 mL) and potassium carbonate (65 μL, 0.1M) made up to 0.5 mL with water. The P6 vial is agitated and the solution transferred to the V-vial by suction. The V-vial is heated to 110° C. for 20 minutes under a flow of nitrogen (0.2 L/min) then cooled to room temperature.

To the dried [¹⁸F]fluoride and kryptofix mixture is added 2-(3-(4-(4-(2-methoxyphenyl)piperazin-1-yl)butyl)-2,4-dioxoimidazolidin-1-yl)ethyl 4-methylbenzenesulfonate (2 mg) in acetonitrile (1 mL). The resulting solution is heated at 80° C.-100° C. for 10-20 min, and then cooled to room temperature. To the reaction vial is added water (1.5 mL) and the mixture loaded on to preparative HPLC for purification. The isolated HPLC fraction is diluted into water (20 mL) and then formulated for administration.

Compounds that were synthesized following procedures that were similar or identical as described those in Examples 3 include the following:

| Structure | Name | [M + H]⁺ |
|---|---|---|
| 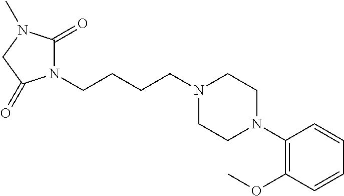 [SMILES: COc1ccccc1N1CCN(CCCCN2C(=O)CN(C)C2=O)CC1] | 3-(4-(4-(2- methoxyphenyl)piperazin- 1-yl)butyl)-1- methylimidazolidine-2,4- dione | 361.4 |
| 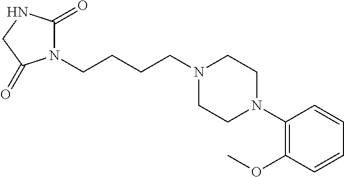 [SMILES: COc1ccccc1N1CCN(CCCCN2C(=O)CNC2=O)CC1] | 3-(4-(4-(2- methoxyphenyl)piperazin- 1-yl)butyl)imidazolidine- 2,4-dione | 347.4 |
| 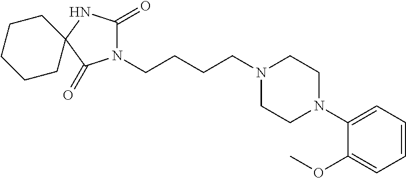 [SMILES: COc1ccccc1N1CCN(CCCCN2C(=O)NC3(CCCCC3)C2=O)CC1] | 3-(4-(4-(2- methoxyphenyl)piperazin- 1-yl)butyl)-1,3- diazaspiro[4.5]decane- 2,4-dione | 415.4 |
| 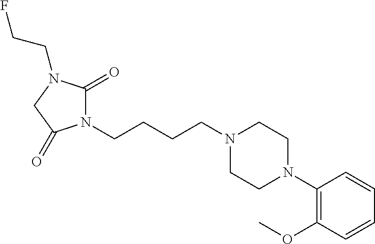 [SMILES: COc1ccccc1N1CCN(CCCCN2C(=O)CN(CCF)C2=O)CC1] | 1-(2-fluoroethyl)-3-(4-(4-(2- methoxyphenyl)piperazin- 1-yl)butyl)imidazolidine- 2,4-dione | 393.4 |
| 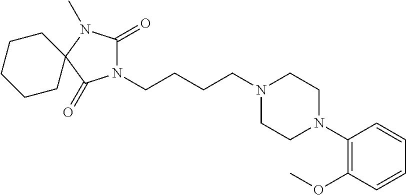 [SMILES: COc1ccccc1N1CCN(CCCCN2C(=O)N(C)C3(CCCCC3)C2=O)CC1] | 3-(4-(4-(2- methoxyphenyl)piperazin- 1-yl)butyl)-1-methyl-1,3- diazaspiro[4.5]decane- 2,4-dione | 429.5 |
| 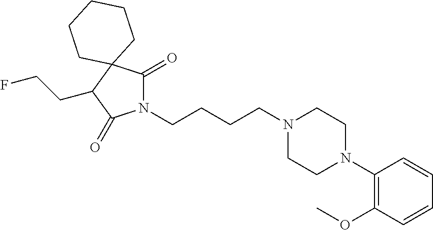 [SMILES: COc1ccccc1N1CCN(CCCCN2C(=O)C(CCF)C3(CCCCC3)C2=O)CC1] | 1-(2-fluoroethyl)-3-(4-(4-(2- methoxyphenyl)piperazin- 1-yl)butyl)-1,3- diazaspiro[4.5]decane- 2,4-dione | 461.5 |
| 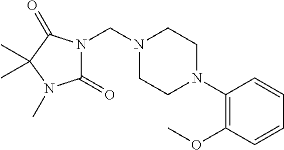 [SMILES: COc1ccccc1N1CCN(CN2C(=O)N(C)C(C)(C)C2=O)CC1] | 3-((4-(2- methoxyphenyl)piperazin- 1-yl)methyl)-1,5,5- trimethylimidazolidine- 2,4-dione | 347.3 |

### Example 4

### Scheme 4 Shows a Method for Making Radiolabeled Compounds of Formula (I) for Preparation of 4-(2-fluoroethyl)-2-(4-(4-(2-methoxyphenyl)piperazin-1-yl)butyl)-1,2,4-triazine-3,5(2H,4H)-dione

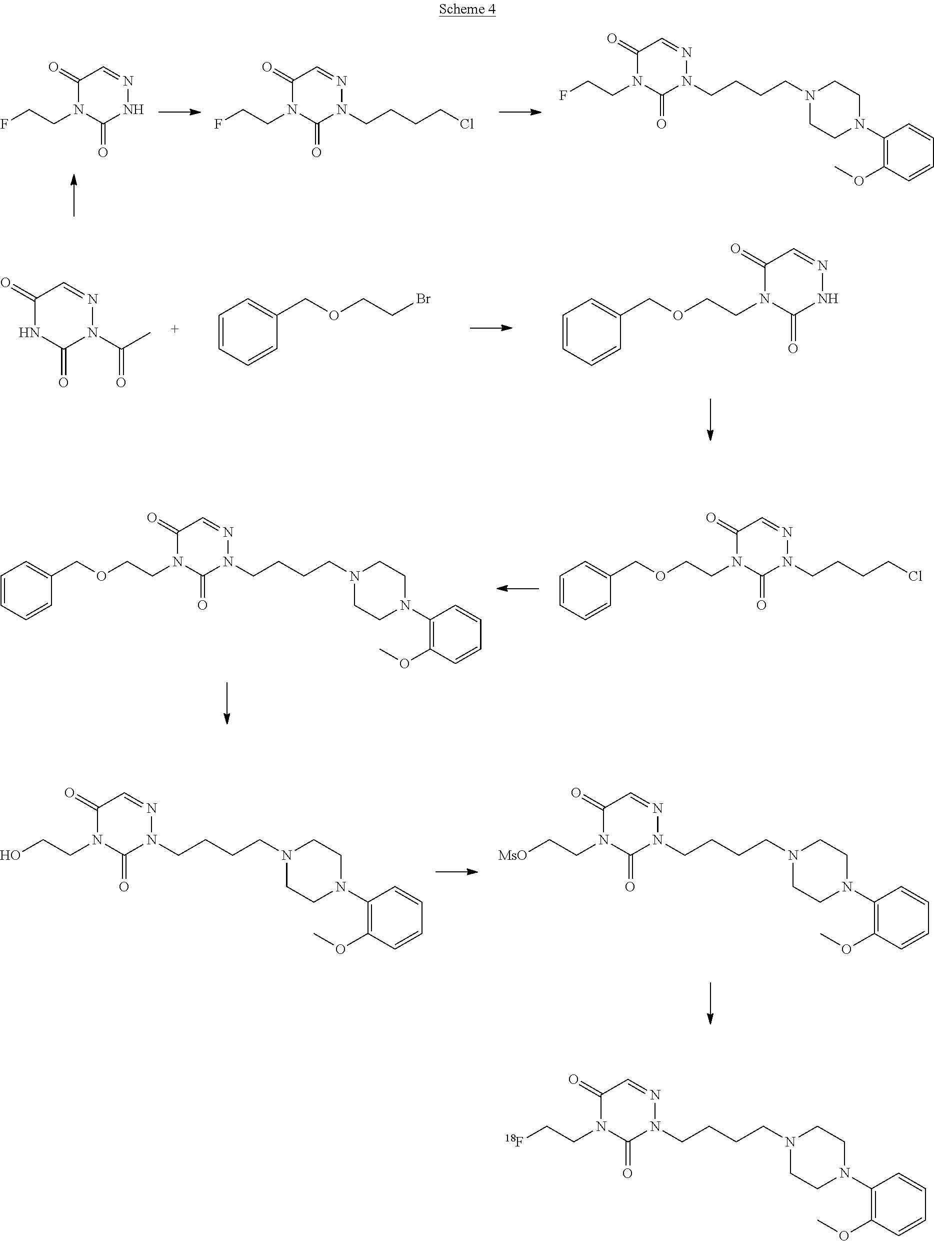

### Preparation of 4-(2-fluoroethyl)-1,2,4-triazine-3,5(2H,4H)-dione

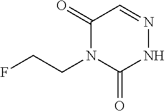 [SMILES: O=c1cn[nH]c(=O)n1CCF]

To a suspension of sodium hydride (0.131 g, 5.46 mmol) in dry DMF (7.5 mL) in a 50 mL round-bottomed flask was added a solution of 2-acetyl-1,2,4-triazine-3,5(2H,4H)-dione (0.77 g, 4.96 mmol) in dry DMF (7.5 mL) and the solution stirred under a balloon of N2 for 1 h. Fluoroethyl tosylate (1.2 g, 5.50 mmol) was added and the mixture stirred at ambient overnight. Evaporated most of the solvent and added ethanol (15 mL) and para-toluenesulfonic acid (0.1 g) and refluxed 2 h. Allowed to cool overnight. Evaporated to dryness and added water (50 mL) and extracted with dichloromethane (4×25 mL). Combined organics were washed with brine and phase separated and evaporated to give an oil (820 mg). Purified by column chromatography on silica gel (50 g) eluting with 0.5-10% methanol-dichloromethane over 20 CV to give the product as an impure viscous oil (176 mg, 22%).

¹H and ¹³C are consistent with slightly impure product.

### Preparation of 2-(4-chlorobutyl)-4-(2-fluoroethyl)-1,2,4-triazine-3,5(2H,4H)-dione

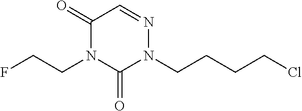 [SMILES: O=c1cnn(CCCCCl)c(=O)n1CCF]

4-(2-Fluoroethyl)-1,2,4-triazine-3,5(2H,4H)-dione (159 mg, 1 mmol) was dissolved in dry DMF (3 mL) and a solution of lithium hexamethyldisilazide (1.5 mL, 1N in hexanes) was added. After 10 mins at ambient temperature, 1-bromo-4-chlorobutane (0.17 mL, 260 mg, 1.5 mmol) was added and the mixture stirred at ambient temperature for 16 h. Diluted with water (15 mL) and extracted with ethyl acetate (3×5 mL). Combined organics were washed with water (3 mL), brine (3 mL), dried over sodium sulfate, filtered and evaporated to give 224 mg crude product. Purified by column chromatography on silica gel (10 g) eluting with 0.5-10% methanol-dischloromethane to give the product as a colourless oil (180 mg, 72%).

LCMS calcd for C₉H₁₃ClFN₃O₂: 249.1; found: 250.1 [M+H]⁺.

¹H, ¹³C and ¹⁹F NMR are consistent with the product structure.

### Preparation of 4-(2-fluoroethyl)-2-(4-(4-(2-methoxyphenyl)piperazin-1-yl)butyl)-1,2,4-triazine-3,5(2H,4H)-dione

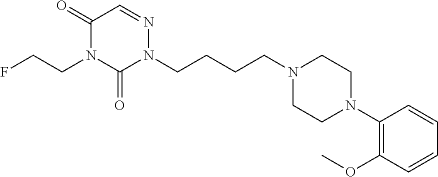 [SMILES: COc1ccccc1N1CCN(CCCCn2ncc(=O)n(CCF)c2=O)CC1]

2-(4-chlorobutyl)-4-(2-fluoroethyl)-1,2,4-triazine-3,5(2H,4H)-dione (90 mg, 0.36 mmol) and 1-(2-methoxyphenyl)piperazine (69.3 mg, 0.360 mmol) were dissolved in 1-butanol (2.5 mL) and triethylamine (0.3 mL) was added. The mixture was heated at 145° C. for 16 h. Evaporated to dryness and partitioned between water and ethyl acetate (20:20 mL), washing water with more ethyl acetate (2×10 mL). The combined organics were washed with brine and dried over sodium sulfate, filtered and evaporated to give crude product, which was purified by chromatography on silica gel (10 g) eluting with 0-5% methanol-dichloromethane.

LCMS calcd for C₂₀H₂₈FN₅O₃: 405.2; found: 406.1 [M+H]⁺.

¹H, ¹³C and ¹⁹F NMR consistent with product structure.

### Preparation of 4-(2-(Benzyloxy)ethyl)-1,2,4-triazine-3,5(2H,4H)-dione

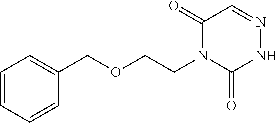 [SMILES: O=c1cn[nH]c(=O)n1CCOCc1ccccc1]

2-Acetyl-1,2,4-triazine-3,5(2H,4H)-dione (1 g, 6.45 mmol) was dissolved in DMF (10 mL) and added to a suspension of sodium hydride (310 mg, 60% dispersion in oil, 7.75 mmol) in DMF (10 mL) and the mixture stirred for 45 mins before addition of bromoethoxymethylbenzene (1.733 g, 8.06 mmol). The reaction was stirred for 48 h at ambient temperature. Diluted with water (80 mL) and extracted with ethyl acetate (3×30 mL). Combined organics were washed with water (30 mL), brine (30 mL), dried over sodium sulfate, filtered and evaporated. TLC (1:1 ethyl acetate:petrol) showed 1 major spot at Rf 0.6. Purified by chromatography on silica gel (50 g) eluting with 10-100% ethyl acetate-petrol gradient over14 CV. Appropriate fractions were evaporated to the product as a viscous oil (383 mg, 24%).

LCMS calc for C₁₂H₁₃N₃O₃:247.1; found 246.2 [M−H]⁻ in ES−.

¹H NMR (300 MHz, CDCl₃): δ 3.74 (2H, t, J=5.5 Hz, N—CH₂), 4.18 (2H, t, J=5.5 Hz, O—CH₂), 4.53 (2H, s, PhCH₂), 7.20-7.38 (5H, m, phenyl-H) and 9.70 (1H, br, NH). ¹³C NMR (75 MHz, CDCl₃): δ 39.2 (N—CH₂), 65.8 (CH₂—O), 72.7 (CH₂Ph), 127.7 and 128.4 (phenyl-C-2-5), 135.3 (N═CH), 137.8 (phenyl-C-1), 149.1 (N—C═O) and 156.0 (N—C(═O)NH).

### Preparation of 4-(2-(benzyloxy)ethyl)-2-(4-chlorobutyl)-1,2,4-triazine-3,5(2H,4H)-dione

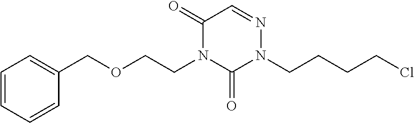 [SMILES: O=c1cnn(CCCCCl)c(=O)n1CCOCc1ccccc1]

4-(2-(Benzyloxy)ethyl)-1,2,4-triazine-3,5(2H,4H)-dione (383 mg, 1.549 mmol) was dissolved in DMF (5 mL) and added to a suspension of sodium hydride (80 mg, 60% dispersion in oil, 2.0 mmol) in DMF (5 mL) and the mixture stirred for 45 mins before addition of 1-bromo-4-chlorobutane (279 mg, 1.626 mmol). The reaction was stirred for 48 h at ambient temperature. Diluted with water (60 mL) and extracted with ethyl acetate (3×25 mL). Combined organics were washed with water (25 mL), brine (20 mL), dried over sodium sulfate, filtered and evaporated. TLC (1:1 ethyl acetate:petrol) showed 1 major spot at Rf 0.6. Purified by chromatography on silica gel (50 g) in 10-100% ethyl acetate-petrol gradient over 14 CV. Appropriate fractions evaporated to give the product as an oil (383 mg, 73%).

¹H NMR (300 MHz, CDCl₃): δ 1.75-1.97 (4H, m, CH₂—CH₂), 3.54 (2H, t, J=6.5 Hz, CH₂N-2), 3.74 (2H, t, J=5.5 Hz, CH₂N-4), 3.98 (2H, t, J=6.5 Hz, CH₂—Cl), 4.20 (2H, t, J=5.5 Hz, CH₂—O), 7.20-7.40 (5H, phenyl-H). ¹³C NMR (CDCl₃): δ 25.6 (CH₂—CH₂), 29.4 (CH₂—CH₂), 39.8 (CH₂—N-2), 44.3 (CH₂—N-4), 51.0 (CH₂—Cl), 66.0 (CH₂—O), 72.7 (CH₂-Ph), 127.8 and 128.5 (phenyl-C2-C6), 134.3 (CH═N), 138.0 (phenyl-C1), 148.7 (N—C═O) and 156.1 (NC(═O)N).

### Preparation of 4-(2-(benzyloxy)ethyl)-2-(4-(4-(2-methoxyphenyl)piperazin-1-yl)butyl)-1,2,4-triazine-3,5(2H,4H)-dione

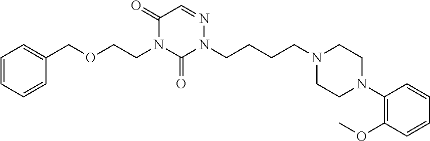 [SMILES: COc1ccccc1N1CCN(CCCCn2ncc(=O)n(CCOCc3ccccc3)c2=O)CC1]

4-(2-(benzyloxy)ethyl)-2-(4-chlorobutyl)-1,2,4-triazine-3,5(2H,4H)-dione (383 mg, 1.13 mmol) and 1-(2-methoxyphenyl)piperazine (217 mg, 1.13 mmol) were dissolved in 1-butanol (15 mL) and triethylamine (1.5 mL) added. The mixture was heated at 145° C. for 18 h. Cooled and evaporated then partitioned between dichloromethane-water (20:20 mL), separated with phase separator and evaporated. TLC (5% methanol-dichloromethane) showed 2 spots (major at Rf 0.3, and minor at Rf 0.8). Purified by column chromatography on silica gel (50 g) in 0.5-10% methanol-dichloromethane over 15 CV. Evaporated to give the product as a gum (284 mg, 60%).

LCMS calc for C₂₇H₃₅N₅O₄: 493.3; found 494.3 [M+H]⁺ in ES+.

¹H NMR (300 MHz, CDCl₃): δ 1.50-1.61 (2H, m, CH₂—CH₂), 1.72-1.84 (2H, m, CH₂—CH₂), 2.44 (2H, t, J=7.5 Hz, CH₂N), 2.58-2.72 (4H, br, pip-CH₂), 3.00-3.18 (4H, br, pip-CH₂), 3.74 (2H, t, J=5.5 Hz, CH₂—N), 3.85 (3H, s, OCH₃), 3.98 (2H, t, J=7.0 Hz, OCH₂CH₂N), 4.20 (2H, t, J=5.5 Hz, CH₂—O), 4.53 (2H, s, PhCH₂), 6.83-7.03 (4H, m, Ar—H), 7.23-7.35 (5H, phenyl-H) and 7.36 (1H, s, N═CH). ¹³C NMR (CDCl₃): δ 3.7 (CH₂—CH₂), 26.2 (CH₂—CH₂), 39.6 (CH₂—N-2), 50.6 (pip-CH₂), 51.7 (CH₂—N), 53.4 (pip-CH₂), 55.3 (O—CH₃), 58.0 (CH₂—N-4), 65.8 (CH₂—O), 72.6 (CH₂-Ph), 111.1 (methoxyphenyl-C-3), 118.1, 120.9, 122.9 (methoxyphenyl-C-4,5,6), 127.7 and 128.3 (phenyl-C2-C6), 133.9 (CH═N), 137.8 (phenyl-C1), 141.2 (methoxyphenyl-C-2), 148.6 (N—C═O), 152.2 (methoxyphenyl-C-1) and 156.0 (NC(═O)N).

### Preparation of 4-(2-hydroxyethyl)-2-(4-(4-(2-methoxyphenyl)piperazin-1-yl)butyl)-1,2,4-triazine-3,5(2H,4H)-dione

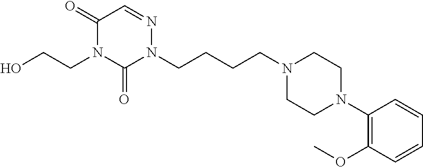 [SMILES: COc1ccccc1N1CCN(CCCCn2ncc(=O)n(CCO)c2=O)CC1]

4-(2-(benzyloxy)ethyl)-2-(4-(4-(2-methoxyphenyl)piperazin-1-yl)butyl)-1,2,4-triazine-3,5(2H,4H)-dione (284 mg, 0.575 mmol) was dissolved in methanol (11 mL) and passed through the H-Cube hydrogenator using a 10% Pd/C catalyst at 20 bar and 70° C. Solvent was evaporated and the product purified by chromatography on silica gel (10 g) eluting with 0.5-10% methanol-dichloromethane over 25 CV. Evaporation of fractions gave the product (72 mg, 31%).

LCMS calc for C₂₀H₂₉N₅O₄: 403.2; found 404.1 [M+H]⁺ in ES+.

¹H NMR (300 MHz, CDCl₃): δ 1.65-1.89 (4H, br m, CH₂—CH₂), 2.65 (2H, t, J=7.5 Hz, CH₂N), 2.89 (4H, br s, pip-CH₂), 3.19 (4H, br s, pip-CH₂), 3.85 (3H, s, OCH₃), 3.88 (2H, t, J=5.0 Hz, CH₂—N), 4.03 (2H, t, J=6.5 Hz, OCH₂CH₂N), 4.16 (2H, t, J=5.0 Hz, CH₂—O), 6.82-6.96 (4H, m, Ar—H), 6.97-7.06 (1H, m, Ar—H) and 7.41 (1H, s, N═CH). ¹³C NMR (CDCl₃): δ 22.3 (CH₂—CH₂), 25.8 (CH₂—CH₂), 43.1 (CH₂—N), 49.3 (pip-CH₂), 50.9 (CH₂—N), 52.8 (pip-CH₂), 55.3 (O—CH₃), 57.4 (CH₂—N4), 59.8 (CH₂—O), 111.1 (methoxyphenyl-C3), 118.4, 121.0, 123.4 (methoxyphenyl-C-4,5,6), 134.4 (CH═N), 140.3 (methoxyphenyl-C-2), 149.4 (N—C═O), 152.0 (NC(═O)N) and 156.4 (methoxyphenyl-C1).

### Preparation of precursor 2-(2-(4-(4-(2-Methoxyphenyl)piperazin-1-yl)butyl)-3,5-dioxo-2,3-dihydro-1,2,4-triazin-4(5H)-yl)ethyl methanesulfonate

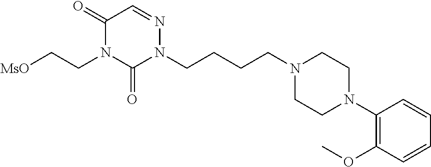 [SMILES: COc1ccccc1N1CCN(CCCCn2ncc(=O)n(CCOS(C)(=O)=O)c2=O)CC1]

4-(2-hydroxyethyl)-2-(4-(4-(2-methoxyphenyl)piperazin-1-yl)butyl)-1,2,4-triazine-3,5(2H,4H)-dione (45 mg, 0.11 mmol) is dissolved in dichloromethane (2 mL) and triethylamine (0.2 mL) added followed by methanesulfonyl chloride (13 mg, 0.11 mmol). The reaction mixture is stirred at ambient temperature overnight, diluted with dichloromethane and washed with dilute aqueous potassium carbonate solution, dried over sodium sulfate and evaporated. The product is isolated by chriomatography on silica gel to give pure 2-(2-(4-(4-(2-methoxyphenyl)piperazin-1-yl)butyl)-3,5-dioxo-2,3-dihydro-1,2,4-triazin-4(5H)-yl)ethyl methanesulfonate.

### Preparation of [¹⁸F]4-(2-Fluoroethyl)-2-(4-(4-(2-methoxyphenyl)piperazin-1-yl)butyl)-1,2,4-triazine-3,5(2H,4H)-dione

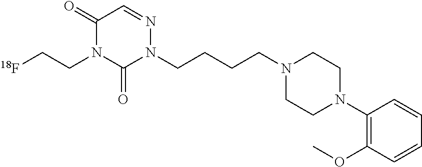 [SMILES: COc1ccccc1N1CCN(CCCCn2ncc(=O)n(CC[18F])c2=O)CC1]

[¹⁸F]fluoride is transferred from a P6 vial into a 3 mL V-vial by suction. To the P6 vial is added a pre-prepared solution of kryptofix 2.2.2 (5 mg, 1.3×10⁻⁵ mol) in acetonitrile (0.5 mL) and potassium carbonate (65 μL, 0.1M) made up to 0.5 mL with water. The P6 vial is agitated and the solution transferred to the V-vial by suction. The V-vial is heated to 110° C. for 20 min under a flow of nitrogen (0.2 L/min) then cooled to room temperature.

To the dried [¹⁸F]fluoride and kryptofix mixture is added 2-(2-(4-(4-(2-methoxyphenyl)piperazin-1-yl)butyl)-3,5-dioxo-2,3-dihydro-1,2,4-triazin-4(5H)-yl)ethyl methanesulfonate (2 mg) in acetonitrile (1 mL). The resulting solution is heated at 80° C.-100° C. for 10-20 min, and then cooled to room temperature. To the reaction vial is added water (1.5 mL) and the mixture loaded on to preparative HPLC for purification. The isolated HPLC fraction is diluted into water (20 mL) and then formulated for administration.

### Example 5

### Scheme 5 Shows a Method for Making Radiolabeled Compounds of Formula (I) for Preparation of 1-(2-(7-(2-Fluoroethoxy)-1,2,3,4-tetrahydronaphthalen-2-yl)ethyl)-4-(pyridin-2-yl)piperazine

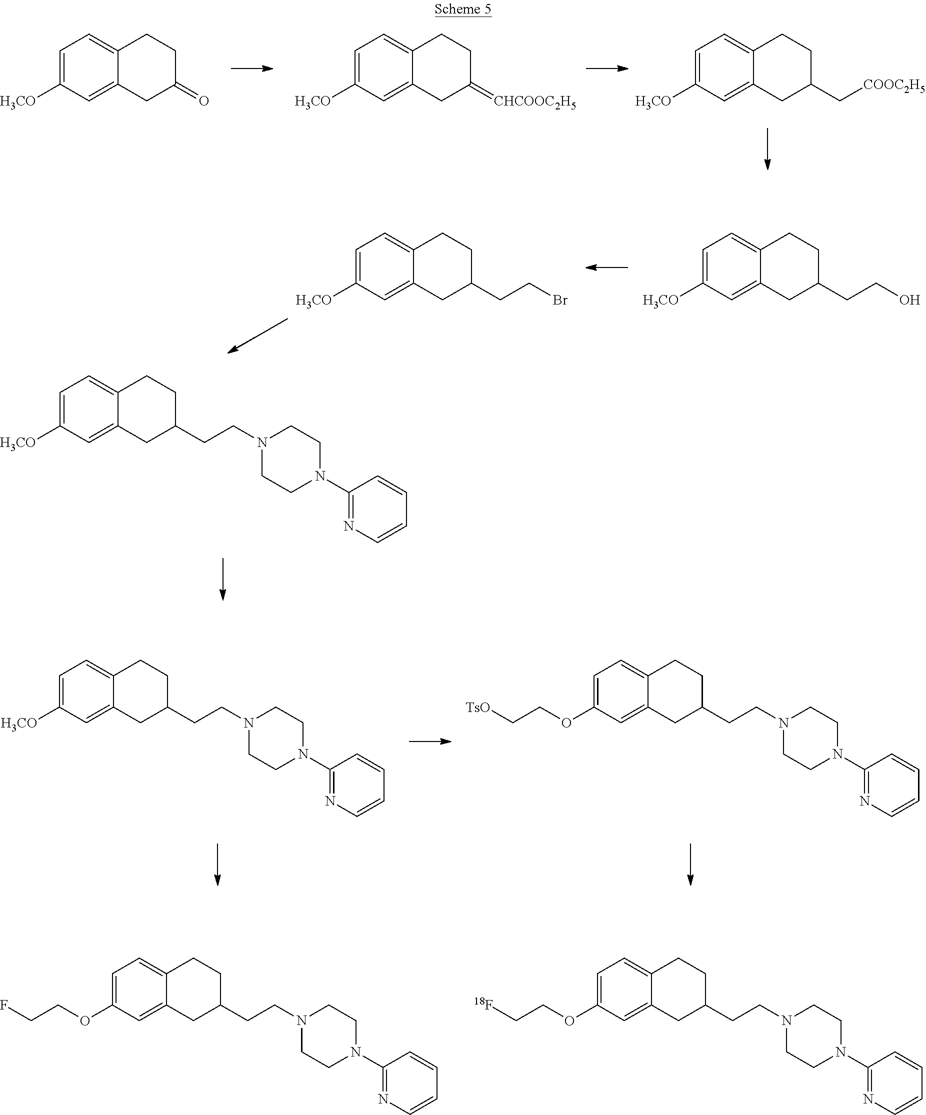

### Preparation of ethyl 2-(7-methoxy-3,4-dihydronaphthalen-2(1H)-ylidene)acetate

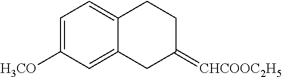

To a solution of triethylphosphoacetate (6.2 mL, 31.24 mmol) in 1,2-dimethoxyethane (100 ml) was added sodium hydride (1.25 g, 60% in oil, 31.24 mmol) in portions at room temperature and the mixture was stirred for 1 h at the same temperature. After addition of 7-methoxy-2-tetralone (5.0 g, 26.40 mmol), the mixture was stirred for 3 h at room temperature, poured onto ice water, and extracted with ethyl acetate (2×30 mL). The combined organic phases were dried over sodium sulfate, filtered and concentrated in vacuum to afford an oil, which was purified by chromatography on silica gel (hexane:ethyl actetate 10:1) to give the product as a colorless liquid (6.82 g, 97%).

LCMS calcd for C₁₅H₁₈O₃=246.1; found 247.11[M=H]⁺.

¹H-NMR (400 MHz, CDCl₃) δ=7.04 (1H, d, J=8 Hz), 6.68 (1H, d, J=8 Hz), 6.61 (1H, s), 6.33 (1H, s), 4.22-4.15 (2H, m), 3.80 (3H, s), 3.23 (2H, s), 2.80 (2H, t, J=8H), 2.36 (2H, t, J=8 Hz), 1.29 (3H, t, J=8 Hz).

### Preparation of ethyl 7-methoxy-1,2,3,4-tetrahydro-2-naphthalenecarboxylate

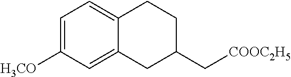

Ethyl 7-methoxy-3,4-dihydro-2-naphthalenecarboxylate (4.0 g, 16.25 mmol) was dissolved in methanol (20 mL) and then 10% Pd—C (400 mg, 10% w/w) added to the mixture. Hydrogenation reaction was then done using the Parr at normal temperature and pressure. The reaction mixture was then filtered through Celite, evaporated under reduced pressure and purified by chromatography on silica gel eluting with hexane:ethyl acetate 10:1 to obtain the product as a colorless liquid (3.0 g, 75%),

LCMS calcd for C₁₅H₂₀O₃=248.1; found 249.1 [M+H]⁺.

¹H-NMR (400 MHz, CDCl₃) δ=7.00 (1H, d, J=12 Hz), 6.70 (1H, d, J=8 Hz), 6.61 (1H, s), 4.22-4.10 (2H, m), 3.78 (3H, s), 2.89 (1H, dd, J1=4 Hz, J2=16 Hz), 2.81-2.75 (2H, m), 2.55-2.45 (1H, m), 2.37 (2H, d, J=4 Hz), 2.27 (1H, bs), 1.96 (1H, d, J=16 Hz), 1.53-1.41 (1H, m), 1.32-1.25 (3H, m).

### Preparation of 2-(7-Methoxy-1,2,3,4-tetrahydronaphthalen-2-yl)ethanol

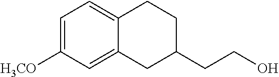 [SMILES: COc1ccc2c(c1)CC(CCO)CC2]

To an oven dried round bottom 2-neck flask was added lithium aluminum hydride (0.61 g, 16.1 mmol) in anhydrous tetrahydrofuran (20 mL) and the mixture cooled in an ice-bath. Ethyl 7-methoxy-1,2,3,4-tetrahydro-2-naphthalenecarboxylate (2.0 g, 8.1 mmol) was added to the reaction mixture keeping at 0° C. The reaction mixture was then allowed to stir at room temperature for 3 h and quenched with ice-cold water (20 ml). The mixture was filtered using Celite and the solvent was evaporated under reduced pressure, partitioned between dichloromethane and water. The combined organic layers were then dried over anhydrous sodium sulfate, filtered and evaporated to give the product (1.75 g, 54%).

LCMS calcd for C₁₃H₁₈O₂=206.1; found 205.1 ([M−H]⁻.

¹H-NMR: (500 MHz, CDCl₃) δ=7.01 (1H, d, J=10 Hz), 6.70 (1H, dd, J=10 Hz), 6.63 (1H, d, J=3 Hz), 3.86-3.75 (5H, m), 2.95-2.70 (3H, m), 2.47 (1H, m), 2.00-1.82 (2H, m), 1.79-1.56 (3H, m), 1.51-1.36 (2H, m).

### Preparation of 2-(2-Bromoethyl)-7-methoxy-1,2,3,4-tetrahydronaphthalene

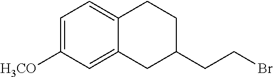 [SMILES: COc1ccc2c(c1)CC(CCBr)CC2]

Anhydrous toluene (17 mL) was added to 2-(7-Methoxy-1,2,3,4-tetrahydronaphthalen-2-yl)ethanol (1.75 g, 8.5 mmol) in a round bottom flask, followed by addition of pyridine (0.112 ml) at 0° C. Phosphorus tribromide (2.2 ml) was then added into the reaction mixture and heated at 70° C. for 12 h. The reaction mixture was then poured into water (20 ml) and extracted with ethyl acetate (3×15 mL). The combined organic layers were dried over sodium sulfate, filtered and evaporated under reduced pressure to give the desired product (940 mg, 42%).

LCMS calcd for C₁₃H₁₇BrO=268.1; found 269.4 [M+H]⁺.

¹H-NMR: (300 MHz, CDCl₃) δ=7.01 (1H, d, J=3, 6 Hz), 6.71 (1H, dd, J=9 Hz), 6.63 (1H, d), 3.79 (3H, s), 3.55 (2H, t, J=9 Hz), 3.01-2.70 (4H, m), 2.60-2.34 (2H, m), 2.05-1.90 (4H, m), 1.55-1.33 (2H, m).

### Preparation of 1-(2-(7-Methoxy-1,2,3,4-tetrahydronaphthalen-2-yl)ethyl)-4-(pyridine-2-yl)piperazine

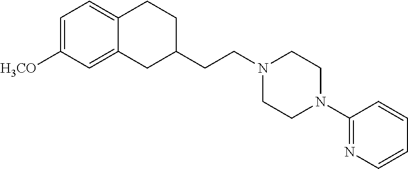 [SMILES: COc1ccc2c(c1)CC(CCN1CCN(c3ccccn3)CC1)CC2]

2-(2-Bromoethyl)-7-methoxy-1,2,3,4-tetrahydronaphthalene (940 mg, 3.4 mmol) was dissolved in acetonitrile (9.4 ml) in an oven dried round bottom flask. potassium carbonate (0.965 mg, 6.9 mmol) was then added followed by 1-(pyridin-2-yl)piperazine (0.855 mg, 5.2 mmol). The reaction mixture was allowed to stir at 80° C. for 12 h. Quenched by addition of water (15 ml) and extracted with ethyl acetate (4×20 mL). The combined organic layers were dried over anhydrous sodium sulfate, filtered and evaporated under reduced pressure. The crude reaction mass was then purified by chromatography on silica gel eluting with 40% ethyl acetate in hexane to give the desired product (1.2 g, 100%).

LCMS calcd for C₂₂H₂₉N₃O=351.2; found 352.4 ([M+H]⁺.

¹H-NMR: (500 MHz, CDCl₃) δ=8.17 (1H, s), 7.50 (1H, s), 7.01 (1H, d, J=10 Hz), 6.65 (4H, m), 3.79 (3H, s), 3.59 (4H, s), 2.75 (2H, bs), 2.60 (4H, bs), 2.51 (3H, bs), 1.94 (1H, bs), 1.77 (2H, bs), 1.62 (2H, bs), 1.42 (1H, s).

### Preparation of 7-(2-(4-(Pyridin-2-yl)piperazin-1-yl)ethyl)-5,6,7,8-tetrahydronaphthalen-2-ol

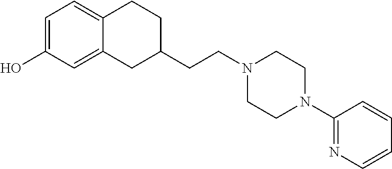 [SMILES: Oc1ccc2c(c1)CC(CCN1CCN(c3ccccn3)CC1)CC2]

1-(2-(7-methoxy-1,2,3,4-tetrahydronaphthalen-2-yBethyl)-4-(pyridin-2-yl)piperazine (1.2 g, 3.4 mmol) was dissolved in anhydrous dichloromethane (12 mL) and cooled to −78° C. Boron tribromide (931 mg, 3.7 mmol) was then added to the reaction mixture and then stirred and allowed to come to room temperature for 12 h. The reaction mixture was then quenched by addition of water. The aqueous layer was basified by addition of saturated sodium bicarbonate solution and extracted with dichloromethane (3×20 mL). The combined organic extracts were dried over anhydrous sodium sulfate, filtered and evaporated under reduced pressure to give the desired product (300 mg, 27%).

LCMS calcd for C₂₁H₂₇N₃O=337.2; found 338.4 [M+H]⁺.

### Preparation of 1-(2-(7-(2-Fluoroethoxy)-1,2,3,4-tetrahydronaphthalen-2-yl)ethyl)-4-(pyridin-2-yl)piperazine

7-(2-(4-(Pyridin-2-yl)piperazin-1-yBethyl)-5,6,7,8-tetrahydronaphthalen-2-ol (1 mmol) was dissolved in anhydrous dimethylformamide/acetonitrile (10 mL) and cesium carbonate (1.1 mmol) added. The whole reaction mass was allowed to stir for about 5 minutes then fluoroethyl tosylate (1.1 mmol) was added to. The reaction was heated at 55° C. for 12 h, quenched with water and extracted with ethyl acetate. The product was purified by chromatography on silica gel eluting with hexane and ethyl acetate (50%) to give the desired compound in a yield of 16%.

LCMS Calcd for C₂₃H₃₀FN₃O=383.2; found 384.4 [M+H]⁺.

¹H-NMR (500 MHz, CDCl₃) δ=8.22 (1H, d, J=5 Hz), 7.51 (1H, t, J=5 Hz), 7.02 (1H, d, J=5 Hz), 6.76-6.60 (m, 4H), 4.81 (1H, t, J=5 Hz), 4.72 (1H, t, J=5 Hz), 4.23 (1H, t, J=5 Hz), 4.17 (1H, t, J=5 Hz), 3.59 (4H, t, J=5 Hz), 2.90-2.68 (3H, m) 2.62 (4H, t, J=5 Hz), 2.57-2.43 (3H, m), 1.96 (1H, d, J=10 Hz), 1.85-1.73 (bs, 2H), 1.50-1.36 (m, 2H).

### Preparation of precursor 2-(7-(2-(4-(pyridin-2-yl)piperazin-1-yl)ethyl)-5,6,7,8-tetrahydronaphthalen-2-yloxy)ethyl 4-methylbenzenesulfonate

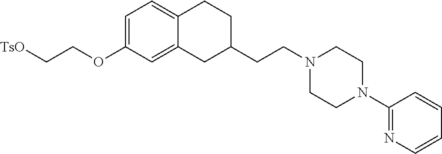 [SMILES: Cc1ccc(S(=O)(=O)OCCOc2ccc3c(c2)CC(CCN2CCN(c4ccccn4)CC2)CC3)cc1]

7-(2-(4-(Pyridin-2-yl)piperazin-1-yl)ethyl)-5,6,7,8-tetrahydronaphthalen-2-ol (1 mmol) was dissolved in anhydrous dimethylformamide/acetonitrile (10 mL) and cesium carbonate (1.1 mmol) added. The whole reaction mass was allowed to stir for about 5 minutes then fluoroethyl tosylate (1.1 mmol) was added to. The reaction was heated at 55° C. for 12 h, quenched with water and extracted with ethyl acetate. The product was purified by chromatography on silica gel eluting with hexane and ethyl acetate (50%) to give the desired compound in a yield of 8%.

LCMS calcd for C₃₀H₃₇N₃O₄S=535.3; found 536.2 [M+H]⁺.

¹H-NMR (300 MHz, CDCl₃) δ=8.19 (1H, dd, J1=3 Hz, J2=6 Hz), 7.52-7.45 (1H, m), 6.94 (1H, d, J=9 Hz), 6.68-6.60 (2H, m), 6.55 (1H, dd, J1=3 Hz, J2=6 Hz), 6.48 (1H, d, J=3 Hz), 4.37-4.32 (2H, m), 4.14-4.08 (2H, m), 3.57 (4H, t, J=3 Hz), 2.85-2.68, (3H, m), 2.60 (4H, t, J=3 Hz), 2.55-2.36 (3H, m), 1.99-1.87 (2H, m), 1.66-1.56 (2H, m), 1.48-1.36 (1H, m).

### Preparation of [¹⁸F]1-(2-(7-(2-Fluoroethoxy)-1,2,3,4-tetrahydronaphthalen-2-yl)ethyl)-4-(pyridin-2-yl)piperazine

Potassium bicarbonate (0.7 mg in 50 μL H₂O) was added to kryptofix (5.0 mg) and anhydrous acetonitrile (0.50 mL) in a 3 mL Wheaton vial equipped with a stirrer vane. [¹⁸F]-fluoride (aq., 358 MBq in ˜50 μL) was added to the vial, and heated to 110° C. under a stream of N₂ to azeotropically dry the fluoride. Two further portions of anhydrous acetonitrile (2×0.5 mL) were added and similarly dried. The reaction vial was cooled to room temperature, and the tosylate precursor (2.0 mg) in anhydrous acetonitrile (200 μL) was added. The reaction was stirred at 90° C. for 5 min. The reaction was diluted with acetonitrile (0.6 mL) and H₂O (1.0 mL) and loaded to a semi-preparative HPLC system. The product was collected using a manual switch, and was diluted with H₂O to a total volume of 20 mL, and loaded to a tC18 Light Sep-pak cartridge (primed with 1 mL ethanol and 2 mL H₂O). The product was eluted with ethanol (0.5 mL) and diluted with phosphate buffered saline (5.5 mL). Yield=63 MBq (17.6%). RCP >99%. SA=3 GBq/μmol.

| | Semi-preparative | Analytical |
|---|---|---|
| Column | ACE-5 C18, 10 × 100 mm | Phenomenex Luna C18, |
| | | 5μ, 4.6 × 150 mm |
| Eluant | A = H₂O | A = H₂O |
| | B = MeOH | B = MeOH |
| Gradient | 0-25 min 20-95% B | 0-1 min 50% B |
| | 25-28 min 95-20% B | 1-15 min 50-95% B |
| | | 15-20 min 95% B |
| Flow rate | 3.0 | 1.0 |
| (mL/min) | | |
| Retention | 24.7 | 14.0 |
| time (min) | | |

Compounds that were synthesized following procedures that were similar or identical as described those in Example 5 include the following:

| Structure | Name | [M + H]⁺ |
|---|---|---|
| 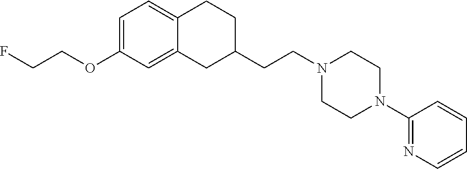 [SMILES: FCCOc1ccc2c(c1)CC(CCN1CCN(c3ccccn3)CC1)CC2] | 1-(2-(7-(2- fluoroethoxy)- 1,2,3,4- tetrahydronaphthalen- 2-yl)ethyl)-4- (pyridin-2- yl)piperazine | 384.4 |
| 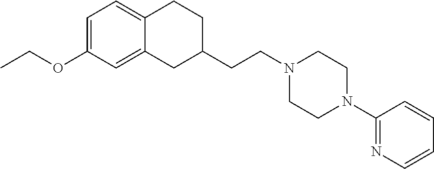 [SMILES: CCOc1ccc2c(c1)CC(CCN1CCN(c3ccccn3)CC1)CC2] | 1-(2-(7-ethoxy- 1,2,3,4- tetrahydronaphthalen- 2-yl)ethyl)-4- (pyridin-2- yl)piperazine | 366.4 |
| 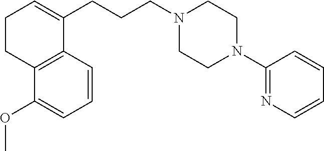 [SMILES: COc1cccc2c1CCC=C2CCCN1CCN(c2ccccn2)CC1] | 1-(3-(5-methoxy-3,4- dihydronaphthalen-1- yl)propyl)-4-(pyridin- 2-yl)piperazine | 364.4 |
| 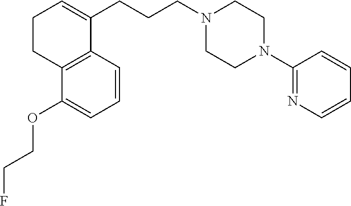 [SMILES: FCCOc1cccc2c1CCC=C2CCCN1CCN(c2ccccn2)CC1] | 1-(3-(5-(2- fluoroethoxy)-3,4- dihydronaphthalen-1- yl)propyl)-4-(pyridin- 2-yl)piperazine | 396.5 |
| 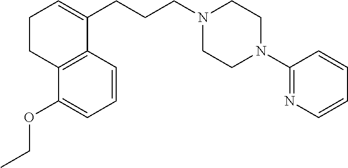 [SMILES: CCOc1cccc2c1CCC=C2CCCN1CCN(c2ccccn2)CC1] | 1-(3-(5-ethoxy-3,4- dihydronaphthalen-1- yl)propyl)-4-(pyridin- 2-yl)piperazine | 378.5 |
| 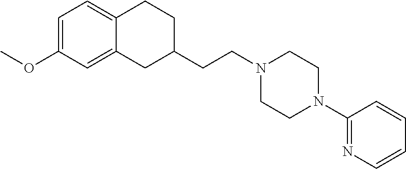 [SMILES: COc1ccc2c(c1)CC(CCN1CCN(c3ccccn3)CC1)CC2] | 1-(2-(7-methoxy- 1,2,3,4- tetrahydronaphthalen- 2-yl)ethyl)-4- (pyridin-2- yl)piperazine | 352.4 |
| 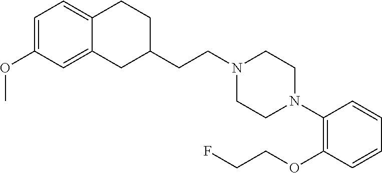 [SMILES: COc1ccc2c(c1)CC(CCN1CCN(c3ccccc3OCCF)CC1)CC2] | 1-(2-(2- fluoroethoxy)phenyl)- 4-(2-(7-methoxy- 1,2,3,4- tetrahydronaphthalen- 2-yl)ethyl)piperazine | 413.4 |
| 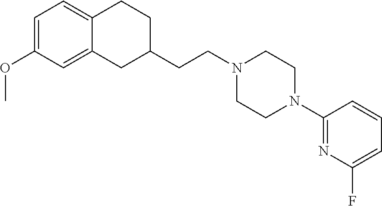 [SMILES: COc1ccc2c(c1)CC(CCN1CCN(c3cccc(F)n3)CC1)CC2] | 1-(6-fluoropyridin-2- yl)-4-(2-(7-methoxy- 1,2,3,4- tetrahydronaphthalen- 2-yl)ethyl)piperazine | 370.4 |
| 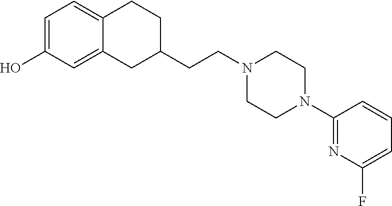 [SMILES: Oc1ccc2c(c1)CC(CCN1CCN(c3cccc(F)n3)CC1)CC2] | 7-(2-(4-(6- fluoropyridin-2- yl)piperazin-1- yl)ethyl)-5,6,7,8- tetrahydronaphthalen- 2-ol | 356.3 |

### Example 6

### Scheme 6 Shows a Method for Making Radiolabeled Compounds of Formula (I) for Preparation of 1-(4-(3-(2-Fluoroethoxy)phenyl)cyclohexyl)-4-(2-fluorophenyl)piperazine

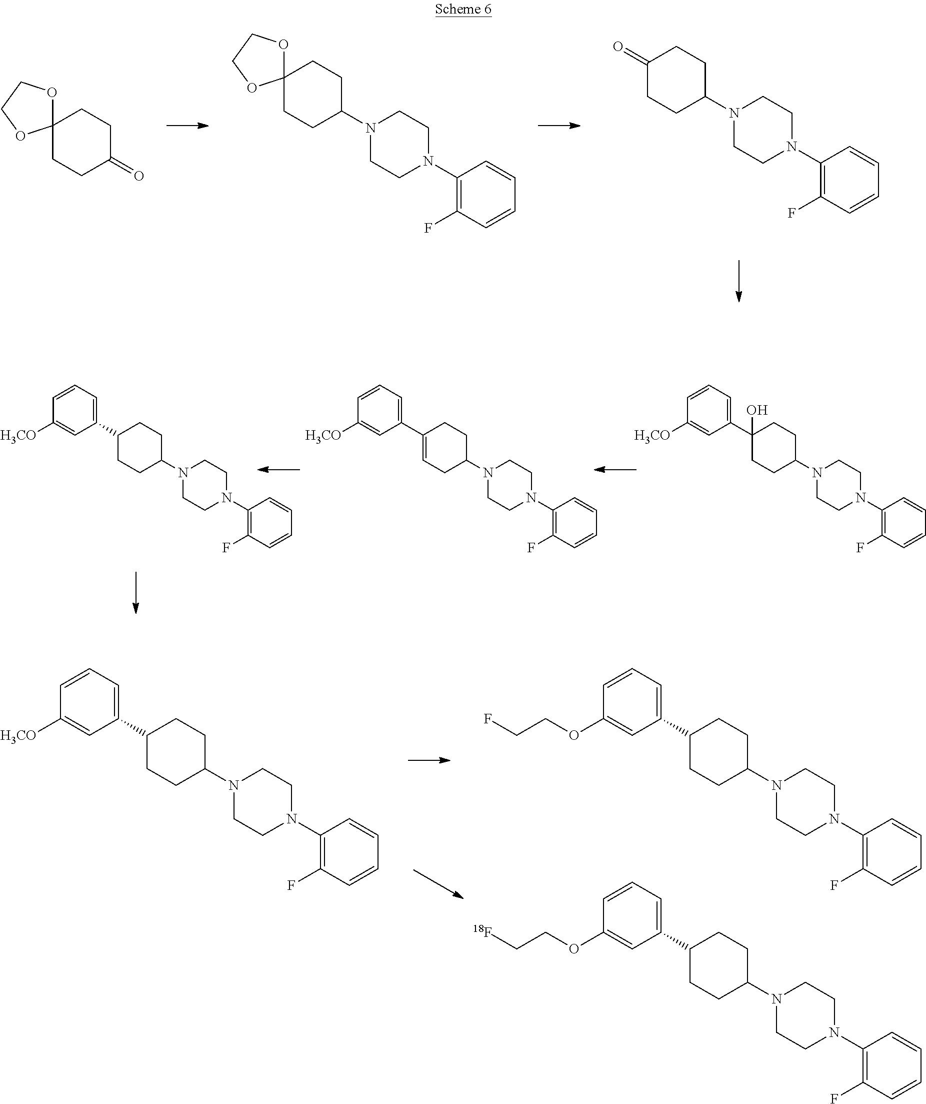

### Preparation of 1-(2-Fluorophenyl)-4-(1,4-dioxaspiro[4.5]decan-8-yl)piperazine

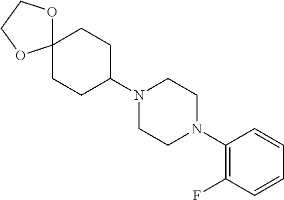 [SMILES: Fc1ccccc1N1CCN(C2CCC3(CC2)OCCO3)CC1]

To 1,4-cyclohexanedione-monoethylene ketal (5 g, 32 mmol) suspended in toluene, 1-(2-fluorophenyl)piperazine (6.95 g, 38.4 mmol) and p-toluenesulfonicacid (0.61 g, 3.26 mmol) were added. The reaction mixture was refluxed overnight using a Dean-Stark apparatus. The solvent was distilled out and 150 ml of anhydrous tetrahydrofuran was added to the resulting solid enamine. Sodium triacetoxyborohydride (8.102 g; 38.2 mmol) was added in portions. Acetic acid (2.74 ml; 48 mmol) was also added and the reaction mixture was stirred overnight at room temperature. Tetrahydrofuran was distilled out to give a brown colored residue. 1N aqueous sodium hydroxide was added to this residue to pH9. It was then extracted with ethyl acetate. The organic layer was separated, dried over sodium sulfate, filtered and evaporated out to give the product as a brown solid (11 g, 98%)

LCMS calcd for C₁₈H₂₅FN₂O₂=320.2; found 321.2 [M+H]⁺.

¹H NMR: (300 MHz, CDCl₃) δ=7.1-6.9 (4H, m), 3.93 (4H, s), 3.24 (4H, t, J=6 Hz), 3.03 (4H, t, J=6 Hz), 2.85 (1H, m), 1.45-1.85 (8H, m).

### Preparation of 4-(4-(2-Fluororophenyl)piperazine-1-yl)cyclohexanone

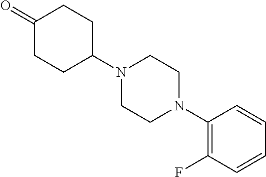 [SMILES: O=C1CCC(N2CCN(c3ccccc3F)CC2)CC1]

3N aqueous hydrochloric acid was added dropwise to a solution of 1-(2-fluorophenyl)-4-(1,4-dioxaspiro[4.5]decan-8-yl)piperazine (11 g, 34.26 mmol) in methanol (100 ml) at room temperature over a period of half an hour. The reaction mixture was stirred overnight at room temperature. Solvents were distilled out to give a crude brown colored residue. To this residue, 20% sodium carbonate solution was added to pH 9. This was then extracted with dichloromethane. The organic layer was separated, dried over sodium sulfate and evaporated to give the crude product. This was purified by chromatography on silica gel eluting with 5% methanol in dichloromethane to give the pure product (7.6 g, 80%).

LCMS calcd for C₁₆H₂₁FN₂O=276.2: found 277.1 [M+H]⁺.

¹H-NMR (300 MHz, CDCl₃) δ=7.04-6.82 (4H, m), 3.07 (4H, t, J=6 Hz), 2.78 (4H, t, J=6 Hz), 2.76-2.52 (1H, m), 2.51-2.39 (2H, m), 2.34-2.20 (2H, m), 2.09-1.76 (4H, m).

### Preparation of 4-(4-(2-fluorophenyl)piperazin-1-yl)-1-(3-methoxyphenyl)cyclohexanol

To 4-(4-(2-fluororophenyl)piperazine-1-yl)cyclohexanone (2.0 g, 7.24 mmol) in anhydrous tetrahydrofuran, 3-methoxyphenylmagnesium bromide (14.47 mL, 14.47 mmol) was added dropwise over a period of 20 minutes and the reaction mixture was refluxed overnight. The reaction mixture was cooled to room temperature and solvent was evaporated to give a brownish mass. To this, a cooled saturated solution of ammonium chloride (25 mL) was added. The product was then extracted into diethylether. The organic layer was separated, dried over sodium sulfate and evaporated to give crude 4-[1-(3-methoxyphenyl)cyclohexanehydroxy-4-yl]-1-(2-fluorophenyl)piperazine (3.36 g).

LCMS calcd for C₂₄H₂₉F₂N₂O₂=384.2; found 385.2 [M+H]⁺.

### Preparation of 1-(2-fluorophenyl)-4-(3′-methoxy-2,3,4,5-tetrahydro-[1,1′-biphenyl]-4-yl)piperazine

The crude 4-[1-(3-methoxyphenyl)cyclohexanehydroxy-4-yl]-1-(2-fluorophenyl)piperazine was taken in a 100 ml round bottom flask to which 20% aqueous sulfuric acid solution (50 ml) was added dropwise. The reaction mixture was heated at 70° C. overnight. The reaction mixture was basified using 10% sodium hydroxide solution to pH 8-9 and then extracted using dichloromethane. The organic layer was separated, dried over sodium sulfate and evaporated to give the crude product. This was purified by chromatography on silica gel eluting with 80% ethyl acetate in hexane to give the pure product (1.3 g, 50%).

LCMS calcd for C₂₃H₂₇FN₂O=366.2; found 367.2 [M+H]⁺.

¹H NMR (300 MHz, CDCl₃) δ=7.22 (1H, d, J=3 Hz), 7.1-6.9 (6H, m), 6.78 (1H, dd, J1=3 Hz, J2=9 Hz), 6.09 (1H, t, J=3 Hz), 3.82 (3H, s), 3.17 (4H, t, J=6 Hz), 2.91-2.78 (4H, m), 2.62-2.41 (2H, m), 2.39-2.12 (2H, m), 1.73-1.54 (3H, m).

### Preparation of trans-1-(2-Fluorophenyl)-4-(4-(3-methoxyphenyl)cyclohexyl)piperazine

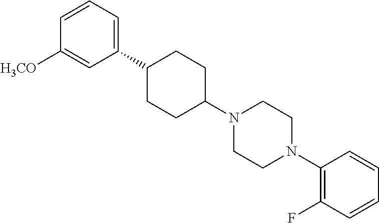

1-(2-fluorophenyl)-4-(3′-methoxy-2,3,4,5-tetrahydro-[1,1′-biphenyl]-4-yl)piperazine (1.3 g, 3.53 mmol) was dissolved in a 1:1 mixture of methanol and ethyl acetate (100 ml). The mixture was subjected to hydrogenation in the Parr shaker using 40 psi pressure and 200 mg 10% Pd—C catalyst overnight. The mixture was filtered through a Celite bed; the solution was then evaporated. The isomers were separated by chromatography on silica gel eluting with 10% ethyl acetate in hexane to isolate the cis-isomer and 15% ethyl acetate in hexane giving the required trans-isomer (190 mg, 14%).

LCMS calcd for C₂₃H₂₉FN₂O=368.2; found 369.2 [M+H]⁺.

¹H NMR (300 MHz; CDCl₃) δ=7.20-7.10 (1H, m), 7.04-6.82 (4H, m), 6.77-6.63 (3H, m), 3.73 (s, 3H), 3.08 (4H, t, J=6 Hz), 2.76 (4H, t, J=6 Hz), 2.48-2.30 (2H, m), 2.07-1.89 (4H, m), 1.54-1.32 (4H, m).

### Preparation for trans-3-(4-(4-(2-Fluorophenyl)piperazin-1-yl)cyclohexyl)phenol

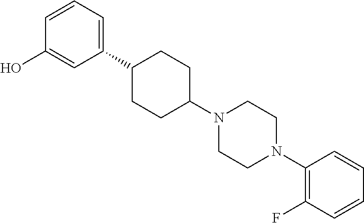

trans-1-(2-fluorophenyl)-4-(4-(3-methoxyphenyl)cyclohexyl)piperazine (190 mg, 0.51 mmol) was dissolved in dichloromethane (10 mL) and the reaction mixture was cooled to −78° C. Boron tribromide (0.14 g, 0.56 mmol) was added and the mixture was allowed to stir at same temperature for half an hour. The reaction mixture was allowed to stir overnight at room temperature and then washed with saturated sodium bicarbonate solution to pH8. The organic layer was separated, dried over sodium sulfate and evaporated to give the product (130 mg, 78%). This was used directly for the next step.

LCMS calcd for C₂₂H₂₇FN₂O=354.2; found=355.2 [M+H]⁺.

### Preparation of trans-4-(3-(2-Fluoroethoxy)phenyl)cyclohexyl)-4-(2-fluorophenyl)piperazine

To crude trans-3-(4-(4-(2-fluorophenyl)piperazin-1-yl)cyclohexyl)phenol (120 mg, 0.338 mmol) dissolved in 1:1 acetonitrile and dimethylformamide (10 mL), cesium carbonate (0.165 g, 0.506 mmol) was added and the reaction mixture was stirred at room temperature for 5 minutes. Fluoroethyl tosylate (0.111 g, 0.508 mmol) was then added and the reaction mixture was stirred overnight at 55° C. Acetonitrile was distilled out and water (20 mL) was added, the aqueous layer was extracted with dichloromethane (3×10 mL), washed thoroughly with water and dried over sodium sulfate and evaporated to give the crude product. This was purified by chromatography on silica gel eluting with 15% ethyl acetate in hexane to give the product (60 mg, 46%.

LCMS calcd for C₂₄H₃₀F₂N₂O=400.2; found 401.2 [M+H]⁺.

¹H NMR: (500 MHz; CDCl₃) δ=7.18-7.12 (1H, m), 7.02-6.84 (4H, m), 6.79-6.72 (2H, m), 6.7-6.66 (1H, m), 4.73 (1H, t, J=5 Hz), 4.64 (1H, t, J=5 Hz), 4.17 (1H, t, J=5 Hz); 4.11 (1H, t, J=5 Hz), 3.07 (4H, t, J=5 Hz), 2.74 (4H, t, J=5 Hz), 2.45-2.30 (2H, m), 2.05-1.89 (4H, m), 1.51-1.31 (4H, m).

### Preparation of [¹⁸F]trans-1-(4-(3-(2-Fluoroethoxy)phenyl)cyclohexyl)-4-(2-fluorophenyl)piperazine

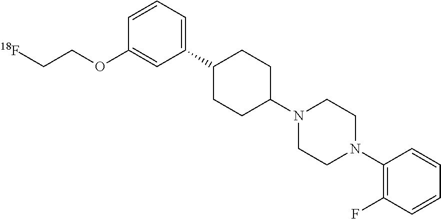

trans-1-(2-fluorophenyl)-4-(4-(3-methoxyphenyl)cyclohexyl)piperazine (5 mg) in DMF (0.1 mL) is stirred in the presence of cesium carbonate for 10 min, when dried [¹⁸F]fluoroethyl tosylate (prepared as previously described) was eluted into the reaction vial with DMF (0.4 mL) and the reaction mixture was stirred at 120° C. for 10 min. The crude reaction mixture was diluted into water (3 mL) and purified by preparative HPLC and formulated with ethanol and phosphate buffered saline for administration.

Compounds that were synthesized following procedures that were similar or identical as described those in Example 6 include the following:

| Structure | Name | [M + H]⁺ |
|---|---|---|
| 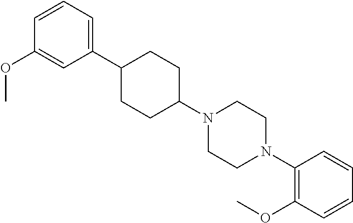 [SMILES: COc1cccc(C2CCC(N3CCN(c4ccccc4OC)CC3)CC2)c1] | 1-(2-methoxyphenyl)-4-(4-(3- methoxyphenyl)cyclohexyl)piperazine | 381.5 |
| 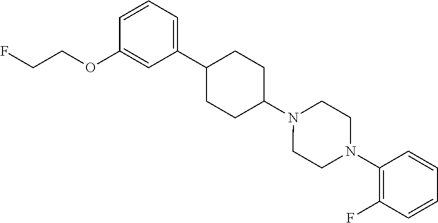 [SMILES: FCCOc1cccc(C2CCC(N3CCN(c4ccccc4F)CC3)CC2)c1] | 1-(4-(3-(2- fluoroethoxy)phenyl)cyclohexyl)-4- (2-fluorophenyl)piperazine | 401.4 |
| 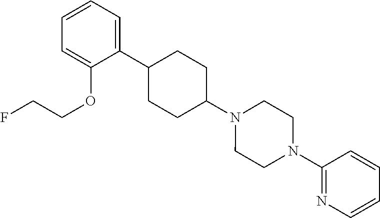 [SMILES: FCCOc1ccccc1C1CCC(N2CCN(c3ccccn3)CC2)CC1] | 1-(4-(2-(2- fluoroethoxy)phenyl)cyclohexyl)-4- (pyridin-4-yl)piperazine | 384.4 |
| 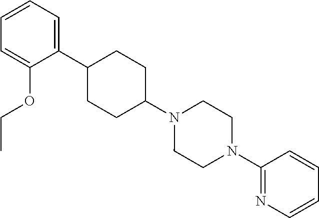 [SMILES: CCOc1ccccc1C1CCC(N2CCN(c3ccccn3)CC2)CC1] | 1-(4-(2-ethoxyphenyl)cyclohexyl)-4- (pyridin-2-yl)piperazine | 366.4 |
| 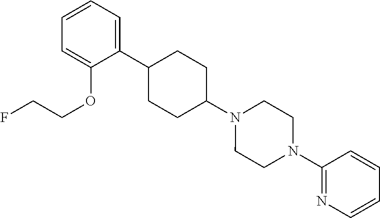 [SMILES: FCCOc1ccccc1C1CCC(N2CCN(c3ccccn3)CC2)CC1] | 1-(4-(2-(2- fluoroethoxy)phenyl)cyclohexyl)-4- (pyridin-2-yl)piperazine | 384.4 |
| 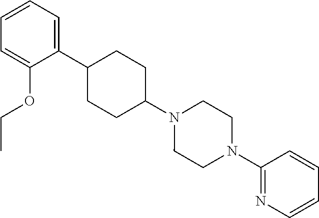 [SMILES: CCOc1ccccc1C1CCC(N2CCN(c3ccccn3)CC2)CC1] | 1-(4-(2-ethoxyphenyl)cyclohexyl)-4- (pyridin-2-yl)piperazine | 366.4 |
| 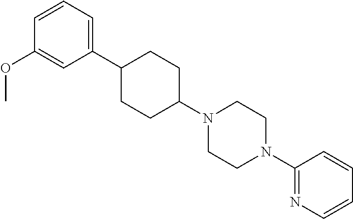 [SMILES: COc1cccc(C2CCC(N3CCN(c4ccccn4)CC3)CC2)c1] | 1-(4-(3-methoxyphenyl)cyclohexyl)- 4-(pyridin-2-yl)piperazine | 352.4 |
| 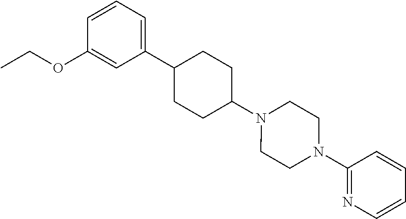 [SMILES: CCOc1cccc(C2CCC(N3CCN(c4ccccn4)CC3)CC2)c1] | 1-(4-(3-ethoxyphenyl)cyclohexyl)-4- (pyridin-2-yl)piperazine | 366.4 |
| 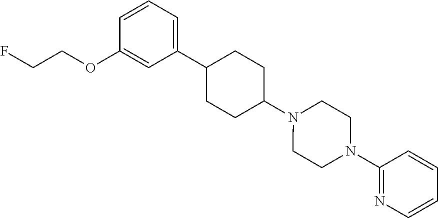 [SMILES: FCCOc1cccc(C2CCC(N3CCN(c4ccccn4)CC3)CC2)c1] | 1-(4-(3-(2- fluoroethoxy)phenyl)cyclohexyl)-4- (pyridin-2-yl)piperazine | 384.4 |
| 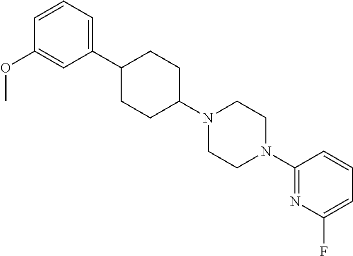 [SMILES: COc1cccc(C2CCC(N3CCN(c4cccc(F)n4)CC3)CC2)c1] | 1-(4-(3-ethoxyphenyl)cyclohexyl)-4- (6-fluoropyridin-2-yl)piperazine | 384.4 |
| 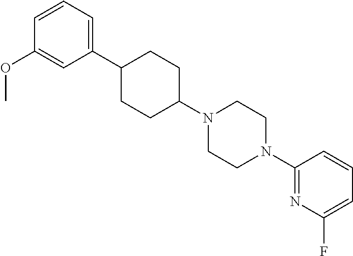 [SMILES: COc1cccc(C2CCC(N3CCN(c4cccc(F)n4)CC3)CC2)c1] | 1-(6-fluoropyridin-2-yl)-4-(4-(3- methoxyphenyl)cyclohexyl)piperazine | 370.4 |
| 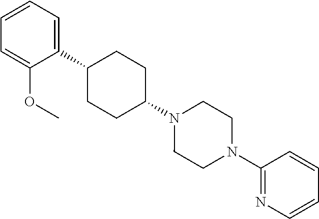 [SMILES: COc1ccccc1[C@H]1CC[C@@H](N2CCN(c3ccccn3)CC2)CC1] | cis-1-(4-(2- methoxyphenyl)cyclohexyl)-4- (pyridin-2-yl)piperazine | 352.4 |
| 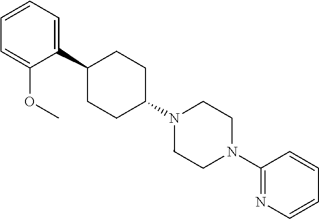 [SMILES: COc1ccccc1[C@H]1CC[C@H](N2CCN(c3ccccn3)CC2)CC1] | trans-1-(4-(2- methoxyphenyl)cyclohexyl)-4- (pyridin-2-yl)piperazine | 352.4 |
| 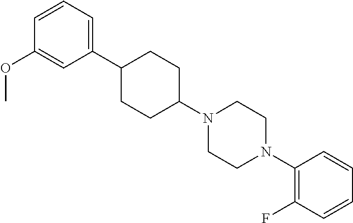 [SMILES: COc1cccc(C2CCC(N3CCN(c4ccccc4F)CC3)CC2)c1] | 1-(2-fluorophenyl)-4-(4-(3- methoxyphenyl)cyclohexyl)piperazine | 369.4 |

### Example 7

### Scheme 7 Shows a Method for Making Radiolabeled Compounds of Formula (I) for Preparation of 2-(5-(4-(6-fluoropyridin-2-yl)piperazin-1-yl)pentyl)-4-methyl-1,2,4-triazine-3,5(2H,4H)-dione

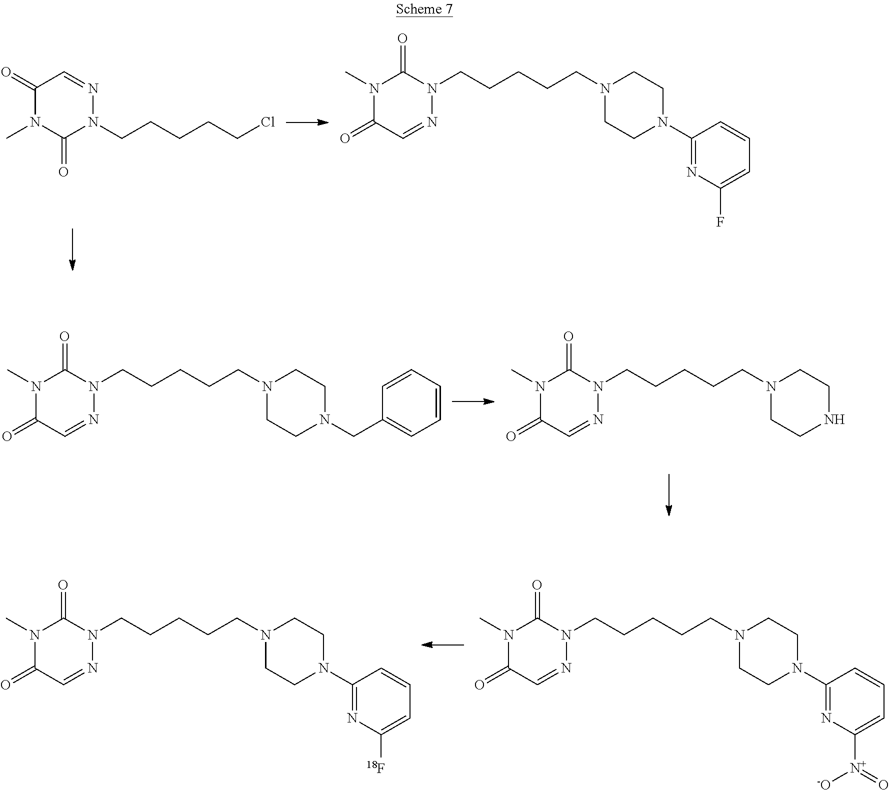

### Preparation of 2-(5-chloropentyl)-4-methyl-1,2,4-triazine-3,5(2H,4H)-dione

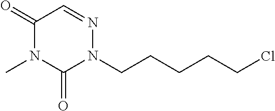

To a suspension of 60% NaH (200 mg, 5 mmol) in DMF (5 mL) was added 4-methyl-1,2,4-triazine-3,5(2H,4H)-dione (508 mg, 4 mmol) in DMF (20 mL) dropwiswe at 0° C. After one hour a yellow/orange suspension had formed and to this was added 1-bromo-5-chloropentane (740 mg, 4 mmol) in DMF (15 mL) dropwise. The reaction mixture was allowed to warm to room temperature and then stirred for 18 hours. The reaction was quenched with water (100 mL) and partitioned with EtOAc (100 mL×2). The organics were dried over magnesium sulfate, filtered and concentrated. The yellow oil was purified on silica (50 g) eluting with EtOAc/petrol (5 to 100% EtOAc over 14 CV at a flow of 40 mL/min). The porduct eluted between 5-8 CV and this was concentrated to give a white solid (390 mg, 34%).

¹H NMR (300 MHz, CDCl₃): δ 1.46 (2H, p, J=7.4 Hz, CH₂), 1.70-1.83 (4H, 2×CH₂), 1.73 (2H, p, J=7.7 Hz, CH₂), 2.31 (2H, t, J=7.7 Hz, CH₂), 3.30 (3H, s, NCH₃), 3.51 (2H, t, J=6.7 Hz, CH₂), 3.95 (2H, t, J=7.4 Hz, CH₂), 7.17-7.31 (5H, m, ArH), and 7.36 (1H, s, N═CH); ¹³C NMR (75 MHz, CDCl₃): δ 23.6, 26.9, 27.3, 31.9, 44.6, 51.5, 133.7, 148.7, and 156.1.

### Preparation of 2-(5-(4-(6-fluoropyridin-2-yl)piperazin-1-yl)pentyl)-4-methyl-1,2,4-triazine-3,5(2H,4H)-dione

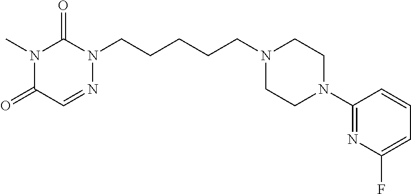 [SMILES: Cn1c(=O)cnn(CCCCCN2CCN(c3cccc(F)n3)CC2)c1=O]

2-(5-chloropentyl)-4-methyl-1,2,4-triazine-3,5(2H,4H)-dione (136 mg, 0.587 mmol) and 1-(6-fluoropyridin-2-yl)piperazine (106 mg, 0.585 mmol) were dissolved in 1-butanol (6 ml) and triethylamine (0.6 mL) added. The mixture was refluxed for 24 h and allowed to cool. Evaporated to dryness, dissolved in dichloromethane (10 mL and washed with water and separated. Evaporated and purified by chromatography on silica gel (10 g) eluting with 0-10% methanol-dichloromethane over 20 CV to give the product as a sticky gum (75 mg, 34%).

¹NMR (300 MHz, CDCl₃) δ 1.29-1.41 (2H, m, CH₂CH₂CH₂), 1.49-1.61 (2H, m, CH₂—CH₂CH₂—Npip), 1.70-1.81 (2H, m, CH₂CH₂N), 2.35 (2H, t, J=7.5 Hz, CH₂—Npip), 2.48 (4H, t, J=5.0 Hz, pip-CH₂N), 3.31 (3H, s, NCH₃), 3.50 (4H, t, J=5.0 Hz, pip-CH₂N), 3.96 (2H, t, J=7.5 Hz, CH₂N), 6.13 (1H, dd, J=7.5 Hz, 2.5 Hz, pyridyl-H5), 6.38 (1H, dd, J=8.5 Hz, 2.5 Hz, pyridyl-H3), 7.36 (1H, s, CH═N) and 7.49 (1H, q, J=8.0 Hz, pyridyl-H4). ¹³C NMR (75 MHz, CDCl₃) δ 24.3 (CH2—CH₂CH₂), 26.3 (CH₂CH₂CH₂), 26.9 (CH₃—N), 28.0 (CH₂CH₂CH₂), 44.8 (2×CH₂-pip), 51.7 (CH₂—Npip), 52.8 (2×CH₂-pip), 58.3 (CH₂—N), 95.9 (d, J=37 Hz, pyridyl-C5), 102.5 (d, J=5 Hz, pyridyl-C-3), 133.6 (CH═N), 141.7 (d, J=8 Hz, pyridyl-C4), 148.7 (C—(C═O)—N), 156.1 (N—((C═O)—N), 158.3 (d, J=15 Hz, pyridyl-C2) and 162.7 (d, J=234 Hz, pyridyl-C6-F). ¹⁹F NMR (CDCl₃) δ−68.4.

### Preparation of 2-(5-(4-benzylpiperazin-1-yl)pentyl)-4-methyl-1,2,4-triazine-3,5(2H,4H)-dione

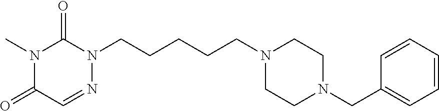 [SMILES: Cn1c(=O)cnn(CCCCCN2CCN(Cc3ccccc3)CC2)c1=O]

To a solution of 2-(5-chloropentyl)-4-methyl-1,2,4-triazine-3,5(2H,4H)-dione (200 mg, 0.86 mmol) and TRIETHYLAMINE (0.15 g, 0.2 mL, 1.5 mmol) was added 1-benzyl-piperazine (200 mg, 1.1 mmol) at ambient temperature. The reaction mixture was partitioned between water (25 mL) and DICHLOROMETHANE (25 mL). The organics were dried and concentrated. They were purified on silica (50 g) eluting with EtOAc/Methanol (5% Methanol for 1 CV then to 10% Methanol over 12 CV at a flow of 40 mL/min). The product eluted between ˜6-14 CV. These fractions were concentrated to give a clear oil (140 mg, 43%).

¹H NMR (300 MHz, CDCl₃): δ 1.32 (2H, p, J=7.7 Hz, CH₂), 1.51 (2H, p, J=7.7 Hz, CH₂), 1.73 (2H, p, J=7.7 Hz, CH₂), 2.31 (2H, t, J=7.7 Hz, CH₂), 2.66 (4H, brs, pip 2×CH₂), 3.11 (4H, brs, pip CH₂), 3.30 (3H, s, NCH₃), 3.48 (2H, s, PhCH₂), 3.94 (2H, t, J=7.7 Hz, CH₂), 7.17-7.31 (5H, m, ArH), and 7.35 (1H, s, N═CH); ¹³C NMR (75 MHz, CDCl₃): δ 24.3, 26.3, 26.8, 27.9, 51.7, 53.1, 53.3, 58.3, 63.0, 126.9, 128.1, 129.1, 133.6, 138.0, 148.7, and 156.1.

### Preparation of 4-methyl-2-(5-(piperazin-1-yl)pentyl)-4,5-dihydro-1,2,4-triazin-3(2H)-one

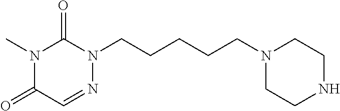 [SMILES: Cn1c(=O)cnn(CCCCCN2CCNCC2)c1=O]

A solution of 2-(5-(4-benzylpiperazin-1-yl)pentyl)-4-methyl-1,2,4-triazine-3,5(2H,4H)-dione (140 mg, 0.38 mmol) in acetic acid (8 mL) was passed over 20% Pd(OH)₂ with a flow of 1 mL/min with 80 bar hydrogen and 80 C using a H-cube hydrogenator. This led to quantitative removal of the benzyl protecting group giving an oil (107 mg, quantitative).

¹H NMR (300 MHz, CDCl₃): δ 1.29 (2H, p, J=7.3 Hz, CH₂), 1.49 (2H, p, J=7.3 Hz, CH₂), 1.71 (2H, p, J=7.3 Hz, CH₂), 2.38 (2H, t, J=7.3 Hz, CH₂), 2.66 (4H, brs, pip 2×CH₂), 3.11 (4H, brs, pip CH₂), 3.29 (3H, s, NCH₃), 3.91 (2H, t, J=7.1 Hz, CH₂), and 7.34 (1H, s, N═CH); ¹³C NMR (75 MHz, CDCl₃): δ 22.1, 23.9, 25.4, 26.8, 27.8, 42.9, 49.5, 51.5, 57.5, 105.4, 125.1, 128.1, 128.9, 133.6, 148.7, 156.1, and 176.6.

### Preparation of precursor 4-methyl-2-(5-(4-(6-nitropyridin-2-yl)piperazin-1-yl)pentyl)-1,2,4-triazine-3,5(2H,4H)-dione

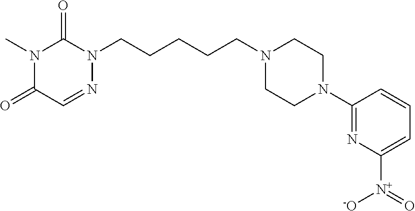

To a solution of 4-methyl-2-(5-(piperazin-1-yl)pentyl)-4,5-dihydro-1,2,4-triazin-3(2H)-one (107 mg, 0.38 mmol) in MeCN (3 mL) was added Hunig's base (0.13 g, 1 mmol) and 2-chloro-6-nitropyridine (0.060 g, 0.38 mmol). This was heated in the CEM microwave at 130 C for 30 minutes. The reaction mixture was concentrated to give a brown oil. This was separated on silica (40 g) eluting with Methanol/EtOAC (3% to 5% Methanol over 16 CV at a flow of 40 mL/min). The product eluted between ˜9 to 12 CV to give a yellow oil (40 mg, 26%).

¹H NMR (300 MHz, CDCl₃): δ 1.38 (2H, p, J=7.7 Hz, CH₂), 1.58 (2H, p, J=7.7 Hz, CH₂), 1.78 (2H, p, J=7.7 Hz, CH₂), 2.40 (2H, t, J=7.4 Hz, CH₂), 2.55 (4H, t, J=5.2 Hz, pip 2×CH₂), 3.34 (3H, s, NCH₃), 3.66 (4H, t, J=5.2 Hz, pip CH₂), 3.98 (2H, t, J=7.3 Hz, CH₂), 6.89 (1H, d, J=8.6 Hz, ArH), 7.42 (1H, s, N═CH), 7.43 (1H, d, J=7.7 Hz, ArH), and 7.68 (1H, t, J=7.7 HZ, ArH); ¹³C NMR (75 MHz, CDCl₃): δ 24.3, 26.2, 26.9, 28.0, 44.6, 51.8, 52.7, 58.2, 105.4, 111.7, 133.7, 140.1, 156.0, and 158.5.

### Preparation of [¹⁸F]2-(5-(4-(6-fluoropyridin-2-yl)piperazin-1-yl)pentyl)-4-methyl-1,2,4-triazine-3,5(2H,4H)-dione

[¹⁸F]-fluoride (8.95 GBq) was transferred to the GE Tracerlab FX and trapped on a conditioned QMA cartridge. The [¹⁸F]-fluoride was then eluted as the Kryptofix (K222) complex using a solution formed of Kryptofix (10.0 mg) in MeCN (2 ml) and 0.1 M K₂CO₃ (100 μl) into the reaction vessel. Azeotropic drying of the [¹⁸F]-fluoride commenced at 100° C. for 30 minutes under a stream of N₂ gas. The dried [¹⁸F]-fluoride/K222 vial was then cooled to 30° C. and N₂ flow ceased. 4-Methyl-2-(5-(4-(6-nitropyridin-2-yl)piperazin-1-yl)pentyl)-1,2,4-triazine-3,5(2H,4H)-dione (1.0 mg) in DMSO (1 ml) was added to the reaction vessel which was then sealed and heated to 150° C. for 10 minutes. The crude reaction was diluted with H₂0 (2.5 ml) and transferred to a semi-preparative HPLC system (Phenomenex Luna C8 250×10 mm, 5 um, A=0.8% TRIETHYLAMINE adj. to pH 7.6 with H₃PO₄, B=MeCN, 50% B over 20 minutes). The collected fraction was diluted with H₂O (10 ml) and trapped on a tC18 light cartridge (conditioned with EtOH (5 ml) and H₂O (10 ml)). The cartridge was washed with H₂O (4 ml) and eluted with EtOH (500 μl) before formulation with PBS (4.5 ml). The product consisted of 58.3 MBq (n.d.c yield 0.65% at time of formulation) [¹⁸F]AH114666 was analysed by RP HPLC (A=0.8% TRIETHYLAMINE, adj. to pH 7.6 with H₃PO₄ B=MeCN; 50% B over 20 mins) and eluted simultaneously with a spike of cold standard at 12.7 minutes. Total synthesis time 71 minutes, specific activity 19 GBq/umol.

### Example 8

### Scheme 8 Shows a Method for Making Radiolabeled Compounds of Formula (I) for Preparation of N-(4-(4-(6-fluoropyridin-2-yl)piperazin-1-yl)butyl)cyclohexanecarboxamide

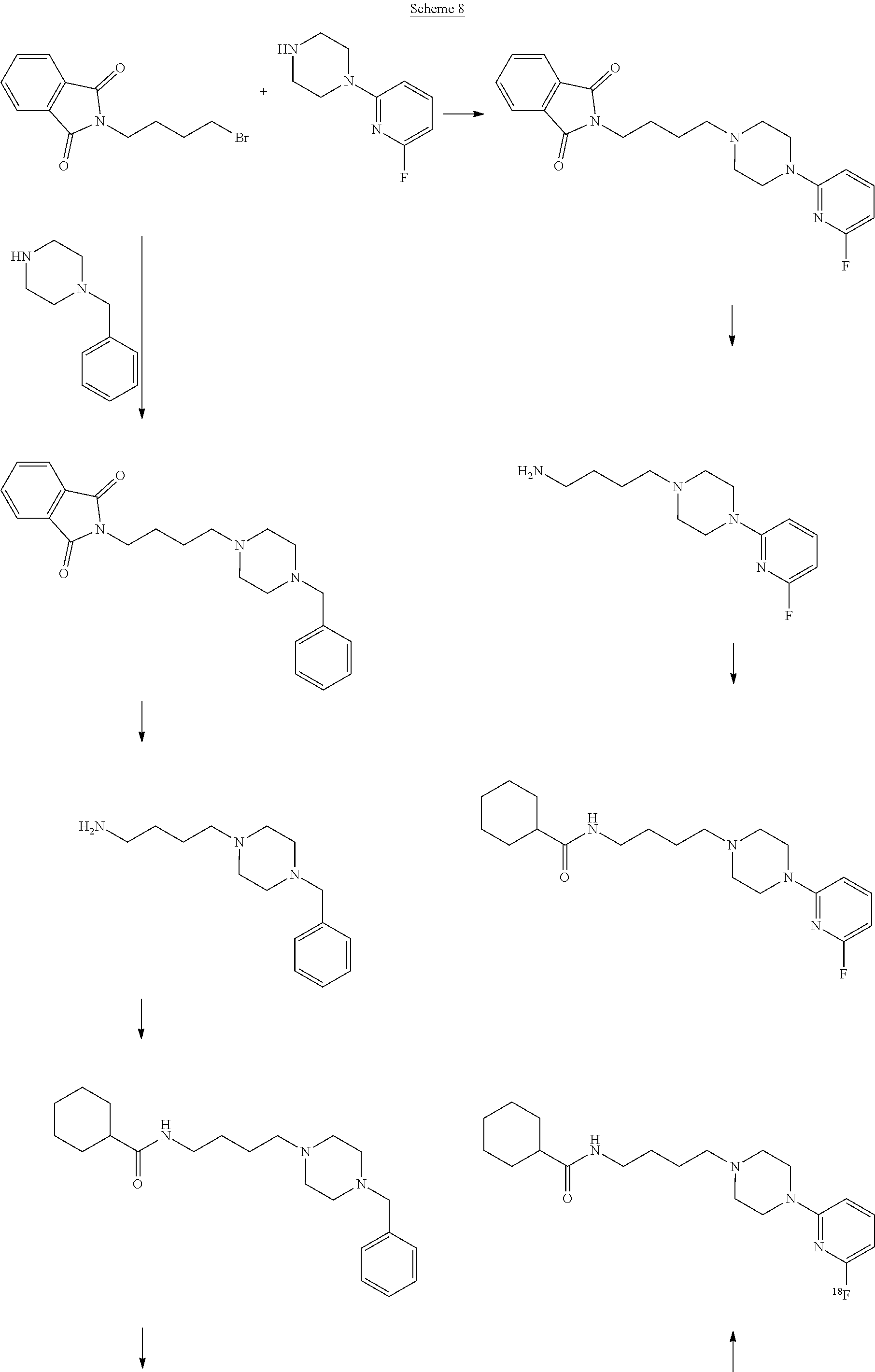

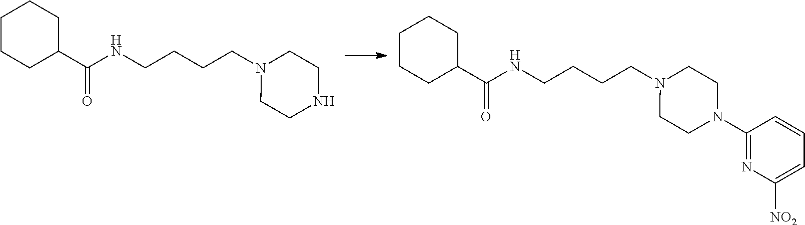 [SMILES: O=C(NCCCCN1CCNCC1)C1CCCCC1 ; O=C(NCCCCN1CCN(c2cccc([N+](=O)[O-])n2)CC1)C1CCCCC1]

### Preparation of 2-(4-(4-(6-fluoropyridin-2-yl)piperazin-1-yl)butyl)isoindoline-1,3-dione

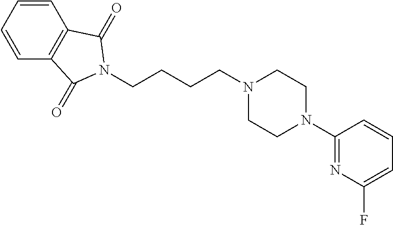 [SMILES: O=C1c2ccccc2C(=O)N1CCCCN1CCN(c2cccc(F)n2)CC1]

A flask was charged with 1-(6-fluoro-pyridin-2-yl)-piperazine (1.60 g, 8.83 mmol), dimethylformamide (30 mL), potassium carbonate (2.44 g, 17.6 mmol) and N-(4-bromobutyl)phthalimide (2.49 g, 8.83 mmol) at ambient temperature under a nitrogen atmosphere then heated to 80° C. for 17 hours. The reaction mixture was filtered and concentrated to give 2-(4-(4-(6-fluoropyridin-2-yl)piperazin-1-yl)butyl)isoindoline-1,3-dione as a slurry (3.38 g, 100%). The product was used without further purification.

LCMS: calcd for C₂₁H₂₃FN₄O₂, 382.2; found 383.05 [M+H]⁺.

### Preparation of 4-(4-(6-fluoropyridin-2-yl)piperazin-1-yl)butan-1-amine

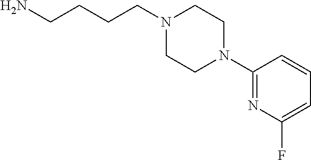 [SMILES: NCCCCN1CCN(c2cccc(F)n2)CC1]

A flask was charged with 2-(4-(4-(6-fluoropyridin-2-yl)piperazin-1-yl)butyl)isoindoline-1,3-dione (3.38 g, 8.83 mmol), ethanol (26 mL) and hydrazine hydrate (0.88 g, 17.66 mmol) at ambient temperature under a nitrogen atmosphere. The mixture was heated to 80° C. for 6 hours leading to the formation of a thick white precipitate. The reaction mixture was cooled to ambient temperature, ethanol (30 mL) added and the white solid removed by filtration. The solid was washed with ethanol (2×30 mL), the filtrates combined and concentrated under reduced pressure to give the product as a yellow slurry (3.34 g, 70% purity by ¹HNMR).

LCMS: calcd for C₁₃H₂₁FN₄, 252.34; found 253 [M+H]⁺.

¹³C and ¹H NMR were consistent with the structure.

### Preparation of N-(4-(4-(6-fluoropyridin-2-yl)piperazin-1-yl)butyl)cyclohexanecarboxamide

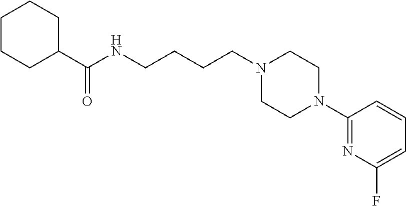 [SMILES: O=C(NCCCCN1CCN(c2cccc(F)n2)CC1)C1CCCCC1]

Triethylamine (154 μl, 1.11 mmol), and cyclohexanecarbonyl chloride (148 μl, 1.11 mmol) were added to 4-(4-(6-fluoropyridin-2-yl)piperazin-1-yl)butan-1-amine (400 mg, 70% purity, 1.11 mmol) in dichloromethane (20 mL) and the reaction mixture stirred at ambient temperature for 1 hour. The reaction mixture was washed with 10% aqueous potassium carbonate solution (10 mL), the organic layer dried over magnesium sulfate, filtered and concentrated under reduced pressure. The residue was purified twice by chromatography on silica gel eluting with a 0-5% gradient of 1% triethylamine/methanol and dichloromethane to give the product as a yellow solid (85 mg, 21%).

LCMS: calcd for C₂₀H₃₁FN₄O, 362.3; found 363.1 [M+H]⁺.

HPLC (UV 254 nm) analysis showed 96% purity.

¹H, ¹⁹F and ¹³C NMR were consistent with the structure.

### Preparation of 2-(4-(4-benzylpiperazin-1-yl)butyl)isoindoline-1,3-dione

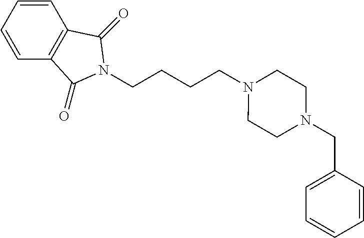 [SMILES: O=C1c2ccccc2C(=O)N1CCCCN1CCN(Cc2ccccc2)CC1]

N-(4-bromobutyl)phthalimide (2.0 g, 7.09 mmol) was dissolved in acetonitrile (8 mL) and 1-benzylpiperazine (1.24 mL, 7.09 mmol) then diidopropylethylamine (1.24 mL, 7.09 mmol) were added at ambient temperature. The reaction mixture was split into four 10 mL tubes and heated at 130° C. for 30 min in the microwave oven. TLC showed all starting materials had disappeared and a major product had formed. The reaction mixtures were combined and evaporated to dryness then purified by chromatography on silica gel eluting with 0-5% methanol in dichloromethane gradient. The fractions containing the product were combined and evaporated to dryness under reduced pressure to give the product as a pale orange oil (2.47 g, 92%).

LCMS: calcd for C₂₃H₂₇N₃O₂, 377.2; found 378.1 [M+H]⁺.

¹H and ¹³C NMR were consistent with the structure.

### Preparation of 4-(4-benzylpiperazin-1-yl)butan-1-amine

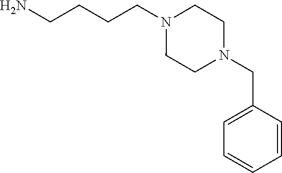 [SMILES: NCCCCN1CCN(Cc2ccccc2)CC1]

A flask was charged with 2-(4-(4-benzylpiperazin-1-yl)butyl)isoindoline-1,3-dione (2.46 g, 6.52 mmol), ethanol (30 mL) and hydrazine hydrate (0.6 mL, 13.0 mmol) and the reaction mixture was heated at 80° C. for 18 h. The reaction mixture was cooled to ambient temperature and the white solid removed by filtration. The solid was washed with ethanol, the filtrates combined and concentrated under reduced pressure to give the product as an off-white solid (1.61 g, 100%).

LCMS: calcd for C₁₅H₂₅N₃, 247.20; found 248.14 [M+H]⁺.

¹H and ¹³C NMR were consistent with the structure.

### Preparation of N-(4-(4-benzylpiperazin-1-yl)butyl)cyclohexanecarboxamide

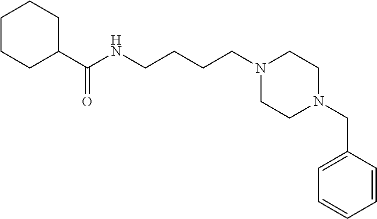 [SMILES: O=C(NCCCCN1CCN(Cc2ccccc2)CC1)C1CCCCC1]

Triethylamine (511 μl, 3.67 mmol), and cyclohexanecarbonyl chloride (491 μl, 3.67 mmol) were added to 4-(4-benzyl-piperazin-1-yl)-butylamine (605 mg, 62% pure, 1.52 mmol) in dichloromethane (25 mL). The reaction was stirred at ambient temperature for 1 hour. The reaction mixture was evaporated to dryness and the residue purified by chromatography on silica gel eluting with 0-10% methanol in dichloromethane to give the product as a pale yellow oil (337 mg, 62%).

LCMS calcd for C₂₂H₃₅N₃O, 357.28; found 358.15 [M+H]⁺.

¹H and ¹³C NMR were consistent with the structure.

### Preparation of N-(4-(piperazin-1-yl)butyl)cyclohexanecarboxamide

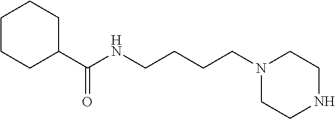 [SMILES: O=C(NCCCCN1CCNCC1)C1CCCCC1]

N-(4-(4-Benzylpiperazin-1-yl)butyl)cyclohexanecarboxamide (194 mg, 0.54 mmol) was dissolved in acetic acid (10 mL) and run through a 20% Pd(OH)₂ cartridge at 1 mL/min acetic acid, 80 bar pressure and 80° C. using an H-Cube hydrogenator. The eluent containing the product was evaporated to dryness and the residue dissolved in dichloromethane then washed with 10% aqueous potassium carbonate. The organic layer was separated and evaporated under reduced pressure to give the product as a colourless oil (77 mg, 53%).

LCMS: calcd for C₁₅H₂₉N₃O 267.2; found 268.2 [M+H]⁺.

¹H and ¹³C NMR were consistent with the structure.

### Preparation of N-(4-(4-(6-nitropyridin-2-yl)piperazin-1-yl)butyl)cyclohexanecarboxamide

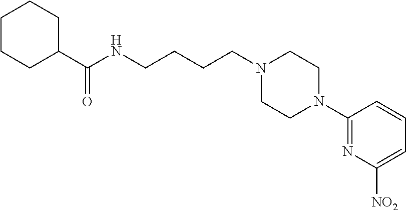 [SMILES: O=C(NCCCCN1CCN(c2cccc([N+](=O)[O-])n2)CC1)C1CCCCC1]

N-(4-(piperazin-1-yl)butyl)cyclohexanecarboxamide (166 mg, 0.62 mmol), and 2-chloro-6-nitropyridine (98.6 mg, 0.62 mmol) were dissolved in dry acetonitrile (8 ml) in 2×10 mL microwave tubes and 6 drops diisopropylethylamine added to each. The mixture was stirred during heating to dissolve and heated in microwave at 120° C. for 40 min. Combined and evaporated. Partitioned between dichloromethane and water and separated. Evaporated and purified by chromatography on silica gel (10 g) eluting with 0.5-10% methanol in ethyl acetate to give incomplete separation of product from a chloro impurity. Further purified by semi-preparative HPLC eluting with methanol-water gradient to give the product (35 mg, 15%).

LCMS calc'd for C₁₇H₂₃N₇O₄: 389.2; found 390.1 [M+H]⁺.

¹H NMR (300 MHz, d6-DMSO): δ 1.50-1.63 (2H, m, CH₂CH₂), 1.73-1.86 (2H, m, CH₂CH₂), 2.42 (2H, t, J=7.5 Hz, CH₂N), 2.53 (4H, t, J=5.0 Hz, pip-CH₂), 3.33 (3H, s, N—CH₃), 3.65 (4H, t, J=5.0 Hz, pip-CH₂), 4.01 (2H, t, J=7.0 Hz, CH₂—N), 6.89 (1H, d, J=8.5 Hz, pyridylC3-H), 7.39 (1H, s, N═CH), 7.43 (1H, d, J=7.5 Hz, pyridylC5-H) and 7.68 (1H, t, J=8.0 Hz, pyridylC4-H). ¹³C NMR (75 MHz, d6-DMSO): δ 23.6 (CH₂CH₂), 26.1 (CH₂CH₂), 26.9 (N—CH₃), 44.7 (pip-CH₂), 51.6 (N—CH₂), 52.7 (pip-CH₂), 57.9 (NCH₂), 105.4 (pyridyl-C5), 111.7 (pyridyl-C3), 133.8 (N═CH), 140.1 (pyridyl-C4), 148.8 (NMe-C═O), 155.8 (pyridyl-C2), 156.2 (MeN-C(═O)N) and 157.7 (C—NO₂).

Compounds that were synthesized following procedures that were similar or identical as described those in Example 8 include the following:

| Structure | Name | [M + H]⁺ |
|---|---|---|
| 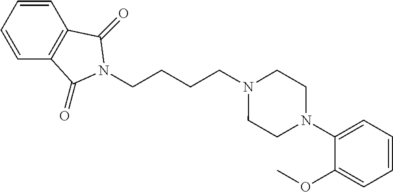 [SMILES: COc1ccccc1N1CCN(CCCCN2C(=O)c3ccccc3C2=O)CC1] | NAN-190 | 394.4 |
| 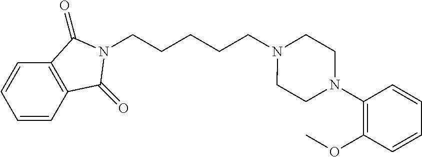 [SMILES: COc1ccccc1N1CCN(CCCCCN2C(=O)c3ccccc3C2=O)CC1] | 2-(5-(4-(2- methoxyphenyl)piperazin- 1-yl)pentyl)isoindoline- 1,3-dione | 408.4 |
| 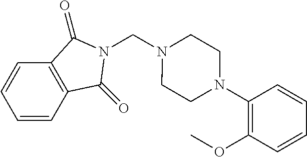 [SMILES: COc1ccccc1N1CCN(CN2C(=O)c3ccccc3C2=O)CC1] | 2-((4-(2- methoxyphenyl)piperazin- 1-yl)methyl)isoindoline- 1,3-dione | 352.3 |
| 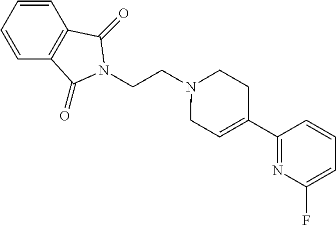 [SMILES: O=C1c2ccccc2C(=O)N1CCN1CC=C(c2cccc(F)n2)CC1] | 2-(2-(6-fluoro-5′,6′- dihydro-[2,4′-bipyridin]- 1′(2′H)- yl)ethyl)isoindoline-1,3- dione | 352.6 |
| 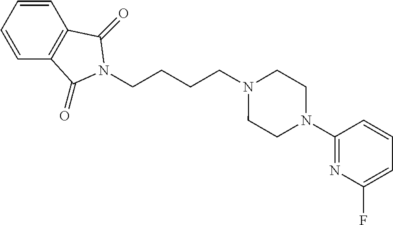 [SMILES: O=C1c2ccccc2C(=O)N1CCCCN1CCN(c2cccc(F)n2)CC1] | 2-(4-(4-(6-fluoropyridin- 2-yl)piperazin-1- yl)butyl)isoindoline-1,3- dione | 383.3 |
| 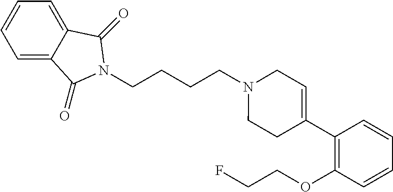 [SMILES: O=C1c2ccccc2C(=O)N1CCCCN1CC=C(c2ccccc2OCCF)CC1] | 2-(4-(4-(2-(2- fluoroethoxy)phenyl)-5,6- dihydropyridin-1(2H)- yl)butyl)isoindoline-1,3- dione | 423.3 |
| 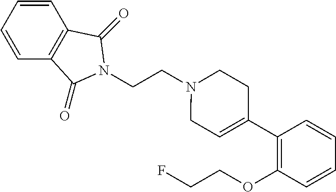 [SMILES: O=C1c2ccccc2C(=O)N1CCN1CC=C(c2ccccc2OCCF)CC1] | 2-(2-(4-(2-(2- fluoroethoxy)phenyl)-5,6- dihydropyridin-1(2H)- yl)ethyl)isoindoline-1,3- dione | 395.3 |

### Example 9

### Scheme 9 Shows a Method for Making Radiolabeled Compounds of Formula (I) for Preparation of (3r,5r,7r)-N-(4-(4-(6-fluoropyridin-2-yl)piperazin-1-yl)butyl)adamantane-1-carboxamide

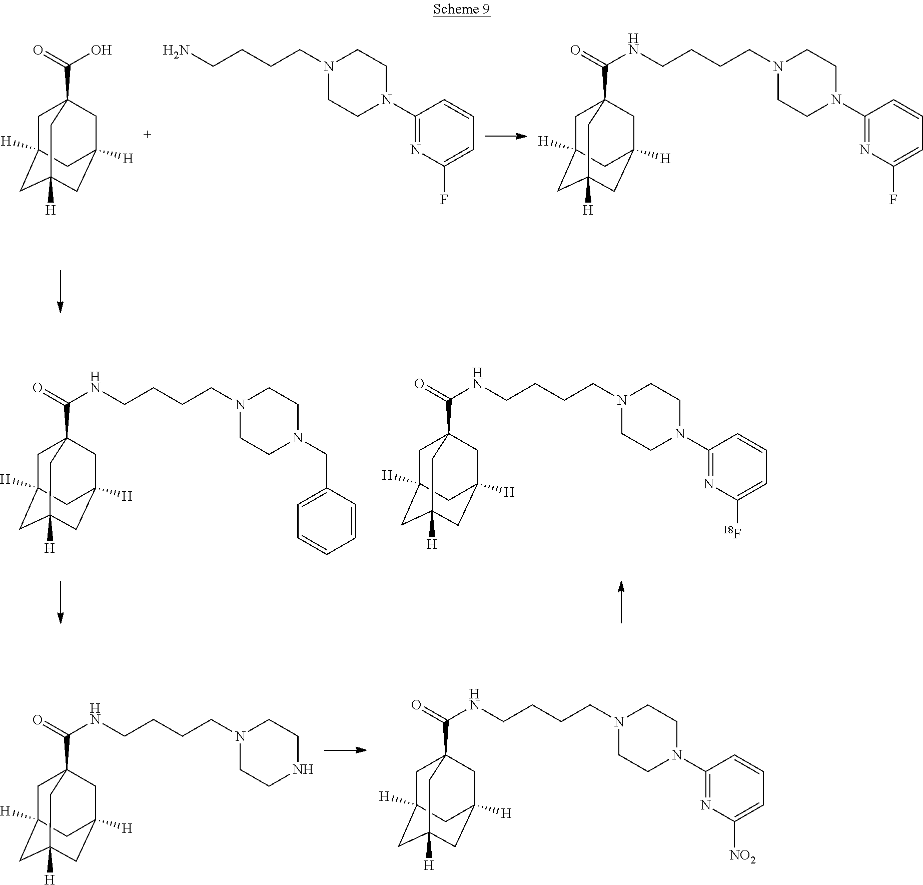

### Preparation of (3r,5r,7r)-N-(4-(4-(6-fluoropyridin-2-yl)piperazin-1-yl)butyl)adamantane-1-carboxamide

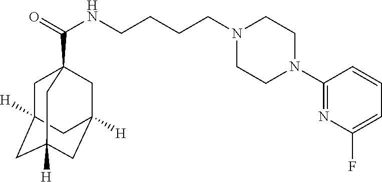 [SMILES: O=C(NCCCCN1CCN(c2cccc(F)n2)CC1)[C@]12C[C@H]3C[C@H](C[C@H](C3)C1)C2]

Triethylamine (77 μl, 0.55 mmol), and adamantanecarbonyl chloride (110 mg, 0.55 mmol) were added to 4-(4-(6-fluoropyridin-2-yl)piperazin-1-yl)butan-1-amine (200 mg, 0.55 mmol) in dichloromethane (10 mL). The reaction was stirred at ambient temperature for 1 hour. The reaction mixture was concentrated and purified by chromatography on silica gel eluting with 1% NEt₃ in methanol and dichloromethane to give the product as a yellow oil (119 mg, 52%).

LCMS: calcd for C₂₄H₃₅FN₄O, 414.3; found 415.2 [M+H]⁺.

HPLC (UV 254 nm) analysis showed 96% purity.

¹H and ¹³C NMR were consistent with the structure.

### Preparation of (3r,5r,7r)-N-(4-(4-benzylpiperazin-1-yl)butyl)adamantane-1-carboxamide

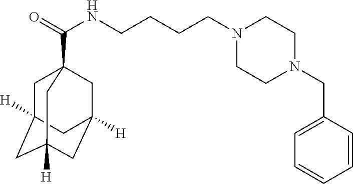 [SMILES: O=C(NCCCCN1CCN(Cc2ccccc2)CC1)[C@]12C[C@H]3C[C@H](C[C@H](C3)C1)C2]

Triethylamine (377 μl, 2.71 mmol) and adamantanecarbonyl chloride (403 mg, 2.03 mmol) were added to 4-(4-benzyl-piperazin-1-yl)-butylamine (500 mg, 1.35 mmol) in dichloromethane (20 mL). The reaction was stirred at ambient temperature for 2.5 hours. 10% aqueous potassium carbonate was added to the reaction mixture and the mixture shaken vigorously then filtered through a phase separator. The dichloromethane layer was evaporated under reduced pressure and the yellow oil obtained purified by chromatography on silica gel to give the product (480 mg, 86%).

LCMS: calcd for C₂₆H₃₉N₃O, 409.3; found 410.2 [M+H]⁺.

¹H and ¹³C NMR were consistent with the structure.

### Preparation of (3r,5r,7r)-N-(4-(piperazin-1-yl)butyl)adamantane-1-carboxamide

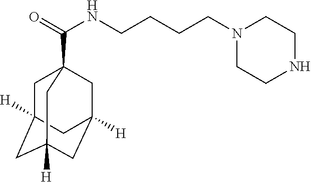 [SMILES: O=C(NCCCCN1CCNCC1)[C@]12C[C@H]3C[C@H](C[C@H](C3)C1)C2]

(3r,5r,7r)-N-(4-(4-Benzylpiperazin-1-yl)butyl)adamantane-1-carboxamide (464 mg, 1.13 mmol) was dissolved in acetic acid (20 ml) and 10% Pd/C (60 mg) was added. Under a nitrogen atmosphere, cyclohexene (1 mL) was added and the reaction mixture heated at 60° C. for 18 h. The reaction mixture was cooled to room temperature and filtered through celite. TLC indicated a non-UV-active spot on the baseline that was revealed using ninhydrin. The filtrate was evaporated under reduced pressure to give a yellow oil which was dissolved in dichloromethane and washed with 10% aqueous potassium carbonate to remove residual acetic acid. The organic layer was evaporated to dryness to give the product as a yellow oil (328 mg, 91%).

LCMS: calcd for C₁₉H₃₃N₃O, 319.3; found 320.0 [M+H]⁺.

¹H and ¹³C NMR were consistent with the structure.

### Preparation of (3r,5r,7r)-N-(4-(4-(6-nitropyridin-2-yl)piperazin-1-yl)butyl)adamantane-1-carboxamide

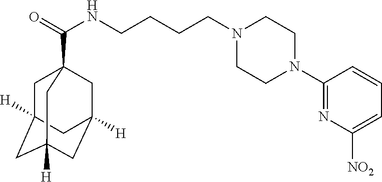 [SMILES: O=C(NCCCCN1CCN(c2cccc([N+](=O)[O-])n2)CC1)[C@]12C[C@H]3C[C@H](C[C@H](C3)C1)C2]

(3r,5r,7r)-N-(4-(piperazin-1-yl)butyl)adamantane-1-carboxamide (328 mg, 1.03 mmol) and 2-chloro-6-nitropyridine (163 mg, 1.03 mmol) were dissolved in dry acetonitrile (5 ml) in a 10 mL microwave tube and diisopropylethylamine (358 μL, 2.06 mmol) added. The reaction mixture was stirred and then heated in a microwave oven at 120° C. for 30 min. The reaction mixture was evaporated under reduced pressure to give a brown residue which was taken up in ethyl acetate (15 mL) and washed with 10% aqueous potassium carbonate (10 mL). The organic layer was filtered through a phase separator and evaporated under reduced pressure. The residue was purified by chromatography on silica gel to give the product eluting with 0-10% methanol-ethyl acetate gradient over 12 CV followed by semi-preparative HPLC using a gradient of 50-95% methanol in water over 20 min @21 ml/min (Gemini C18, 110 Å, 150×21.2 mm, 5 μm; Rt 17.3 min) to give the product as a yellow solid (155 mg, 34%).

LCMS: calcd for C₂₄H₃₅N₅O₃, 441.3; found 442.2 [M+H]⁺.

The product was 100% pure by HPLC (UV254 nm).

¹H and ¹³C NMR were consistent with the structure.

### Preparation of [¹⁸F](3r,5r,7r)-N-(4-(4-(6-fluoropyridin-2-yl)piperazin-1-yl)butyl)adamantane-1-carboxamide

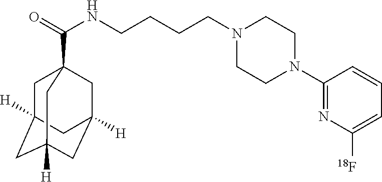 [SMILES: O=C(NCCCCN1CCN(c2cccc([18F])n2)CC1)[C@]12C[C@H]3C[C@H](C[C@H](C3)C1)C2]

The Kryptofix/potassium carbonate solution (molar ratio 2:1, MeCN/H₂O 96:4, 2 mL) and precursor (3r,5r,7r)-N-(4-(4-(6-nitropyridin-2-yl)piperazin-1-yl)butyl)adamantane-1-carboxamide (2.0 mg in 0.5 mL DMF and 0.1 mL DMSO) were added to vials 1 and 3, respectively of the Tracer Lab synthesizer. Vials 5, 7, 8 and 9 were filled with H₂O (2 mL), H₂O (1 mL), MeCN (0.5 mL), H₂O (5 mL) respectively. The target water containing ¹⁸F⁻ was passed through a pre-conditioned QMA cartridge where the ¹⁸F⁻ was trapped. The ¹⁸F⁻ was released from the QMA cartridge and carried into the reactor by passing the Kryptofix/potassium carbonate solution from vial 1 through the cartridge. The mixture was dried at 100° C. for 30 min. The precursor solution from vial 3 was added to the dried [K/K.2.2.2]⁺¹⁸F⁻ complex. The reaction mixture was heated for 20 min at 150° C. After reaction the crude was diluted with water from vial 5 and transferred to the round bottomed flask containing H₂O (10 mL), then transferred to a pre-conditioned SPE cartridge. The SPE was washed with the water from vial 9 and then the crude was eluted into a vial with acetonitrile and water (from vial 8 and 7). The crude was further diluted with H₂O (0.5 mL) before injected on the semi-preparative HPLC system. The mobile phase used for purification was: A: 10 mM Na₂HPO₄ B: MeCN. Flow rate=3.0 mL/min Gradient: 45-65% over 10 min, then 65-95% over 25 min. Column: Phenomenex Luna 100×10 mm, 5 micron, C18. The product fraction (Rₜ=12.4 min) was collected and diluted with H₂O (5 mL) and passed through a pre-conditioned C18 Light SPE cartridge. The purified product was trapped on the cartridge and washed with H₂O (1 mL) and eluted with EtOH (0.5 mL) and phosphate buffer (4.5 mL)

The formulated product was finally analyzed on the analytical HPLC system before being released to biology. Analytical HPLC conditions: A: H₂O B: MeCN. Gradient: 50-95% over 15 min. Flow rate=1 mL/min Column: Phenomenex Luna 150×4.6 mm, C18. Rₜ=9.1 min.

Starting with 7.5 GBq gave 776 MBq formulated [¹⁸F] (3r,5r,7r)-N-(4-(4-(6-fluoropyridin-2-yl)piperazin-1-yl)butyl)adamantane-1-carboxamide after a total synthesis time of 134 min. RCP: >99%. RCY: 10%. SRA: 21 GBq/μmol. Amount cold compound: 0.24 μg/mL.

### Example 10

### Scheme 10 Shows a Method for Making Radiolabeled Compounds of Formula (I) for Preparation of N-(3-(4-(6-Fluoropyridin-2-yl)piperazin-1-yl)propyl)cyclohexanecarboxamide

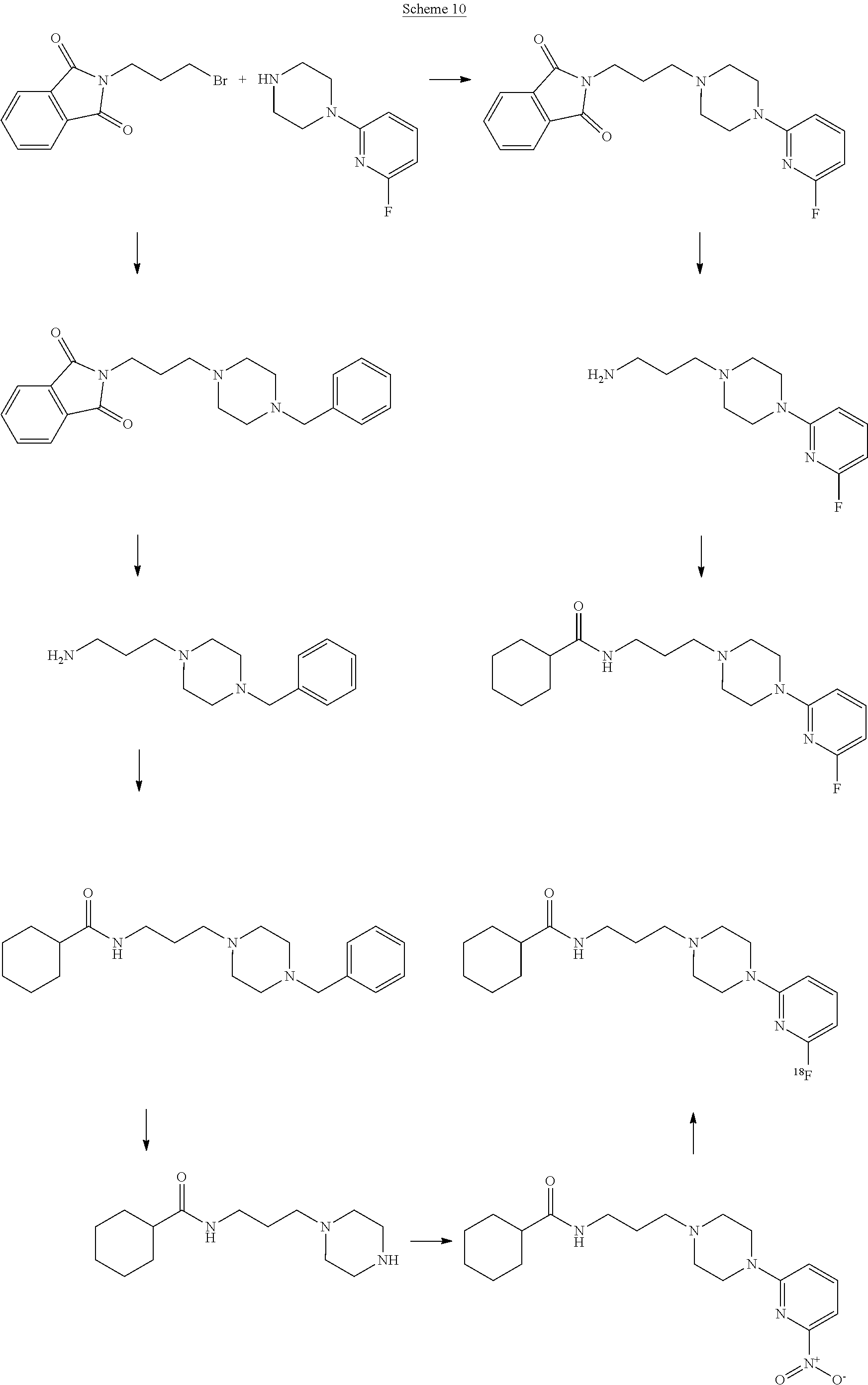

### Preparation of 2-(3-(4-(6-fluoropyridin-2-yl)piperazin-1-yl)propyl)isoindoline-1,3-dione

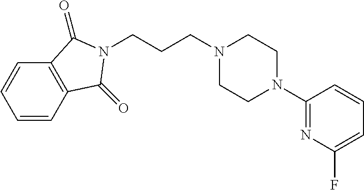 [SMILES: O=C1c2ccccc2C(=O)N1CCCN1CCN(c2cccc(F)n2)CC1]

A flask was charged with 1-(6-fluoro-pyridin-2-yl)-piperazine (1.20 g, 6.62 mmol), DMF (22 mL), potassium carbonate (1.83 g, 13.24 mmol) and N-(4-bromopropyl)-phthalimide (1.77 g, 6.62 mmol) under a nitrogen atmosphere then heated to 80° C. for 20 hours. The reaction mixture was filtered and concentrated to give the product as a slurry (2.44 g, 100%).

LCMS: calcd for C₂₀H₂₁FN₄O₂, 368.26; found 369.1 [M+H]⁺.

### Preparation of 3-(4-(6-fluoropyridin-2-yl)piperazin-1-yl)propan-1-amine

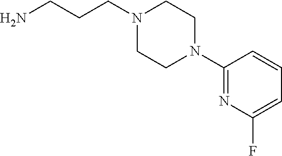 [SMILES: NCCCN1CCN(c2cccc(F)n2)CC1]

A flask was charged with 2-(3-(4-(6-fluoropyridin-2-yl)piperazin-1-yl)propyl)-isoindoline-1,3-dione (2.44 g, 6.62 mmol), ethanol (19 mL) and hydrazine hydrate (0.64 mL, 13.24 mmol) under a nitrogen atmosphere. The reaction mixture was heated to 80° C. for 4 hours then cooled to ambient temperature. Ethanol (30 mL) was added and the white solid removed by filtration. The solid was washed with ethanol (2×30 mL), the filtrates combined and concentrated under reduced pressure to give the product as yellow slurry (1.83 g, 100%).

¹H and ¹³C NMR were consistent with the structure.

### Preparation of N-(3-(4-(6-fluoropyridin-2-yl)piperazin-1-yl)propyl)cyclohexanecarboxamide

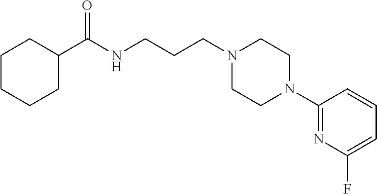 [SMILES: O=C(NCCCN1CCN(c2cccc(F)n2)CC1)C1CCCCC1]

TEA (77 μl, 0.55 mmol), and cyclohexanecarbonyl chloride (74 μl, 0.55 mmol) were added to 3-(4-(6-fluoropyridin-2-yl)piperazin-1-yl)propan-1-amine (175 mg, 0.55 mmol) in dichloromethane (10 mL). The reaction was stirred at ambient temperature for 1 hour. The reaction mixture was concentrated and purified by chromatography on silica gel eluting with 1% triethylamine in methanol and dichloromethane to give the product as a light brown solid (90 mg, 47%).

LCMS: calcd for C₁₉H₂₉FN₄O, 348.2; found 349.2 [M+H]⁺.

HPLC analysis showed 98% purity by UV (254 nm).

¹H and ¹³C NMR were consistent with the structure.

### Preparation of 2-(3-(4-Benzylpiperazin-1-yl)propyl)isoindoline-1,3-dione

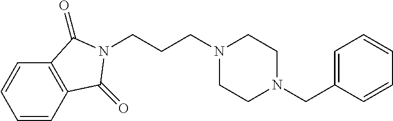 [SMILES: O=C1c2ccccc2C(=O)N1CCCN1CCN(Cc2ccccc2)CC1]

N-(3-bromopropyl)phthalimide (1.9 g, 7.09 mmol) was dissolved in acetonitrile (7 mL) and 1-benzylpiperazine (1.24 mL, 7.09 mmol) then diisopropylethylamine (1.24 mL, 7.09 mmol) were added at ambient temperature. The reaction mixture was split into two 10 mL tubes and heated at 130° C. for 15 min in the microwave oven. The reaction mixtures were combined, evaporated to dryness and the product purified by chromatography on silica gel eluting with 0-5% methanol-dichloromethane gradient to give the product as a pale yellow oil (2.03 g, 79%).

LCMS: calcd for C₂₂H₂₅N₃O₂, 363.2; found 364.1 [M+H]⁺.

¹H and ¹³C NMR were consistent with the structure.

### Preparation of 3-(4-Benzylpiperazin-1-yl)propan-1-amine

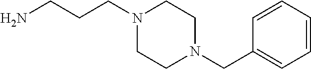 [SMILES: NCCCN1CCN(Cc2ccccc2)CC1]

A flask was charged with 2-(3-(4-benzylpiperazin-1-yl)propyl)isoindoline-1,3-dione (2.01 g, 5.53 mmol), ethanol (25 mL) and hydrazine hydrate (0.51 mL, 11.06 mmol) and the reaction mixture was heated at 80° C. for 18 h leading to the formation of a white solid. The reaction mixture was cooled to ambient temperature and the white solid removed by filtration. The solid was washed with ethanol and the filtrate was concentrated at reduced pressure to give the product as a white solid (0.51 mg, 39%).

LCMS: calcd for C₁₄H₂₃N₃, 233.2; found 234.1 [M+H]⁺.

¹H and ¹³C NMR were consistent with the structure.

### Preparation of N-(3-(4-Benzylpiperazin-1-yl)propyl)cyclohexanecarboxamide

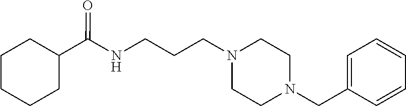 [SMILES: O=C(NCCCN1CCN(Cc2ccccc2)CC1)C1CCCCC1]

To a solution of 3-(4-benzyl-piperazin-1-yl)-propylamine (500 mg, 1.67 mmol) in dichloromethane (20 mL) was added cyclohexanecarbonyl chloride (335 μl, 2.51 mmol) and triethylamine (466 μl, 3.34 mmol). The reaction was stirred at ambient temperature for 1 hour. TLC showed the formation of a main product of Rf 0.16 (MeOH/DCM 0.5:9.5; revealed with ninhydrin). The reaction mixture was evaporated to dryness and the residue was purified by chromatography on silica gel eluting with a 0-10% methanol in dichloromethane gradient to give the product as a light brown gum (340 mg, 59%).

LC-MS: calcd for C₂₁H₃₃N₃O, 343.3; found 344.1 [M+H]⁺.

¹H and ¹³C NMR were consistent with the structure.

### Preparation of N-(3-(piperazin-1-yl)propyl)cyclohexanecarboxamide

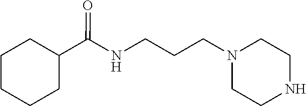 [SMILES: O=C(NCCCN1CCNCC1)C1CCCCC1]

N-(3-(4-benzylpiperazin-1-yl)propyl)cyclohexanecarboxamide (333 mg, 0.97 mmol) was dissolved in acetic acid (20 ml) and 6N hydrochloric acid (1 mL) added followed by 10% Pd/C (100 mg). The reaction mixture was shaken for 4 h at 60° C. under 30 psi H₂ atmosphere using a Parr hydrogenator. The reaction mixture was filtered through Celite and the celite washed twice with acetic acid (2×5 mL). TLC showed the formation of a new product revealed with ninhydrin as a dark pink spot on the TLC baseline. The filtrate was evaporated under reduced pressure to give a green solid. 10% aqueous potassium carbonate (20 mL) was added to the residue and the product extracted with dichloromethane (20 mL×3). The organic layers were combined and washed with brine, dried over magnesium sulfate, filtered and evaporated under reduced pressure to give the product as a yellow gum (128 mg, 52%).

LC-MS: calcd for C₁₄H₂₇N₃O, 253.2; found 254.2 [M+H]⁺.

¹H and ¹³C NMR were consistent with the structure.

### Preparation of N-(3-(4-(6-Nitropyridin-2-yl)piperazin-1-yl)propyl)cyclohexanecarboxamide

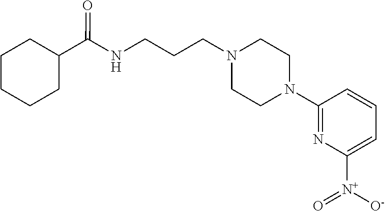 [SMILES: O=C(NCCCN1CCN(c2cccc([N+](=O)[O-])n2)CC1)C1CCCCC1]

N-(3-(piperazin-1-yl)propyl)cyclohexanecarboxamide (128 mg, 0.50 mmol), and 2-chloro-6-nitropyridine (79 mg, 0.50 mmol) were dissolved in dry acetonitrile (8 ml) in a 10 mL microwave tube and diisopropylethylamine (87 μL, 0.50 mmol) added. The mixture was stirred and then heated in the microwave oven at 120° C. for 60 min. TLC (5% MeOH-EtOAc) showed the presence of 2 close running products of Rf 0.16 and 0.10. The reaction mixture was evaporated under reduced pressure to give a brown residue that was purified by chromatography on silica gel eluting with a 0-10% methanol in ethyl acetate gradient. The product was further purified by semi-preparative HPLC using a gradient of 50-95% methanol in water over 20 min @18 ml/min (Gemini C18, 110 Å, 150×21.2 mm, 5 μm; Rt 10.8 min) to give the product (37 mg, 19%).

LCMS: calcd for C₁₉H₂₉N₅O₃, 375.2; found 376.1 [M+H]⁺.

¹H and ¹³C NMR were consistent with the structure.

Purity was 96% by HPLC (UV 254 nm).

### Preparation of [¹⁸F]N-(3-(4-(6-fluoropyridin-2-yl)piperazin-1-yl)propyl)cyclohexanecarboxamide

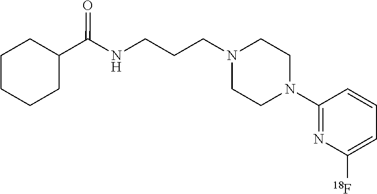 [SMILES: O=C(NCCCN1CCN(c2cccc([18F])n2)CC1)C1CCCCC1]

The synthesis was performed manually, shielded by a lead castle. Potassium carbonate (50 μL, 0.10 M) was added to kryptofix (5.0 mg) and anhydrous acetonitrile (0.50 mL) in a 3 mL Wheaton vial equipped with a stirrer vane. [¹⁸F]-fluoride (aq., approx. 250 μL, 344 MBq) was added to the vial, and heated to 110° C. under a stream of N₂ to azeotropically dry the fluoride. Two further portions of anhydrous acetonitrile (2×0.5 mL) were added and similarly dried. The reaction vial was cooled to room temperature, and N-(3-(4-(6-nitropyridin-2-yl)piperazin-1-yl)propyl)cyclohexanecarboxamide (1.0 mg) in mixed DMF (250 μL)/DMSO (50 μL) was added. The reaction was stirred at 150° C. for 15 min. The reaction was cooled to room temperature, diluted in water (1.5 mL) and loaded to a semi-preparative HPLC system. The product was collected using a manual switch, and diluted in water (18 mL). The product was loaded to a primed (1 mL ethanol, 2 mL water) tC18 Light Sep-pak cartridge, and washed with water (2 mL). The product was eluted in ethanol (0.3 mL), and diluted with phosphate buffered saline (2.7 mL). Yield=8.6% (28.5 MBq). RCP >99%. SA=7 GBq/μmol.

| | Semi-preparative | Analytical |
|---|---|---|
| Column | ACE-5 C18 10 × 100 mm | Phenomenex Luna C18, |
| | | 5μ, 4.6 × 150 mm |
| Eluant | A = 0.8% NEt₃ in H₂O, | A = H₂O |
| | adjusted to pH = 7.5 with | B = MeCN |
| | H₃PO₄ | |
| | B = MeOH | |
| Gradient | 0-1 min 35% B | 0-1 min 50% B |
| | 1-30 min 35-60% B | 1-20 min 50-95% B |
| | 30-40 min 60% B | 20-25 min 95% B |
| | 40-41 min 60-95% B | |
| Flow rate | 3.0 | 1.0 |
| (mL/min) | | |
| Retention | 36.3 | 4.9 (peak shifted by several |
| time (min) | | min on repeat injections) |

Compounds that were synthesized following procedures that were similar or identical as described those in Examples 8-10, 15, and 16 include the following:

| Structure | Name | [M + H]⁺ |
|---|---|---|
| 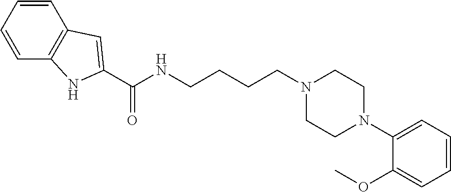 [SMILES: COc1ccccc1N1CCN(CCCCNC(=O)c2cc3ccccc3[nH]2)CC1] | N-(4-(4-(2- methoxyphenyl)piperazin-1- yl)butyl)-1H-indole-2-carboxamide | 407.4 |
| 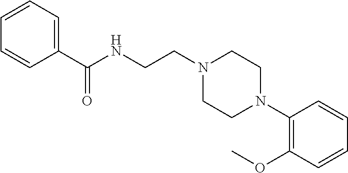 [SMILES: COc1ccccc1N1CCN(CCNC(=O)c2ccccc2)CC1] | N-(2-(4-(2- methoxyphenyl)piperazin-1- yl)ethyl)benzamide | 340.3 |
| 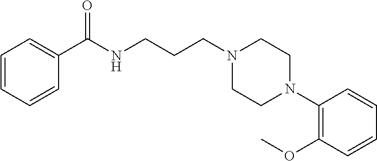 [SMILES: COc1ccccc1N1CCN(CCCNC(=O)c2ccccc2)CC1] | N-(3-(4-(2- methoxyphenyl)piperazin-1- yl)propyl)benzamide | 354.4 |
| 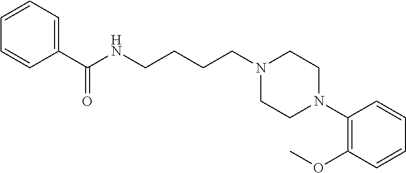 [SMILES: COc1ccccc1N1CCN(CCCCNC(=O)c2ccccc2)CC1] | N-(4-(4-(2- methoxyphenyl)piperazin-1- yl)butyl)benzamide | 368.4 |
| 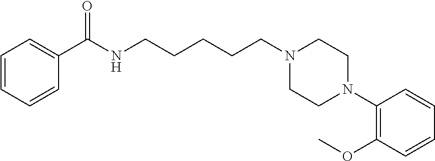 [SMILES: COc1ccccc1N1CCN(CCCCCNC(=O)c2ccccc2)CC1] | N-(5-(4-(2- methoxyphenyl)piperazin-1- yl)pentyl)benzamide | 382.4 |
| 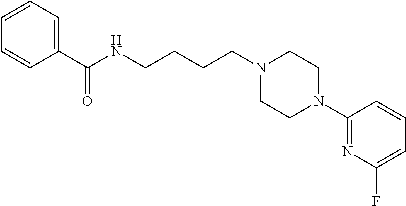 [SMILES: O=C(NCCCCN1CCN(c2cccc(F)n2)CC1)c1ccccc1] | N-(4-(4-(6-fluoropyridin-2- yl)piperazin-1-yl)butyl)benzamide | 357.4 |
| 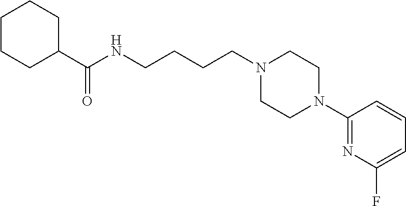 [SMILES: O=C(NCCCCN1CCN(c2cccc(F)n2)CC1)C1CCCCC1] | N-(4-(4-(6-fluoropyridin-2- yl)piperazin-1- yl)butyl)cyclohexanecarboxamide | 363.4 |
| 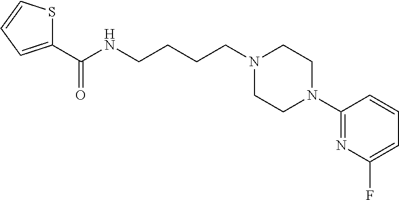 [SMILES: O=C(NCCCCN1CCN(c2cccc(F)n2)CC1)c1cccs1] | N-(4-(4-(6-fluoropyridin-2- yl)piperazin-1-yl)butyl)thiophene- 2-carboxamide | 362.3 |
| 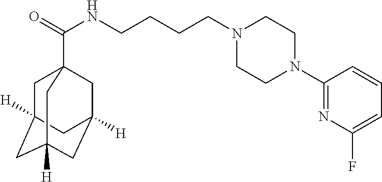 [SMILES: O=C(NCCCCN1CCN(c2cccc(F)n2)CC1)C12C[C@H]3C[C@@H](C1)C[C@@H](C2)C3] | (3r,5r,7r)-N-(4-(4-(6-fluoropyridin- 2-yl)piperazin-1- yl)butyl)adamantane-1- carboxamide | 415.5 |
| 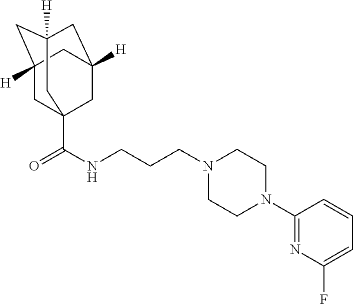 [SMILES: O=C(NCCCN1CCN(c2cccc(F)n2)CC1)C12C[C@H]3C[C@@H](C1)C[C@@H](C2)C3] | (3r,5r,7r)-N-(3-(4-(6-fluoropyridin- 2-yl)piperazin-1- yl)propyl)adamantane-1- carboxamide | 401.4 |
| 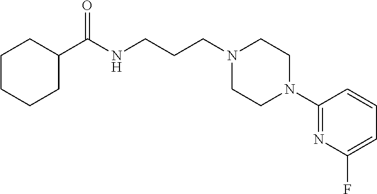 [SMILES: O=C(NCCCN1CCN(c2cccc(F)n2)CC1)C1CCCCC1] | N-(3-(4-(6-fluoropyridin-2- yl)piperazin-1- yl)propyl)cyclohexanecarboxamide | 349.4 |
| 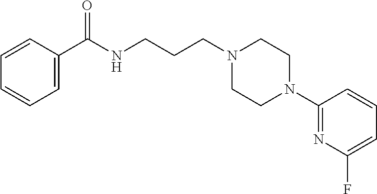 [SMILES: O=C(NCCCN1CCN(c2cccc(F)n2)CC1)c1ccccc1] | N-(3-(4-(6-fluoropyridin-2- yl)piperazin-1-yl)propyl)benzamide | 343.3 |
| 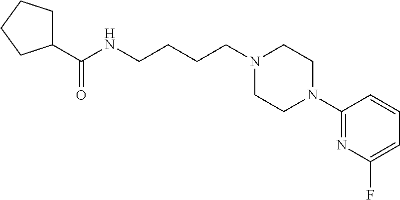 [SMILES: O=C(NCCCCN1CCN(c2cccc(F)n2)CC1)C1CCCC1] | N-(4-(4-(6-fluoropyridin-2- yl)piperazin-1- yl)butyl)cyclopentanecarboxamide | 349.4 |
| 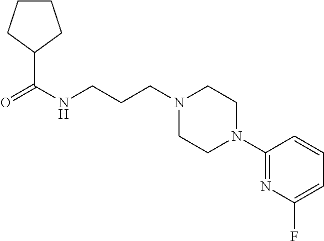 [SMILES: O=C(NCCCN1CCN(c2cccc(F)n2)CC1)C1CCCC1] | N-(3-(4-(6-fluoropyridin-2- yl)piperazin-1- yl)propyl)cyclopentanecarboxamide | 335.3 |
| 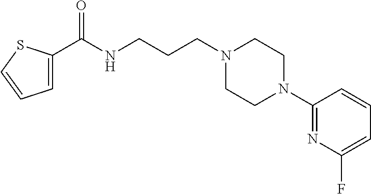 [SMILES: O=C(NCCCN1CCN(c2cccc(F)n2)CC1)c1cccs1] | N-(3-(4-(6-fluoropyridin-2- yl)piperazin-1-yl)propyl)thiophene- 2-carboxamide | 349.3 |
| 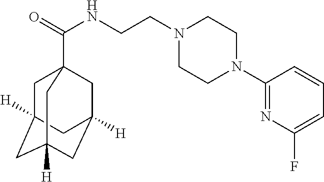 [SMILES: O=C(NCCN1CCN(c2cccc(F)n2)CC1)C12C[C@H]3C[C@@H](C1)C[C@@H](C2)C3] | (3r,5r,7r)-N-(2-(4-(6-fluoropyridin- 2-yl)piperazin-1- yl)ethyl)adamantane-1- carboxamide | 387.4 |
| 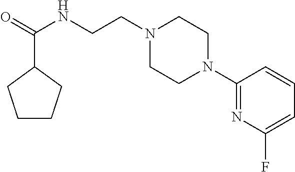 [SMILES: O=C(NCCN1CCN(c2cccc(F)n2)CC1)C1CCCC1] | N-(2-(4-(6-fluoropyridin-2- yl)piperazin-1- yl)ethyl)cyclopentanecarboxamide | 321.3 |
| 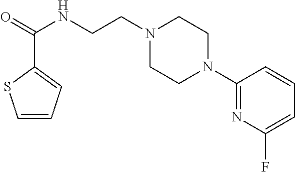 [SMILES: O=C(NCCN1CCN(c2cccc(F)n2)CC1)c1cccs1] | N-(2-(4-(6-fluoropyridin-2- yl)piperazin-1-yl)ethyl)thiophene- 2-carboxamide | 335.3 |
| 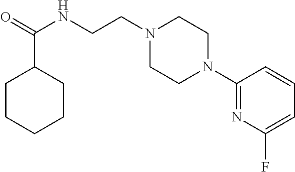 [SMILES: O=C(NCCN1CCN(c2cccc(F)n2)CC1)C1CCCCC1] | N-(2-(4-(6-fluoropyridin-2- yl)piperazin-1- yl)ethyl)cyclohexanecarboxamide | 335.3 |
| 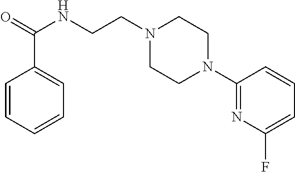 [SMILES: O=C(NCCN1CCN(c2cccc(F)n2)CC1)c1ccccc1] | N-(2-(4-(6-fluoropyridin-2- yl)piperazin-1-yl)ethyl)benzamide | 329.3 |
| 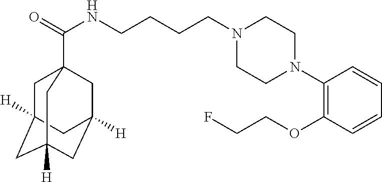 [SMILES: O=C(NCCCCN1CCN(c2ccccc2OCCF)CC1)C12C[C@H]3C[C@@H](C1)C[C@@H](C2)C3] | (3r,5r,7r)-N-(4-(4-(2-(2- fluoroethoxy)phenyl)piperazin-1- yl)butyl)adamantane-1- carboxamide | 458.5 |
| 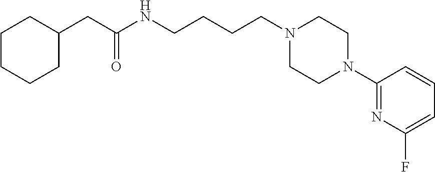 [SMILES: O=C(CC1CCCCC1)NCCCCN1CCN(c2cccc(F)n2)CC1] | 2-cyclohexyl-N-(4-(4-(6- fluoropyridin-2-yl)piperazin-1- yl)butyl)acetamide | 377.4 |
| 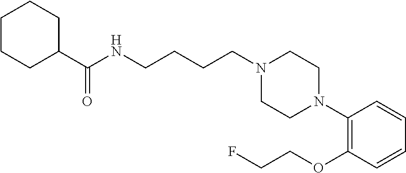 [SMILES: O=C(NCCCCN1CCN(c2ccccc2OCCF)CC1)C1CCCCC1] | N-(4-(4-(2-(2- fluoroethoxy)phenyl)piperazin-1- yl)butyl)cyclohexanecarboxamide | 406.2 |
| 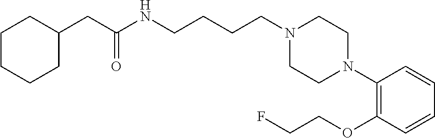 [SMILES: O=C(CC1CCCCC1)NCCCCN1CCN(c2ccccc2OCCF)CC1] | 2-cyclohexyl-N-(4-(4-(2-(2- fluoroethoxy)phenyl)piperazin-1- yl)butyl)acetamide | 420.2 |
| 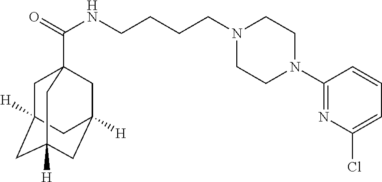 [SMILES: O=C(NCCCCN1CCN(c2cccc(Cl)n2)CC1)C12C[C@H]3C[C@@H](C1)C[C@@H](C2)C3] | (3r,5r,7r)-N-(4-(4-(6-chloropyridin- 2-yl)piperazin-1- yl)butyl)adamantane-1- carboxamide | 431.9 |
| 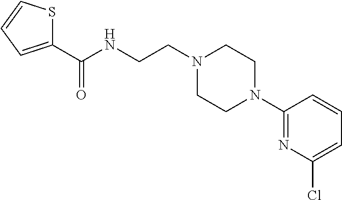 [SMILES: O=C(NCCN1CCN(c2cccc(Cl)n2)CC1)c1cccs1] | N-(2-(4-(6-chloropyridin-2- yl)piperazin-1-yl)ethyl)thiophene- 2-carboxamide | 351.8 |
| 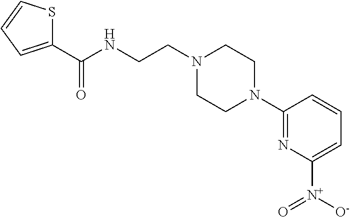 [SMILES: O=C(NCCN1CCN(c2cccc([N+](=O)[O-])n2)CC1)c1cccs1] | N-(2-(4-(6-nitropyridin-2- yl)piperazin-1-yl)ethyl)thiophene- 2-carboxamide | 362.4 |
| 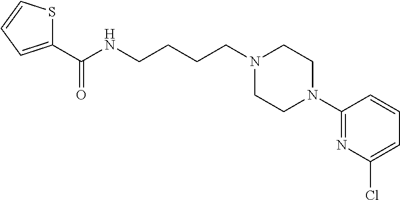 [SMILES: O=C(NCCCCN1CCN(c2cccc(Cl)n2)CC1)c1cccs1] | N-(4-(4-(6-chloropyridin-2- yl)piperazin-1-yl)butyl)thiophene- 2-carboxamide | 379.9 |
| 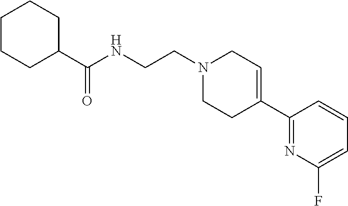 [SMILES: O=C(NCCN1CC=C(c2cccc(F)n2)CC1)C1CCCCC1] | N-(2-(6-fluoro-5′,6′-dihydro-[2,4′- bipyridin]-1′(2′H)- yl)ethyl)cyclohexanecarboxamide | 332.3 |
| 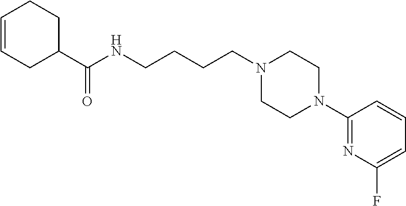 [SMILES: O=C(NCCCCN1CCN(c2cccc(F)n2)CC1)C1CC=CCC1] | N-(4-(4-(6-fluoropyridin-2- yl)piperazin-1-yl)butyl)cyclohex-3- enecarboxamide | 361.3 |
| 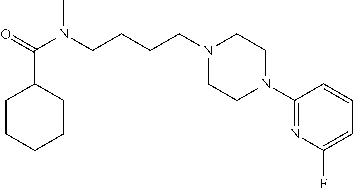 [SMILES: CN(CCCCN1CCN(c2cccc(F)n2)CC1)C(=O)C1CCCCC1] | N-(4-(4-(6-fluoropyridin-2- yl)piperazin-1-yl)butyl)-N- methylcyclohexanecarboxamide | 377.3 |
| 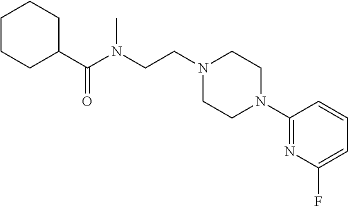 [SMILES: CN(CCN1CCN(c2cccc(F)n2)CC1)C(=O)C1CCCCC1] | N-(2-(4-(6-fluoropyridin-2- yl)piperazin-1-yl)ethyl)-N- methylcyclohexanecarboxamide | 348.9 |
| 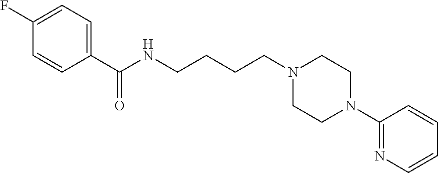 [SMILES: O=C(NCCCCN1CCN(c2ccccn2)CC1)c1ccc(F)cc1] | 4-fluoro-N-(4-(4-(pyridin-2- yl)piperazin-1-yl)butyl)benzamide | 357.2 |
| 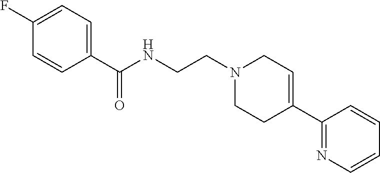 [SMILES: O=C(NCCN1CC=C(c2ccccn2)CC1)c1ccc(F)cc1] | 4-fluoro-N-(2-(4-(pyridin-2- yl)piperazin-1-yl)ethyl)benzamide | 329.3 |
| 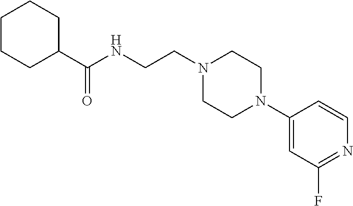 [SMILES: O=C(NCCN1CCN(c2ccnc(F)c2)CC1)C1CCCCC1] | N-(2-(4-(2-fluoropyridin-4- yl)piperazin-1- yl)ethyl)cyclohexanecarboxamide | 335.3 |
| 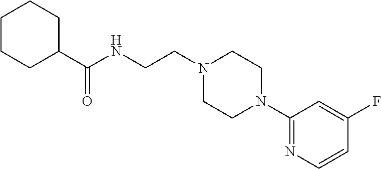 [SMILES: O=C(NCCN1CCN(c2cc(F)ccn2)CC1)C1CCCCC1] | N-(2-(4-(4-fluoropyridin-2- yl)piperazin-1- yl)ethyl)cyclohexanecarboxamide | 335.3 |
| 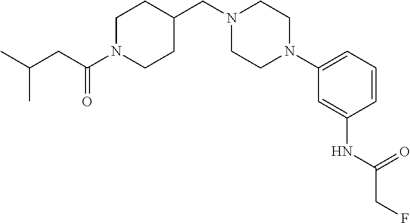 [SMILES: CC(C)CC(=O)N1CCC(CN2CCN(c3cccc(NC(=O)CF)c3)CC2)CC1] | 2-fluoro-N-(3-(4-((1-(3- methylbutanoyl)piperidin-4- yl)methyl)piperazin-1- yl)phenyl)acetamide | 419.4 |

### Example 11

### Scheme 11 Shows a Method for Making Radiolabeled Compounds of Formula (I) for Preparation of 2-((4-(2-(2-fluoroethoxy)phenyl)piperazin-1-yl)methyl)benzo[d]thiazole

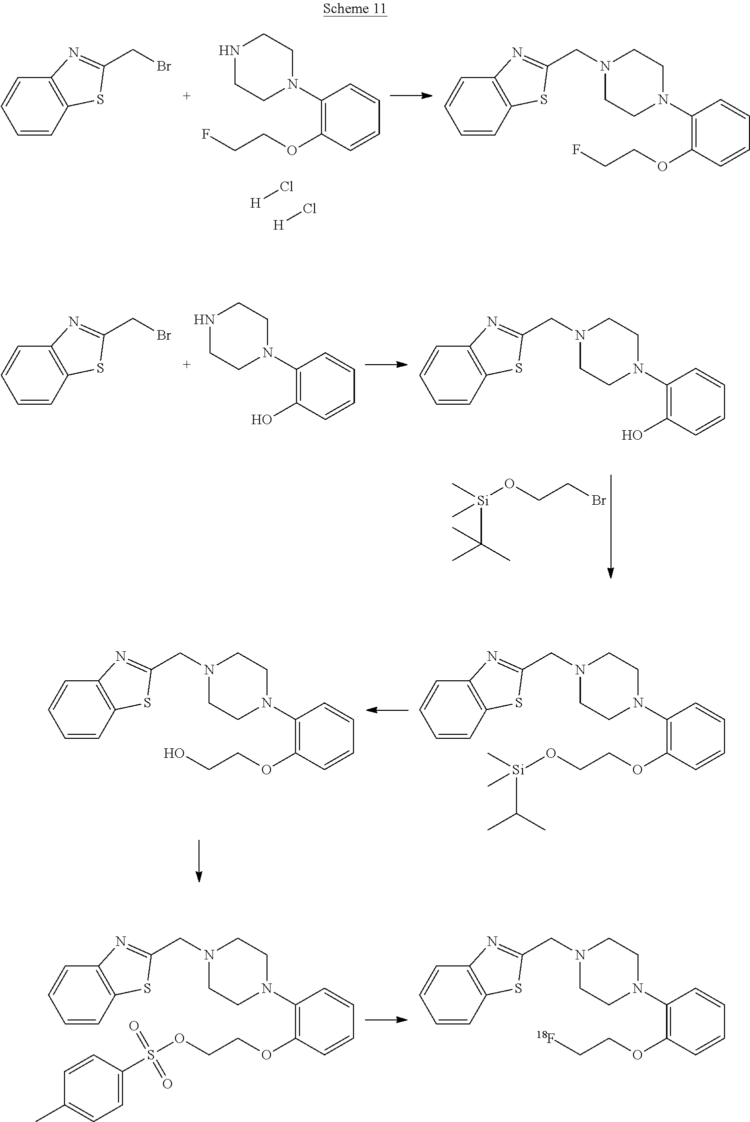

### Preparation of 2-((4-(2-(2-Fluoroethoxy)phenyl)piperazin-1-yl)methyl)benzo[d]thiazole

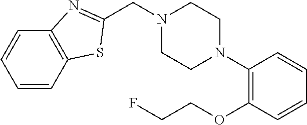 [SMILES: FCCOc1ccccc1N1CCN(Cc2nc3ccccc3s2)CC1]

2-(bromomethyl)benzo[d]thiazole (100 mg, 0.438 mmol) and 1-(2-(2-fluoroethoxy)phenyl)piperazine dihydrochloride (130 mg, 0.438 mmol) were dissolved in acetonitrile (2.5 ml) in a 10 mL microwave tube and approx 8 drops triethylamine added from a glass pippette. The mixture was heated to 100° C. for a total of 30 mins when almost all starting material was consumed. Evaporated solvent and added dichloromethane (25 mL) and washed with water, separating with a phase separator. Evaporated and purified by chormatography on silica gel (10 g) eluting with 10-75% EtOAc-petrol over 30 CV. Product peak fractions (9-12) were evaporated to give the product (100 mg, 61%) as a colourless viscous oil.

LCMS calculated for C₂₀H₂₂FN₃OS 371.2; found 372.1 [M+H]⁺.

¹H NMR (300 MHz, CDCl₃): δ 2.78-2.91 (4H, br, pip-CH₂), 3.10-3.28 (4H, br, pip-CH₂), 4.03 (2H, s, CH₂—N), 4.25 (2H, dt, J=28.0 Hz & 4.0 Hz, OCH₂CH₂F), 4.75 (2H, dt, J=47.0 Hz & 4.0 Hz, CH₂F), 6.82-6.89 (1H, m, phenyl-H), 6.94-7.00 (3H, m, phenyl-H), 7.33-7.49 (2H, m, benzo-C5,6-H), 7.87 (1H, dm, J=8.0 Hz, benzo-C7-H) and 7.98 (1H, dm, J=8.0 Hz, benzo-C4-H). ¹³C NMR (75 MHz, CDCl₃): δ 50.5 (pip-CH₂), 53.7 (pip-CH₂), 60.4 (CH₂—N), 67.6 (d, J=24.5 Hz, OCH₂), 81.9 (d, J=169.5 Hz, CH₂F), 113.7 (phenyl-C3), 118.5, 121.7, 122.1, 122.7, 122.8, 124.8 and 125.8 (aryl-CH), 135.4 (aryl-C—S), 141.9 (phenyl-CO), 151.0 (aryl-C—N), 153.3 (phenyl-C1) and 172.1 (N═C—S).

### Preparation of 2-(4-(Benzo[d]thiazol-2-ylmethyl)piperazin-1-yl)phenol

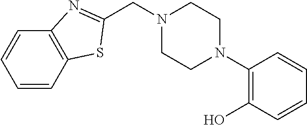 [SMILES: Oc1ccccc1N1CCN(Cc2nc3ccccc3s2)CC1]

To a suspension of 2-(bromomethyl)benzo[d]thiazole (0.175 g, 0.76 mmol) and diisopropylethyl amine (0.125 g, 1 mmol) was added 2-(piperazin-1-yl)phenol (0.150 g, 0.84 mmol) and acetonitrile (2 mL). The resultant mixture was heated at 50° C. for 20 minutes in a CEM microwave reactor. The mixture was then cooled to room temperature, and the solvent was evaporated at reduced pressure. The crude mixture was diluted in dichloromethane (3 mL) and purified by chromatography on silica gel (50 g) eluting with ethyl acetate at a flow of 40 mL/min. The product eluted between 1.5-2.5 CV. This yielded an off-white solid (185 mg, 75%).

LCMS: m/z calcd for C₁₈H₁₉N₃OS 325.1; found, 326.0 [M+H]⁺.

¹H NMR (300 MHz, CDCl₃): δ 2.86 (4H, brs, pip), 2.97 26 (4H, brs, pip), 4.04 (2H, s, NCH₂C═N), 7.80-7.53 (6H, m, ArH), and 8.80-9.05 (2H, m, ArH); ¹³C NMR (75 MHz, CDCl₃): δ 52.5, 54.0, 60.2, 114.1, 120.0, 121.4, 121.7, 122.8, 124.9, 125.9, 126.5, 135.1, 138.8, 151.4, 153.3, and 171.5.

### Preparation of 2-((4-(2-(2-(tert-Butyldimethylsilyloxy)ethoxy)phenyl)piperazin-1-yl)methyl)benzo[d]thiazole

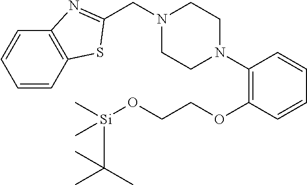 [SMILES: CC(C)(C)[Si](C)(C)OCCOc1ccccc1N1CCN(Cc2nc3ccccc3s2)CC1]

To a suspension of 2-(4-(benzo[d]thiazol-2-ylmethyl)piperazin-1-yl)phenol (0.2 g, 0.61 mmol) and cesium carbonate (0.500 g, 1.5 mmol) in acetonitrile (5 mL) was added (2-bromoethoxy)(tert-butyl)dimethylsilane (0.239 g, 1.0 mmol) and triethylamine (0.073 g, 0.73 mmol) was heated at 60° C. for 18 hours. The reaction mixture was concentrated and partitioned between dichloromethane (50 mL) and water (50 mL). The organics were dried and concentrated. This material was dissloved in dichloromethane (3 mL) and separated on silica gel (50 g) eluting with 25% ethyl acetate in petrol for 2 CV then ethyl acetate (25-100%) in petrol over 10 CV at a flow of 40 mL/min. The product eluted between 4-6 CV. This was concentrated to yield a yellow oil (100 mg, 34%).

LC-MS: m/z calcd for C₂₆H₃₇N₃O₂SSi 483.2; found, 484.1 [M+H]⁺.

¹H NMR (300 MHz, CDCl₃): δ 0.07 (6H, s, 2×SiCH₃), 0.88 (9H, s, SiC(CH₃)₃), 2.83 (4H, brs, pip), 3.19 (4H, brs, pip), 3.98 (2H, t, J=4.9 Hz), 4.03 (2H, s, NCH₂C═N), 4.08 (2H, t, J=4.9 Hz), 6.82-7.01 (4H, m, ArH), 7.36 (1H, dt, J=1.2 and 8 Hz, ArH), 7.46 (1H, dt, J=1.2 and 8 Hz, ArH), 7.87 (1H, d, J=8 Hz, ArH), and 7.98 (1H, d, J=8 Hz, ArH); ¹³C NMR (75 MHz, CDCl₃): δ 52.5, 54.0, 60.2, 114.1, 120.0, 121.4, 121.7, 122.8, 124.9, 125.9, 126.5, 135.1, 138.8, 151.4, 153.3, and 171.5;

### Preparation of 2-(2-(4-(Benzo[d]thiazol-2-ylmethyl)piperazin-1-yl)phenoxy)ethanol

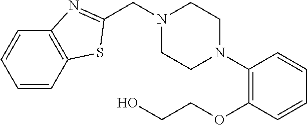 [SMILES: OCCOc1ccccc1N1CCN(Cc2nc3ccccc3s2)CC1]

To 2-((4-(2-(2-(tert-Butyldimethylsilyloxy)ethoxy)phenyl)piperazin-1-yl)methyl)benzo[d]thiazole (0.08 g, 0.17 mmol) was added tertabutylammonium fluoride in THF (1 mmol, 1 mL of a 1 M solution). The reaction was stirred for 10 minutes then partitioned between dichloromethane (10 mL) and 1N hydrochloric acid (10 mL). The organics were dried and concentrated. The crude material dissloved in dichloromethane (3 mL) and separated on silica (10 g) eluting with 30% ethyl acetate in petrol for 5 CV then ethyl acetate (30-100%) in petrol over 15 CV then held for 8 CV at a flow of 30 mL/min. The product eluted between 19-27 CV. This was concentrated to yield a white solid (51 mg, 81%).

LC-MS: m/z calcd for C₂₀H₂₃N₃O₂S 369.2; found, 370.1 [M+H]⁺.

¹H NMR (300 MHz, CDCl₃): δ 2.85 (4H, t, J=4.9 Hz, pip), 3.16 (4H, t, J=4.9 Hz, pip), 3.68 (2H, t, J=4.6 Hz), 4.02 (2H, s, NCH₂C═N), 4.16 (2H, t, J=4.9 Hz), 6.97-7.05 (4H, m, ArH), 7.35 (1H, dt, J=1.2 and 8 Hz, ArH), 7.44 (1H, dt, J=1.2 and 8 Hz, ArH), 7.86 (1H, d, J=8 Hz, ArH), and 7.98 (1H, d, J=8 Hz, ArH); ¹³C NMR (75 MHz, CDCl₃): δ 51.4, 53.4, 60.1, 60.6, 74.4, 118.9, 119.0, 121.6, 122.8, 123.4, 124.3, 124.8, 125.8, 135.3, 143.7, 152.0, 153.3, and 171.9.

### Preparation of 2-(2-(4-(benzo[d]thiazol-2-ylmethyl)piperazin-1-yl)phenoxy)ethyl 4-methyl-benzenesulfonate

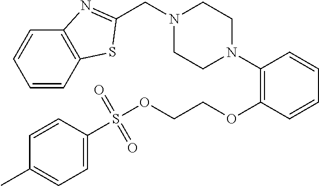 [SMILES: Cc1ccc(S(=O)(=O)OCCOc2ccccc2N2CCN(Cc3nc4ccccc4s3)CC2)cc1]

To a solution of 2-(2-(4-(benzo[d]thiazol-2-ylmethyl)piperazin-1-yl)phenoxy)ethanol (0.051 g, 0.14 mmol) in dichloromethane (3 mL) and triethylamine (0.073 g, 0.73 mmol) was added tosyl chloride (0.029 g, 0.15 mmol) at 0° C. The resultant mixture was allowed to warm to room temperature and was stirred for 18 hours. The solvent was evaporated at reduced pressure. The crude mixture was diluted in dichloromethane (3 mL) and purified by chromatography on silica gel (10 g) eluting with 25% ethyl acetatae in petrol for 2 CV up to 100% ethyl acetate over 18 CV at a flow of 30 mL/min. The product eluted between 8-12 CV. This yielded a white solid (28 mg, 38%).

LC-MS: m/z calcd for C₂₇H₂₉N₃O₄S₂ 523.2; found, 524.1 [M+H]⁺.

¹H NMR (300 MHz, CDCl₃): δ 2.39 (3H, s, PhCH₃), 2.76 (4H, t, J=4.9 Hz, pip), 3.12 (4H, t, J=4.9 Hz, pip), 4.00 (2H, s, NCH₂C═N), 4.21 (2H, t, J=4.6 Hz), 4.39 (2H, t, J=4.9 Hz), 6.73-6.79 (1H, m, ArH), 6.89-7.00 (3H, m, ArH), 7.29 (2H, d, J=6 Hz, ArH), 7.37 (1H, dt, J=1.2 and 8 Hz, ArH), 7.46 (1H, dt, J=1.2 and 8 Hz, ArH), 7.78 (2H, d, J=6 Hz, ArH), 7.88 (1H, d, J=8 Hz, ArH), and 7.99 (1H, d, J=8 Hz, ArH).

### Preparation of [¹⁸F]2-((4-(2-(2-Fluoroethoxy)phenyl)piperazin-1-yl)methyl)benzo[d]thiazole

The Kryptofix/potassium carbonate solution (molar ratio 2:1, MeCN/H₂O 96:4, 2 mL) and precursor 2-(2-(4-(benzo[d]thiazol-2-ylmethyl)piperazin-1-yl)phenoxy)ethyl 4-methylbenzenesulfonate (2.0 mg in 0.6 mL acetonitrile) were added to vials 1 and 3, respectively of the Tracer Lab synthesizer. Vials 5, 7, 8 and 9 were filled with H₂O (2 mL), H₂O (1 mL), MeCN (0.5 mL), H₂O (5 mL) respectively. The target water containing ¹⁸F⁻ was passed through a pre-conditioned QMA cartridge where the ¹⁸F⁻ was trapped. The ¹⁸F⁻ was released from the QMA cartridge and carried into the reactor by passing the Kryptofix/potassium carbonate solution from vial 1 through the cartridge. The mixture was dried at 100° C. for 30 min. The precursor solution from vial 3 was added to the dried [K/K.2.2.2]⁺¹⁸F⁻ complex. The reaction mixture was heated for 30 min at 78° C. After reaction the crude was diluted with water from vial 5 and transferred to the round bottomed flask containing H₂O (10 mL), then transferred to a pre-conditioned SPE cartridge. The SPE was washed with the water from vial 9 and then the crude was eluted into a vial with acetonitrile and water (from vial 8 and 7). The crude was further diluted with H₂O (0.5 mL) before injected on the semi-preparative HPLC system. The mobile phase used for purification was: A: H₂O B: MeCN. Flow rate=3.0 mL/min. Gradient: 50-95% over 20 min. Column: Phenomenex Luna 100×10 mm, 5 micron, C18. The product fraction (Rt=12.1 min) was collected and diluted with H₂O (5 mL) and passed through a pre-conditioned C18 Light SPE cartridge. The purified product was trapped on the cartridge and washed with H₂O (1 mL) and eluted with ethanol (1.0 mL) and phosphate buffer (9 mL)

The formulated product was finally analyzed on the analytical HPLC system before being released to biology. Analytical HPLC conditions: A:H₂O B:MeCN. Gradient: 50-95% over 20 min. Flow rate=1 mL/min Column: Phenomenex Luna 150×4.6 mm, C18. Rt=11.0 min.

Starting with 25.9 GBq gave 251 MBq formulated [¹⁸F]GEH120270 after a total synthesis time of 167 min. RCP: >99%. RCY: 1%. SRA: 66 GBq/mmol. Amount cold compound: 0.14 μg/mL.

### Example 12

### Scheme 12 Shows a Method for Making Radiolabeled Compounds of Formula (I) for Preparation of 2-(4-(4-(2-(2-fluoroethoxy)phenyl)piperazin-1-yl)butyl)-4-methyl-1,2,4-triazine-3,5(2H,4H)-dione

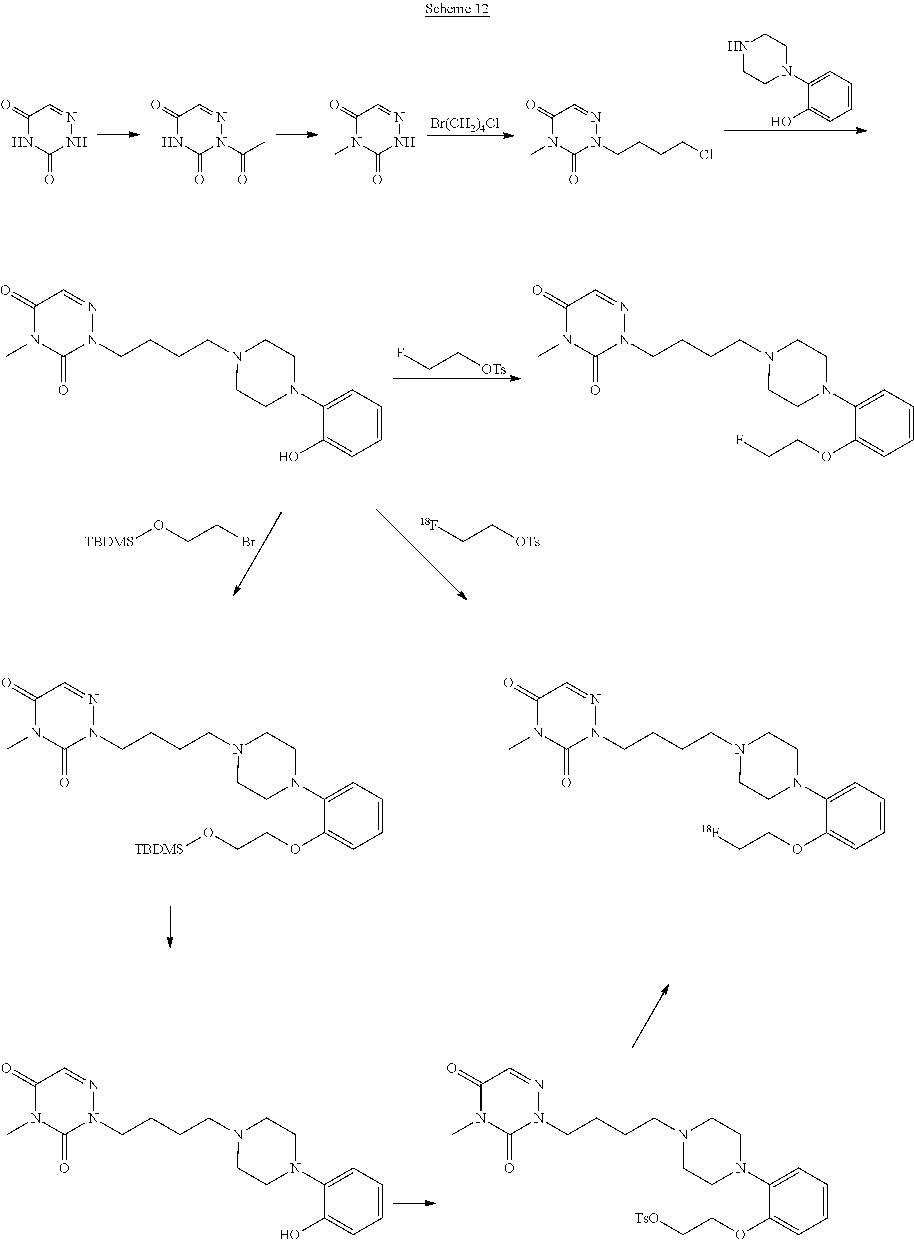

### Preparation of 2-Acetyl-1,2,4-triazine-3,5(2H,4H)-dione

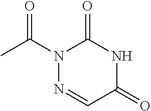

6-Azauracil (10 g, 89.0 mmol) was added to acetic anhydride (60 mL) and heated to reflux for 120 mins when a clear solution resulted. After cooling, the solution was evaporated to dryness then toluene (150 mL) was added, thoroughly mixed and co-evaporated. The resultant solid was triturated with toluene (200 mL) and filtered to give a white solid that was dried in the vacuum oven at 55° C. 2-Acetyl-1,2,4-triazine-3,5(2H,4H)-dione was isolated as a white solid (11.55 g, 84%).

¹H NMR (300 MHz, DMSO-d6) δ 2.48 (3H, s, CH₃), and 7.67 (1H, s, N═CH). ¹³C NMR (75 MHz, DMSO-d6) δ 25.4 (CH₃), 137.2 (N═CH), 146.4 (NHC(═O)NH), 156.2 (NHC(═O)CH), and 169.4 (CH₃C═O).

### Preparation of 4-Methyl-1,2,4-triazine-3,5(2H,4H)-dione

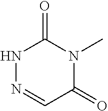

2-Acetyl-1,2,4-triazine-3,5(2H,4H)-dione (10 g, 64.5 mmol) was added portionwise over 40 min to a suspension of sodium hydride (3.09 g, 60 wt % suspension in oil, 77.4 mmol) in DMF (160 mL) and stirred vigorously for 45 min. Iodomethane (4.41 mL, 10.1 g, 70.9 mmol) was added and the whole mixture stirred at ambient temperature for 66 h under a dry nitrogen atmosphere. A slight ppt remained but this disappeared on warming to evaporate the solvent. The residue was dissolved in EtOH (200 mL) and para-toluenesulfonic acid monohydrate (1.4 g, ˜10 mol %) was added and the yellow solution refluxed for 2 h before allowing to cool. Solvent was evaporated and toluene (275 mL) added with heating. After standing overnight, solid was filtered off. The crude solid was suspended in EtOAc (250 mL) and stirred overnight to dissolve out sodium iodide, then filtered off to give 4-methyl-1,2,4-triazine-3,5(2H,4H)-dione (4.9 g, 60%).

¹H NMR (300 MHz, DMSO-d6) δ 3.10 (3H, s, CH₃), and 7.48 (1H, s, CH). ¹³C NMR (75 MHz, DMSO-d6) δ 25.6 (CH₃), 134.4 (CH), 149.4 (NHC(═O)CH), and 156.5 (NHC(═O)NH).

### Preparation of 2-(4-chlorobutyl)-4-methyl-1,2,4-triazine-3,5(2H,4H)-dione

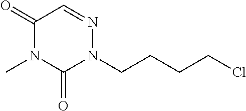

A solution of 4-methyl-1,2,4-triazine-3,5(2H,4H)-dione (4.5 g, 35.4 mmol) in dry DMF (40 mL) was added slowly to a suspension of sodium hydride (1.56 g, 60% dispersion in oil, 38.9 mmol) in dry DMF (15 mL). The mixture was stirred for 1.5 h under a balloon of dry nitrogen. To the yellow suspension was added 1-bromo-4-chlorobutane (6.67 g, 4.48 mL, 38.9 mmol) and the mixture allowed to stir at ambient temperature overnight when tlc (70:30 toluene:EtOAc) showed a single spot at Rf 0.5. Water (200 mL) was added and the product extracted with diethyl ether (3×70 mL). The combined organics were washed with brine (40 mL), dried over Na₂SO₄, filtered and evaporated to a yellow oil (8.92 g). The product was isolated after chromatography on 120 g silica gel eluting with EtOAc-petrol (5-85% over 12 CV) with the main peak eluting at 30% EtOAc. Combined fractions were evaporated to give 2-(4-chlorobutyl)-4-methyl-1,2,4-triazine-3,5(2H,4H)-dione as a clear viscous oil (5.65 g, 73%).

¹H NMR (300 MHz, DMSO-d6) δ 1.72-1.88 (4H, m, 2×CH₂), 3.14 (3H, s, CH₃), 3.66 (2H, t, J=6.5 Hz CH₂N), 3.91 (2H, t, J=6.5 Hz CH₂Cl), and 7.57 (1H, s, CH). ¹³C NMR (75 MHz, DMSO-d6) δ 25.1 (CH₂), 26.5 (CH₂), 28.9 (CH₃), 45.0 (CH₂N), 50.1 (CH₂Cl) 134.0 (CH), 148.6 (NHC(═O)CH), and 156.1 (NHC(═O)NH).

### Preparation of 2-(4-(4-(2-hydroxyphenyl)piperazin-1-yl)butyl)-4-methyl-1,2,4-triazine-3,5(2H,4H)-dione

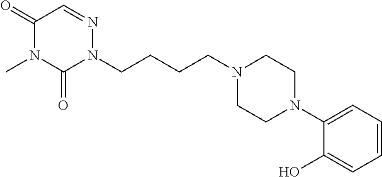 [SMILES: Cn1c(=O)cnn(CCCCN2CCN(c3ccccc3O)CC2)c1=O]

Triethylamine (5 mL) was added to a solution of 2-(4-chlorobutyl)-4-methyl-1,2,4-triazine-3,5(2H,4H)-dione (1.83 g, 8.4 mmol) and 1-(2-hydroxyphenyl)piperazine (1.5 g, 8.4 mmol) in 1-butanol (50 mL) and the mixture heated to reflux for 14 h. On cooling, the solvent was evaporated and the residue was taken up in ether (500 mL) and washed with water (2×50 mL), brine (50 mL), dried (MgSO4) filtered and evaporated to give a yellow solid (1.38 g). Tlc (5% MeOH-DICHLOROMETHANE) showed one main spot at Rf 0.5. The product was isolated after chromatography on 50 g silica gel eluting with 1-10% MeOH-DICHLOROMETHANE gradient and the main peak coming off at 8%. Evaporation of appropriate fractions gave the product as a pale grey solid (1.25 g, 41%).

LC-MS: calcd for C₁₈H₂₅N₅O₃ 359.2; found, 360.1 (M+H)⁺.

¹H NMR (300 MHz, CDCl₃) δ 1.52-1.62 (2H, m, CH₂), 1.75-1.87 (2H, m, CH₂), 2.45 (2H, t, J=6.5 Hz CH₂N), 2.53-2.70 (4H, br, 2×CH₂N), 2.90 (4H, t, J=5.0 Hz, 2×CH₂N), 3.34 (3H, s, CH₃), 4.02 (2H, t, J=7.0 Hz CH₂N), 6.85 (1H, dt, J=7.5 & 2.0 Hz, phenyl-C4-H or -C5-H), 6.93 (1H, dd, J=8.0 & 1.0 Hz phenyl-C3-H or -C6-H), 7.07 (1H, dt, J=8.0 & 1.5 Hz phenyl-C4-H or -C5-H), 7.16 (1H, dd, J=7.5 & 1.5 Hz, phenyl-C3-H or C6-H), and 7.39 (1H, s, N═CH). ¹³C NMR (75 MHz, CDCl₃) δ 23.8 (CH₂), 26.2 (CH₂), 27.0 (CH₃), 51.7 (CH₂N), 52.5 (CH₂N), 53.9 (CH₂N), 58.0 (CH₂N), 114.0, 120.0, 121.4, 126.6 (phenyl-C3-C6), 133.8 (N═CH), 138.9 (phenyl-C2-O), 148.9 (NHC(═O)CH), 151.5 (phenyl-C1-N) and 156.2 (NHC(═O)NH).

### Preparation of 2-(4-(4-(2-(2-fluoroethoxy)phenyl)piperazin-1-yl)butyl)-4-methyl-1,2,4-triazine-3,5(2H,4H)-dione

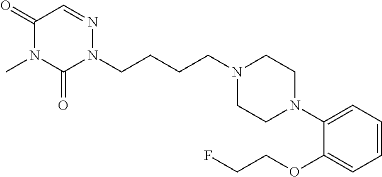 [SMILES: Cn1c(=O)cnn(CCCCN2CCN(c3ccccc3OCCF)CC2)c1=O]

2-(4-(4-(2-hydroxyphenyl)piperazin-1-yl)butyl)-4-methyl-1,2,4-triazine-3,5(2H,4H)-dione (180 mg, 0.5 mmol) and fluoroethyl tosylate (171 mg, 0.78 mmol) were dissolved in dry acetonitrile (10 mL) and cesium carbonate (180 mg, 1.30 mmol) was added. The mixture was stirred vigorously under a dry nitrogen atmosphere heating at reflux for 3 h when LC-MS showed almost complete absence of starting material. The mixture was filtered, washing with a little acetonitrile and solvent evaporated. Diethyl ether (25 mL) and water (30 mL) were added to the residue and the layers separated. The aqueous layer was extracted with ether (2×30 mL) and the combined organics washed with brine, dried (Na₂SO₄), filtered and evaporated to give a crude product (275 mg). 2-(4-(4-(2-(2-fluoroethoxy)phenyl)piperazin-1-yl)butyl)-4-methyl-1,2,4-triazine-3,5(2H,4H)-dione was isolated by chromatography on 10 g silica gel eluting with 0.5-10% MeOH-DICHLOROMETHANE over 28 CV as a sticky gum (110 mg, 54%) after removing solvent residues under high vacuum.

LC-MS: calcd for C₁₈H₂₅FN₅O₃ 405.2; found, 406.2 (M+H)⁺.

¹H NMR (300 MHz, CDCl₃) δ 1.51-1.64 (2H, m, CH₂), 1.74-1.86 (2H, m, CH₂), 2.43 (2H, t, J=7.5 Hz CH₂N), 2.55-2.71 (4H, br, 2×pip-CH₂), 3.04-3.22 (4H, br, 2×pip-CH₂), 3.34 (3H, s, CH₃), 4.02 (2H, t, J=7.0 Hz, CH₂N), 4.20 (1H, m, OCH₂), 4.30 (1H, m, OCH₂), 4.69 (1H, m, CH₂F), 4.85 (1H, m, CH₂F), 6.81-6.88 (1H, m, phenyl-H), 6.92-6.99 (3H, m, phenyl-H), and 7.39 (1H, s, N═CH). ¹³C NMR (75 MHz, CDCl₃) δ 23.8 (CH₂), 26.2 (CH₂), 27.0 (CH3), 50.5 (CH₂N), 51.8 (CH₂N), 53.5 (CH₂N), 58.1 (CH₂N), 67.5 (d, J=21 Hz, CH₂O), 82.0 (d, J=165 Hz, CH₂F), 113.6, 118.4, 122.1, 122.7 (phenyl-C3-C6), 133.7 (N═CH), 142.0 (phenyl-C2-O), 148.9 (NHC(═O)CH), 151.0 (phenyl-C1-N), 156.3 (NHC(═O)NH).

### Preparation of [¹⁸F]2-(4-(4-(2-(2-fluoroethoxy)phenyl)piperazin-1-yl)butyl)-4-methyl-1,2,4-triazine-3,5(2H,4H)-dione

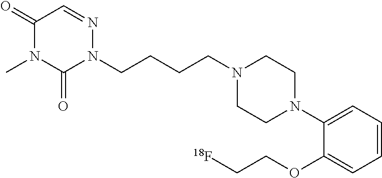 [SMILES: Cn1c(=O)cnn(CCCCN2CCN(c3ccccc3OCC[18F])CC2)c1=O]

### Preparation of [¹⁸F]fluoroethyl tosylate

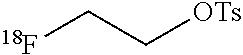 [SMILES: Cc1ccc(S(=O)(=O)OCC[18F])cc1]

[¹⁸F]fluoride was transferred from a P6 vial into a 3 mL V-vial by suction. To the P6 vial was added a pre-prepared solution of kryptofix 2.2.2 (5 mg, 1.3×10⁻⁵ mol) in MeCN (0.5 mL) and K₂CO₃ (65 μL, 0.1M) made up to 0.5 mL with water. The P6 vial was agitated and the solution transferred to the V-vial by suction. The V-vial was heated to 110° C. for 20 min under a flow of nitrogen (0.2 L/min) then cooled to room temperature.

To the dried [¹⁸F]fluoride and kryptofix mixture was added ethanediol ditosylate (6 mg, 1.3×10⁻⁵ mol) in MeCN (1 mL). The resulting yellow solution was heated at 80° C. for 10 min, and then cooled to room temperature. To the reaction vial was added water (1.5 mL) and the mixture loaded on to preparative HPLC for purification. The isolated HPLC fraction was diluted into water (20 mL) and then loaded onto a Waters tC18-light Sep Pak cartridge. The cartridge was then dried on a high pressure nitrogen line for 20 min.

### Coupling with 2-(4-(4-(2-hydroxyphenyl)piperazin-1-yl)butyl)-4-methyl-1,2,4-triazine-3,5(2H,4H)-dione

2-(4-(4-(2-hydroxyphenyl)piperazin-1-yl)butyl)-4-methyl-1,2,4-triazine-3,5(2H,4H)-dione (5 mg, 1.4×10⁻⁵ mol) in DMF (0.1 mL) was stirred in the presence of Cs₂CO₃ (14 mg, 4.3×10⁻⁵ mol) for 10 min, when the dried [¹⁸F]fluoroethyl tosylate was eluted into the reaction vial with DMF (0.4 mL) and the reaction mixture was stirred at 120° C. for 10 min (colour change: pale yellow to brown). The crude reaction mixture was diluted into water (3 mL) and purified by preparative HPLC (see FIGS. 1 and 2).

### Formulation

The isolated HPLC fraction was diluted into water (15 mL) and trapped on a pre-treated Waters tC18 light Sep Pak and then eluted with ethanol (0.5 mL) into a pre-weighed vial containing PBS (0.5 mL). The ethanol was removed in vacuo to give the formulated product.

### Prep HPLC

Purification of [¹⁸F]fluoroethyl tosylate

| | |
|---|---|
| HPLC Column | Hichrom Ace 5, C18, 10 × 100 mm & 10 × 10 mm guard |
| Solvent | A = 50 mM ammonium acetate, B = Methanol |
| Gradient | 50-80% B over 20 min |
| Flow | 3 ml/min |
| UV | 254 nM |

Purification of [18F]2-(4-(4-(2-(2-fluoroethoxy)phenyl)piperazin-1-yl)butyl)-4-methyl-1,2,4-triazine-3,5(2H,4H)-dione

| | |
|---|---|
| HPLC Column | Hichrom Ace 5, C18, 10 × 100 mm & 10 × 10 mm guard |
| Solvent | A = 50 mM ammonium acetate, B = Methanol |
| Gradient | 50% B isocratic 20 min |
| Flow | 4 ml/min |
| UV | 254 nM |

A ‘specific activity’ was calculated on the basis of UV peak area of the species that elute with the product, giving a figure of between 4-5 GBq/μmol.

### Example 13

### Preparation for Making the Radiolabled Compounds of Formula (I) by a 1-Pot 2-Step Synthesis

A Kryptofix/potassium carbonate solution (molar ratio 2:1, MeCN/H₂O 96:4, 2 mL) was added to the target water containing ¹⁸F⁻ and dried at 110° C. for 30 min under N₂-gas. A solution of ethane-1,2-diyl bis(4-methylbenzenesulfonate) (6-7 mg) in DMF (0.2 mL) was added to the residue containing the complex [K/K2.2.2]⁺¹⁸F⁻ and the mixture was heated to 70° C. in a closed vessel for 15 min. The crude product from step 1 was analyzed on the analytical HPLC system. Analytical HPLC conditions: A:H₂O B:MeCN. Gradient: 20-95% over 15 min. Flow rate=1.5 mL/min. Column: Phenomenex Luna 250×4.6 mm, 5 micron, C18. Rₜ=11.2 min. Incorporation yield 71%. Cesium carbonate (7.2 mg) was added to 2-(4-(benzo[d]thiazol-2-ylmethyl)piperazin-1-yl)phenol (1 mg) in DMF (0.2 mL). The mixture was added to the vessel containing 2-[¹⁸F]fluoroethyl 4-methylbenzenesulfonate and heated at 150° C. for 15 min. The crude product was analyzed on the analytical HPLC system using same conditions as above. Rₜ=15.01 min. Incorporation yield 8-32%.

### Example 14

### Preparation of 3-(4-(4-(4-(2-fluoroethoxy)phenyl)piperazin-1-yl)butyl)-1H-indole

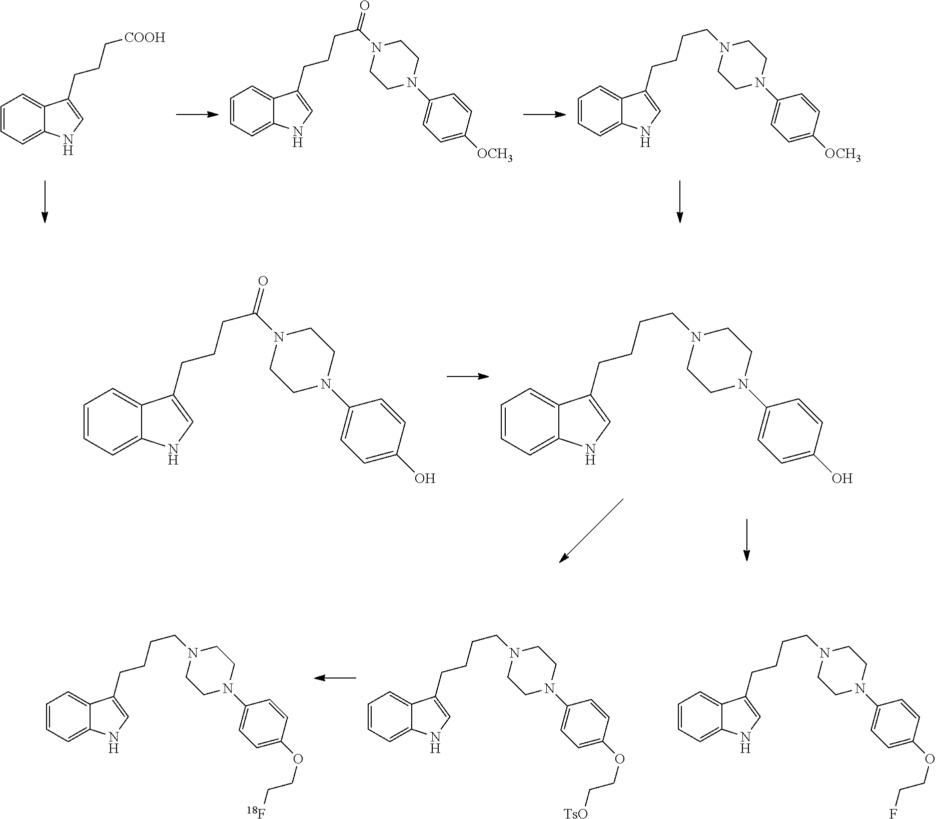

### Preparation of 4-(1H-Indol-3-yl)-1-(4-(4-methoxyphenyl)piperazin-1-yl)butan-1-one

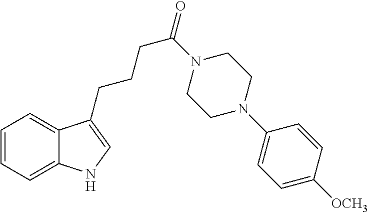 [SMILES: COc1ccc(N2CCN(C(=O)CCCc3c[nH]c4ccccc34)CC2)cc1]

Indole-3-butyric acid (1.0 g, 4.92 mmol) was taken in an oven dried round bottomed flask and dissolved in dry tetrahydrofuran (10 mL) under inert atmosphere. 1-Ethyl-3-(3-dimethylaminopropyl) carbodiimide (1.17 g, 6.15 mmol) was added to the reaction mixture at 0° C. and then stirred at same temperature for 2 h followed by slow addition of a tetrahydrofuran (10 mL) solution of 1-(4-methoxyphenyl)piperazine (1.04 g, 5.41 mmol), 1-hydroxybenzotriazole (332 mg, 2.46 mmol), triethylamine (2.3 mL, 12.3 mmol). Reaction mixture was then stirred for 24 h at room temperature, quenched with water and then extracted with dichloromethane. Crude product was then purified by chromatography on silica gel eluting with ethyl acetate to give the product as a pale brown gum in 33% yield.

LCMS: calcd for C₂₃H₂₇N₃O₂=377.2; found 378.1 [M+H]⁺.

¹H-NMR (300 MHz, CDCl₃) δ=8.19 (1H, bs, NH), 7.62 (1H, d, J=9 Hz), 7.35 (1H, d, J=9 Hz), 7.19 (1H, t, J=6 Hz), 7.11 (1H, t, J=6 Hz), 7.00 (1H, d, J=3 Hz), 6.85 (4H, d, J=6 Hz), 3.78 (3H, s), 3.49 (2H, t, J=6 Hz), 3.01 (2H, t, J=6 Hz), 2.94 (2H, t, J=6 Hz), 2.86 (2H, t, J=6 Hz), 2.43 (2H, t, J=6 Hz), 2.16-2.05 (3H, m).

### Preparation of 3-(4-(4-(4-methoxyphenyl)piperazin-1-yl)butyl)-1H-indole

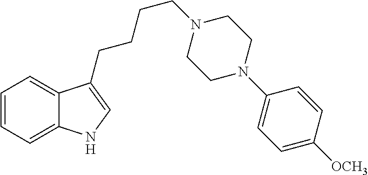 [SMILES: COc1ccc(N2CCN(CCCCc3c[nH]c4ccccc34)CC2)cc1]

To an oven dried round bottom 2-neck flask, 0.236 g (6.21 mmol) of lithium aluminum hydride was dissolved in anhydrous tetrahydrofuran (25 mL) at 0° C. A tetrahydrofuran (25 mL) solution of 4-(1H-indol-3-yl)-1-(4-(4-methoxyphenyl)piperazin-1-yl)butan-1-one (1.57 g, 4.15 mmol) was added to the reaction mixture kept at 0° C. The reaction mixture was then heated to reflux for 12 h and quenched with ice-cold water (20 mL). The mixture was filtered using Celite bed and the solvent was evaporated under reduced pressure. The residue was partitioned between dichloromethane and water. The combined organic layers were then dried over anhydrous sodium sulfate. The solvent was evaporated under reduced pressure and then purified by chromatography on silica gel eluting with 50% ethyl acetate in hexane to give the product as a yellow colored solid (618 mg, 41%),

LCMS: calcd for C₂₃H₂₉N₃O=363.2; found 364.1 [M⁺H]⁺.

¹H-NMR: (300 MHz, CDCl₃) δ=8.12 (1H, bs, NH), 7.64 (1H, d, J=9 Hz), 7.36 (1H, d, J=6 Hz), 7.25-7.10 (2H, m), 7.01-6.81 (5H, m), 3.80 (3H, s), 3.13 (4H, t, J=6 Hz), 2.82 (2H, t, J=6 Hz), 2.65 (4H, t, J=6 Hz), 2.48 (2H, t, J=9 Hz), 1.84-1.63 (4H, m).

### Preparation of 4-(4-(4-(1H-indol-3-yl)butyl)piperazin-1-yl)phenol

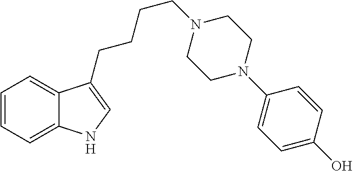 [SMILES: Oc1ccc(N2CCN(CCCCc3c[nH]c4ccccc34)CC2)cc1]

3-(4-(4-(4-Methoxyphenyl)piperazin-1-yl)butyl)-1H-indole (610 mg, 1.68 mmol) was dissolved in anhydrous dichloromethane (10 mL) in a round bottom flask. The reaction mass was then cooled to −78° C. Boron tribromide (175 μl, 1.85 mmol) was then added to the reaction mixture at the same temperature and then allowed to come to room temperature and stirred for 12 h. The reaction mixture was then quenched by addition of water. The aqueous layer was basified by saturated sodium bicarbonate solution and extracted with dichloromethane (3×20 mL). The combined organic extracts were dried over anhydrous sodium sulfate, evaporated under reduced pressure to give the desired product (100 mg, 17%), which was used directly in the next step with no further purification.

LCMS: calcd for C₂₂H₂₇N₃O=349; found 350.2 [M⁺H]⁺.

### Preparation of 3-(4-(4-(4-(2-Fluoroethoxy)phenyl)piperazin-1-yl)butyl)-1H-indole

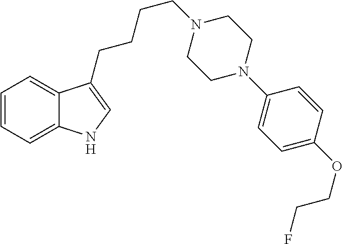 [SMILES: FCCOc1ccc(N2CCN(CCCCc3c[nH]c4ccccc34)CC2)cc1]

4-(4-(4-(1H-Indol-3-yl)butyl)piperazin-1-yl)phenol (30 mg, 0.085 mmol) was taken in a oven dried round bottomed flask and dissolved in dry acetonitrile and dimethylformamide (10 mL, 1:1) under inert atmosphere. Cesium carbonate (50 mg, 0.127 mmol) was then added to the reaction mixture followed by slow addition of acetonitrile solution (5 mL) of fluoroethyl tosylate (40 mg, 0.127 mmol) over a period of 10 minutes. Reaction mixture was then stirred for 12 h at 50° C. The reaction mass was quenched with water and extracted with ethyl acetate. Crude product was then purified by chromatography on silica gel eluting with 5% methanol in dichloromethane to give the desired compound in 76% yield.

LCMS: calcd for C₂₄H₃₀N₃OF=395.2; found 396.1 [M+H]⁺.

¹H-NMR (500 MHz, CDCl₃) δ=8.27 (1H, bs, NH), 7.60 (1H, d, J=10 Hz), 7.34 (1H, d, J=10 Hz), 7.17 (1H, t, J=10 Hz), 7.10 (1H, t, J=10 Hz), 6.97 (1H, bs), 6.91-6.85 (4H, m), 4.77 (1H, t, J=5 Hz), 4.67 (1H, t, J=5 Hz), 3.11 (4H, t, J=5 Hz), 2.79 (2H, t, J=5 Hz), 2.62 (4H, t, J=5 Hz), 2.45 (2H, t, J=5 Hz), 1.75 (2H, p, J=10 Hz), 1.68-1.61 (5H, m).

### Preparation of 1-(4-(4-hydroxyphenyl)piperazin-1-yl)-4-(1H-indol-3-yl)butan-1-one

Indole-3-butyric acid (1500 mg, 7.38 mmol) was taken in an oven dried round bottomed flask and dissolved in dry tetrahydrofuran (20 mL) under inert atmosphere. 1-Ethyl-3-(3-dimethylaminopropyl)carbodiimide (1.41 g, 7.38 mmol) was added to the reaction mixture at 0° C. and then stirred at same temperature for 2 h followed by slow addition of a tetrahydrofuran solution (10 mL) of 4-hydroxy phenylpiperazine (1.31 g, 7.38 mmol). Reaction mixture was then stirred for 24 h at room temperature, quenched with water and then extracted with dichloromethane. Crude product was then washed with hexane to give desired product as a white solid (2.38 g, 88%).

LCMS: calcd for C₂₂H₂₅N₃O₂=363.2; found 364.2 [M+H]+.

¹H-NMR (300 MHz, d₆-DMSO) δ=10.77 (1H, bs, OH), 8.89 (1H, bs, NH), 7.52 (1H, d, J=6 Hz), 7.33 (1H, d, J=9 Hz), 7.12 (1H, bs), 7.06 (1H, t, J=6 Hz), 6.96 (1H, t, J=6 Hz), 6.79 (2H, d, J=9 Hz), 6.66 (2H, d, J=9 Hz), 3.58 (2H, bs), 3.50 (2H, bs), 3.36 (1H, s), 2.88 (3H, bs), 2.72 (2H, t, J=6 Hz), 2.39 (2H, t, J=6 Hz), 1.96-1.92 (2H, m).

### Preparation of 2-(4-(4-(4-(1H-Indol-3-yl)butyl)piperazin-1-yl)phenoxy)ethyl 4-methylbenzenesulfonate

4-(4-(4-(1H-indol-3-yl)butyl)piperazin-1-yl)phenol (260 mg, 0.744 mmol) was taken in a oven dried round bottomed flask and dissolved in dry acetonitrile (10 mL) under inert atmosphere. Cesium carbonate (363 mg, 1.116 mmol) was then added to the reaction mixture followed by slow addition of an acetonitrile solution (5 mL) of ethylene ditosylate (302 mg, 0.814 mmol) over a period of 30 minutes. Reaction mixture was then stirred for 12 h at 50° C. The reaction mass was quenched with water and extracted with ethyl acetate. Crude product was then purified by chromatography on silica gel eluting with 5% methanol in dichloromethane to give the desired compound in a yield of 74%.

LCMS: calcd for C₃₁H₃₇N₃O₄S=547.3; found 548.2 [M+H]⁺.

¹H-NMR (300 MHz, CDCl₃) δ=8.11 (1H, bs, NH), 7.81 (2H, d, J=9 Hz), 7.61 (1H, d, J=6 Hz), 7.37-7.30 (3H, m), 7.21-7.07 (2H, m), 6.97 (1H, bs), 6.84 (2H, d, J=9 Hz), 6.72 (2H, d, J=9 Hz), 4.33 (2H, t, J=6 Hz), 4.08 (2H, t, J=6 Hz), 3.10 (4H, t, J=6 Hz), 2.79 (2H, t, J=6 Hz), 2.61 (4H, t, J=6 Hz), 2.49-2.42 (5H, m), 1.81-1.58 (4H, m).

### Preparation of [¹⁸F]3-(4-(4-(4-(2-Fluoroethoxy)phenyl)piperazin-1-yl)butyl)-1H-indole

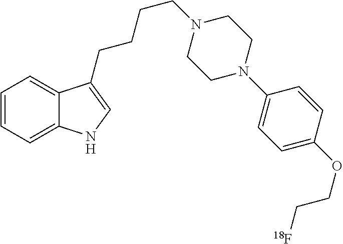 [SMILES: [18F]CCOc1ccc(N2CCN(CCCCc3c[nH]c4ccccc34)CC2)cc1]

[¹⁸F]-fluoride (975 MBq) was transferred to the GE TracerLab FX and trapped on a conditioned QMA cartridge. The [¹⁸F]-fluoride was then eluted as the Kryptofix (K222) complex using a solution formed of Kryptofix (10.0 mg) in acetonitrile (2 ml) and 13.0 mg/ml potassium hydrogen carbonate (100 μl) into the reaction vessel. Azeotropic drying of the [¹⁸F]-fluoride commenced at 100° C. for 30 minutes under a stream of N₂ gas. The dried [¹⁸F]-fluoride/K222 vial was then cooled to 50° C. and N₂ flow ceased. 2-(4-(4-(4-(1H-indol-3-yl)butyl)piperazin-1-yl)phenoxy)ethyl 4-methylbenzenesulfonate (1.7 mg) in acetonitrile (1 ml) was added to the reaction vessel which was then sealed and heated to 90° C. for 10 minutes. The crude reaction was diluted with H₂O (2.5 ml) [¹⁸F]3-(4-(4-(4-(2-Fluoroethoxy)phenyl)piperazin-1-yl)butyl)-1H-indole was purified by preparative RP HPLC (Phoenomenex Luna C18, 150× 10 mm, 5 μm, A=10 mM H₃PO₄, B=MeCN; 5%-90% B over 20 mins). The peak collected was trapped on a tC18 light cartridge (conditioned with ethanol (5 ml), H₂0 (10 ml)) and eluted with ethanol (500 μl) followed by formulation with PBS (4.5 ml). Formulated) [¹⁸F]3-(4-(4-(4-(2-Fluoroethoxy)phenyl)piperazin-1-yl)butyl)-1H-indole=133.2 MBq total synthesis time 72 minutes, 14% non-decay corrected yield, specific activity 2.5 GBq/μmol, RCP 91%.

Compounds that were synthesized following procedures that were similar or identical as described those in Example 14 include the following:

| Structure | Name | [M + H]⁺ |
|---|---|---|
| 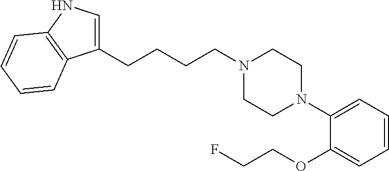 [SMILES: FCCOc1ccccc1N1CCN(CCCCc2c[nH]c3ccccc23)CC1] | 3-(4-(4-(2-(2- fluoroethoxy)phenyl)piperazin- 1-yl)butyl)-1H-indole | 396.4 |

### Example 15

### Preparation of N-(2-(4-(6-Fluoropyridin-2-yl)piperazin-1-yl)ethyl)cyclohexanecarboxamide

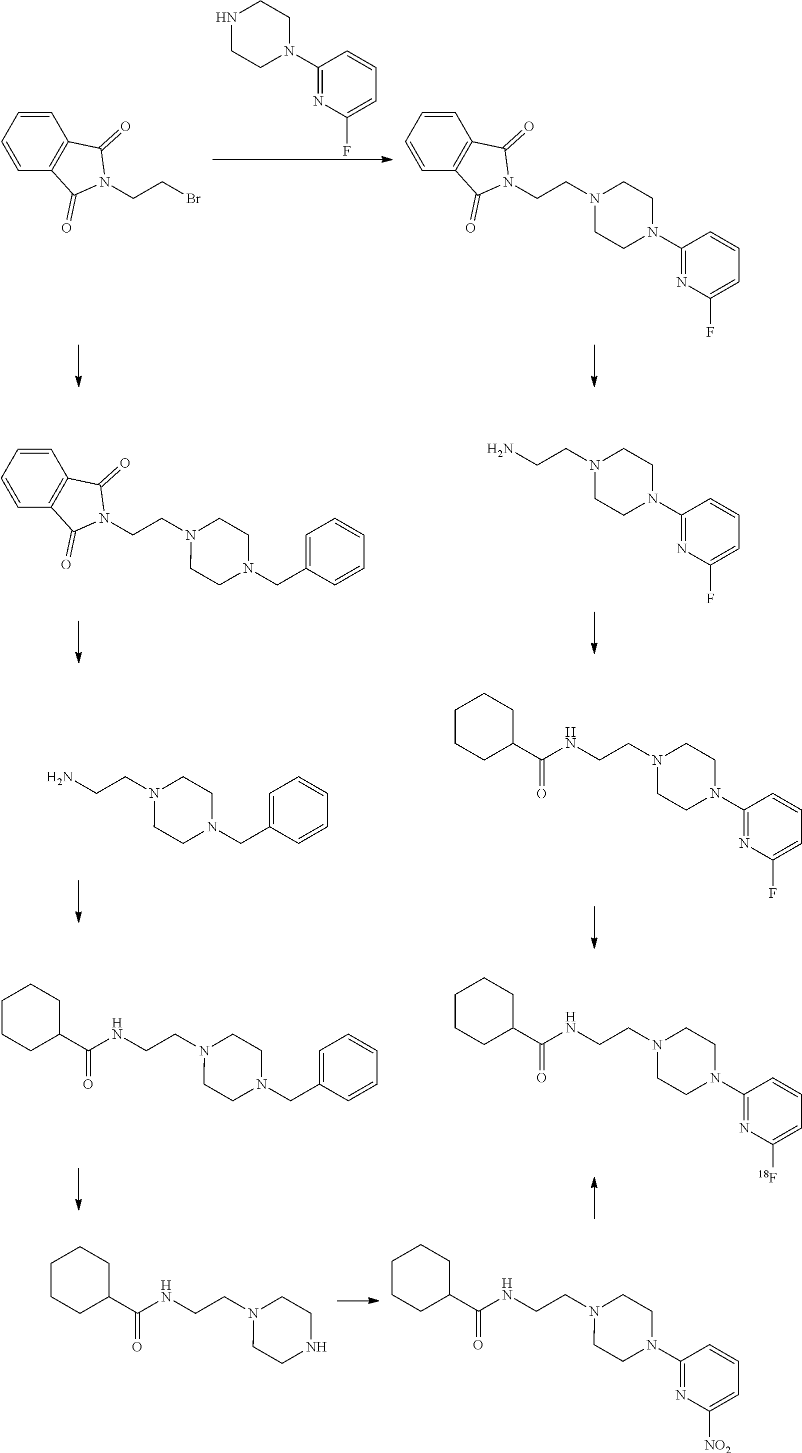

### Preparation of 2-(2-(4-(6-fluoropyridin-2-yl)piperazin-1-yl)ethyl)isoindoline-1,3-dione

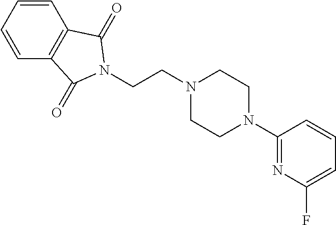 [SMILES: O=C1c2ccccc2C(=O)N1CCN1CCN(c2cccc(F)n2)CC1]

A 10 mL microwave tube was charged with 1-(6-fluoro-pyridin-2-yl)-piperazine (0.61 g, 3.37 mmol), N-(4-bromoethyl)-phthalimide (0.85 g, 3.34 mmol), acetonitrile (5 mL) then diidopropylethylamine (0.59 mL, 3.37 mmol) and heated at 120° C. in the microwave oven for 40 min. The reaction mixture was concentrated to give a brown solid. This was taken up in ethyl acetate (20 mL) and washed with 10% aqueous potassium carbonate (15 mL). The aqueous layer was washed with ethyl acetate (20 mL), the organic layers combined and filtered through a phase separator and evaporated under reduced pressure to give a light brown solid. Purification by chromatography on silica gel eluting with 0-10% methanol in dichloromethane gradient over 15 CV gave the product (1.16 g, 98%).

LCMS: calcd for C₁₉H₁₉FN₄O₂, 354.2; found 355.1 [M+H]⁺.

¹H and ¹³C NMR were consistent with the structure.

### Preparation of 2-(4-(6-Fluoropyridin-2-yl)piperazin-1-yl)ethanamine

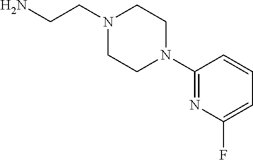 [SMILES: NCCN1CCN(c2cccc(F)n2)CC1]

A flask was charged 2-(2-(4-(6-fluoropyridin-2-yl)piperazin-1-yl)ethyl)isoindoline-1,3-dione (1.16 g, 3.27 mmol), ethanol (10 mL) and hydrazine hydrate (300 μL, 6.55 mmol) and the reaction mixture was heated to 80° C. for 3 hours then cooled to ambient temperature. The white solid was stirred in ethanol and removed by filtration. The solid was washed with ethanol and the filtrates were concentrated under reduced pressure to give the product as a yellow oil (747 mg, 86%).

LCMS: calcd for C₁₁H₁₇FN₄, 224.1; found 225.0 [M+H]⁺.

¹H and ¹³C NMR were consistent with the structure.

### Preparation of N-(2-(4-(6-Fluoropyridin-2-yl)piperazin-1-yl)ethyl)cyclohexanecarboxamide

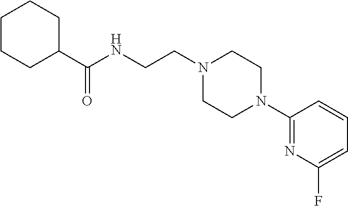 [SMILES: O=C(NCCN1CCN(c2cccc(F)n2)CC1)C1CCCCC1]

Triethylamine (66 μl, 0.47 mmol), and cyclohexanecarbonyl chloride (95 μl, 0.71 mmol) were added to 2-(4-(6-fluoropyridin-2-yl)piperazin-1-yl)ethanamine (125 mg, 85% pure, 0.47 mmol) in dichloromethane (7 mL). The reaction was stirred at ambient temperature for 18 hours. The reaction mixture was stirred with 10% aqueous potassium carbonate (10 mL) and the organic layer filtered through a phase separator. The organic layer was concentrated and purified twice by chromatography on silica gel (10 g) eluting with a 0-5% gradient of 1% triethylamine in methanol and dichloromethane over 25 CV to give the product as an off white solid. The product was purified further by semi-preparative HPLC using a gradient of 50-95% methanol in water over 20 min @ 21 ml/min (column: Phenomenex Gemini C18, 110 Å, 150×21.2 mm, 5 μm; Product Rt 10.6 min). This gave the pure product (39 mg, 25%).

LC-MS: calcd for C₁₈H₂₇FN₄O 334.2; found 335.1 [M+H]⁺, 357.1 [M+Na]⁺.

¹H and ¹³C NMR were consistent with the structure.

Purity measured as 100% by HPLC (UV 254 nm).

### Preparation of 2-(2-(4-Benzylpiperazin-1-yl)ethyl)isoindoline-1,3-dione

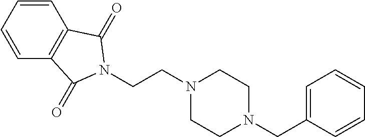 [SMILES: O=C1c2ccccc2C(=O)N1CCN1CCN(Cc2ccccc2)CC1]

N-(2-bromoethyl)phthalimide (1.80 g, 7.09 mmol) was dissolved in acetonitrile (7 mL) and 1-benzylpiperazine (1.24 mL, 7.09 mmol) then diisopropylethylamine (1.24 mL, 7.09 mmol) were added at ambient temperature. The reaction mixture was split into two 10 mL tubes and heated at 130° C. for 15 min in the microwave oven. The reaction mixtures were combined, evaporated to dryness then purified by chromatography on silica gel eluting with a 0-5% methanol in dichloromethane gradient to give the product as a pale orange oil (2.44 g, 98%).

LCMS ES⁺: C₂₁H₂₃N₃O₂ calc. 349.2; found 350.1 [M+H]⁺.

¹H and ¹³C NMR were consistent with the structure.

### Preparation of 2-(4-Benzylpiperazin-1-yl)ethanamine

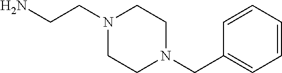 [SMILES: NCCN1CCN(Cc2ccccc2)CC1]

A flask was charged with 2-(2-(4-benzylpiperazin-1-yl)ethyl)isoindoline-1,3-dione (2.44 g, 6.98 mmol), ethanol (25 mL) and hydrazine monohydrate (0.64 mL, 13.96 mmol) at ambient temperature. The reaction mixture was heated at 80° C. for 18 h leading to the formation of a white solid. The reaction mixture was then cooled to ambient temperature and the white solid removed by filtration. The solid was washed with ethanol and the filtrate was concentrated under reduced pressure to give the product as a yellow gum (1.58 g, 100%).

LCMS: calcd for C₁₃H₂₁N₃, 219.2; found 220.1 [M+H]⁺.

¹H and ¹³C NMR were consistent with the structure.

### Preparation of N-(2-(4-Benzylpiperazin-1-yl)ethyl)cyclohexanecarboxamide

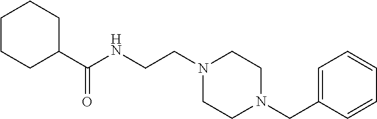 [SMILES: O=C(NCCN1CCN(Cc2ccccc2)CC1)C1CCCCC1]

To a solution of 2-(4-benzylpiperazin-1-yl)ethanamine (408 mg, 1.71 mmol) in dichloromethane (20 mL) was added cyclohexanecarbonyl chloride (229 μl, 1.71 mmol) and triethylamine (477 μl, 3.42 mmol). The reaction mixture was stirred at ambient temperature for 1 hour and was evaporated to dryness. The residue was taken up in ethyl acetate, filtered, loaded on silica cartridge and chromatographed eluting with a 0-10% methanol in ethyl acetate gradient to give the product as a light pink solid (293 mg, 52%).

LCMS: calcd for C₂₀H₃₁N₃O, 329.3; found 330.1 [M+H]⁺.

¹H and ¹³C NMR were consistent with the structure.

### Preparation of N-(2-(piperazin-1-yl)ethyl)cyclohexanecarboxamide

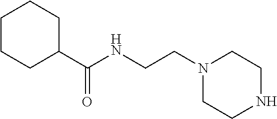 [SMILES: O=C(NCCN1CCNCC1)C1CCCCC1]

N-(2-(4-benzylpiperazin-1-yl)ethyl)cyclohexanecarboxamide (270 mg, 0.82 mmol) was dissolved in acetic acid (16 ml) and 6N HCl (1 mL) added followed by 10% Pd/C (80 mg). The mixture was placed under 30 psi H₂ atmosphere in the Parr hydrogenator and was shaken for 3 h at 60° C. The reaction mixture was filtered through Celite and the Celite washed with acetic acid (2×5 mL). TLC showed the formation of a new product revealed using ninhydrin as a pink spot on the TLC base line. The filtrate was evaporated under reduced pressure to give a brown gum. dichloromethane (20 mL) was added and the organic layer was washed with 10% aqueous potassium carbonate (20 mL) then the aqueous layer was extracted with more dichloromethane (20 mL). The organic layers were combined and washed with brine (10 mL), dried over magnesium sulfate, filtered and evaporated under reduced pressure to give the product as an off-white gum (119 mg, 61%).

LCMS: calcd for C₁₃H₂₅N₃O, 239.2; found 240.1 [M+H]⁺.

¹H and ¹³C NMR were consistent with the structure.

### Preparation of N-(2-(4-(6-Nitropyridin-2-yl)piperazin-1-yl)ethyl)cyclohexanecarboxamide

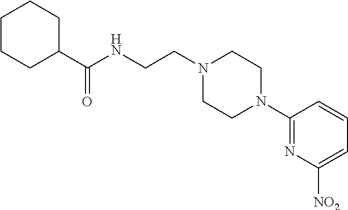 [SMILES: O=C(NCCN1CCN(c2cccc([N+](=O)[O-])n2)CC1)C1CCCCC1]

N-(2-(piperazin-1-yl)ethyl)cyclohexanecarboxamide (114 mg, 0.48 mmol), and 2-chloro-6-nitropyridine (76 mg, 0.48 mmol) were dissolved in dry acetonitrile (5 ml) in a 10 mL microwave tube and diisopropylethylamine (83 μL, 0.48 mmol) added. The mixture was stirred and then heated in the microwave oven at 120° C. for 20 min. The reaction mixture was evaporated under reduced pressure to give a brown residue which was taken up in ethyl acetate (15 mL) and washed with 10% aqueous potassium carbonate (10 mL). The organic layer was filtered through a phase separator and evaporated under reduced pressure. The residue was purified by chromatography on silica gel eluting with a 2-10% methanol in ethyl acetate gradient to give the product (26 mg, 15% yield) as a yellow solid. This was further purified by semi-preparative HPLC using a gradient of 50-95% MeOH in water over 20 min @16 ml/min (Gemini C18, 110 Å, 150×21.2 mm, 5 μm; Rt 11.1 min) to give the pure product (17 mg, 10%).

LCMS: calcd for C₁₈H₂₇N₅O₃, 361.2; found 362.1 [M+H]⁺.

¹H and ¹³C NMR were consistent with the structure.

Purity was 99⁺% by HPLC (UV 254 nm).

### Preparation of [¹⁸F]N-(2-(4-(6-Fluoropyridin-2-yl)piperazin-1-yl)ethyl)cyclohexanecarboxamide

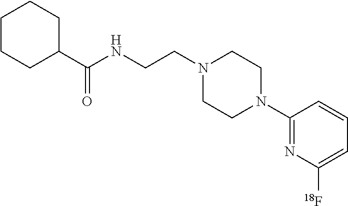 [SMILES: O=C(NCCN1CCN(c2cccc([18F])n2)CC1)C1CCCCC1]

[¹⁸F]-fluoride (11.96 GBq) was transferred to the GE Tracerlab FX and trapped on a conditioned QMA cartridge. The [¹⁸F]-fluoride was then eluted as the Kryptofix complex using a solution formed of Kryptofix (K222) (10.0 mg) in acetonitrile (2 ml) and 0.1M potassium carbonate (80 μl) into the reaction vessel. Azeotropic drying of the [¹⁸F]-fluoride commenced at 100° C. for 30 minutes under a stream of N₂ gas. The dried [¹⁸F]-fluoride/K222 vial was then cooled to 30° C. and N₂ flow ceased. N-(2-(4-(6-nitropyridin-2-yl)piperazin-1-yl)ethyl)cyclohexanecarboxamide (2.3 mg) in DMSO (1 ml) was added to the reaction vessel which was then sealed and heated to 150° C. for 10 minutes. The crude reaction was diluted with H₂0 (10 ml) and trapped on a tC18 light cartridge (conditioned with ethanol (5 ml and H₂O (10 ml)). The cartridge was washed with H₂O (4 ml) and eluted with acetonitrile (500 μl) before dilution with H₂O (2 ml). The crude product consisted of 3.58 GBq, from which a 560 MBq portion was removed and [¹⁸F]N-(2-(4-(6-fluoropyridin-2-yl)piperazin-1-yl)ethyl)cyclohexanecarboxamide was purified by preparative RP HPLC (Phoenomenex Luna C18, 150× 10 mm, 5 μm, A=0.8% triethylamine adj. to pH 7.5 with phosphoric acid, B=MeOH; 55%-85% B over 25 mins). The peak collected at 13.87 minutes (317 MBq) was diluted with H2O (4 ml) trapped on a tC18 light cartridge (conditioned as above) and eluted with EtOH (500 μl) and formulated by the addition of PBS (4.5 ml). Formulated [¹⁸F] N-(2-(4-(6-fluoropyridin-2-yl)piperazin-1-yl)ethyl)cyclohexanecarboxamide=268 MBq total synthesis time 133 minutes, specific activity 141 GBq/μmol, 14% non-decay corrected yield, RCP >99%

### Example 16

### Preparation of N-(4-(4-(2-Methoxyphenyl)piperazin-1-yl)butyl)-1H-indole-2-carboxamide

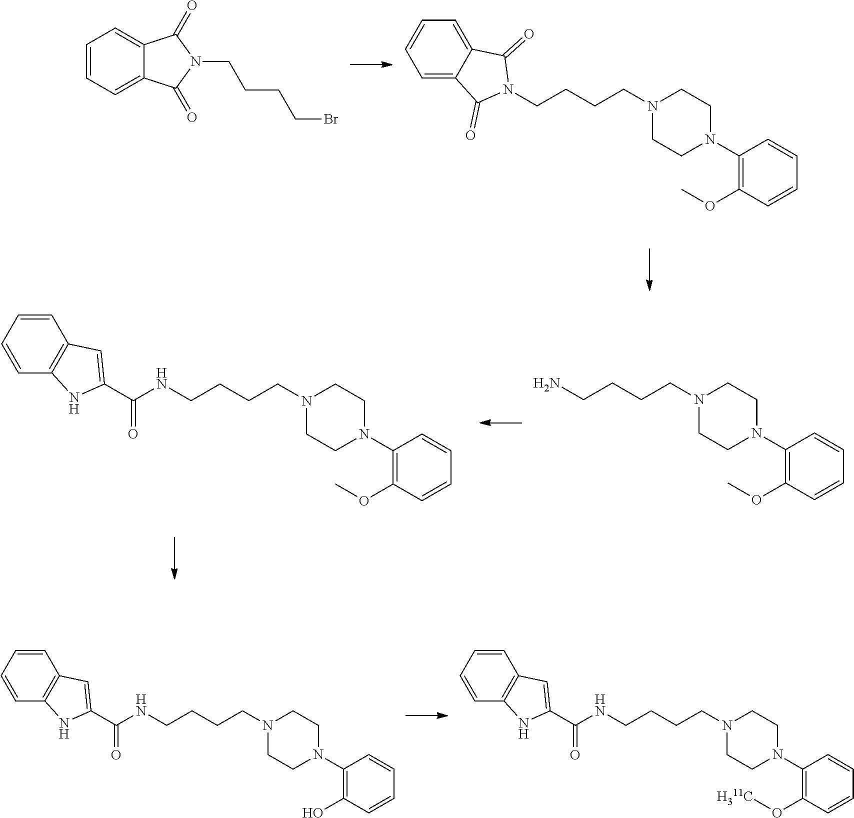 [SMILES: O=C1c2ccccc2C(=O)N1CCCCBr ; COc1ccccc1N1CCN(CCCCN2C(=O)c3ccccc3C2=O)CC1 ; COc1ccccc1N1CCN(CCCCNC(=O)c2cc3ccccc3[nH]2)CC1 ; COc1ccccc1N1CCN(CCCCN)CC1 ; O=C(NCCCCN1CCN(c2ccccc2O)CC1)c1cc2ccccc2[nH]1 ; [11CH3]Oc1ccccc1N1CCN(CCCCNC(=O)c2cc3ccccc3[nH]2)CC1]

### Preparation of 2-(4-(4-(2-Methoxyphenyl)piperazin-1-yl)butyl)isoindoline-1,3-dione

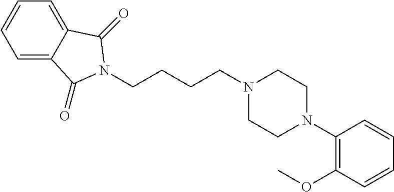 [SMILES: COc1ccccc1N1CCN(CCCCN2C(=O)c3ccccc3C2=O)CC1]

1-(2-Methoxyphenyl)piperazine (680 mg, 3.54 mmol) was taken in an oven dried round bottomed flask and dissolved in dry acetonitrile (15 mL) under inert atmosphere. Diisopropylethylamine (1.23 mL, 7.09 mmol) was then added to the reaction mixture followed by slow addition of an acetonitrile solution (10 mL) of N-(4-bromobutyl) phthalimide (1.0 g, 3.54 mmol) over a period of 10 minutes. Reaction mixture was then stirred for 12 h at room temperature. The reaction mass was quenched with water and extracted with ethyl acetate. Crude product was then purified by chromatography on silica gel eluting with 5% methanol in dichloromethane to give the desired compound (1.18 g, 84%).

LCMS: calcd for C₂₃H₂₇N₃O₃=393.2; found 394.1,

¹H-NMR (300 MHz, CDCl₃) δ=7.88-7.82 (2H, m), 7.73-7.69 (2H, m), 7.03-6.83 (4H, m), 3.86 (3H, s), 3.73 (2H, t, J=9 Hz), 3.08 (4H, bs), 2.66 (4H, bs), 2.48-2.41 (2H, m), 1.80-1.52 (4H, m).

### Preparation of 4-(4-(2-Methoxyphenyl)piperazin-1-yl)butan-1-amine

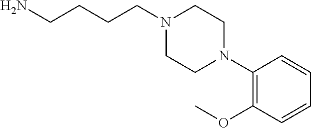 [SMILES: COc1ccccc1N1CCN(CCCCN)CC1]

A flask was charged with 2-(4-(4-(2-methoxyphenyl)piperazin-1-yl)butyl)isoindoline-1,3-dione (1.1 g, 2.79 mmol), ethanol (40 ml), hydrazine monohydrate (1.36 mL, 27.9 mmol) at room temperature under an inert atmosphere. The mixture was heated at 80° C. for 2 h. The reaction mixture was cooled to room temperature and the white precipitate, which was present, was removed by filtration. The solid was washed with ethanol (2×30 mL). The filtrate was concentrated at reduced pressure to obtain the product as a gum (700 mg).

LCMS: Calcd for C₁₅H₂₅N₃O=263.2; found 264.1 [M+H]⁺.

### Preparation of N-(4-(4-(2-Methoxyphenyl)piperazin-1-yl)butyl)-1H-indole-2-carboxamide

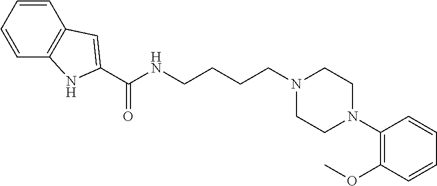 [SMILES: COc1ccccc1N1CCN(CCCCNC(=O)c2cc3ccccc3[nH]2)CC1]

Indole-2-carboxylic acid (428 mg, 2.66 mmol) was taken in an oven dried round bottomed flask and dissolved in dry tetrahydrofuran (10 mL) under inert atmosphere. 1-Ethyl-3-(3-dimethylaminopropyl)carbodiimide (508 mg, 2.66 mmol) was added to the reaction mixture at 0° C. and then stirred at same temperature for 2 h followed by slow addition of a tetrahydrofuran solution (10 mL) of 4-(4-(2-methoxyphenyl)piperazin-1-yl)butan-1-amine (700 mg, 2.66 mmol). Reaction mixture was then stirred for 24 h at room temperature, quenched with water and then extracted with dichloromethane. Crude product was then purified by chromatography on silica gel eluting with ethyl acetate to give the product as a yellow solid in 51% yield.

LCMS: Calcd for C₂₄H₃₀N₄O₂=406.2; found 407.1 [M+H]⁺.

¹H-NMR (300 MHz, CDCl₃) δ=7.66 (1H, d, J=9 Hz), 7.46 (1H, t, J=9 Hz), 7.33-7.25 (2H, m), 7.14 (1H, t, J=9 Hz), 7.08-7.02 (2H, m), 6.97-6.87 (3H, m), 3.88 (3H, s), 3.56 (2H, bs), 3.22 (4H, bs), 2.94 (4H, bs), 2.73 (2H, t, J=9 Hz), 1.90-1.70 (4H, m).

### Preparation of N-(4-(4-(2-Hydroxyphenyl)piperazin-1-yl)butyl)-1H-indole-2-carboxamide

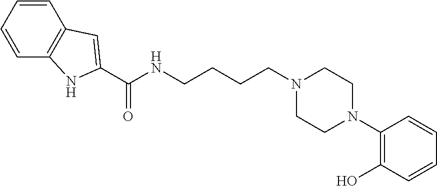 [SMILES: O=C(NCCCCN1CCN(c2ccccc2O)CC1)c1cc2ccccc2[nH]1]

N-(4-(4-(2-Methoxyphenyl)piperazin-1-yl)butyl)-1H-indole-2-carboxamide is dissolved in dichloromethane and treated with boron tribromide at −78° C. to room temperature. The reaction mixture is then quenched by addition of water. The aqueous layer is basified by saturated sodium bicarbonate solution and extracted with dichloromethane. The combined organic extracts are dried over anhydrous sodium sulfate, evaporated under reduced pressure to give the desired product which is purified by chromatography on silica gel.

### Preparation of [¹¹C] 2-(4-(4-(2-Methoxyphenyl)piperazin-1-yl)butyl)isoindoline-1,3-dione

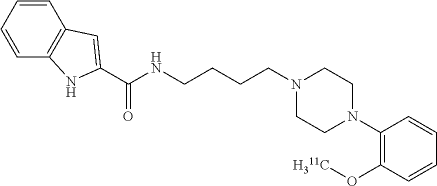 [SMILES: [11CH3]Oc1ccccc1N1CCN(CCCCNC(=O)c2cc3ccccc3[nH]2)CC1]

N-(4-(4-(2-Hydroxyphenyl)piperazin-1-yl)butyl)-1H-indole-2-carboxamide is treated with [¹¹C]iodomethane as previously described and the product purified by hplc.

### Example 17

### Preparation of N-(3-(4-(6-Fluoropyridin-2-yl)piperazin-1-yl)propyl)-4-phenylthiazol-2-amine

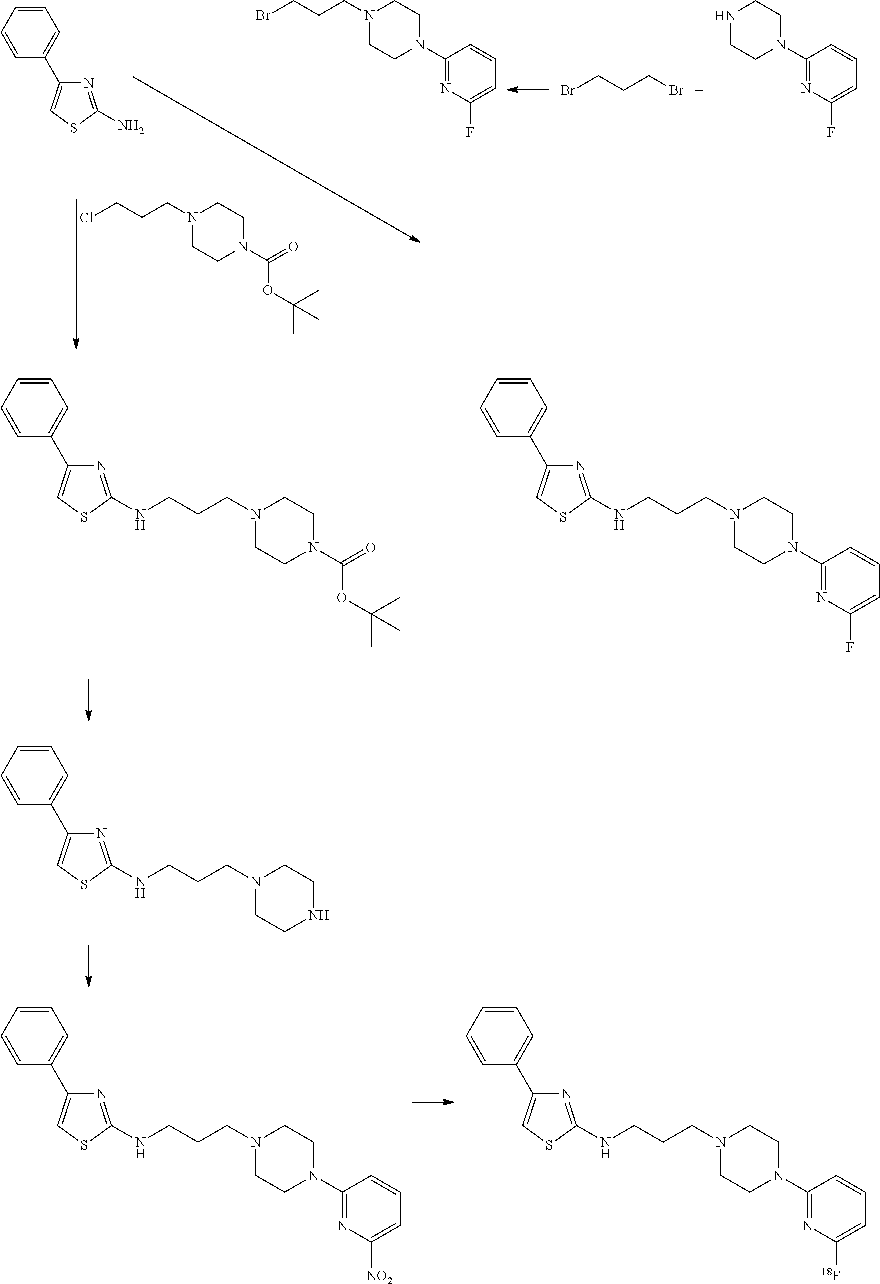

### Preparation of 1-(3-Bromopropyl)-4-(6-fluoropyridin-2-yl)piperazine

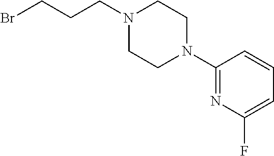 [SMILES: Fc1cccc(N2CCN(CCCBr)CC2)n1]

A mixture of 1-(6-fluoropyridin-2-yl)piperazine (1 eq.), 1,3-dibromopropane (10 eq.) and potassium carbonate (5 eq.) in dry dimethylformamide (50 ml) was heated at 90° C. for 18 h. The solvent was evaporated under reduced pressure and the residue was taken up in ethyl acetate and washed with water (4×50 ml) followed by brine (50 ml). The organic layer was concentrated and the crude product was purified by column chromatography using 40% ethyl acetate in pet. ether.

¹H-NMR (400 MHz, CDCl₃) δ 2.08 (m, 2H), 2.54 (m, 6H), 3.48-3.58 (m, 6H), 6.17 (m, 1H), 6.40 (m, 1H), 7.53 (m, 1H).

### Preparation of N-(3-(4-(6-Fluoropyridin-2-yl)piperazin-1-yl)propyl)-4-phenylthiazol-2-amine

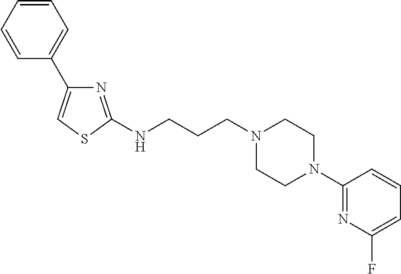 [SMILES: Fc1cccc(N2CCN(CCCNc3nc(-c4ccccc4)cs3)CC2)n1]

To a solution of 1-(3-bromopropyl)-4-(6-fluoropyridin-2-yl)piperazine (1 eq.) and 4-phenylthiazol-2-amine (2 eq.) in dimethylformamide, cesium carbonate (1.5 eq.) was added and the resulting mixture was heated at 90° C. for 18 h under nitrogen atmosphere. After completion of the reaction, the mixture was diluted with ethyl acetate and washed with water followed by brine. The organic layer was concentrated and the crude product was purified by column chromatography.

¹H and ¹³C NMR were consistent with the structure.

### Preparation of tert-Butyl 4-(3-((4-phenylthiazol-2-yl)amino)propyl)piperazine-1-carboxylate

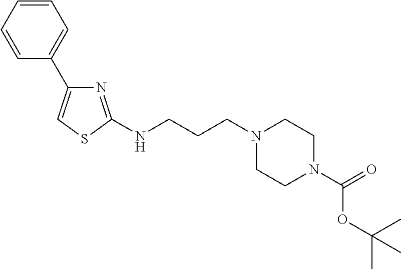 [SMILES: CC(C)(C)OC(=O)N1CCN(CCCNc2nc(-c3ccccc3)cs2)CC1]

To a solution of tert-butyl 4-(3-chloropropyl)piperazine-1-carboxylate (350 mg, 1.3 mmol) in dimethylformamide (10 mL) was added cesium carbonate (0.5 g, 1.5 mmol), and 2-amino-4-phenylthiazole (350 mg, 2 mmol). The mixture was heated at 90° C. for 16 hr. The reaction mixture was diluted in ethyl acetate (50 mL) and washed with water (50 mL) and brine (50 mL). The organics were dried over magnesium sulfate and concentrated. The residue diluted in dichloromethane (2 mL) and crude material was separated on silica (50 g) eluting with petrol/ethyl acetate (10% to 100% ethyl acetate over 14 CV at a flow of 40 mL/min). This gave the product as a yellow oil (150 mg, 29%).

LCMS: calcd for C₂₁H₃₀N₄O₂S 402.2; found 403.2 [M+H]⁺.

### Preparation of 4-Phenyl-N-(3-(piperazin-1-yl)propyl)thiazol-2-amine

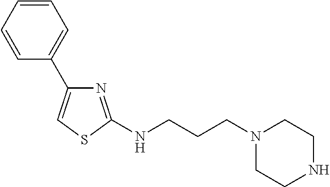 [SMILES: c1ccc(-c2csc(NCCCN3CCNCC3)n2)cc1]

To a solution of tert-butyl 4-(3-((4-phenylthiazol-2-yl)amino)propyl)piperazine-1-carboxylate (150 mg, 0.34 mmol) in dichloromethane (2 mL) was added trifluoroacetic acid (1 mL). The reaction mixture efforvesced and after fifteen minutes the reaction was complete. The reaction was diluted with dichloromethane (50 mL) and washed with saturated aqueous potassium carbonate (20 mL). The organics were dried and concentrated to yield a viscous orange oil (100 mg, 89%).

LCMS: calcd for C₁₆H₂₂N₄S 302.2; found m/z 303.2 [M+H]⁺.

### Preparation of N-(3-(4-(6-Nitropyridin-2-yl)piperazin-1-yl)propyl)-4-phenylthiazol-2-amine

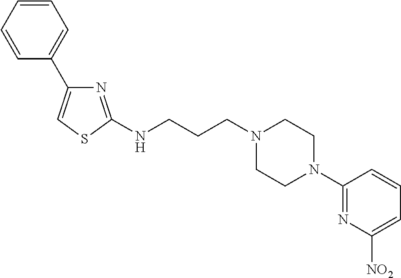 [SMILES: O=[N+]([O-])c1cccc(N2CCN(CCCNc3nc(-c4ccccc4)cs3)CC2)n1]

4-Phenyl-N-(3-(piperazin-1-yl)propyl)thiazol-2-amine, (0.1 g, 0.33 mmol) was dissolved in acetonitrile (6 mL) and 2-chloro-6-nitropyridine (0.06 g, 0.33 mmol) then diisopropylethylamine (0.1 mL, 0.6 mmol) were added at ambient temperature. The reaction mixture was split into two 10 mL microwave glass tubes then heated at 130 C for 26 min in the microwave oven. The reaction mixture was concentrated to give a brown oil. This was purified by chromatography on silica (40 g) eluting with 2.5% methanol in ethyl acetate over 16 CV at a flow of 40 mL/min). The product eluted between 7-10 CV and when concentrated yielded a yellow solid (32 mg, 23%).

LCMS: calcd for C₂₁H₂₄N₆O₂S 424.2; found m/z 425.2 (M+H)⁺.

### Preparation of [¹⁸F]N-(3-(4-(6-Fluoropyridin-2-yl)piperazin-1-yl)propyl)-4-phenylthiazol-2-amine

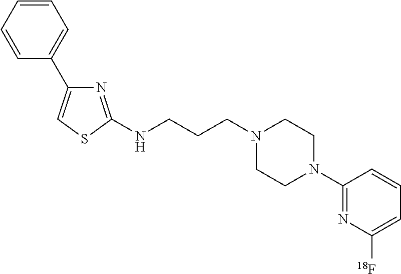 [SMILES: [18F]c1cccc(N2CCN(CCCNc3nc(-c4ccccc4)cs3)CC2)n1]

N-(3-(4-(6-Nitropyridin-2-yl)piperazin-1-yl)propyl)-4-phenylthiazol-2-amine is treated with [18F]fluoride as described previously and the [¹⁸F]N-(3-(4-(6-Fluoropyridin-2-yl)piperazin-1-yl)propyl)-4-phenylthiazol-2-amine produced is purified by hplc and formulated in ethanol and phosphate buffered saline.

### Example 18

### Preparation of 1-cyclohexyl-N-(4-(4-(6-fluoropyridin-2-yl)piperazin-1-yl)butyl)methane-sulfonamide

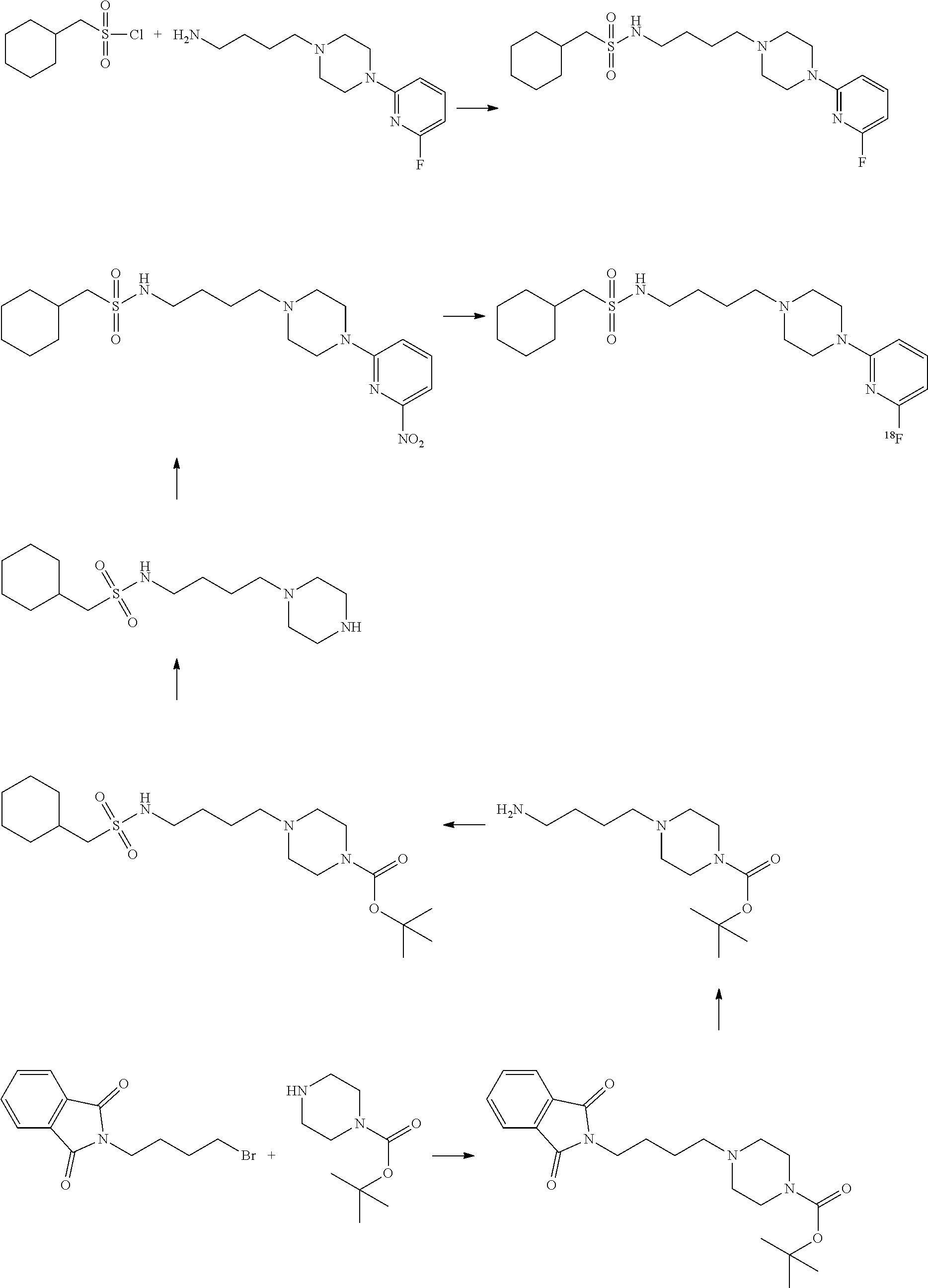

### Preparation of 1-Cyclohexyl-N-(4-(4-(6-fluoropyridin-2-yl)piperazin-1-yl)butyl)methane-sulfonamide

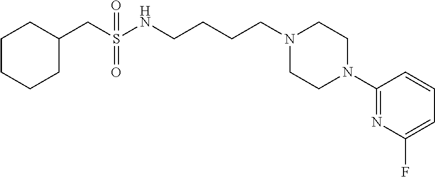 [SMILES: O=S(=O)(CC1CCCCC1)NCCCCN1CCN(c2cccc(F)n2)CC1]

To a flask charged with 4-(4-(6-fluoropyridin-2-yl)piperazin-1-yl)butan-1-amine (0.125 g, 0.5 mmol), dichloromethane (10 mL) and triethylamine (0.15 g, 14.8 mmol) was added cylohexylmethane sulfonyl chloride (100 mg, 0.55 mmol) at 0° C. under a nitrogen atmosphere. The mixture were allowed to warm to ambient temperature. After 18 hours the mixture was concentrated and partitioned between water (10 mL) and dichloromethane (10 mL). The organics were dried and concentrated. The crude material was loaded onto silica (10 g) and eluted with 10% methanol in ethyl acetate. This yielded a pale pink solid upon concentration (90 mg, 44%).

LCMS: calcd for C₂₀H₃₃FN₄O₂S 412.2; found 413.1 [M+H]⁺.

### Preparation of 1-Cyclohexyl-N-(4-(4-(6-nitropyridin-2-yl)piperazin-1-yl)butyl)methane-sulfonamide (Method A)

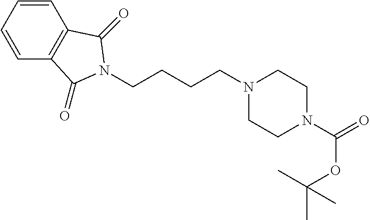 [SMILES: CC(C)(C)OC(=O)N1CCN(CCCCN2C(=O)c3ccccc3C2=O)CC1]

N-(2-Bromobutyll)-phthalimide (2.82 g, 10 mmol) was dissolved in acetonitrile (6 mL) and 1-Boc-piperazine (1.82 g, 10 mmol) then diisopropylethylamine (1.87 mL, 10.74 mmol) were added at ambient temperature. The reaction mixture was split into two 10 mL microwave glass tubes then heated at 130 C for 15 min in the microwave oven. TLC (ethyl acetate/petrol 1:1) showed a small amount of starting materials still present and a major product. The reaction mixture was evaporated to dryness and the yellow residue was dissolved in dichloromethane (20 mL) and washed with 10% aqueous potassium carbonate (30 mL), then the aqueous layer was washed twice with dichloromethane (10 mL). The organic layers were combined then evaporated to dryness to give a yellow oil which was purified by chromatography on silica gel (50 g) using 5-100% ethyl acetate in petrol gradient to at a flow of 40 mL/min to give the product as a white solid 3.1 g (80%).

LCMS: calcd for C₂₁H₂₉N₃O₄ 387.2; found 388.2 [M+H]⁺.

### Preparation of tert-Butyl 4-(4-aminobutyl)piperazine-1-carboxylate

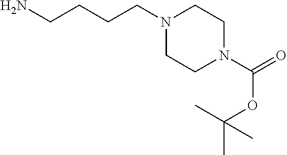 [SMILES: CC(C)(C)OC(=O)N1CCN(CCCCN)CC1]

tert-Butyl 4-(4-(1,3-dioxoisoindolin-2-yl)butyl)piperazine-1-carboxylate (2.50 g, 6.5 mmol), ethanol (130 mL) and hydrazine monohydrate (1.8 g, 36 mmol) at ambient temperature under a nitrogen atmosphere. The mixture was heated to 90 C for 18 hours. The reaction mixture was cooled to ambient temperature and the white precipitate which was present was removed by filtration. The solid was washed with ethanol (3×30 mL). The filtrate was concentrated at reduced pressure to give the product as a white solid (1.66 g, quantitative).

¹H (300 MHz, CD₃OD): δ 1.45 (9H, s, C(CH₃)₃), 1.56 (4H, p, J=3.7 Hz, CH₂), 2.39 (6H, m, CH₂ and 2×pip CH₂), 2.76 (2H, t, J=6.7 Hz, CH₂), 3.43 (4H, brt, J=4.3 Hz, 2×pip CH₂); ¹³C (75 MHz, CD₃OD): δ 24.8, 28.6, 29.8, 41.7, 53.9, 59.2, 81.3 and 156.3

### Preparation of tert-Butyl 4-(4-(cyclohexylmethylsulfonamido)butyl)piperazine-1-carboxylate

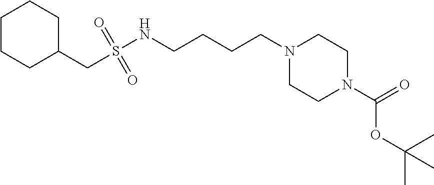 [SMILES: CC(C)(C)OC(=O)N1CCN(CCCCNS(=O)(=O)CC2CCCCC2)CC1]

tert-Butyl 4-(4-aminobutyl)piperazine-1-carboxylate (0.18 g, 0.70 mmol) was dissolved in dichloromethane (2 mL) then diisopropylethylamine (0.072 g 0.55 mmol) and cyclohexylmethanesulfonyl chloride (0.1 g, 0.51 mmol) were added at ambient temperature. The reaction mixture was left at RT for 18 hr. The mixture was dilited and partitioned between dichloromethane (20 mL) and water (20 mL). The organics were dried and concentrated. The crude mixture was purified on silic (10 g) eliting with 2.5% methanol/dichloromethane at a flow of 30 mL/min. The product was a glassy solid (55 mg, 18%).

¹H (CDCl₃, 300 MHz): δ1.09 (2H, pentamer), 1.25 (2H, pentamer), 1.42 (9H, s, C(CH₃)₃), 1.66 (6H, m), 1.88 (3H, m), 2.38 (6H, m), 2.96 (2H, d), 3.03 (2H, t, J=6.1 Hz, CH₂), 3.45 (4H, t, J=7.9 Hz, 2×pip CH₂), and 6.66 (1H, brs, NH); ¹³C (CDCl₃, 75 MHz): δ 24.4, 25.8, 28.3, 28.4, 33.0, 33.7, 43.0, 52.8, 58.0, 59.0, 79.9, and 154.6.

### Preparation of 1-Cyclohexyl-N-(4-(piperazin-1-yl)butyl)methanesulfonamide

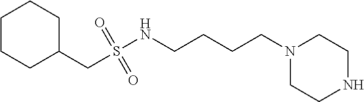 [SMILES: O=S(=O)(CC1CCCCC1)NCCCCN1CCNCC1]

To a solution of tert-butyl 4-(4-(cyclohexylmethylsulfonamido)butyl)piperazine-1-carboxylate (55 mg, 0.13 mmol) in dichloromethane (2 mL) was added trifluoroacetic acid (0.5 mL). The reaction mixture efforvesced and after fifteen minutes the reaction was complete. The reaction was diluted with dichloromethane (20 mL) and washed with saturated aqueous potassium carbonate (10 mL). The organics were dried and concentrated to yield the product as a pale yellow oil (40 mg, 97%).

¹H (CDCl₃, 300 MHz): δ1.09 (2H, pentamer), 1.25 (2H, pentamer), 1.63 (6H, m), 1.95 (3H, m), 2.36 (2H, t), 2.42 (4H, brs), 2.85 (2H, d), 2.93 (4H, t), and 3.07 (2H, t); ¹³C (CDCl₃, 75 MHz): δ 24.5, 25.8, 25.9, 29.2, 331, 33.8, 43.2, 54.3, 58.7, and 59.0.

### Preparation of 1-Cyclohexyl-N-(4-(4-(6-nitropyridin-2-yl)piperazin-1-yl)butyl)methane-sulfonamide

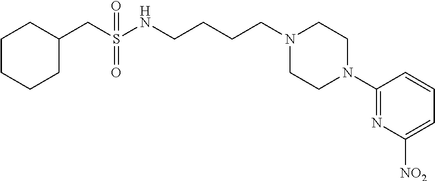 [SMILES: O=[N+]([O-])c1cccc(N2CCN(CCCCNS(=O)(=O)CC3CCCCC3)CC2)n1]

1-cyclohexyl-N-(4-(piperazin-1-yl)butyl)methanesulfonamide (40 mg, 0.33 mmol) was dissolved in acetonitrile (2 mL) and 2-chloro-6-nitropyridine (20 mg, 0.33 mmol) then diisopropylethylamine (0.1 mL, 0.6 mmol) were added at ambient temperature. The reaction mixture was at 130° C. for 40 min in the CEM microwave oven. The reaction mixture was concentrated to give a brown oil. This was purified by chromatography on silica gel (10 g) eluting with 2.5% methanol in ethyl acetate at a flow of 30 mL/min. The product eluted between 6 to 8 CV. These fractions were concentrated to give a yellow oil (14 mg, 26%). Further purification was performed by preparative HPLC eluting with 50 to 95% methanol in water over 20 min (Gemini column 150×21.2 mm at a flow of 20 mL/min).

LCMS: calcd for C₂₀H₃₃N₅O₄S 439.2; found 440.2 [M+H]⁺.

¹H (300 MHz, CDCl₃): δ 1.07 (2H, pentamer), 1.29 (2H, pentamer), 1.68 (7H, brm), 1.91 (4H, brm), 2.43 (2H, t), 2.60 (4H, t), 2.85 (2H, d), 3.10 (2H, t), 3.73 (4H, t), 6.91 (1H, d), 7.43 (1H, d), 7.69 (1H, t).

### Preparation of 1-Cyclohexyl-N-(4-(4-(6-nitropyridin-2-yl)piperazin-1-yl)butyl)methane-sulfonamide (Method B)

### 2-(4-(piperazin-1-yl)butyl)isoindoline-1,3-dione

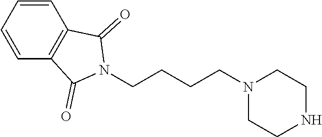 [SMILES: O=C1c2ccccc2C(=O)N1CCCCN1CCNCC1]

tert-Butyl 4-(4-(1,3-dioxoisoindolin-2-yl)butyl)piperazine-1-carboxylate (0.40 g, 1 mmol) was dissolved in dichloromethane (1 mL) and trifluoroacetic acid (1.0 mL, 9 mmol) was added at room temperature. The mixture was stirred until the efforvescence ceased. The reaction mixture was concentrated to dryness and placed under vacuum. This yielded a yellow oil 0.28 g (quantitative) which was used without further purification.

LCMS: calcd for C₁₆H₂₁N₃O₂ 287.2; found 288.2 [M+H]⁺.

### Preparation of 2-(4-(4-(6-Nitropyridin-2-yl)piperazin-1-yl)butyl)isoindoline-1,3-dione

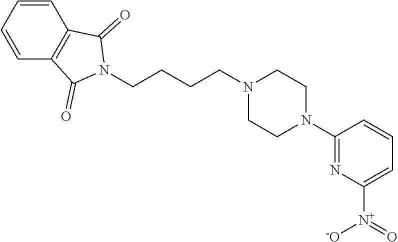

2-(4-(piperazin-1-yl)butyl)isoindoline-1,3-dione, (0.3 g, 1 mmol) was dissolved in acetonitrile (6 mL) and 2-chloro-6-nitropyridine (0.17 g, 1 mmol) then diisopropylethylamine (0.5 mL, 30 mmol) were added at ambient temperature. The reaction mixture was split into two 10 mL microwave glass tubes then heated at 130° C. for 26 min in the microwave oven. The reaction mixture was concentrated to give a brown oil. This was purified by chromatography on silica gel (40 g) eluting with a 3-5% methanol in ethyl acetate gradient over 16 CV at a flow of 40 mL/min). The product eluted between ˜4 to 5 CV, one of the fractions was concentrated to give a yellow solid (80 mg, 16%).

¹H (300 MHz, CDCl₃): δ 1.53-1.80 (4H, m, 2×CH₂), 2.41 (2H, t, J=7.7 Hz, CH₂), 2.53 (4H, t, J=5.2 Hz, pip 2×CH₂), 3.64 (4H, t, J=4.9 Hz, pip CH₂), 3.73 (2H, t, J=6.7 Hz, CH₂), 6.89 (1H, d, J=8.3 Hz, ArH), 7.43 (1H, d, J=7.4 Hz, ArH), and 7.68 (1H, t, J=8.6 HZ, ArH), 7.71-7.73 (2H, m, Phth), 7.83-7.86 (2H, m, Phth); ¹³C (75 MHz, CDCl₃): δ 24.0, 26.5, 37.8, 44.7, 52.7, 57.9, 105.4, 111.7, 123.2, 132.1, 133.9, 140.1, 155.8, 157.8, and 168.4.

### Preparation of 4-(4-(6-Nitropyridin-2-yl)piperazin-1-yl)butan-1-amine

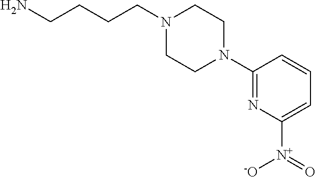

To a solution of 2-(4-(4-(6-nitropyridin-2-yl)piperazin-1-yl)butyl)isoindoline-1,3-dione (75 mg, 0.18 mmol) in ethanol (5 mL) was added hydrazine monohydrate (0.1 g, 2 mmol). The mixture was heated at 90° C. for 18 hr. The reaction mixture was cooled and concentrated. The residue was partitioned between dichloromethane (20 mL) and 10% aqueous potassium carbonate (20 mL). The dichloromethane layer was dried and concentrated to give the product as a yellow solid (50 mg, quantitative).

¹H (300 MHz, CDCl₃): δ 1.37 (4H, m, 2×CH₂), 2.35 (4H, t, J=7 Hz, pip 2×CH₂), 2.52 (4H, t, J=5 Hz, pip CH₂), 2.71 (2H, t, J=7 Hz, NH₂), 3.63 (4H, t, J=5 Hz, pip CH₂), 6.89 (1H, d, J=8.6 Hz, ArH), 7.41 (1H, d, J=7.3 Hz, ArH), and 7.66 (1H, t, J=8.0 HZ, ArH); ¹³C (75 MHz, CDCl₃): δ 24.2, 28.4, 31.3, 41.8, 44.7, 52.7, 58.3, 105.3, 111.7, 140.1, 155.8, 157.7.

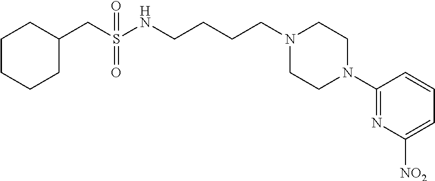 [SMILES: O=[N+]([O-])c1cccc(N2CCN(CCCCNS(=O)(=O)CC3CCCCC3)CC2)n1]

To a solution of 4-(4-(6-nitropyridin-2-yl)piperazin-1-yl)butan-1-amine (20 mg, 0.072 mmol) in dichloromethane (2 mL) was added triethylamine (20 mg, 0.2 mmol) and cyclohexylmethanesulfonyl chloride (20 mg, 0.1 mmol) at ambient temperature under a nitrogen atmosphere for 4 hours. After evaporation of the solvent the product was purified by chromatography on silica gel (10 g) eluting with 2.5% methanol in ethyl acetate at a flow of 30 mL/min. This was concentrated to give a yellow solid. Further purification was performed by preparative HPLC eluting with 50 to 95% methanol in water over 20 min (Gemini column 150×21.2 mm at a flow of 20 mL/min). This yielded the product as a yellow solid (14 mg, 44%).

LCMS: calcd for C₂₀H₃₃N₅O₄S 439.2; found 440.2 [M+H]⁺.

¹H (300 MHz, CDCl₃): δ 1.07 (2H, pentamer), 1.29 (2H, pentamer), 1.68 (7H, brm), 1.91 (4H, brm), 2.43 (2H, t), 2.60 (4H, t), 2.85 (2H, d), 3.10 (2H, t), 3.73 (4H, t), 6.91 (1H, d), 7.43 (1H, d), 7.69 (1H, t).

Compounds that were synthesized following procedures that were similar or identical as described those in Example 18 include the following:

| Structure | Name | [M + H]⁺ |
|---|---|---|
| 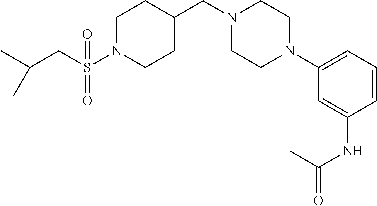 [SMILES: CC(=O)Nc1cccc(N2CCN(CC3CCN(S(=O)(=O)CC(C)C)CC3)CC2)c1] | N-(3-(4-((1- (isobutylsulfonyl)piperidin-4- yl)methyl)piperazin-1- yl)phenyl)acetamide | 437.5 |
| 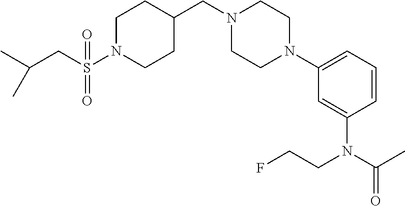 [SMILES: CC(=O)N(CCF)c1cccc(N2CCN(CC3CCN(S(=O)(=O)CC(C)C)CC3)CC2)c1] | N-(2-fluoroethyl)-N-(3-(4-((1- (isobutylsulfonyl)piperidin-4- yl)methyl)piperazin-1- yl)phenyl)acetamide | 483.5 |
| 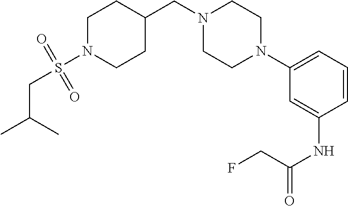 [SMILES: CC(C)CS(=O)(=O)N1CCC(CN2CCN(c3cccc(NC(=O)CF)c3)CC2)CC1] | 2-fluoro-N-(3-(4-((1- (isobutylsulfonyl)piperidin-4- yl)methyl)piperazin-1- yl)phenyl)acetamide | 455.5 |
| 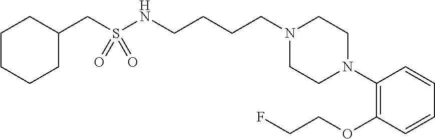 [SMILES: O=S(=O)(CC1CCCCC1)NCCCCN1CCN(c2ccccc2OCCF)CC1] | 1-cyclohexyl-N-(4-(4-(2-(2- fluoroethoxy)phenyl)piperazin-1- yl)butyl)methanesulfonamide | 456.1 |
| 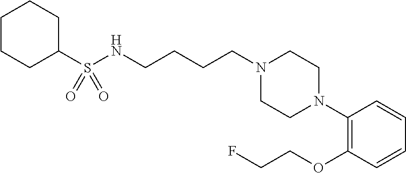 [SMILES: O=S(=O)(NCCCCN1CCN(c2ccccc2OCCF)CC1)C1CCCCC1] | N-(4-(4-(2-(2- fluoroethoxy)phenyl)piperazin-1- yl)butyl)cyclohexanesulfonamide | 442.1 |
| 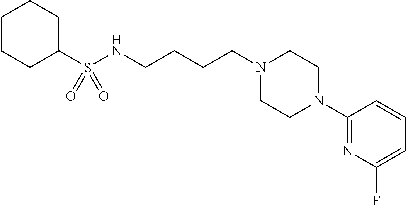 [SMILES: O=S(=O)(NCCCCN1CCN(c2cccc(F)n2)CC1)C1CCCCC1] | N-(4-(4-(6-fluoropyridin-2- yl)piperazin-1- yl)butyl)cyclohexanesulfonamide | 399.0 |
| 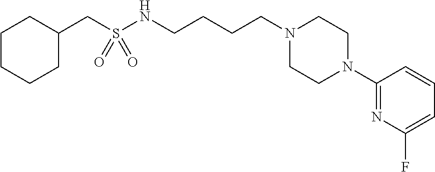 [SMILES: O=S(=O)(CC1CCCCC1)NCCCCN1CCN(c2cccc(F)n2)CC1] | 1-cyclohexyl-N-(4-(4-(6- fluoropyridin-2-yl)piperazin-1- yl)butyl)methanesulfonamide | 413.1 |

### Example 19

### Preparation of 1-(2-Fluoroethyl)-3-(4-(4-(2-methoxyphenyl)piperazin-1-yl)butyl)-imidazolidine-2,4-dione

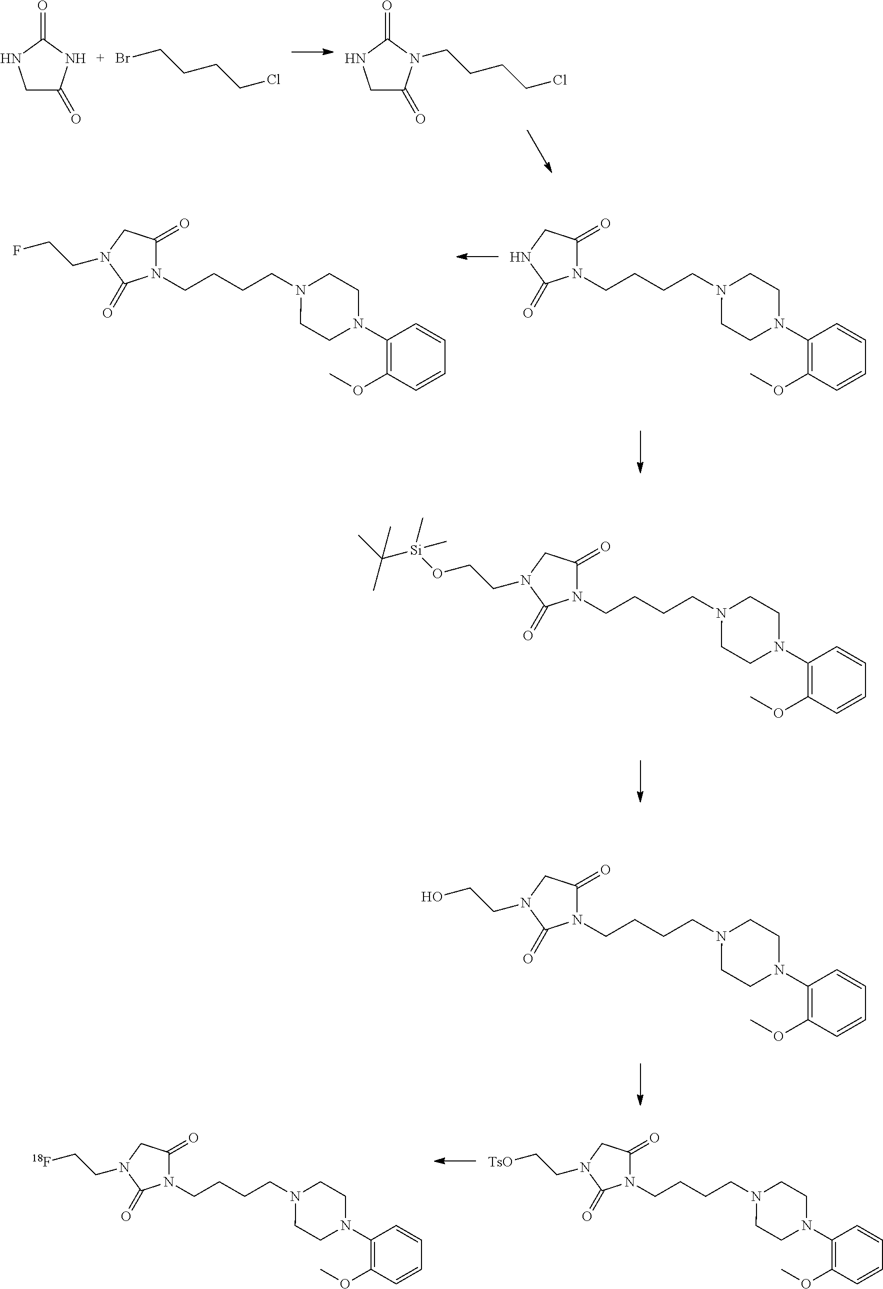

### Preparation of 3-(4-Chlorobutyl)imidazolidine-2,4-dione

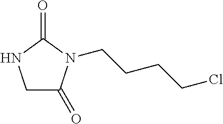 [SMILES: O=C1CNC(=O)N1CCCCCl]

To hydantoin (1.06 g, 10.5 mmol) in dimethylformamide (20 mL) at 50° C. was added a 60% suspension of sodium hydride (420 mg, 10.5 mmol). After stirring for 60 minutes 1-bromo-4-chlorobutane (4.5 g, 26.3 mmol) was added, and the mixture stirred for 18 hours. The reaction was quenched with 1 N hydrochloric acid (20 mL, 20 mmol) and concentrated to give a yellow oil. This was purified on silica gel (40 g) eluting with ethyl acetate at a flow of 40 mL/min. The product eluted in fractions 3-7. These were concentrated to give the product as a pale orange solid (1.9 g, 95%).

¹H (CDCl₃, 300 MHz): δ 1.65 (4H, m), 3.36 (2H, t), 3.62 (2H, t), 3.89 (2H, s), and 8.02 (1H, s).

### Preparation of 3-(4-(4-(2-Methoxyphenyl)piperazin-1-yl)butyl)imidazolidine-2,4-dione

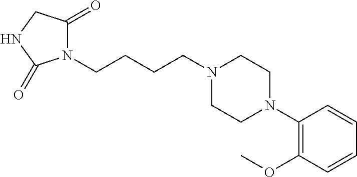 [SMILES: COc1ccccc1N1CCN(CCCCN2C(=O)CNC2=O)CC1]

To 3-(4-chlorobutyl)imidazolidine-2,4-dione (1.10 g, 5.7 mmol) in acetone (100 mL) was added potassium carbonate (2.5 g, 18 mmol) and 1-(2-methoxyphenyl)piperazine (1.0 g, 5.2 mmol). The mixture was heated at reflux for 24 hours. The mixture was cooled, filtered and concentrated at reduced pressure. The crude material was purified by chromatography on silica gel (40 g) eluting with 15% methanol in dichloromethane at a flow of 40 mL/min. The product eluted between ˜4-8 CV. These fractions were concentrated at reduced pressure to yield the product as a white solid (780 mg, 43%).

LCMS: calcd for C₁₈H₂₆N₄O₃ 346.2; found 347.0 [M+H]⁺.

### Preparation of 1-(2-Fluoroethyl)-3-(4-(4-(2-methoxyphenyl)piperazin-1-yl)butyl)imidazolidine-2,4-dione

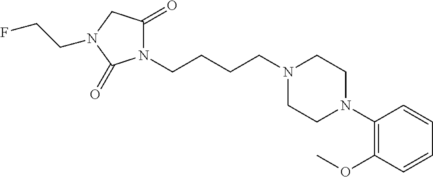 [SMILES: COc1ccccc1N1CCN(CCCCN2C(=O)CN(CCF)C2=O)CC1]

To 3-{4-[4-(2-methoxyphenyl)piperazin-1-yl]butyl}imidazolidine-2,4-dione (100 mg, 0.29 mmol) in dimethylformamide (1 mL) was added sodium hydride (12 mg, 0.3 mmol) and fluoroethyltosylate (63 mg, 0.29 mmol). The mixture was stirred at room temperature for 30 minutes. The mixture was filtered and concentrated at reduced pressure. The crude material was purified by chromatography on on silica gel (40 g) eluting with 5% methanol in ethyl acetate for 5 CV then a 5-15% methanol in ethyl actetate gradient over 15 CV at a flow of 30 mL/min. The product eluted between 2-6 CV. These fractions were concentrated at reduced pressure to yield the product as an off white solid (30 mg, 29%).

¹H (CDCl₃, 300 MHz): δ 1.67 (4H, m), 2.56 (2H, t), 2.79 (4H, brm), 3.18 (4H, brm), 3.55 (2H, t), 3.70 (2H, dt), 3.84 (3H, s), 4.01 (2H, s), 4.60 (2H, dt), and 6.81-7.05 (4H, m).

### Preparation of 1-(2-(tert-Butyldimethylsilyloxy)ethyl)-3-(4-(4-(2-methoxyphenyl)piperazin-1-yl)butyl)imidazolidine-2,4-dione

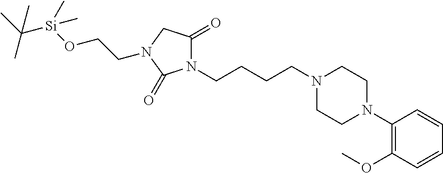 [SMILES: COc1ccccc1N1CCN(CCCCN2C(=O)CN(CCO[Si](C)(C)C(C)(C)C)C2=O)CC1]

To a solution of 3-(4-chlorobutyl)imidazolidine-2,4-dione in dimethylformamide is added sodium hydride (1.1 equivalent) and (2-bromoethoxy)(tert-butyl)dimethylsilane (1 equivalent). The reaction mixture is stirred at reflux for 18 hours. The reaction is partitioned between dichloromethane and water. The organics are dried and concentrated. The reaction mixture is purified by chromatography on silica gel eluting with 0-10% gradient of methanol in ethyl acetate.

### Preparation of 1-(2-Hydroxyethyl)-3-(4-(4-(2-methoxyphenyl)piperazin-1-yl)butyl)imidazolidine-2,4-dione

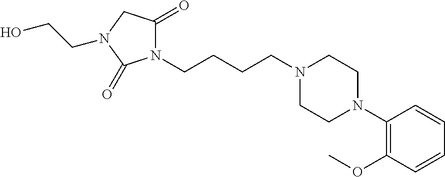 [SMILES: COc1ccccc1N1CCN(CCCCN2C(=O)CN(CCO)C2=O)CC1]

To 1-(2-(tert-butyldimethylsilyloxy)ethyl)-3-(4-(4-(2-methoxyphenyl)piperazin-1-yl)butyl)imidazolidine-2,4-dione is added 1M tetrabutylammonium fluoride in tetrahydrofuran. The reaction is partitioned between dichloromethane and water. The organics are dried and concentrated. The reaction mixture is purified by chromatography on silica gel eluting with 5% methanol in ethyl acetate.

### Preparation of 2-(3-(4-(4-(2-Methoxyphenyl)piperazin-1-yl)butyl)-2,4-dioxoimidazolidin-1-yl)ethyl 4-methylbenzenesulfonate

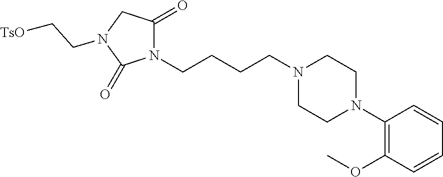 [SMILES: COc1ccccc1N1CCN(CCCCN2C(=O)CN(CCOS(=O)(=O)c3ccc(C)cc3)C2=O)CC1]

To a solution of 1-(2-hydroxyethyl)-3-(4-(4-(2-methoxyphenyl)piperazin-1-yl)butyl)imidazolidine-2,4-dione in dichloromethane is added triethylamine (1.1 equivalent) and tosyl chloride (1 equivalent) at 0° C. The reaction is partitioned between dichloromethane and water. The organics are dried and concentrated. The reaction mixture is purified by chromatography on silica gel eluting with 5% methanol in ethyl acetate.

### Preparation of [¹⁸F]1-(2-Fluoroethyl)-3-(4-(4-(2-methoxyphenyl)piperazin-1-yl)butyl)imidazolidine-2,4-dione

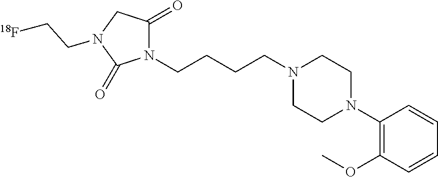 [SMILES: COc1ccccc1N1CCN(CCCCN2C(=O)CN(CC[18F])C2=O)CC1]

2-(3-(4-(4-(2-Methoxyphenyl)piperazin-1-yl)butyl)-2,4-dioxoimidazolidin-1-yl)ethyl 4-methylbenzenesulfonate is treated with [18F]fluoride in acetonitrile at 90° C. for between 5 and 10 mins. The product is isolated by hplc purification and formulated in ethanol with phosphate buffered saline.

### Example 20

### Preparation of 2-(4-(6-Fluoro-5′,6′-dihydro-[2,4′-bipyridin]-1′(2′H)-yl)butyl)-4-methyl-1,2,4-triazine-3,5(2H,4H)-dione

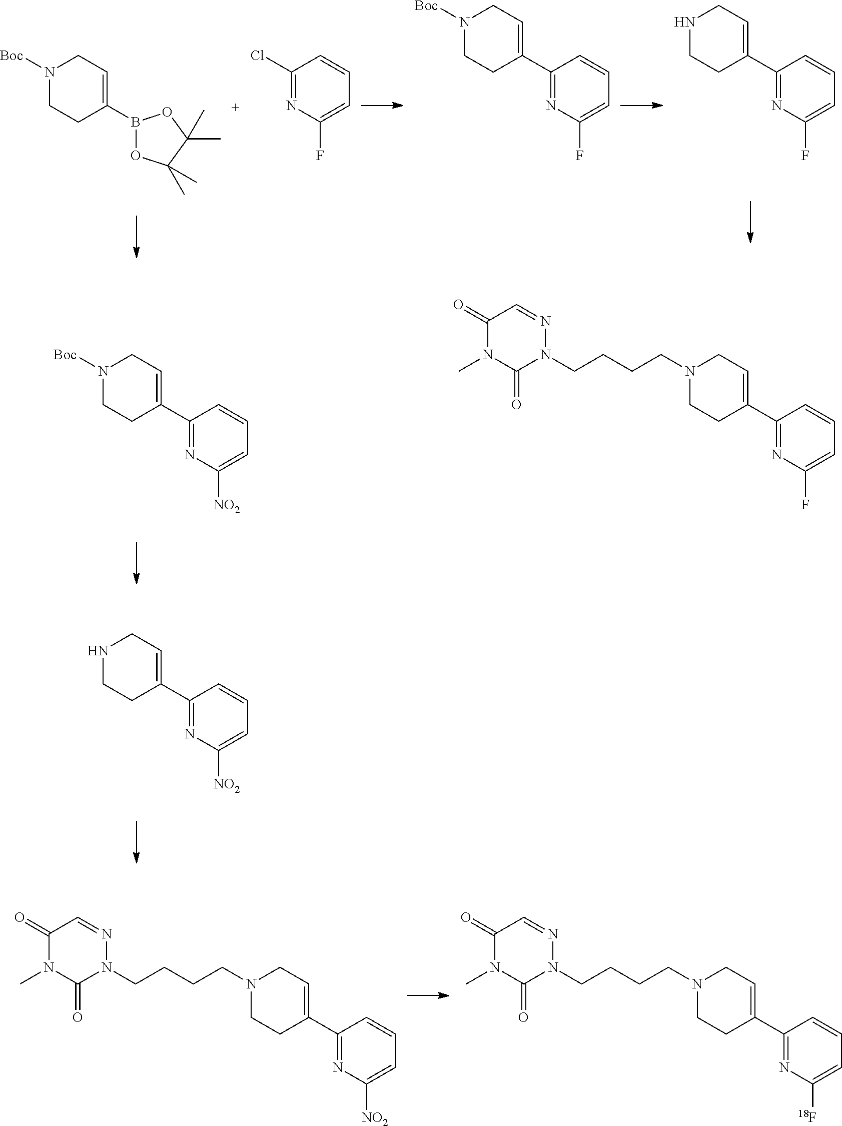

### Preparation of tert-Butyl 6-fluoro-5′,6′-dihydro-[2,4′-bipyridine]-1′(2′H)-carboxylate

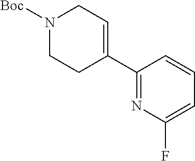 [SMILES: CC(C)(C)OC(=O)N1CC=C(c2cccc(F)n2)CC1]

A mixture of tert-butyl 4-(4,4,5,5-tetramethyl-1,3,2-dioxaborolan-2-yl)-5,6-dihydropyridine-1(2H)-carboxylate (1 g, 3.24 mmol, potassium carbonate (1.34 g, 9.71 mmol), PdCl₂dppf (0.16 g, 0.20 mmol) and 2-chloro-6-fluoropyridine (0.44 g, 3.40 mmol) in anhydrous dimethylformamide (5 mL) was heated at 80° C. for 18 hours. The reaction mixture was cooled to room temperature, filtered through Celite and the resulting solution was partitioned between ethyl acetate and brine. The organic portion was separated, dried over magnesium sulphate, filtered and evaporated to dryness under reduced pressure to afford a dark brown oil. This oil was purified by silica gel chromatography eluting with 0-50% ethyl acetate in petroleum ether (50 g, 27 CV, 40 mL/min) afford tert-butyl 6-fluoro-5′,6′-dihydro-[2,4′-bipyridine]-1′(2′H)-carboxylate as a colourless oil (254 mg, 28%).

¹H NMR (300 MHz, CDCl₃): δ: 1.43 (9H, s, (CH₃)₃), 2.53 (2H, bs, CH₂), 3.58 (2H, t, NCH₂), 4.07 (2H, m, NCH₂), 6.65 (1H, p, C═CH), 6.72 (1H, dd, CH_{Ar}), 7.16 (1H, dd, CH_{Ar}), 7.69 (1H, q, CH_{Ar}). ¹³C NMR (75 MHz, CDCl₃): δ: 25.6, 28.3, 40.1, 43.7, 79.6, 107.4, 115.7, 125.7, 133.7, 141.3, 154.7, 161.1, 164.3. MS expected for C₁₅H₁₉FN₂O₂: 278.14; found: 223.14 (M−C(CH₃)₃]⁺.

### Preparation of 6-Fluoro-1′,2′,3′,6′-tetrahydro-2,4′-bipyridine

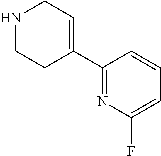 [SMILES: Fc1cccc(C2=CCNCC2)n1]

4-(6-Fluoropyridin-2-yl)-5,6-dihydropyridine-1(2H)-carboxylate (258 mg, 0.93 mmol) was dissolved in dichloromethane (1.5 mL). To this solution was added trifluoroacetic acid (1.5 mL) and was stirred at ambient temperature for 20 mins. The reaction mixture was quenched with 1.5 mL saturated potassium carbonate solution, partitioned between saturated aqueous potassium carbonate and dichloromethane and separated. The aqueous phase was washed again with ethyl acetate (3 mL) and the organic portion was collected. The combined organic portions were evaporated to dryness under reduced pressure to afford 6-fluoro-1′,2′,3′,6′-tetrahydro-2,4′-bipyridine as a pale orange oil (155 mg, 94%).

LCMS; calcd for C₁₀H₁₁FN₂: 178.1; found: 179.2 [M+H]⁺.

¹H NMR (300 MHz, CDCl₃): δ: 2.61 (2H, s), 3.19 (2H, s), 3.66 (2H, s), 5.81 (1H, s), 6.72 (1H, s), 6.77 (1H, dd), 7.20 (1H, d), 7.71 (1H, q). ¹³C NMR (75 MHz, CDCl₃): δ: 24.6, 42.0, 44.0, 107.8, 115.8, 125.4, 133.8, 141.4, 161.2, 164.3.

### Preparation of 2-(4-(6-fluoro-5′,6′-dihydro-[2,4′-bipyridin]-1′(2′H)-yl)butyl)-4-methyl-1,2,4-triazine-3,5(2H,4H)-dione

2-(4-Chlorobutyl)-4-methyl-1,2,4-triazine-3,5(2H,4H)-dione (67 mg, 0.31 mmol), 6-fluoro-1′,2′,3′,6′-tetrahydro-2,4′-bipyridine (50 mg, 0.28 mmol), potassium carbonate (116 mg, 0.84 mmol) and sodium iodide (8 mg, 20 mol %) were dissolved in anhydrous acetonitrile (0.5 mL) in a microwave vial and stirred in the microwave at 120° C. for 2 hours. The reaction mixture was filtered and evaporated to dryness under reduced pressure. The residue was purified by silica gel chromatography eluting with 75-100% ethyl acetate in petroleum ether (10 g, 25 CV. 30 mL/min) to afford 2-(4-(6-fluoro-5′,6′-dihydro-[2,4′-bipyridin]-1′(2′H)-yl)butyl)-4-methyl-1,2,4-triazine-3,5(2H,4H)-dione as a yellow oil (31 mg, 31%).

LCMS: calcd for C₁₈H₂₂FN₅O₂ 359.2; found: 360.2 [M+H]⁺.

¹H NMR (300 MHz, CDCl₃): δ: 1.60 (2H, p), 1.81 (2H, p), 2.50 (2H, t), 2.61 (2H, m), 2.79 (2H, t), 3.19 (2H, dd), 3.33 (3H, s), 4.01 (2H, t), 6.72 (1H, m), 6.75 (1H, dd), 7.19 (1H, dd), 7.38 (1H, s), 7.71 (1H, q). ¹³C NMR (75.5 MHz, CDCl₃): δ: 24.1, 26.2, 26.5, 26.9, 50.1, 51.7, 53.2, 57.6, 107.2, 115.7, 126.9, 133.4, 133.8, 141.2, 148.8, 156.2, 156.3, 164.4.

### Preparation of tert-Butyl 6-nitro-5′,6′-dihydro-[2,4′-bipyridine]-1′(2′H)-carboxylate

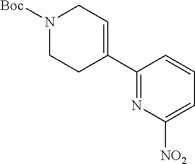 [SMILES: CC(C)(C)OC(=O)N1CC=C(c2cccc([N+](=O)[O-])n2)CC1]

A mixture of tert-butyl 4-(4,4,5,5-tetramethyl-1,3,2-dioxaborolan-2-yl)-5,6-dihydropyridine-1(2H)-carboxylate (3.24 mmol) potassium carbonate (1.34 g, 9.71 mmol), PdCl₂dppf (0.16 g, 0.20 mmol) and 2-chloro-6-nitropyridine (0.44 g, 3.40 mmol) in anhydrous dimethylformamide (5 mL) is heated at 80° C. for 18 hours. The reaction mixture is cooled to room temperature, filtered through Celite and the resulting solution is partitioned between ethyl acetate and brine. The organic portion is separated, dried over magnesium sulphate, filtered and evaporated to dryness under reduced pressure to afford a crude product. This is purified by chromatography on silica gel eluting with 0-50% ethyl acetate in petroleum ether (50 g) to afford tert-butyl 6-nitro-5′,6′-dihydro-[2,4′-bipyridine]-1′(2′H)-carboxylate.

### Preparation of 6-Nitro-1′,2′,3′,6′-tetrahydro-2,4′-bipyridine

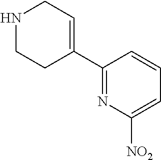 [SMILES: O=[N+]([O-])c1cccc(C2=CCNCC2)n1]

4-(6-Nitropyridin-2-yl)-5,6-dihydropyridine-1(2H)-carboxylate (1 mmol) is dissolved in dichloromethane (1.5 mL). To this solution is added trifluoroacetic acid (1.5 mL) and is stirred at ambient temperature for 20 mins. The reaction mixture is quenched with 1.5 mL saturated potassium carbonate solution, partitioned between saturated aqueous potassium carbonate and dichloromethane and separated. The aqueous phase is washed again with ethyl acetate (3 mL) and the organic portion is collected. The combined organic portions are evaporated to dryness under reduced pressure to afford 6-nitro-1′,2′,3′,6′-tetrahydro-2,4′-bipyridine.

### Preparation of 4-Methyl-2-(4-(6-nitro-5′,6′-dihydro-[2,4′-bipyridin]-1′(2′H)-yl)butyl)-1,2,4-triazine-3,5(2H,4H)-dione

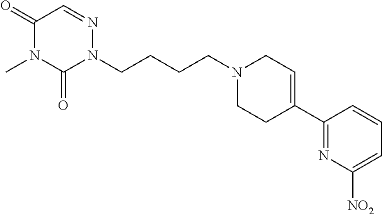 [SMILES: Cn1c(=O)cnn(CCCCN2CC=C(c3cccc([N+](=O)[O-])n3)CC2)c1=O]

2-(4-Chlorobutyl)-4-methyl-1,2,4-triazine-3,5(2H,4H)-dione (67 mg, 0.31 mmol), 6-nitro-1′,2′,3′,6′-tetrahydro-2,4′-bipyridine (0.28 mmol), potassium carbonate (116 mg, 0.84 mmol) and sodium iodide (8 mg, 20 mol %) are dissolved in anhydrous acetonitrile (0.5 mL) in a microwave vial and stirred in the microwave at 120° C. for 2 hours. The reaction mixture is filtered and evaporated to dryness under reduced pressure. The residue is purified by silica gel chromatography eluting with 75-100% ethyl acetate in petroleum ether (10 g, 25 CV. 30 mL/min) to afford 2-(4-(6-nitro-5′,6′-dihydro-[2,4′-bipyridin]-1′(2′H)-yl)butyl)-4-methyl-1,2,4-triazine-3,5(2H,4H)-dione.

### Preparation of [¹⁸F]2-(4-(6-fluoro-5′,6′-dihydro-[2,4′-bipyridin]-1′(2′H)-yl)butyl)-4-methyl-1,2,4-triazine-3,5(2H,4H)-dione

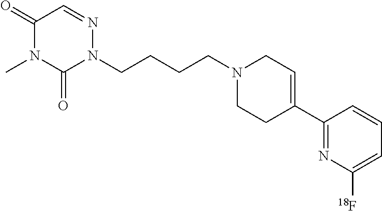 [SMILES: Cn1c(=O)cnn(CCCCN2CC=C(c3cccc([18F])n3)CC2)c1=O]

2-(4-(6-Nitro-5′,6′-dihydro-[2,4′-bipyridin]-1′(2′H)-yl)butyl)-4-methyl-1,2,4-triazine-3,5(2H,4H)-dione is treated with [18F]fluoride in acetonitrile at 150° C. for 10-20 mins. The product is purified by hplc and formulated in ethanol with phosphate buffered saline.

### Example 21

### 1-(3-(4-(2-fluoroethyl)-1H-1,2,3-triazol-1-yl)propyl)-4-(pyridin-2-yl)piperazine

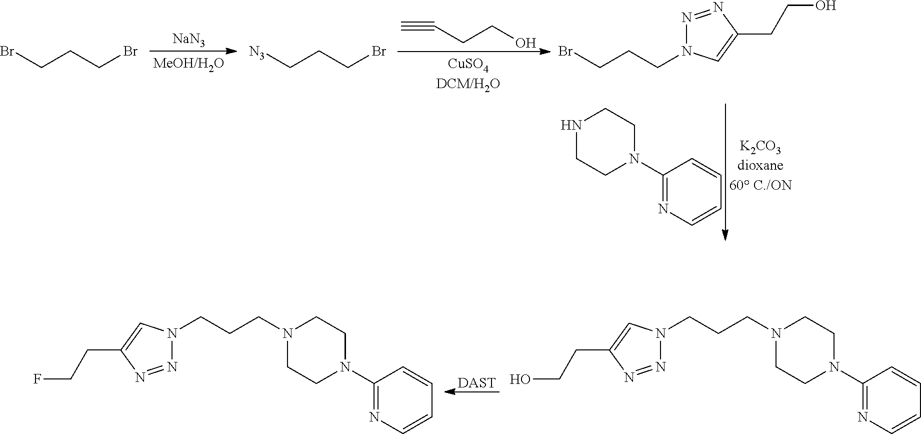

1,3-Dibromobutane (6.21 g, 30.76 mmol) and sodium azide (1 g, 15.38 mmol) were dissolved in methanol (10 ml) and water (1 ml) and heated at 60° C. overnight. The progress of the reaction was monitored by TLC. After completion of the reaction, the solvents were evaporated under reduced pressure and the crude product was used directly in the next step.

To a solution of 3-butyn-1-ol (1.5 g, 21.4 mmol) and 3-azido-1-bromopropane (3.17 g, 19.46 mmol) in DCM (5 ml) and water (5 ml) were added copper(II) sulphate pentahydrate (242 mg, 0.97 mmol) and sodium ascorbate (540 mg, 2.72 mmol). The resulting solution was stirred overnight at room temperature. The reaction mixture was diluted with DICHLOROMETHANE (10 ml) and water (10 ml). The organic layer was separated, dried over sodium sulphate and concentrated. The residue was purified by flash column chromatography (hexane/EtOAc, 2:1) to give 2-(1-(3-bromopropyl)-1H-1,2,3-triazol-4-yl)ethanol (220 mg).

MS: calcd for C₇H₁₂BrN₃O 233.0; found 234.0 (M+H)⁺.

To a solution of 2-pyridinyl piperazine (77 mg, 0.47 mmol) in 1,4-dioxane (8 ml), potassium carbonate (130 mg, 0.94 mmol) and 2-(1-(3-bromopropyl)-1H-1,2,3-triazol-4-yl)ethanol (220 mg, 0.94 mmol) was added and heated at 60° C. overnight. TLC analysis showed the completion of the reaction. The reaction mixture was filtered and washed with 1,4-dioxane (2×10 ml). The filtrates were concentrated and the crude product was purified by column chromatography using 5% methanol in dichloromethane to give 2-(1-(3-(4-(pyridin-2-yl)piperazin-1-yl)propyl)-1H-1,2,3-triazol-4-yl)ethanol (100 mg).

¹H NMR (400 MHz, CDCl₃) δ 2.15 (m, 2H), 2.43 (t, 2H), 2.56 (brs, 4H), 2.96 (t, 2H), 3.56 (brs, 4H), 3.96 (t, 2H), 4.45 (t, 2H), 6.64 (m, 1H), 7.43 (s, 1H), 7.49 (m 1H), 8.19 (m, 1H).

MS: calcd for C₁₆H₂₄N₆O 316.2; found 317.1 (M+H)⁺: 316.

2-(1-(3-(4-(pyridin-2-yl)piperazin-1-yl)propyl)-1H-1,2,3-triazol-4-yl)ethanol (50 mg, 0.15 mmol) in dichloromethane (5 ml) was added to a solution of DAST (76 mg, 0.47 mmol) in dichloromethane (5 ml) at −78° C. After the addition the reaction mixture was stirred at RT for 1 hr. The progress of the reaction was monitored by TLC. After completion of the reaction, saturated aqueous sodium bicarbonate solution (10 ml) was added and extracted with dichloromethane (3×10 ml). The combined organic layers were dried over sodium sulphate and concentrated. The crude material was purified by silica gel column chromatography using 2% methanol in dichloromethane to afford the impure fluoro compound, which was purified by preparative HPLC to give 1-(3-(4-(2-fluoroethyl)-1H-1,2,3-triazol-1-yl)propyl)-4-(pyridin-2-yl)piperazine (10 mg).

¹H NMR (400 MHz, DMSO) δ 2.13 (m, 2H), 2.40 (t, 2H), 2.54 (brs, 4H), 3.11 (t, 1H), 3.18 (t, 1H), 3.55 (brs, 4H), 4.45 (t, 2H), 4.65 (t, 1H), 4.76 (t, 1H), 6.62-6.66 (m, 1H), 7.46-7.51 (m, 2H), 8.18-8.20 (m, 1H).

MS: calcd for C₁₆H₂₃FN₆ 318.2; found 319.1 (M+H)⁺.

Compounds that were synthesized following procedures that were similar or identical as described those in Example 21 include the following:

| Structure | Name | [M + H]⁺ |
|---|---|---|
| 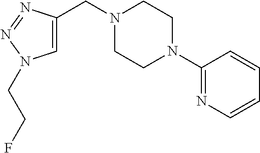 [SMILES: FCCn1cc(CN2CCN(c3ccccn3)CC2)nn1] | 1-((1-(2-fluoroethyl)-1H- 1,2,3-triazol-4- yl)methyl)-4-(pyridin-2- yl)piperazine | 291.2 |
| 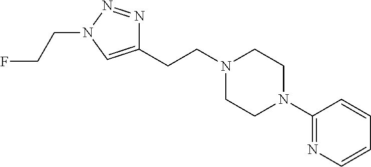 [SMILES: FCCn1cc(CCN2CCN(c3ccccn3)CC2)nn1] | 1-(2-(1-(2-fluoroethyl)- 1H-1,2,3-triazol-4- yl)ethyl)-4-(pyridin-2- yl)piperazine | 305.3 |
| 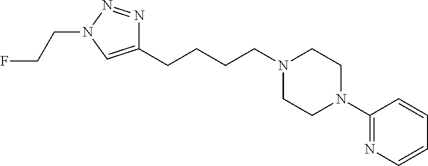 [SMILES: FCCn1cc(CCCCN2CCN(c3ccccn3)CC2)nn1] | 1-(4-(1-(2-fluoroethyl)- 1H-1,2,3-triazol-4- yl)butyl)-4-(pyridin-2- yl)piperazine | 333.3 |
| 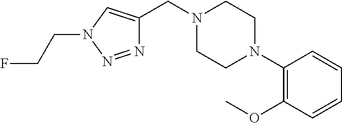 [SMILES: COc1ccccc1N1CCN(Cc2cn(CCF)nn2)CC1] | 1-((1-(2-fluoroethyl)-1H- 1,2,3-triazol-4- yl)methyl)-4-(2- methoxyphenyl)piperazine | 320.3 |
| 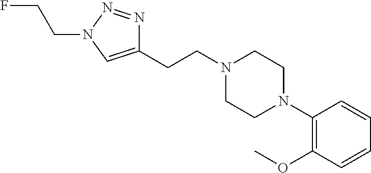 [SMILES: COc1ccccc1N1CCN(CCc2cn(CCF)nn2)CC1] | 1-(2-(1-(2-fluoroethyl)- 1H-1,2,3-triazol-4- yl)ethyl)-4-(2- methoxyphenyl)piperazine | 334.3 |
| 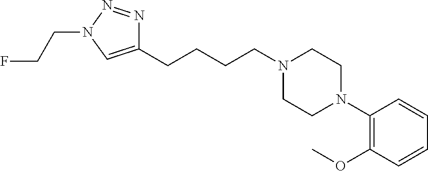 [SMILES: COc1ccccc1N1CCN(CCCCc2cn(CCF)nn2)CC1] | 1-(4-(1-(2-fluoroethyl)- 1H-1,2,3-triazol-4- yl)butyl)-4-(2- methoxyphenyl)piperazine | 362.4 |
| 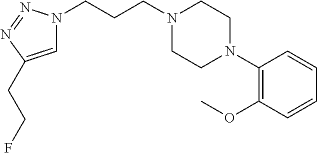 [SMILES: COc1ccccc1N1CCN(CCCn2cc(CCF)nn2)CC1] | 1-(3-(4-(2-fluoroethyl)- 1H-1,2,3-triazol-1- yl)propyl)-4-(2- methoxyphenyl)piperazine | 348.3 |
| 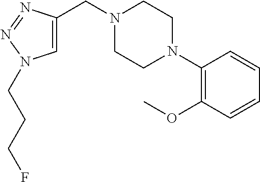 [SMILES: COc1ccccc1N1CCN(Cc2cn(CCCF)nn2)CC1] | 1-((1-(3-fluoropropyl)- 1H-1,2,3-triazol-4- yl)methyl)-4-(2- methoxyphenyl)piperazine | 334.3 |
| 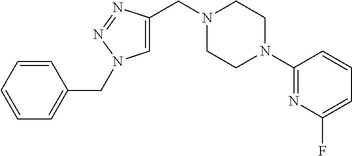 [SMILES: Fc1cccc(N2CCN(Cc3cn(Cc4ccccc4)nn3)CC2)n1] | 1-((1-benzyl-1H-1,2,3- triazol-4-yl)methyl)-4-(6- fluoropyridin-2- yl)piperazine | 353.3 |
| 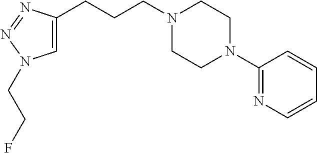 [SMILES: FCCn1cc(CCCN2CCN(c3ccccn3)CC2)nn1] | 1-(3-(1-(2-fluoroethyl)- 1H-1,2,3-triazol-4- yl)propyl)-4-(pyridin-2- yl)piperazine | 319.3 |
| 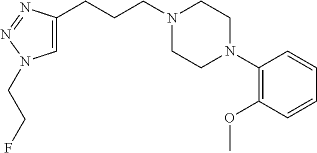 [SMILES: COc1ccccc1N1CCN(CCCc2cn(CCF)nn2)CC1] | 1-(3-(1-(2-fluoroethyl)- 1H-1,2,3-triazol-4- yl)propyl)-4-(2- methoxyphenyl)piperazine | 348.3 |
| 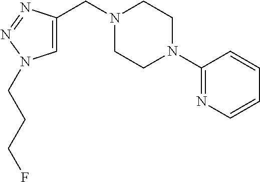 [SMILES: FCCCn1cc(CN2CCN(c3ccccn3)CC2)nn1] | 1-((1-(3-fluoropropyl)- 1H-1,2,3-triazol-4- yl)methyl)-4-(pyridin-2- yl)piperazine | 305.3 |
| 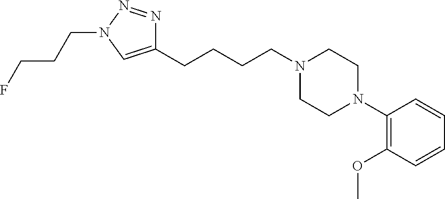 [SMILES: COc1ccccc1N1CCN(CCCCc2cn(CCCF)nn2)CC1] | 1-(4-(1-(3-fluoropropyl)- 1H-1,2,3-triazol-4- yl)butyl)-4-(2- methoxyphenyl)piperazine | 376.4 |
| 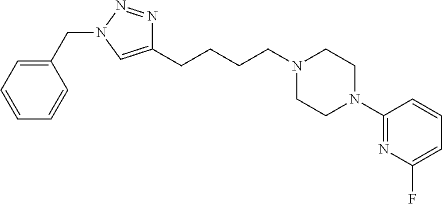 [SMILES: Fc1cccc(N2CCN(CCCCc3cn(Cc4ccccc4)nn3)CC2)n1] | 1-(4-(1-benzyl-1H-1,2,3- triazol-4-yl)butyl)-4-(6- fluoropyridin-2- yl)piperazine | 395.4 |
| 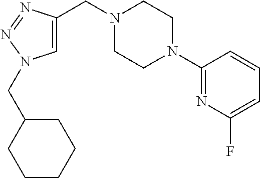 [SMILES: Fc1cccc(N2CCN(Cc3cn(CC4CCCCC4)nn3)CC2)n1] | 1-((1- (cyclohexylmethyl)-1H- 1,2,3-triazol-4- yl)methyl)-4-(6- fluoropyridin-2- yl)piperazine | 359.4 |
| 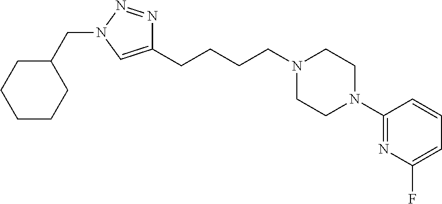 [SMILES: Fc1cccc(N2CCN(CCCCc3cn(CC4CCCCC4)nn3)CC2)n1] | 1-(4-(1- (cyclohexylmethyl)-1H- 1,2,3-triazol-4-yl)butyl)- 4-(6-fluoropyridin-2- yl)piperazine | 401.4 |
| 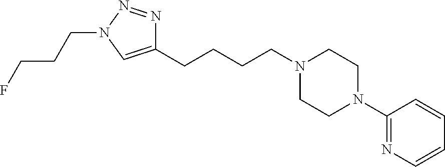 [SMILES: FCCCn1cc(CCCCN2CCN(c3ccccn3)CC2)nn1] | 1-(4-(1-(3-fluoropropyl)- 1H-1,2,3-triazol-4- yl)butyl)-4-(pyridin-2- yl)piperazine | 347.3 |
| 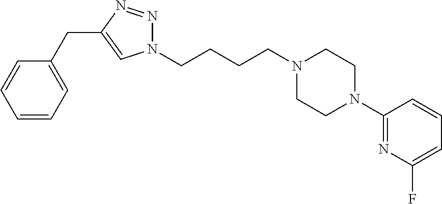 [SMILES: Fc1cccc(N2CCN(CCCCn3cc(Cc4ccccc4)nn3)CC2)n1] | 1-(4-(4-benzyl-1H-1,2,3- triazol-1-yl)butyl)-4-(6- fluoropyridin-2- yl)piperazine | 395.4 |
| 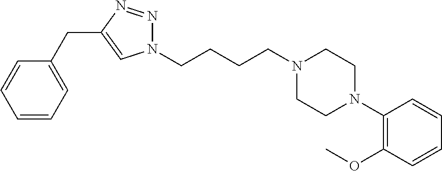 [SMILES: COc1ccccc1N1CCN(CCCCn2cc(Cc3ccccc3)nn2)CC1] | 1-(4-(4-benzyl-1H-1,2,3- triazol-1-yl)butyl)-4-(2- methoxyphenyl)piperazine | 406.4 |
| 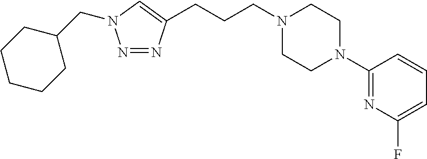 [SMILES: Fc1cccc(N2CCN(CCCc3cn(CC4CCCCC4)nn3)CC2)n1] | 1-(3-(1- (cyclohexylmethyl)-1H- 1,2,3-triazol-4- yl)propyl)-4-(6- fluoropyridin-2- yl)piperazine | 387.4 |
| 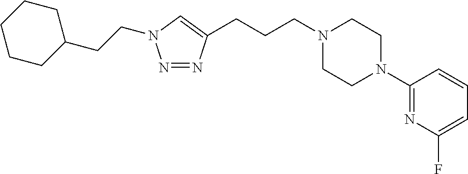 [SMILES: Fc1cccc(N2CCN(CCCc3cn(CCC4CCCCC4)nn3)CC2)n1] | 1-(3-(1-(2- cyclohexylethyl)-1H- yl)propyl)-4-(6- fluoropyridin-2- yl)piperazine | 401.4 |
| 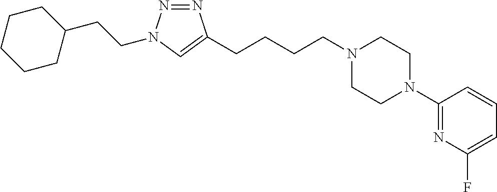 [SMILES: Fc1cccc(N2CCN(CCCCc3cn(CCC4CCCCC4)nn3)CC2)n1] | 1-(4-(1-(2- cyclohexylethyl)-1H- 1,2,3-triazol-4-yl)butyl)- 4-(6-fluoropyridin-2- yl)piperazine | 415.5 |
| 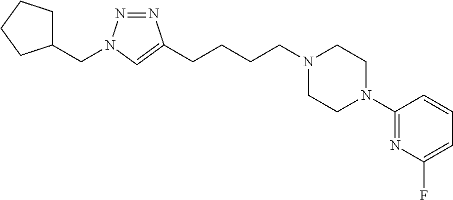 [SMILES: Fc1cccc(N2CCN(CCCCc3cn(CC4CCCC4)nn3)CC2)n1] | 1-(4-(1- (cyclopentylmethyl)-1H- 1,2,3-triazol-4-yl)butyl)- 4-(6-fluoropyridin-2- yl)piperazine | 387.4 |
| 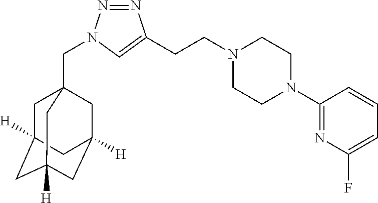 [SMILES: Fc1cccc(N2CCN(CCc3cn(CC45C[C@H]6C[C@@H](C4)C[C@@H](C5)C6)nn3)CC2)n1] | 1-(2-(1-((3r,5r,7r)- adamantan-1-ylmethyl)- 1H-1,2,3-triazol-4- yl)ethyl)-4-(6- fluoropyridin-2- yl)piperazine | 425.5 |
| 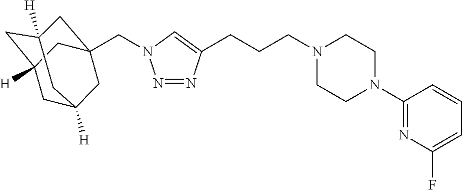 [SMILES: Fc1cccc(N2CCN(CCCc3cn(CC45C[C@H]6C[C@@H](C4)C[C@@H](C5)C6)nn3)CC2)n1] | 1-(3-(1-((3r,5r,7r)- adamantan-1-ylmethyl)- 1H-1,2,3-triazol-4- yl)propyl)-4-(6- fluoropyridin-2- yl)piperazine | 439.5 |
| 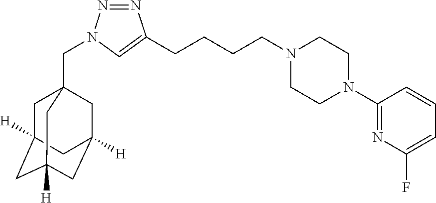 [SMILES: Fc1cccc(N2CCN(CCCCc3cn(CC45C[C@H]6C[C@@H](C4)C[C@@H](C5)C6)nn3)CC2)n1] | 1-(4-(1-((3r,5r,7r)- adamantan-1-ylmethyl)- 1H-1,2,3-triazol-4- yl)butyl)-4-(6- fluoropyridin-2- yl)piperazine | 453.5 |
| 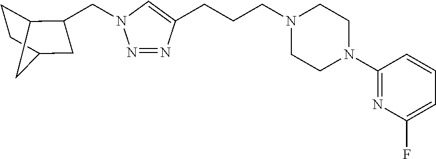 [SMILES: Fc1cccc(N2CCN(CCCc3cn(CC4CC5CCC4C5)nn3)CC2)n1] | 1-(3-(1- (bicyclo[2.2.1]heptan-2- ylmethyl)-1H-1,2,3- triazol-4-yl)propyl)-4-(6- fluoropyridin-2- yl)piperazine | 399.4 |
| 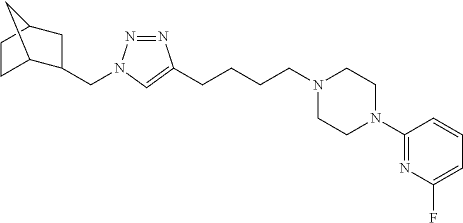 [SMILES: Fc1cccc(N2CCN(CCCCc3cn(CC4CC5CCC4C5)nn3)CC2)n1] | 1-(4-(1-((1R,4S)- bicyclo[2.2.1]heptan-2- ylmethyl)-1H-1,2,3- triazol-4-yl)butyl)-4-(6- fluoropyridin-2- yl)piperazine | 413.4 |
| 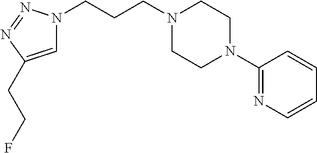 [SMILES: FCCc1cn(CCCN2CCN(c3ccccn3)CC2)nn1] | 1-(3-(4-(2-fluoroethyl)- 1H-1,2,3-triazol-1- yl)propyl)-4-(pyridin-2- yl)piperazine | 319.3 |

### Example 22

### N-((2-(4-fluorophenyl)-2-phenyl-1,3-oxathiolan-5-yl)methyl)-3-phenylpropan-1-amine

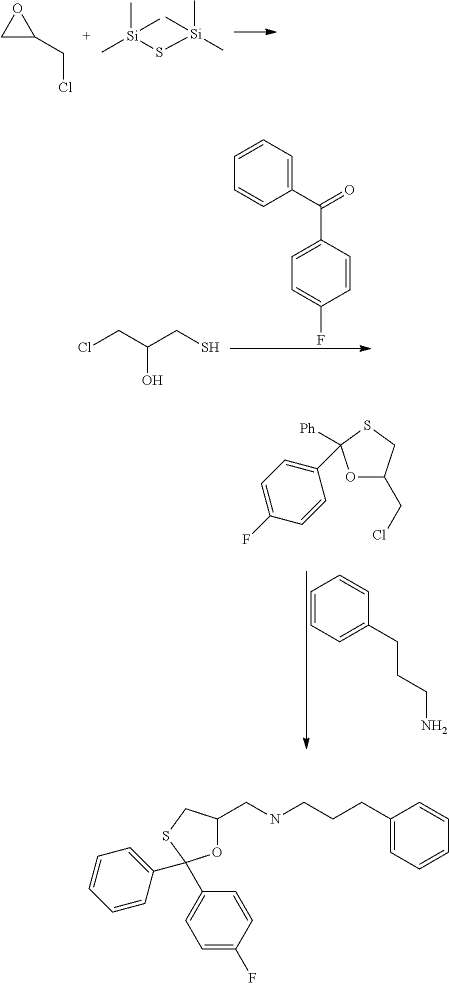

### 1-Chloro-3-mercaptopropan-2-ol

To a solution of epichlorohydrin (0.3 ml, 4.3 mmol) in anhydrous tetrahydrofuran (0.4 ml) cooled at −20° C., 1,1,1,3,3,3-hexamethyldisilathiane (1.17 ml, 5.6 mmol) and TBAF (0.29 g, 1.12 mmol) were added. The reaction mixture was maintained at the same temperature for another 30 minutes. The reaction mixture was quenched with 2 ml of 50% citric acid solution and allowed to stir for another 5 minutes. Diethylether (10 ml) was added and the organic layer was washed with 20% citric acid solution (2×10 ml). The organic layer was separated and dried over anhydrous sodium sulfate, filterd and evaportaed. The crude material was then purified by chromatography on silica gel column to give 1-chloro-3-mercaptopropan-2-ol (177 mg, 33%)

¹H-NMR (500 MHz, CDCl₃) δ=3.85 (1H, m), 3.62 (2H, d, J=5 Hz), 3.06 (1H, bs), 2.76-2.64 (2H, m), 1.50 (1H, t, J=10 Hz).

### 5-(Chloromethyl)-2-(4-fluorophenyl)-2-phenyl-1,3-oxathiolane

A solution of 4-fluorobenzophenone (0.6 g, 3.0 mmol) in toluene (30 ml) was treated with excess of 1-chloro-3-mercaptopropan-2-ol (1.13 g, 9.0 mmol) and a catalytic amount of PTSA. The solution was refluxed with a Dean-Stark apparatus for 24 h to remove water. The reaction was then washed with water (2×20 ml), saturated NaHCO₃ (2×20 ml). The organic layer then extracted with ethyl acetate (2×30 ml) and dried over anhydrous sodium sulfate, filtered and evaporated. The crude product was purified by chromatography on silica gel eluting with isochratic hexane:ethyl acetate (40:60) to give 5-(chloromethyl)-2-(4-fluorophenyl)-2-phenyl-1,3-oxathiolane (535 mg, 57%).

LC-MS: calcd for C₁₆H₁₄ClFOS 308.0; found 309.0 (M+H)⁺.

¹H-NMR (500 MHz, CDCl₃) δ 7.64-7.60 (2H, m), 7.43-7.29 (5H, m), 7.05 (1H, t, J=10 Hz), 6.98 (1H, t, J=5 Hz), 4.46 (1H, m), 3.83-3.71 (2H, m), 3.25 (2H, d, J=5 Hz).

### N-((2-(4-fluorophenyl)-2-phenyl-1,3-oxathiolan-5-yl)methyl)-3-phenylpropan-1-amine

5-(Chloromethyl)-2-(4-fluorophenyl)-2-phenyl-1,3-oxathiolane (200 mg, 0.64 mmol) in 2-methoxyethanol (2 ml), was treated with a 5-fold excess of the 3-phenylpropan-1-amine (0.44 g, 3.2 mmol) and a catalytic amount of potassium iodide. The reaction was then refluxed for 24 h. The solvent was evaporated and the organic layer was washed with water (2×20 ml) and extracted with ethyl acetate (2×30 ml). The organic extract was dried over sodium sulfate, filtered and evaporated. The crude material was then purified by chromatography on silica gel eluting with isochratic hexane:ethyl acetate (50:50) to give N-((2-(4-fluorophenyl)-2-phenyl-1,3-oxathiolan-5-yl)methyl)-3-phenylpropan-1-amine (96 mg, 35%)

LC-MS: calcd for C₂₅H₂₆FNOS=407.2; found 408.2 (M+H)⁺.

¹H-NMR (300 MHz, CDCl₃) δ 7.64-7.56 (2H, m), 7.45-7.16 (10H, m), 7.08-6.91 (2H, m), 4.30 (1H, m), 3.21-3.11 (2H, m), 3.07-2.87 (2H, m), 2.70 (4H, q, J=9 Hz), 1.86 (2H, m).

Compounds that were synthesized following procedures that were similar or identical as described those in Example 22 include the following:

| Structure | Name | [M + H]⁺ |
|---|---|---|
| 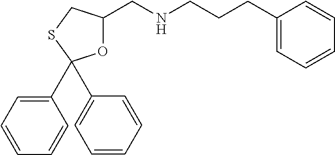 [SMILES: c1ccc(CCCNCC2CSC(c3ccccc3)(c3ccccc3)O2)cc1] | N-((2,2-diphenyl-1,3- oxathiolan-5-yl)methyl)-3- phenylpropan-1-amine | 390.5 |
| 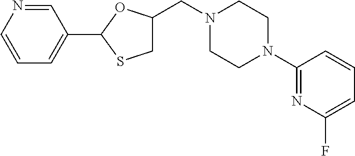 [SMILES: Fc1cccc(N2CCN(CC3CSC(c4cccnc4)O3)CC2)n1] | 1-(6-fluoropyridin-2-yl)-4- ((2-(pyridin-3-yl)-1,3- oxathiolan-5- yl)methyl)piperazine | 361.3 |
| 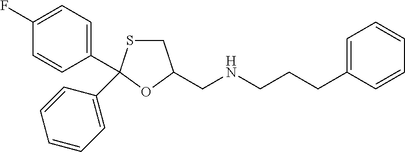 [SMILES: Fc1ccc(C2(c3ccccc3)OC(CNCCCc3ccccc3)CS2)cc1] | N-((2-(4-fluorophenyl)-2- phenyl-1,3-oxathiolan-5- yl)methyl)-3-phenylpropan- 1-amine | 408.4 |
| 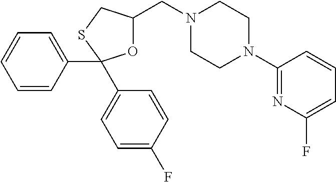 [SMILES: Fc1ccc(C2(c3ccccc3)OC(CN3CCN(c4cccc(F)n4)CC3)CS2)cc1] | 1-((2-(4-fluorophenyl)-2- phenyl-1,3-oxathiolan-5- yl)methyl)-4-(6- fluoropyridin-2-yl)piperazine | 454.4 |
| 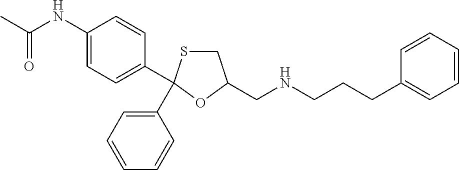 [SMILES: CC(=O)Nc1ccc(C2(c3ccccc3)OC(CNCCCc3ccccc3)CS2)cc1] | N-(4-(2-phenyl-5-(((3- phenylpropyl)amino)methyl)- 1,3-oxathiolan-2- yl)phenyl)acetamide | 447.5 |
| 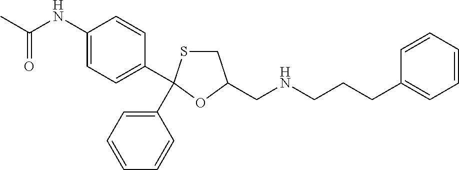 [SMILES: CC(=O)Nc1ccc(C2(c3ccccc3)OC(CNCCCc3ccccc3)CS2)cc1] | N-(4-(2-phenyl-5-(((3- phenylpropyl)amino)methyl)- 1,3-oxathiolan-2- yl)phenyl)acetamide | 447.5 |
| 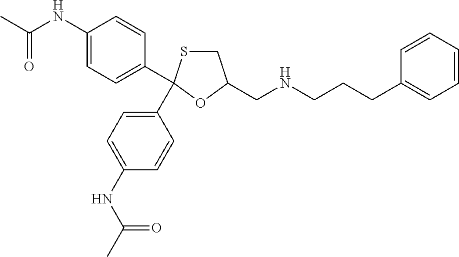 [SMILES: CC(=O)Nc1ccc(C2(c3ccc(NC(C)=O)cc3)OC(CNCCCc3ccccc3)CS2)cc1] | N,N′-((5-(((3- phenylpropyl)amino)methyl)- 1,3-oxathiolane-2,2- diyl)bis(4,1- phenylene))diacetamide | 504.6 |

### Example 23

### In Vitro Binding Assessment of Compounds

For the compounds described herein, the Ki towards the 5-HT1A receptor target measured using a 5-HT_{1A} affinity assay (J. Pharmacol. Exp. Ther. 306(1): 301-309 (2003)). ranged between 0.14-471 nM. In some embodiments, the Ki is from about 0.1 to about 500 nM and preferably from about 0.1 to about 1 nM.

In some cases, the Ki can be from about 1 to about 500 nM, from about 1 to about 10 nM, from about 10 nM to about 100 nM, from about 100 nM or from about 0.1 nM to about 10 nM.

To assess the degree of agonism towards the 5-HT_{1A} receptor, a standard GTPγS assay was used. J. Neurosci. Res. 61(6): 674-685 (2000). Full agonism was determined using serotonin and compound agonism was expressed as a percentage serotonin response. At a compound concentration of 10 nM, the degree of agonism ranged between 2-95%.

For the compounds described herein, the off target binding to D2 receptors was assessed. At 10 nM compound concentration, the % inhibition of 0.7 nM [³H] Spiperone from the D2 receptor ranged between 0-95%, with a preferred range of 0-30%.

For the compounds described herein, the off target binding to adrenergic alpha 1 receptors was assessed. At 10 nM compound concentration, the % inhibition of 0.25 nM [³H] Prazosin from the adrenergic alpha 1 receptor ranged between 0-89%, with a preferred range of 0-55%.

### In Vivo Binding Assessment of Compounds

Biodistributions in conscious rats were performed to assess regional brain distribution of ¹⁸F-radiolabelled compounds. The following brain regions were chosen to represent regions of high, intermediate and low 5-HT_{1A} receptor density; prefrontal cortex, frontal cortex, striata, amygdala, hippocampus, periacquaductal gray (including raphe) and cerebellum. The observed hippocampus:cerebellum ratios at 60 minutes post injection ranged from 0.93-3.5, with a preferred range of 1.3-3.5. The specific binding to the 5-HT_{1A} receptor in vivo was confirmed by reduced radiolabelled compound retention to 5-HT_{1A} receptor rich brain areas following pre-dosing animals with unlabelled WAY100635. The structure of WAY100635 and other standards are given below.

Selected compounds were assessed in vivo for pharmacological effects when administered to conscious rats at 3 mg/kg. This pharmacological challenge confirmed that the degree of agonism correlated with pharmacological effect, with full agonists showing the same pharmacological effect in vivo as 8-OH-DPAT (dosed at 0.3 mg/kg).

| Structure | Name |
|---|---|
| 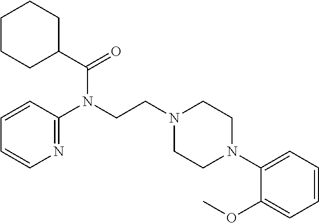 [SMILES: COc1ccccc1N1CCN(CCN(C(=O)C2CCCCC2)c2ccccn2)CC1] | WAY100635 |
| 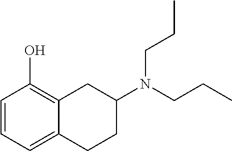 [SMILES: CCCN(CCC)C1CCc2cccc(O)c2C1] | 8-Hydroxy- DPAT |

The invention described and claimed herein is not to be limited in scope by the specific embodiments herein disclosed, since these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A compound of the formula (I):
Z—Y-L₂-N(R₁)-L₁-X(R₂)—Ar (I)
or a pharmaceutically acceptable salt thereof,
wherein:
Ar is -aryl or a 3- to 9-membered aromatic heterocycle, wherein the Ar;
X is —N, —CH—, O, or S;
R₁ is absent, H, Me or with R₂ forms a heterocycloalkyl group
L₁ is a (—CH₂)₂—
L₂ is —(CH₂)ₙ— or —(CH₂)ᵣ-L₃-(CH₂)ₛ— where n is an integer ranging from 1 to 5; r and s are independently integers ranging from 0 to 2
L₃ is a 3-9-membered cycloalkyl or heterocycloalkyl
Y is absent or a bond, S, O, NH, CONH, NHCO or SO₂NH;
Z is selected from a group comprising a 3-to 9-membered aromatic heterocycle, aryl, an alkyl, cycloalkyl or a heterocycloalkyl; and
wherein said compound of formula (I) is not a compound of the formula:

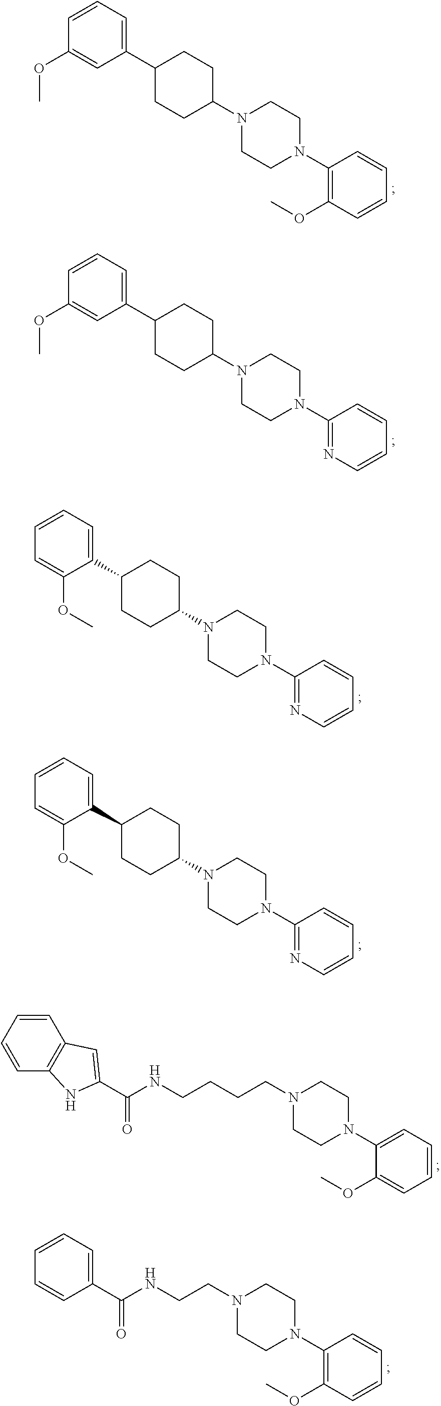 [SMILES: COc1cccc(C2CCC(N3CCN(c4ccccc4OC)CC3)CC2)c1 ; COc1cccc(C2CCC(N3CCN(c4ccccn4)CC3)CC2)c1 ; COc1ccccc1[C@H]1CC[C@@H](N2CCN(c3ccccn3)CC2)CC1 ; COc1ccccc1[C@H]1CC[C@H](N2CCN(c3ccccn3)CC2)CC1 ; COc1ccccc1N1CCN(CCCCNC(=O)c2cc3ccccc3[nH]2)CC1 ; COc1ccccc1N1CCN(CCNC(=O)c2ccccc2)CC1]

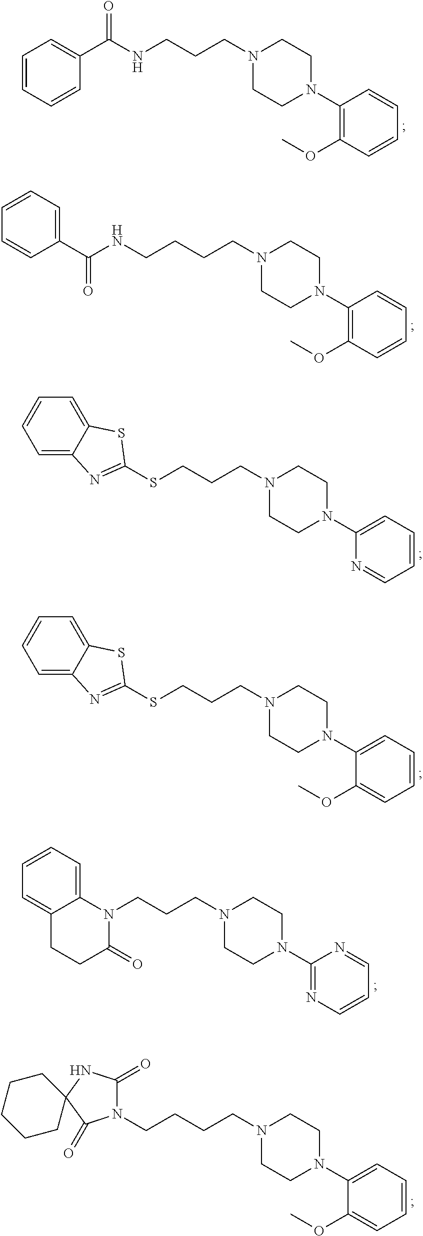 [SMILES: COc1ccccc1N1CCN(CCCNC(=O)c2ccccc2)CC1 ; COc1ccccc1N1CCN(CCCCNC(=O)c2ccccc2)CC1 ; c1ccc(N2CCN(CCCSc3nc4ccccc4s3)CC2)nc1 ; COc1ccccc1N1CCN(CCCSc2nc3ccccc3s2)CC1 ; O=C1CCc2ccccc2N1CCCN1CCN(c2ncccn2)CC1 ; COc1ccccc1N1CCN(CCCCN2C(=O)NC3(CCCCC3)C2=O)CC1]

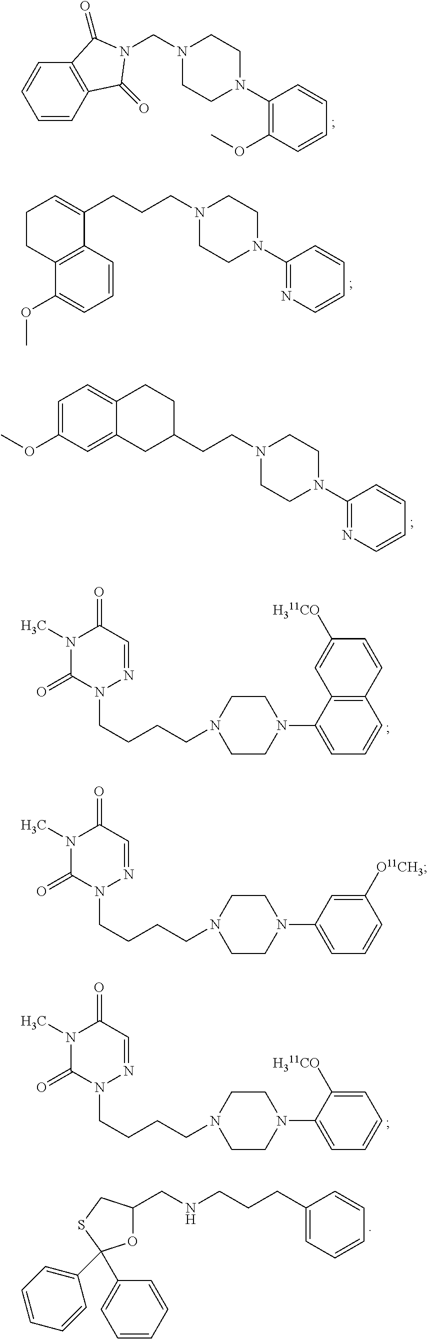

2. The compound of claim 1, wherein the compound is radiolabeled.

3. The compound of claim 1, wherein the compound is not radiolabeled.

4. The compound of claim 1, wherein the compound comprises an ¹⁸F or a ¹¹C atom.

5. The compound of claim 1, wherein an ¹⁸F or an ¹¹C atom is attached directly to Ar.

6. The compound of claim 1, wherein a —OCₙHₘ¹⁸F group or —OC¹¹H₃ group is directly attached to Ar, wherein n is 1 to 4 and m is 2 to 8, respectively.

7. The compound of claim 1, wherein an ¹⁸F or an ¹¹C atom is attached directly to Z or to a suitable group on Z.

8. The compound of claim 1, wherein an ¹⁸F or an ¹¹C atom is attached directly to L₂ or L₃ or to a suitable group on L₂ or L₃.

9. The compound of claim 1 having the formula: 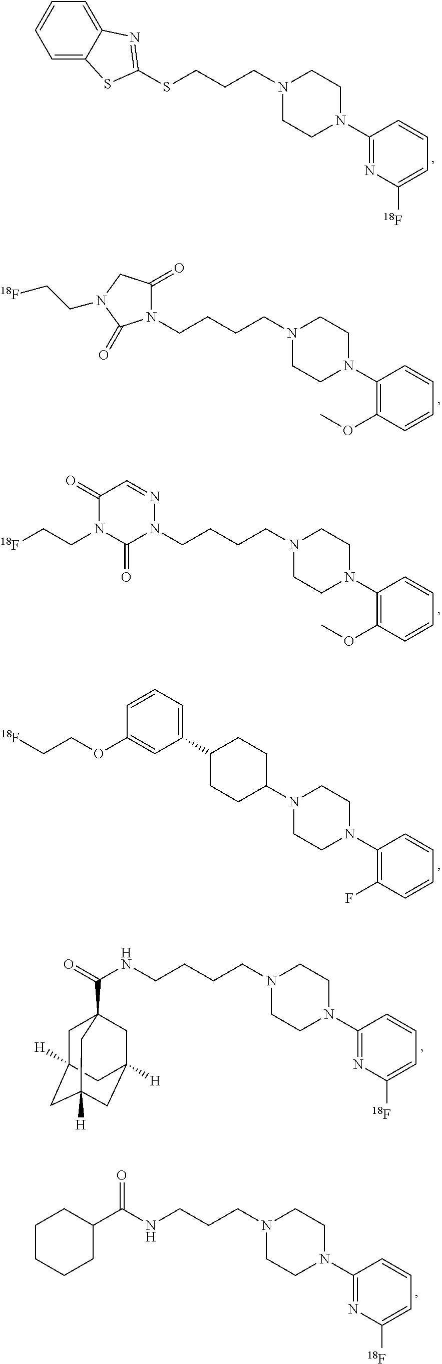 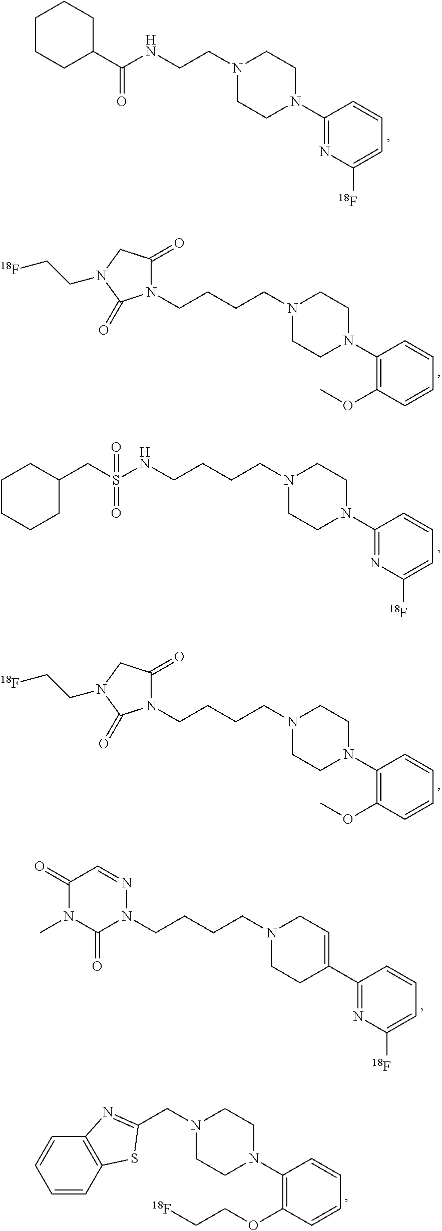 [SMILES: O=C(NCCN1CCN(c2cccc([18F])n2)CC1)C1CCCCC1 ; COc1ccccc1N1CCN(CCCCN2C(=O)CN(CC[18F])C2=O)CC1 ; O=S(=O)(CC1CCCCC1)NCCCCN1CCN(c2cccc([18F])n2)CC1 ; COc1ccccc1N1CCN(CCCCN2C(=O)CN(CC[18F])C2=O)CC1 ; Cn1c(=O)cnn(CCCCN2CC=C(c3cccc([18F])n3)CC2)c1=O ; [18F]CCOc1ccccc1N1CCN(Cc2nc3ccccc3s2)CC1] 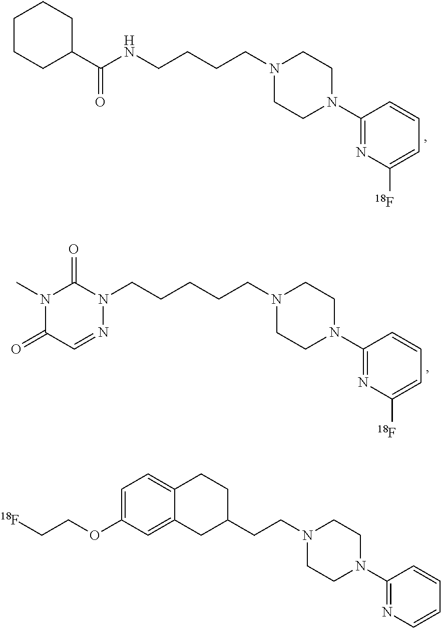 [SMILES: O=C(NCCCCN1CCN(c2cccc([18F])n2)CC1)C1CCCCC1 ; Cn1c(=O)cnn(CCCCCN2CCN(c3cccc([18F])n3)CC2)c1=O ; [18F]CCOc1ccc2c(c1)CC(CCN1CCN(c3ccccn3)CC1)CC2]
or a pharmaceutically acceptable salt thereof.

10. The compound of claim 1 having the formula: 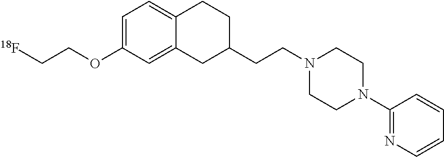 [SMILES: [18F]CCOc1ccc2c(c1)CC(CCN1CCN(c3ccccn3)CC1)CC2]
or a pharmaceutically acceptable salt thereof.

11. A composition comprising the compound of claim 1, or a pharmaceutically acceptable salt thereof, and a physiologically acceptable carrier or vehicle.

12. A method for imaging one or more 5-HT_{1A} receptors in a subject in vivo, the method comprising:
(a) administering to the subject an imaging-effective amount of a compound of claim 2, or a pharmaceutically acceptable salt thereof; and
(b) detecting the radioactive emission of the radiolabel on the compound of claim 2, or salt thereof, following its administration to the subject.

13. The method of claim 12, wherein the radioactive emission is detected using PET or SPECT.

14. The method of claim 12, wherein the radioactive emission is detected in the brain of the subject.

15. The method of claim 12, wherein the subject is known or suspected to have a neurological disorder.

16. The method of claim 15, wherein the neurological disorder is an affective disorder, an anxiety disorder, an eating disorder, an addictive disorder, a sleep disorder, a disease associated with cognitive dysfunction, a neurodegenerative disease, such as stroke; a seizure disorder, a pain disorder; a panic disorder, a disorder of movement, or an obsessive-compulsive disorder.

17. The method of claim 16, wherein the disease associated with cognitive dysfunction is Alzhemer's disease.

18. The method of claim 16, wherein the neurodegenerative disease is stroke.

19. The method of claim 16, wherein the disorder of movement is Parkinson's disease.

20. The method of claim 16, wherein the seizure disorder is epilepsy.

21. The method of claim 16, wherein the affective disorder is depression.

22. The method of claim 12, wherein the compound selectively binds to the 5-HT_{1A} receptor relative to other serotonin receptors.

23. A method for treating a disease associated with abnormal 5-HT_{1A} receptor function comprising administering to the subject in need thereofan effective amount of a compound of claim 1, or a pharmaceutically acceptable salt thereof.

24. A method for treating a neurological or psychiatric disorder in a subject, the method comprising administering to the subject a therapeutically effective amount of a compound as claimed in claim 3 or a pharmaceutically acceptable salt.

25. The method of claim 24, wherein the neurological disorder is Alzheimer's disease.

26. A method for stabilizing the mood of a subject having a mood disorder, the method comprising administering to the subject a therapeutically effective amount of a compound as claimed in claim 1 or a pharmaceutically acceptable salt thereof.

27. The method of claim 26, wherein the mood disorder is bipolar disorder or depression.

28. A compound according to claim 1, or a pharmaceutically acceptable salt thereof, for medical use.

29. Use of a compound according to claim 2 for the manufacture of a radiopharmaceutical for use in a method of in vivo imaging.

30. A method of generating an image of a human or animal body comprising administering a compound according to claim 2 to said body and generating an image of at least a part of said body to which said compound has distributed using PET.

31. A method of monitoring the effect of treatment of a human or animal body with a drug to combat or treat a condition associated with neurological disorder, said method comprising administering to said body a compound of claim 2 and detecting the uptake of said conjugate by cell receptors said administration and detection optionally but preferably being effected before, during and after treatment with said drug.

32. A compound according to claim 3, wherein the compound is for therapeutic use.

33. A method of making a compound of the formula (VII): 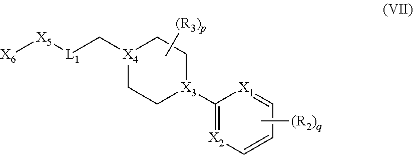
the method comprising:
(i) reacting a compound of the formula 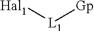
where Hal₁ is a halogen; Gp is a different halogen than Hal₁, an amine or a protected amine; and L₁ is optionally substituted alkyl or optionally substituted cycloalkyl; with an optionally substituted heterocycloalkyl compound to give a compound of the formula: 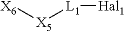
where:
X₅ is a bond; and
X₆ is optionally substituted heterocycloalkyl; and
(ii) reacting the compound of formula 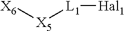
with a compound of the formula: 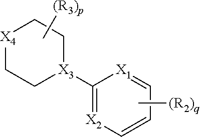
wherein X₁ and X₂ are the same or different, independently one from the other, and each is N or CR₁, where R₁ is H, hydroxy, alkoxy, halo, haloalkyloxy, nitro, haloalkylamido, or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ is N or CR₄, wherein R₄ is H or halogen;
X₄ is N; and
p and q are the same or different, independently one from the other, and each is 0, 1 or 2;
to give a compound of the formula (VII):

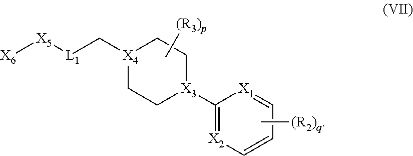

34. A method of making a compound of the formula (VII): 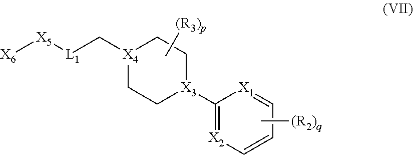
the method comprising:
(i) reacting a compound of the formula 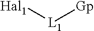
where Hal₁ is a halogen; Gp is a different halogen than Hal₁, an amine or a protected amine; and L₁ is optionally substituted alkyl or optionally substituted cycloalkyl; with a compound of the formula: 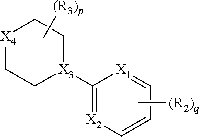
wherein X₁ and X₂ are the same or different, independently one from the other, and each is N or CR₁, where R₁ is H, hydroxy, alkoxy, halo, haloalkyloxy, nitro, haloalkylamido, or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ is N or CR₄, wherein R₄ is H or halogen;
X₄ is N; and
p and q are the same or different, independently one from the other, and each is 0, 1 or 2;
to give a compound of the formula: 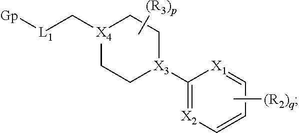
and
(ii) reacting a compound of the formula: 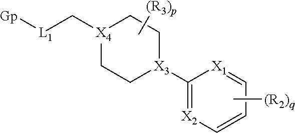
with a compound of the formula: 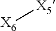
where:
X₅′ comprises a group that reacts with Gp; and
X₆ is optionally substituted heterocycloalkyl;
to give a compound of the formula (VII):

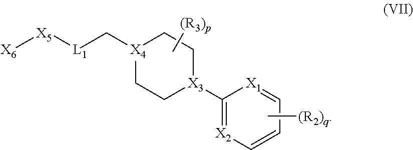

35. The method of claim 34, wherein said group that reacts with Gp comprises a thiol, an amine or a hydroxyl.

36. A method of preparing a compound of formula (VIII): 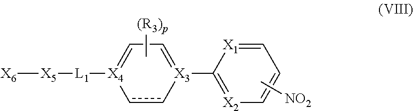
the method comprising reacting a compound of the formula 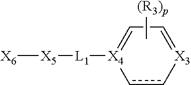
with a compound of the formula 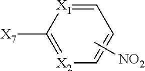
wherein X₁ and X₂ are the same or different, independently one from the other, and each is N or CR₁, where R₁ is H, hydroxy, alkoxy, halo, haloalkyloxy, nitro, haloalkylamido;
X₇ is halo;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

37. A method of preparing a compound of formula (IX): 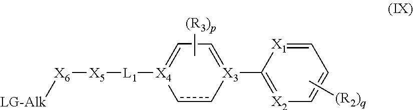
the method comprising reacting a compound of the formula 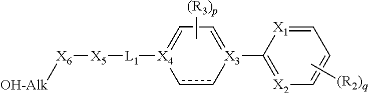
with a reagent which transforms the hydroxyl group attached to Alk into a leaving group;
wherein Alk is an alkyl group;
LG is a leaving group;
X₁ and X₂ are the same or different, independently one from the other, and each is N or CR₁, where R₁ is H, hydroxy, alkoxy, halo, haloalkyloxy, nitro, haloalkylamido or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl;
p and q are the same or different, independently one from the other, and each is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

38. A method of preparing a compound of formula (X): 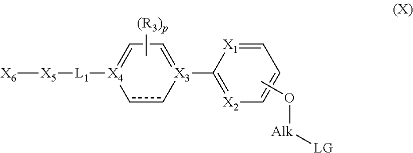
the method comprising reacting a compound of the formula 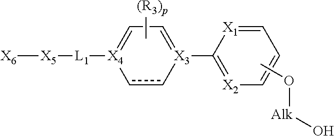
with a reagent which transforms the hydroxyl group attached to Alk into a leaving group;
wherein Alk is an alkyl group;
LG is a leaving group;
wherein X₁ and X₂ are the same or different, independently one from the other, and each is N or CR₁, where R₁ is H, hydroxy, alkoxy, halo, haloalkyloxy, nitro, haloalkylamido;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

39. A compound of formula (VIII): 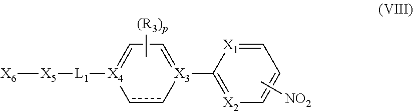
wherein X₁ and X₂ are the same or different, independently one from the other, and each is N or CR₁, where R₁ is H, hydroxy, alkoxy, halo, haloalkyloxy, nitro, haloalkylamido;
X₇ is halo;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

40. A compound of formula (IX): 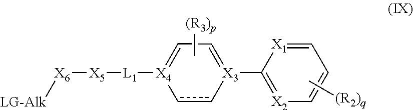
wherein Alk is an alkyl group;
LG is a leaving group;
X₁ and X₂ are the same or different, independently one from the other, and each is N or CR₁, where R₁ is H, hydroxy, alkoxy, halo, haloalkyloxy, nitro, haloalkylamido or R₁ and an R₂ group, together with the carbon atoms to which they are attached, form a ring comprising one or more heterocycles;
R₂ is H, alkoxy, halo, haloalkylamido or nitro;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl;
p and q are the same or different, independently one from the other, and each is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

41. A method of preparing a compound of formula (X): 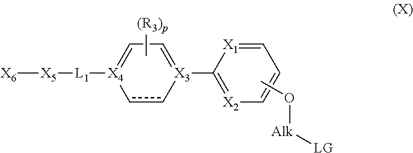
the method comprising reacting a compound of the formula 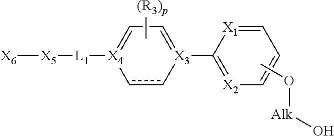
with a reagent which transforms the hydroxyl group attached to Alk into a leaving group;
wherein Alk is an alkyl group;
LG is a leaving group;
wherein X₁ and X₂ are the same or different, independently one from the other, and each is N or CR₁, where R₁ is H, hydroxy, alkoxy, halo, haloalkyloxy, nitro, haloalkylamido;
R₃ is H or halogen or R₁ and R₃, together with the atoms to which they are attached, form a ring comprising one or more heteroatoms;
X₃ and X₄ are the same or different, independently one from the other, and each is N or CR₄, wherein R₄ is H or halogen, where the dashed lines in the ring comprising X₃ and X₄ represent a single or a double bond, with the proviso that when the dashed line between X₃ or X₄ and an adjacent carbon represents a double bond, then R₄ is not present at X₃ or X₄, respectively;
L₁ is optionally substituted alkyl or optionally substituted cycloalkyl; and
p is 0, 1 or 2;
X₅ is a bond, —N(R₅)—C(O)—, —C(O)—N(R₅)—, —N(R₅)—, —S(O)ₓ—, —O—, heterocycloalkyl or heteroaryl, where R₅ is H, aryl or heteroaryl and x is 0, 1 or 2; and
X₆ is halogen, hydroxy, optionally substituted alkyl, optionally substituted aryl, optionally substituted heteroaryl, optionally substituted cycloalkyl, optionally substituted cycloalkylalkyl, optionally substituted cycloalkenyl or optionally substituted heterocycloalkyl.

42. A radiopharmaceutical kit for the preparation of a radiopharmaceutical compound for use in PET, which comprises:
(i) a vessel comprising a compound of one of claims 39-41; and
(ii) means for eluting the vessel with a source of ¹⁸F⁻.

43. The method of claim 42, further comprising:
(iii) an ion-exchange cartridge for removal of excess ¹⁸F⁻.

44. A method for obtaining a diagnostic PET image which comprises the step of using a radiopharmaceutical kit according to claim 42.

45. A method for obtaining a diagnostic PET image which comprises the step of using a cartridge for a radiopharmaceutical kit according to claim 42.

46. A cartridge for a radiopharmaceutical kit for the preparation of a radiopharmaceutical compound for use in PET, which comprises:
(i) a vessel containing a compound of one of claims 39-41; and
(ii) means for eluting the vessel with a source of ¹⁸F⁻.
